(19) United States
(12) Patent Application Publication
(10) Pub. No.: US 2021/0079474 A1
(43) Pub. Date: Mar. 18, 2021

(54) CIRCULAR RNAS FOR THE DIAGNOSIS AND TREATMENT OF BRAIN DISORDERS

(71) Applicant: STC. UNM, Albuquerque, NM (US)

(72) Inventor: Nikolaos MELLIOS, Albuquerque, NM (US)

(21) Appl. No.: 17/050,285

(22) PCT Filed: Apr. 25, 2019
(86) PCT No.: PCT/US2019/029065

§ 371 Date: Oct. 23, 2020

Related U.S. Application Data
(60) Provisional application No. 62/662,294, filed on Apr. 25, 2018.

(51) Int. Cl. C12Q 1/6883

(52) U.S. Cl. CPC C12Q 1/6883; C12Q 2600/178; C12Q 2600/166

(57) ABSTRACT

A plurality of circular RNAs (circRNAs) the expression of which is correlated with brain disorders. The circRNAs are useful for compositions, kits, assays, and methods for the identification, diagnosis, screening, treatment and/or monitoring of brain disorders including psychiatric disorders such as bipolar disorder (BD), schizophrenia (SCZ), depression, Attention-Deficit/Hyperactivity Disorder (ADHD), Obsessive-compulsive disorder (OCD), Anxiety Disorders, etc., and neurodevelopmental disorders such as Autism, Asperger's Syndrome, and other Autism Spectrum Disorders (ASD), pervasive developmental disorders not otherwise specified (PDD-NOS), etc.

### CROSS-REFERENCE TO RELATED APPLICATIONS

The following application claims benefit of U.S. Provisional Application No. 62/662,294, filed Apr. 25, 2018, which is hereby incorporated by reference in its entirety.

### STATEMENT REGARDING GOVERNMENT SPONSORED RESEARCH

This invention was made with Government support under Grant No. 646-681-4888 awarded by The Brain and Behavior Research foundation. The U.S. Government has certain rights in this invention.

### SEQUENCE LISTING

A sequence listing is submitted with the present application. The sequence listing is submitted as a .txt file entitled 7570102SeqLis_ST25.txt and is 6 kb in size. The entire contents of the sequence listing are hereby incorporated by reference.

### BACKGROUND

Bipolar disorder (BD), schizophrenia (SCZ), and depression are multifactorial and heterogeneous psychiatric disorders with an average age of onset during late adolescence to young adulthood that together affect more than 10% of the US population and result in significant socioeconomic burdens (See, e.g., Kessler et al., Prevalence, severity, and comorbidity of 12-month DSM-IV disorders in the National Comorbidity Survey Replication. Arch Gen Psychiatry 62, 617-627 (2005); Merikangas et al., Lifetime and 12-month prevalence of bipolar spectrum disorder in the National Comorbidity Survey replication. Arch Gen Psychiatry 64, 543-552 (2007); Vigo et a., Estimating the true global burden of mental illness. Lancet Psychiatry 3, 171-178 (2016); and Adams et al., A Trillion-Dollar Opportunity: How Brain Research Can Drive Health and Prosperity. Inf. Tech. & Innov. Found. July 2016 pp. 1-31). While many studies have uncovered critical protein-coding genes associated with psychiatric disorders, such as those linked to synaptic plasticity and glutamate neurotransmission (See e.g., Crabtree et al., Synaptic plasticity, neural circuits, and the emerging role of altered short-term information processing in schizophrenia. Front Synaptic Neurosci 6, 28 (2014); Egan et al., Variation in GRM3 affects cognition, prefrontal glutamate, and risk for schizophrenia. Proc Natl Acad Sci USA 101, 12604-12609 (2004); Schloesser et al., Cellular plasticity cascades in the pathophysiology and treatment of bipolar disorder. Neuropsychopharmacology 33, 110-133 (2008); Tsai et al., Abnormal excitatory neurotransmitter metabolism in schizophrenic brains. Arch Gen Psychiatry 52, 829-836 (1995) and; Hashimoto et al., Postsynaptic density: a key convergent site for schizophrenia susceptibility factors and possible target for drug development. Drugs Today (Barc) 43, 645-654 (2007).), the pathogenesis and pathophysiology of psychiatric disorders still remain elusive. On a similar note, autism spectrum disorders (ASD) are devastating neurodevelopmental disorders that affect 1 out of 68 children and result in very significant societal burden (Adams et al). Together, all psychiatric and neurodevelopmental disorders affect more than 15% of the US population and impose a tremendous burden on patients and their families and massive costs that can likely amount up to 8% of the US GDP (Adams et al). The lack of reliable diagnostic tests has resulted in high rates of late or incorrect diagnosis and has further hindered the effort to better diagnose and treat psychiatric disorders. Thus, novel and robust molecular targets and mechanisms need to be discovered to produce clinically viable treatments. Both large and small non-coding RNAs (ncRNAs) have been recently shown to have important regulatory functions with significant implications for brain development, plasticity, and psychiatric disease (See e.g., G. Barry et al., The long non-coding RNA Gomafu is acutely regulated in response to neuronal activation and involved in schizophrenia-associated alternative splicing. Mol Psychiatry 19, 486-494 (2014); Bavamian et al., Dysregulation of miR-34a links neuronal development to genetic risk factors for bipolar disorder. Mol Psychiatry 20, 573-584 (2015); Qureshi et al., Emerging roles of non-coding RNAs in brain evolution, development, plasticity and disease. Nat Rev Neurosci 13, 528-541 (2012); Xu et al., Derepression of a neuronal inhibitor due to miRNA dysregulation in a schizophrenia-related microdeletion. Cell 152, 262-275 (2013) and; Geaghan et al., MicroRNA and Posttranscriptional Dysregulation in Psychiatry. Biol Psychiatry 78, 231-239 (2015)). Understanding the function of ncRNAs has led to the recognition of their ability to orchestrate the activity of complex regulatory pathways, which allows them to link multiple genetic risk factors for polygenic human disorders, such as psychiatric disorders, into functional networks.

Circular RNAs (circRNAs) are a novel category of long ncRNAs is that are derived from the circularization and covalent joining of backspliced exons and/or introns. CircRNAs are particularly enriched in the mammalian brain, yet, with few exceptions, lack the capacity of being translated into protein. The recent application of improved annotation tools following deep sequencing has revealed the existence of tens of thousands of circRNAs in multiple species. Some circRNAs are known to sequester microRNAs (miRNAs) by containing partial complementary sequences and others to associate with RNA-binding proteins (RBPs) and transcription factors. However, the mechanism of action of the overwhelming majority of brain expressed circRNAs remains elusive. Moreover, their significance for psychiatric disorders has not yet been explored, despite the findings that brain-enriched circRNAs are being preferentially derived from genes involved in synaptic plasticity, and the fact that circRNAs are the most resistant to degradation of the RNA species and, thus, ideal for postmortem and biomarker studies.

### SUMMARY

According to an embodiment the present disclosure provides compositions, kits, assays, and methods for the identification, diagnosis, screening, treatment and/or monitoring of brain disorders including, but not necessarily limited to, psychiatric disorders such as bipolar disorder (BD), schizophrenia (SCZ), depression, Attention-Deficit/Hyperactivity Disorder (ADHD), Obsessive-compulsive disorder (OCD), Anxiety Disorders, etc., and neurodevelopmental disorders such as Autism, Asperger's Syndrome, and other Autism Spectrum Disorders (ASD), pervasive developmental disorders not otherwise specified (PDD-NOS), etc. According to a specific embodiment, the present disclosure provides a plurality of circular RNAs (circRNAs) the expression of which is correlated with brain disorders.

### BRIEF DESCRIPTION OF THE DRAWINGS

FIG. 1A shows hierarchical clustering analysis of circRNA array data in 100 OFC RNA samples treated with RNase-R for linear RNA digestion. Example of circRNA array raw image is also shown.

FIG. 1B is a volcano plot showing differential circRNA expression in BD patients vs unaffected Controls (x-axis=relative to control log 2 fold changes; y-axis: negative log 10 of the p-values). Vertical lines correspond to >1.25-fold changes, and the horizontal line represents p<0.05. Example of validated circRNAs are shown in the graph.

FIG. 1C is a volcano plot showing differential circRNA expression in SCZ patients vs unaffected Controls (x-axis=relative to control log 2 fold changes; y-axis: negative log 10 of the p-values). Vertical lines correspond to >1.25-fold changes, and the horizontal line represents p<0.05. Example of validated circRNAs are shown in the graph.

FIG. 2A is a schematic showing the molecular pathways formed by the host genes of altered in BD circRNAs based on ingenuity pathway analysis. Information on molecular expression/interactions/relationships are shown in the graph.

FIG. 2B is a schematic showing the molecular pathways formed by the host genes of altered in SCZ circRNAs based on ingenuity pathway analysis. Information on molecular expression/interactions/relationships are shown in the graph.

FIG. 3A is a schematic of human HOMER1 gene, pre-HOMER1B mRNA isoforms and circHomer1a biogenesis. Complementary sequences in intron 1 and 5 and RNA Binding Proteins (RBPs) are predicted to result in the backsplicing of exons 5 and 2 into a precursor circHomer1a sequence, which is spliced to generate circHomer1a. Primers and probes for detection and shRNA for knockdown of the unique circHomer1a splice junction are shown.

FIG. 3B shows the sequencing validation of the circHomer1a splice junction (SEQ ID. NO. 20) following qRT-PCR. Exon 5 and exon 2 boundaries shown below.

FIG. 3C shows the complete sequence (SEQ ID. No. 1) of the mature (spliced) circHomer1a shown as exon 2 and exon 5 sequences in order. The parts of exons 2 and 5 that create the splice junction (shown also in FIG. 3B) are shown in dashed underline and double underline, respectively. The sequences of the forward (SEQ ID. No. 2) and reverse (SEQ ID. No. 3) PCR primers used to detect circHomer1a are shown and their location within the sequence is shown as underlined (forward primer) and underlined plus italics (reverse primer).

FIG. 4A shows the mean±SEM relative to Control circHomer1a levels (qRT-PCR, normalized to the geometric mean of highly expressed and un-altered circTulp4 and CDR1as) in the OFC of subjects with SCZ and BD.

FIG. 4B shows the mean±SEM relative to Control circCUL4A levels (qRT-PCR, normalized to the geometric mean of highly expressed and un-altered circTulp4 and CDR1as) in the OFC of subjects with SCZ and BD.

FIG. 4C shows reductions in circHomer1a in SCZ and BD OFC via qRT-PCR in RNAseR-treated samples (no normalization, shown as relative to Control Mean±SEM ratios).

FIG. 4D shows RNaseR increases the relative abundance of circHomer1a, whereas poly-A selection depletes circHomer1a expression (Mean±SEM, ***p<0.001, two-tailed one sample t-test). In all graphs individual SCZ, BD, and Control sample values are shown.

FIG. 4E, shows mean±S.E.M relative to the mean of unaffected Controls circADAM22 (normalized to the geometric mean of circTulp4 and CDR1as) calculated as geometric mean of both circRNAs—no additional normalization), expression in BD, SCZ, and Control samples from the OFC. *p<0.05, based on general linear model correcting for RIN, PMI, RI, and brain pH. A schematic showing the multi-exonic nature of circADAM22 is also shown.

FIG. 4F shows mean±S.E.M relative to the mean of unaffected Controls circTulp4/CDR1as calculated as geometric mean of both circRNAs—no additional normalization), expression in BD, SCZ, and Control samples from the OFC. *p<0.05, based on general linear model correcting for RIN, PMI, RI, and brain pH. A schematic showing the multi-exonic nature of circADAM22 is also shown.

In FIGS. 4A-C, E-F **p<0.01, based on a Univariate General Linear Model corrected for RIN, PMI, RI, and brain pH.

FIG. 4G shows the correlation between relative to Control OFC expression of circHomer1a in patients with SCZ and age of onset of SCZ.

FIG. 4H shows the correlation between relative to Control OFC expression of circHomer1a in patients with SCZ and BD and duration of illness.

In FIGS. 4G and H, spearman correlation coefficients (r) and two-tailed p-values are shown in the graphs and individual SCZ and/or BD sample values are shown.

FIG. 5A shows the mean±SEM relative to Control circHomer1a levels (qRT-PCR, normalized to the geometric mean of highly expressed and un-altered circTulp4 and CDR1as) in the DLPFC of subjects with BD and SCZ.

FIG. 5B shows the mean±SEM relative to Control circCUL4A levels (qRT-PCR, normalized to the geometric mean of highly expressed and un-altered circTulp4 and CDR1as) in the DLPFC of subjects with BD and SCZ.

FIG. 5C shows the correlation between relative to Control DLPFC expression of circHomer1a in patients with SCZ and age of onset of SCZ.

FIG. 5D shows the correlation between relative to Control DLPFC expression of circHomer1a in patients with SCZ and BD and duration of illness. In all graphs individual SCZ, BD, and Control sample values are shown.

FIG. 6A is a representative bright-field image from 6-9 month differentiated human embryonic stem cell (hESC)-derived mature mixed neuronal and glial cultures.

FIG. 6B is an immunofluorescence image showing the presence of neurons (MAP2, red) and astrocytes (GFAP, green). Scale bar=100 μm.

FIG. 6C shows relative to Vehicle (N=5) Mean±SEM circHomer1a expression (based on circRNA qRT-PCR, without normalization) following treatment with olanzapine (Olanz, N=3), Haloperidol (Hal, N=4), or valproic acid (VPA, N=3) for 2 days in human 6-9 month differentiated stem cell-derived mixed neuronal and astrocyte cultures. *p<0.05, two-tailed one sample t-test. The number of replicates is also shown within the graph.

FIG. 7A is a schematic showing the location of SCZ-linked SNP rs12516663 inside the circHomer1a precursor.

FIG. 7B shows the mean±SEM relative circHomer1a (normalized to the geometric mean of circTulp4 and CDR1as) levels, in the OFC of BD and SCZ patients carrying one copy of the risk allele for SNP rs12516663 (TC) or only WT alleles (TT). All results are shown as relative to unaffected Control mean ratios. *p<0.05, two-tailed one sample t-test relative to TT. In all three graphs SCZ and BD cases with TT genotype are shown as purple circles, whereas those with TC genotype are shown as pink circles.

FIG. 7C shows the mean±SEM relative circHomer1a (normalized to the geometric mean of circTulp4 and CDR1as) levels, in the DLPFC of BD and SCZ patients carrying one copy of the risk allele for SNP rs12516663 (TC) or only WT alleles (TT).

FIG. 7D is a schematic showing the OFC and the inferior frontal gyms, pars triangularis, whose resting-state functional connectivity is associated with rs12516663.

FIG. 7E is a scatter plot of rs12516663 associated with resting-state functional connectivity (FC) between the OFC and the inferior frontal gyrus, pars triangularis. Corrected SNP values are plotted in x-axis. Correlation p-value shown based on original coding. Individual SCZ, and Control correlations are shown in the graph.

FIG. 8A is a schematic of human HOMER1 gene, pre-HOMER1B mRNA isoforms and circHomer1b biogenesis. Complementary sequences in intron 1 and 3 and RNA Binding Proteins (RBPs) are predicted to result in the backsplicing of exons 3 and 2 into a precursor circHomer1b sequence (not shown in the graph), which is spliced to generate mature circHomer1b (shown in the graph). Primers for detection and shRNA for knockdown of the unique circHomer1b splice junction are shown.

FIG. 8B shows the complete sequence of the mature (spliced) circHomer1b shown as exon2 and exon 3 sequences in order. The parts of exons 2 and 3 that create the splice junction (shown also in a) are shown in dotted underline and double underline, respectively. The sequences of the forward and reverse PCR primers used to detect circHomer1b are shown and their location within the sequence is shown as underlined (forward primer) and underlined plus italics (reverse primer).

FIG. 8C shows the mean±SEM relative to Control circHomer1b levels (qRT-PCR, normalized to the geometric mean of highly expressed and un-altered circTulp4 and CDR1as) in the OFC of subjects with SCZ and BD. A schematic of the exonic nature of circHomer1b is also shown. In all graphs individual SCZ, BD, and Control sample values are shown. ***p<0.001, based on general linear model correcting for RIN, PMI, RI, and brain pH.

FIG. 8C shows the mean±SEM relative to Control circHomer1b levels (qRT-PCR, normalized to the geometric mean of highly expressed and un-altered circTulp4 and CDR1as) in the DLPFC of subjects with SCZ and BD. A schematic of the exonic nature of circHomer1b is also shown.

FIG. 9A shows the mean±SEM relative to the mean of Control neuronal progenitors (NPs) circHomer1a levels in iPSC-derived SCZ patient and Control (N=10 Control and 9 SCZ subjects) NPs and 6 week differentiated neurons. ^{#}0.10>p>0.05, *p<0.05, two-tailed one sample t-test relative to the Control of the same developmental time-point.

FIG. 9B shows the mean±SEM relative to the mean of Control NPs circHomer1a levels in iPSC-derived BD patient and Control (N=3 Control and 4 BD) NPs and 2 week, 4 week, and 6 week differentiated neurons. ^{#}0.10>p>0.05, *p<0.05, two-tailed one sample t-test relative to the Control of the same developmental time-point. In all graphs individual samples are shown.

FIG. 10A is a schematic of circRNA-specific shRNA knockdown design for circHomer1a (same design for human, mouse, rat circHomer1a). The shRNA targeting the circHomer1a splice junction is asymmetrically complimentary with the 5′ of exon 2 and the 3 of exon 5, which participate in the creation of circHomer1a via backsplicing and covalent joining (upper). The same shRNA does not cause have any significant complementarity with either exon 2 and exon 3 when present in any linear Homer1 mRNA to cause degradation or miRNA-like translational inhibition and subsequent decay (only nts 1-6 in the 5′ “seed sequence” of the shRNA are complementary with the 5′ of exon 2).

FIG. 10B shows the mean±SEM relative to scrambled shRNA control (sh-Control) mouse circHomer1a levels after shRNA-mediated circHomer1a knockdown (sh-circHomer1) in mouse OFC. ^{#}0.10<p<0.05, *p<0.05, two-tailed one sample t-test relative to sh-Control mean, circHomer1a was normalized to mouse circTulp4 and Homer1 mRNA isoforms to 18S rRNA, and the number of replicates is shown within the graphs.

FIG. 10C shows the mean±SEM relative to scrambled shRNA control (sh-Control) mouse Homer1 mRNA isoform levels after shRNA-mediated circHomer1a knockdown (sh-circHomer1) in mouse OFC. ^{#}0.10<p<0.05, *p<0.05, two-tailed one sample t-test relative to sh-Control mean, circHomer1a was normalized to mouse circTulp4 and Homer1 mRNA isoforms to 18S rRNA, and the number of replicates is shown within the graphs.

FIG. 10D is an image showing the expression of circHomer1a in mouse neuronal cultures via in situ hybridization with two probes aiming at its splice junction (both probes need to specifically bind for a signal to be generated) and signal amplification with co-immunostaining with mouse SMI312 and HOMER1B/C. Scale bar=50 μm

FIG. 11 depicts pathway analysis of mRNA alterations following in vivo knockdown of mouse circHomer1a in mouse OFC. Schematic showing the molecular pathways formed by the mRNAs altered in following in vivo knockdown of mouse circHomer1a in mouse OFC following RNA sequencing and based on ingenuity pathway analysis. Information on molecular expression/interactions/relationships are shown in the graph.

FIG. 12 depicts pathway analysis of mRNA isoform alterations following in vivo knockdown of mouse circHomer1a in mouse OFC. Schematic showing the molecular pathways formed by the mRNA isoforms altered in following in vivo knockdown of mouse circHomer1a in mouse OFC following deep RNA sequencing and based on ingenuity pathway analysis. Information on molecular expression/interactions/relationships are shown in the graph.

FIG. 13A is a flow chart showing trials are initiated through a lever press (1), which leads to the onset of the pairwise stimuli on a touch sensitive screen (2). Touch of the rewarded stimulus results in delivery of reward in the magazine (3) concomitant with 1 second tone and illumination of the magazine light. Touches at the unrewarded lead to illumination of the house light (4) for 10 seconds for an incorrect response. Error choices are followed by correction trials in which a subsequent initiation led to the stimuli presented in the same spatial orientation until a correct response is made to prevent side-bias and measure perseveration.

FIG. 13B shows behavioral paradigm interventions and recording session's timeline. After training, lentiviral injection with circHomer1 or scrambled control shRNA, and 2 weeks of recovery, discrimination and reversal learning trials were carried out.

FIG. 13C is a graph showing that in vivo knockdown of circHomer1a in mouse OFC increases the number of total errors to reach reversal criterion during a touch-screen reversal learning paradigm. *p<0.05, based on two-tailed one sample t-test compared to sh-Control mean.

FIG. 13D is a graph showing that knockdown of circHomer1a (sh-circHomer1) did not alter reaction time to choose between stimuli (choice) or retrieve a reward (magazine) during reversal learning. All bar graphs represent mean±SEM relative to scrambled shRNA control (sh-Control) values and display the number of replicates within.

FIG. 14A shows the complete sequence of the mature (spliced) circADAM22. The splice junctions are shown in dotted underline and the sequences of the forward and reverse PCR primers are shown as underlined (forward primer) and underlined plus italics (reverse primer).

FIG. 14B shows the complete sequence of the mature (spliced) circCUL4A. The splice junctions are shown dotted underline with the sequence also including the underlined forward primer. The sequences of the forward and reverse PCR primers are shown as underlined (forward primer) and underlined plus italics (reverse primer).

FIG. 14C shows the sequences used for shRNA knockdown and in situ hybridization of circHomer1a(sh-circHomer1) in mouse, human, and rat (see FIGS. 10A-D) for shRNA knockdown design of circHomer1a).

### DETAILED DESCRIPTION

According to an embodiment the present disclosure provides compositions, kits, assays, and methods for the identification, diagnosis, screening, treatment and/or monitoring of brain disorders including, but not necessarily limited to, psychiatric disorders such as bipolar disorder (BD), schizophrenia (SCZ), depression, Attention-Deficit/Hyperactivity Disorder (ADHD), Obsessive-compulsive disorder (OCD), Anxiety Disorders, etc., and neurodevelopmental disorders such as Autism, Asperger's Syndrome, and other Autism Spectrum Disorders (ASD), pervasive developmental disorders not otherwise specified (PDD-NOS), etc. For the purposes of the present disclosure the term “brain disorder(s)” is intended to be an inclusive term which is encompasses a wide range of psychiatric and neurodevelopmental disorders including those identified above.

According to a specific embodiment, the present disclosure provides a plurality of circular RNAs (circRNAs) the expression of which is correlated with brain disorders. CircRNAs are a novel category of long non-coding RNAs (ncRNAs) that are derived from the circularization and covalent joining of backspliced exons and/or introns. CircRNAs are particularly enriched in the mammalian brain (close to 100,000 unique circRNAs have been identified in the human brain), yet, with few exceptions, lack the capacity of being translated into protein. The recent application of improved annotation tools following deep sequencing has revealed the existence of tens of thousands of circRNAs in multiple species. Some circRNAs are known to sequester microRNAs (miRNAs) by containing partial complementary sequences and others to associate with RNA-binding proteins (RBPs) and transcription factors. However, the mechanism of action of the overwhelming majority of brain expressed circRNAs remains elusive. Additional general information about circRNAs can be found, for example, in S. P. Barrett, et al., Circular RNA biogenesis can proceed through an exon-containing lariat precursor. Elife 4, e07540 (2015).

Tables 1-4 provide circRNAs which showed significantly altered expression in individuals diagnosed with specific brain disorders compared to unaffected controls. For the purposes of the present disclosure, the circRNAs provided in Tables 1-4 are collectively referred to herein as “brain disorder-associated circRNA biomarkers.” Specifically, Table 1 provides circRNAs which showed significantly altered expression in human induced pluripotent stem cell (iPSC)-derived neuronal progenitors from patients with early onset schizophrenia vs unaffected controls. Table 2 provides circRNAs which showed significantly altered expression in human induced pluripotent stem cell (iPSC)-derived neurons (6 weeks of differentiation) from patients with early onset schizophrenia vs unaffected controls. Table 3 provides circRNAs which showed significantly altered expression in the orbitofrontal cortex (OFC) of subjects with Bipolar Disorder vs unaffected Controls. Table 4 provides cirRNAs which showed significantly altered expression in the OFC of subjects with Schizophrenia vs unaffected controls. For the purposes of the present disclosure, the term “significantly altered expression” means that the expression of a circRNA in patient samples is significantly lower or higher compared to its expression in unaffected control samples or other disorders determined via two-tailed Student's t-test with p<0.05 as a cutoff p-value for statistical significance.

The circRNAs in Tables 1 and 2 were identified by profiling circRNA expression in 5 early (childhood) onset SCZ and 5 unaffected Control iPSC-derived neuronal progenitor (Table 1) and 6 week differentiated neuronal cultures (Table 2). Samples were gifted by the lab of Dr. Kristen Brennand from Mount Sinai School of Medicine.

The circRNAs in Tables 3 and 4 were identified by profiling circRNA expression in OFC RNA samples from 34 SCZ, 32 BD, and 34 unaffected control subjects from the Stanley Medical Research Institute.

Each of the four Tables shows the unique transcript start and end chromosomal coordinates (tx Start and tx End), the chromosome (Chr) number and strand, the circRNA type, the circRNA alias based on circBase (http://circbase.mdc-berlin.de) or other circRNA databases the relative to Control fold change and uncorrected p-value, and the host gene symbol for significantly altered circRNAs (p<0.05, two-tailed Students t-test).” In the column titled “circRNA type” in Tables 1-4, “exo” refers to exonic, “s/o” refers to “sense overlapping,” “int” refers to intronic, “ing” refers to “intergenic” and “as” refers to antisense.

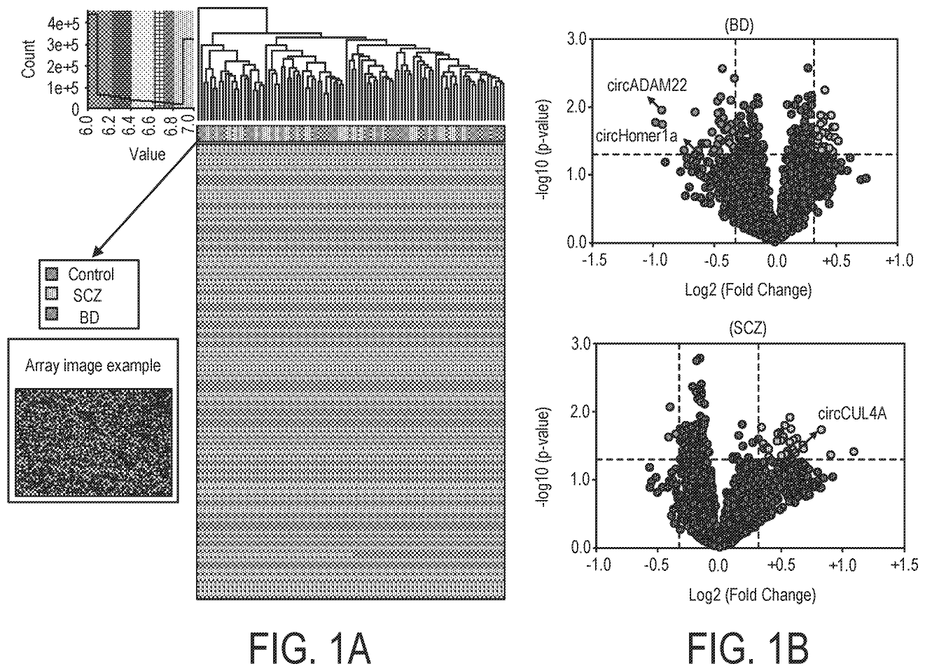

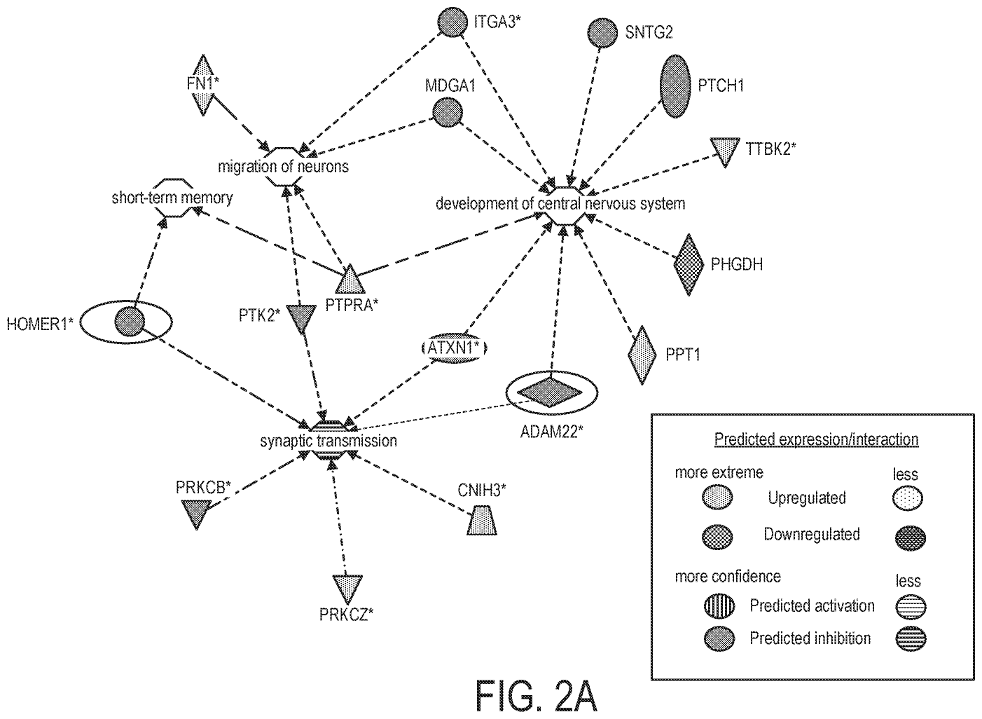

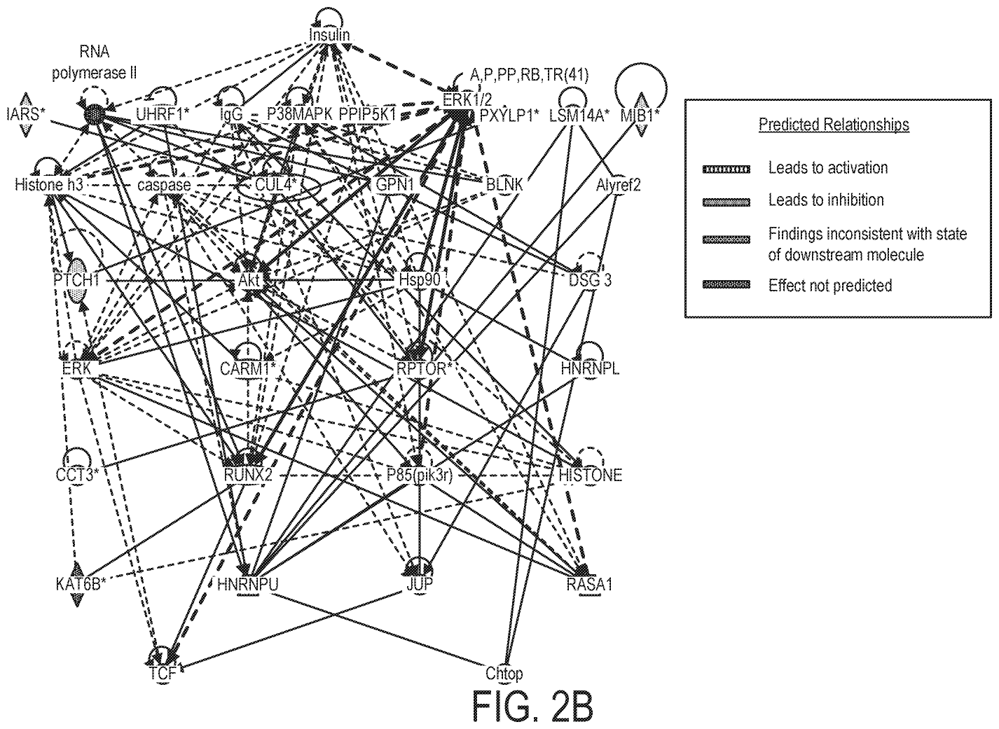

All circRNA array experiments used for Tables 1-4 were performed by employing a commercially available circRNA microarray platform that uses 13,617 circRNA splice junction probes designed based on previous RNA sequencing and circRNA annotation data (Service sold by Arraystar Inc.). Analysis of circRNA changes in postmortem brain samples from the OFC of subjects with BD uncovered a subset of differentially expressed circRNAs (Table 3) a subset of which stemmed from genes related to synaptic transmission, neuronal development and migration, and short term memory (FIGS. 1A-C, 2A-B), whereas altered circRNAs in SCZ (Table 4 and FIGS. 1A-C, 2A-B) were associated, among others, with the mitogen-activated protein kinase (MAPK/ERK) and protein kinase B (PKB/AKT) pathways. Additional methods for identifying these brain disorder-associated circRNA biomarkers are discussed below.

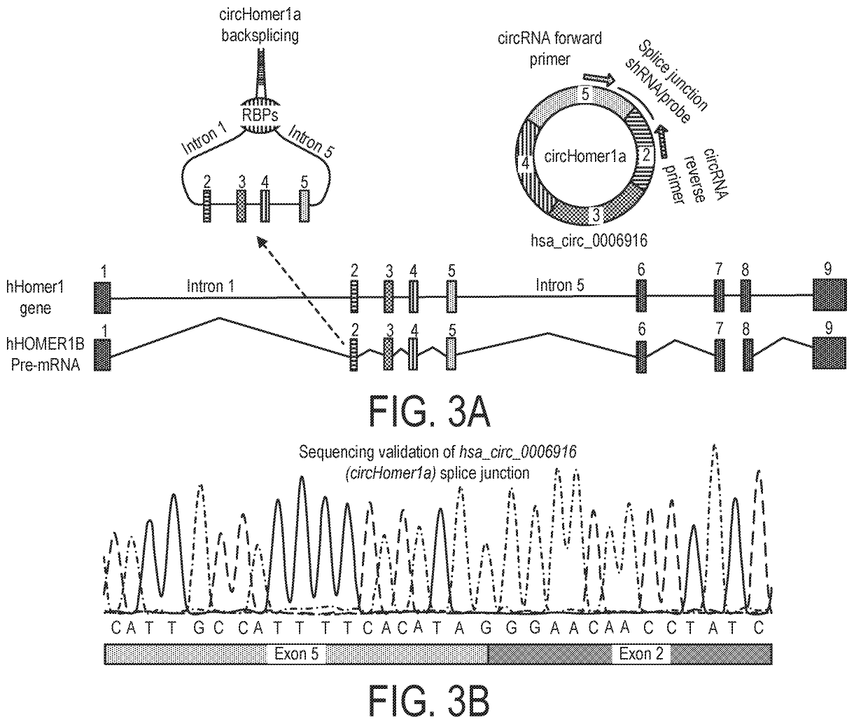

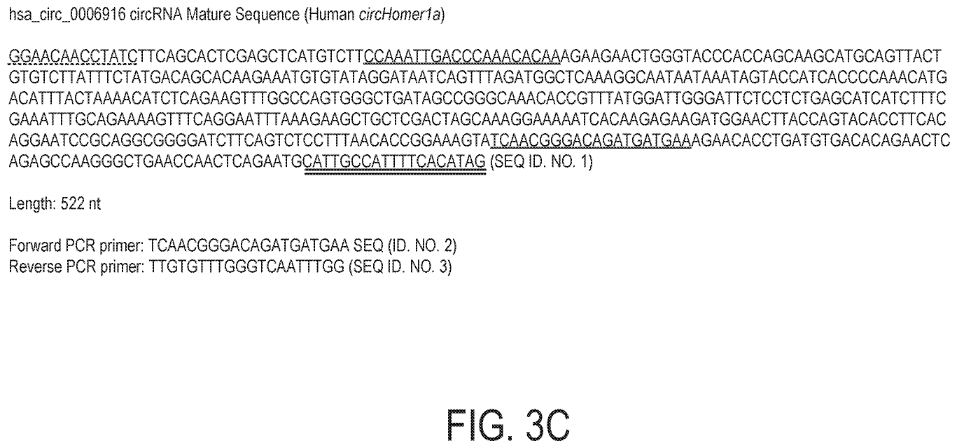

Given that harsh RNAseR treatment can partially digest some circRNAs together with all linear RNAs, thus making circRNA screening semi-quantitative, the present disclosure also provides novel validated and sequence-verified circRNA-specific qRT-PCR primers aimed at the unique circRNA-specific splice junction, which enable the measurement of a subset of dysregulated circRNAs in non RNaseR-treated samples. These primers are shown in FIGS. 3C, 8B, and 14A, B (See also SEQ ID NOS. 2, 3, 5, 6, 8, 9, 11 or 12).

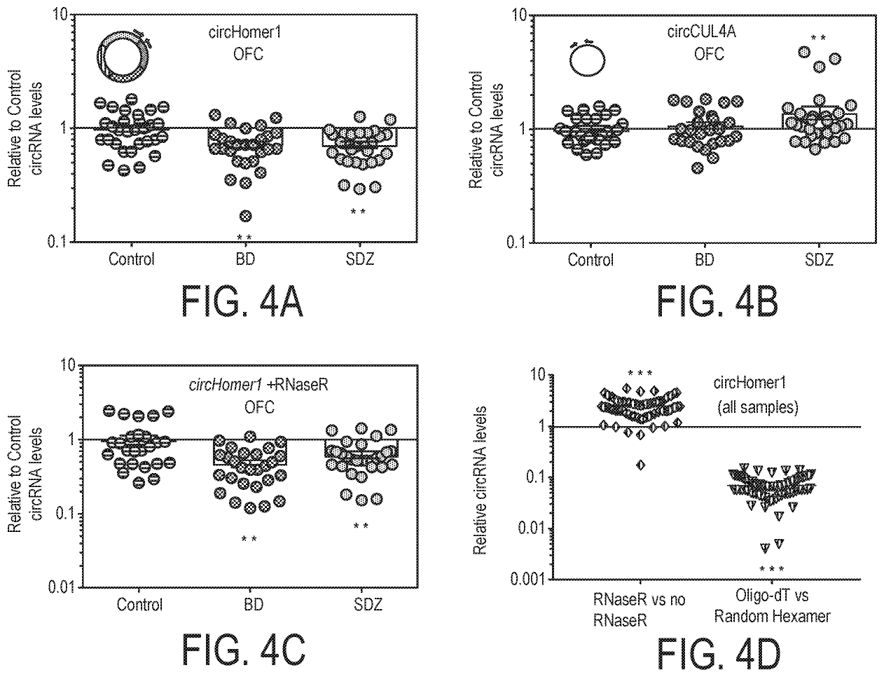

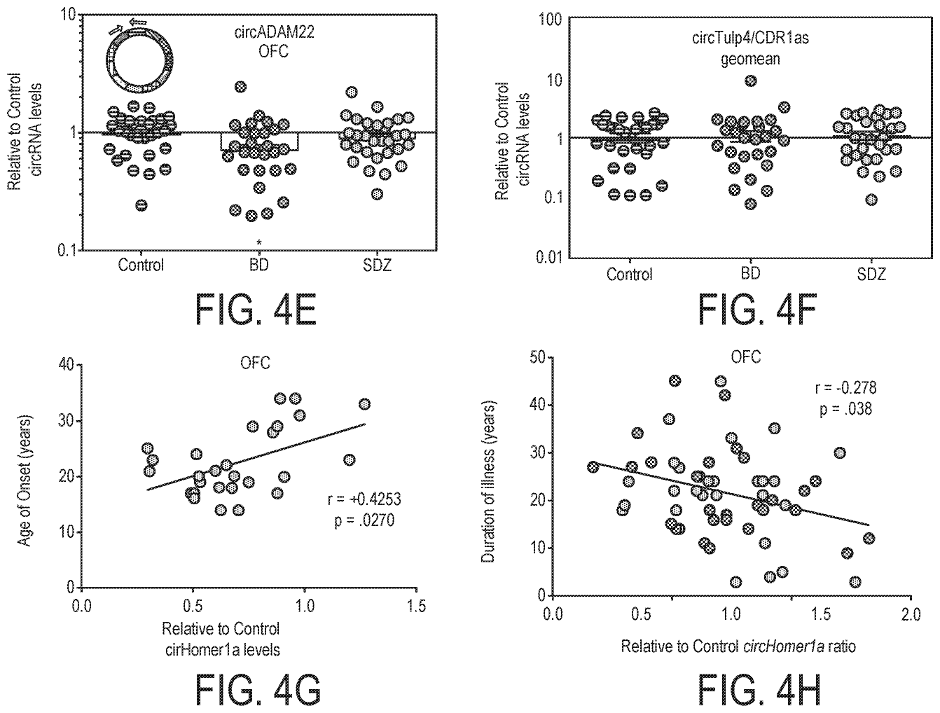

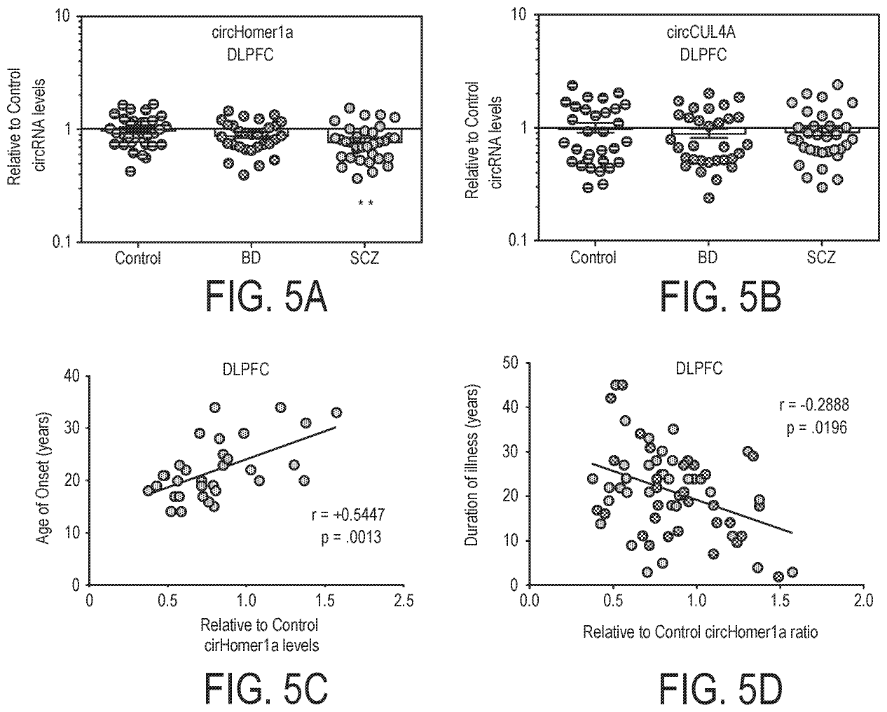

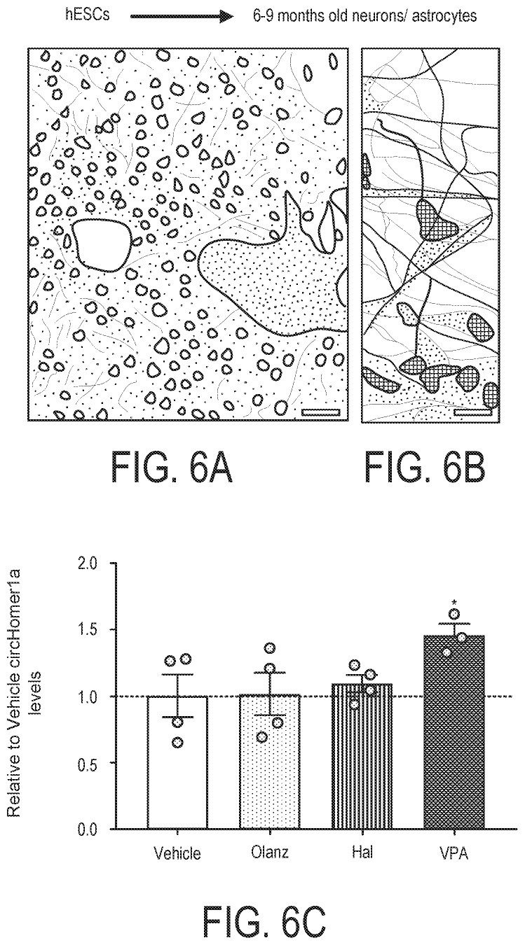

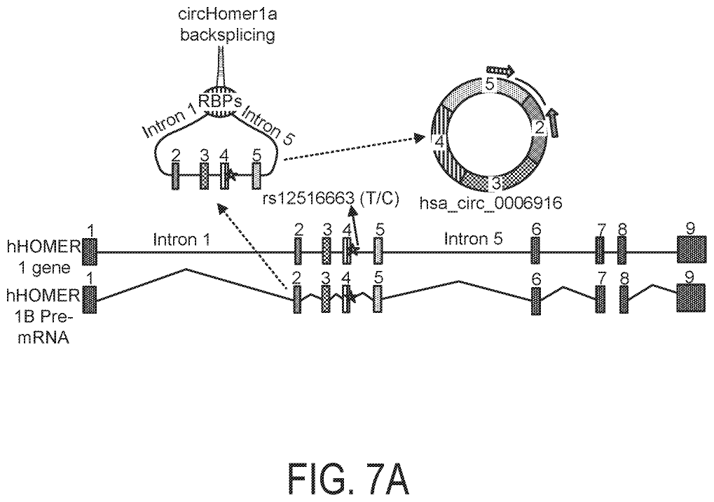

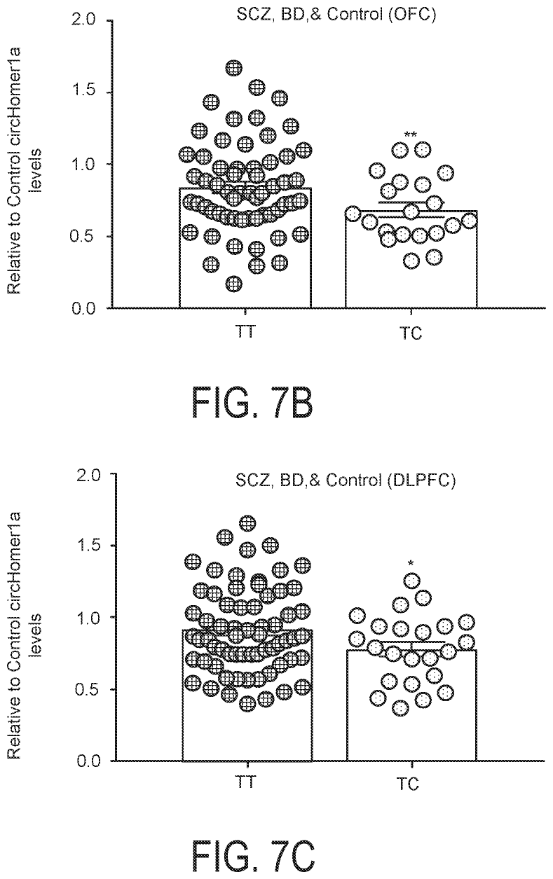

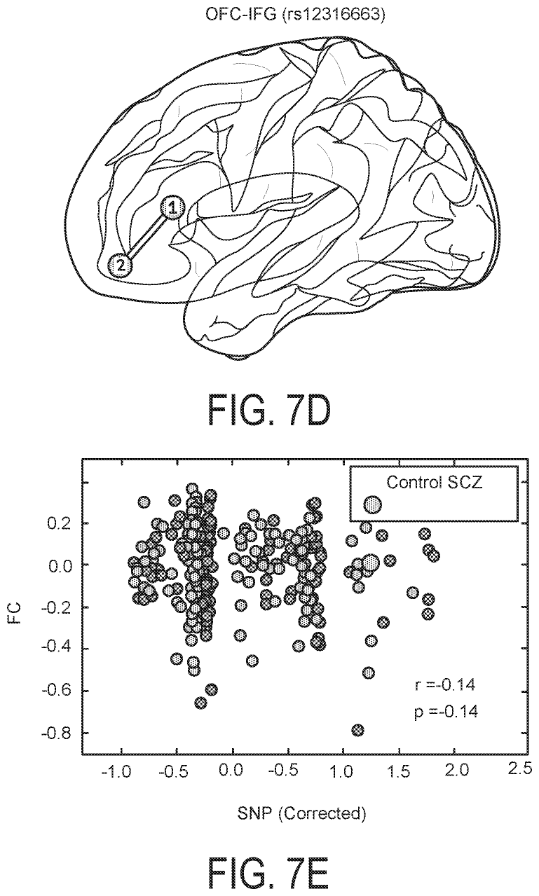

According to a specific embodiment, the present disclosure provides circHomer1a (circRNA alias via circbase and circinteractome databases: hsa_circ_0006916) as a biomarker for brain disorders including, but not necessarily limited to BD and SCZ. The sequence for human circHomer1a is shown in FIG. 3C (SEQ ID. NO. 1). (See also FIG. 7A.) FIG. 3B shows the sequence validation of its unique splice junction. FIG. 3C also shows the location and sequence of the primers used for its detection (See also SEQ ID NOS. 2, 3)). circHomer1a is a circRNA derived from exons 2-5 of Homer protein homolog 1 (HOMER1) (FIG. 3A). circHomer1a is reduced in the OFC and dorsolateral prefrontal cortex (DLPFC) from patients with SCZ and BD and is positively correlated with age of onset of disease for SCZ patients and duration of illness for both SCZ and BD (FIGS. 4A-H, 5A-D). Of note, the changes in circHomer1a expression in the OFC of patients with SCZ and BD were not shown to be significantly affected by multiple postmortem demographics nor duration of antipsychotic treatment (FIGS. 6A-B). The lack of effect of antipsychotic treatment on circHomer1a expression was also validated in stem cell-derived cultures treated with Haloperidol or Olanzapine (FIG. 6B; Valproic acid treatment did increase circHomer1a levels, however), Furthermore, a SCZ-linked single nucleotide polymorphism (rs12516663) is associated with reduced circHomer1a expression in both the OFC and DLPFC and altered local functional connectivity (FIG. 7A-D).

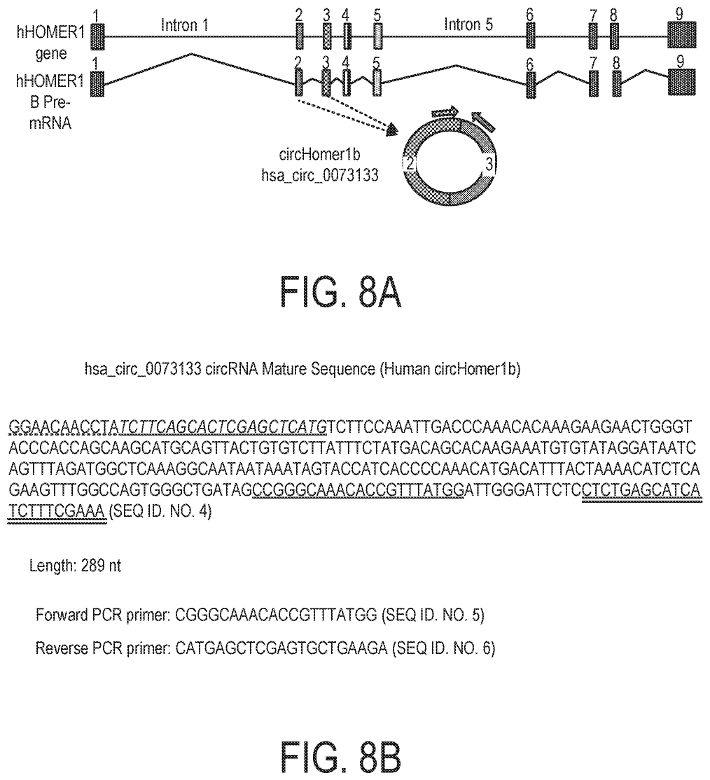

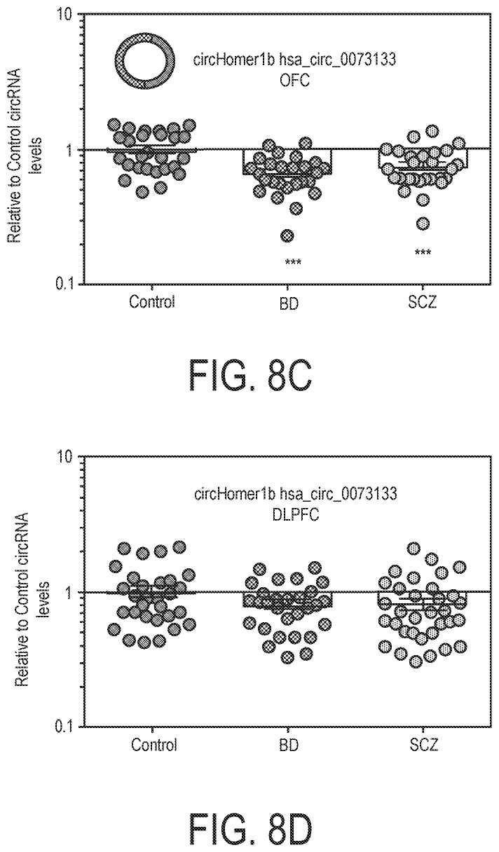

Another smaller circRNA derived from the human HOMER1 gene that is generated from the backsplicing of exons 2 and 3 of HOMER1 pre-mRNA was found to be reduced in the OFC but not DLPFC of subjects with SCZ and BD (FIG. 8A, C, D). FIG. 8B shows the sequence validation of its unique splice junction and the location and sequence of the primers used for its detection (See also SEQ ID NOS. 5, 6)). This circRNAs has been designated as circHomer1b (circRNA alias via circbase and circinteractome databases: hsa_circ_0073133 and chromosomal location chr5:78746812-78752841). (See, SEQ ID. NO. 4.)

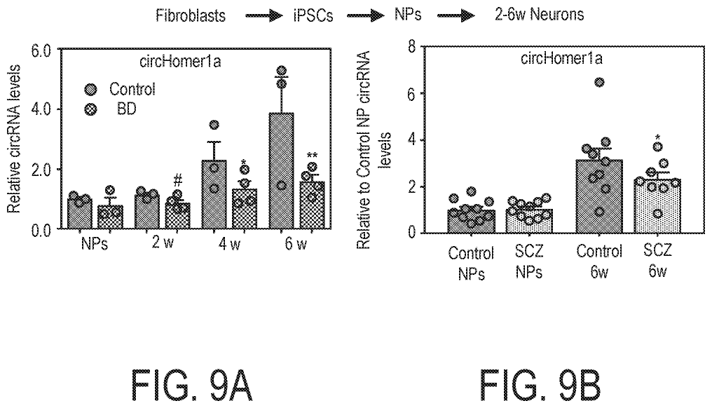

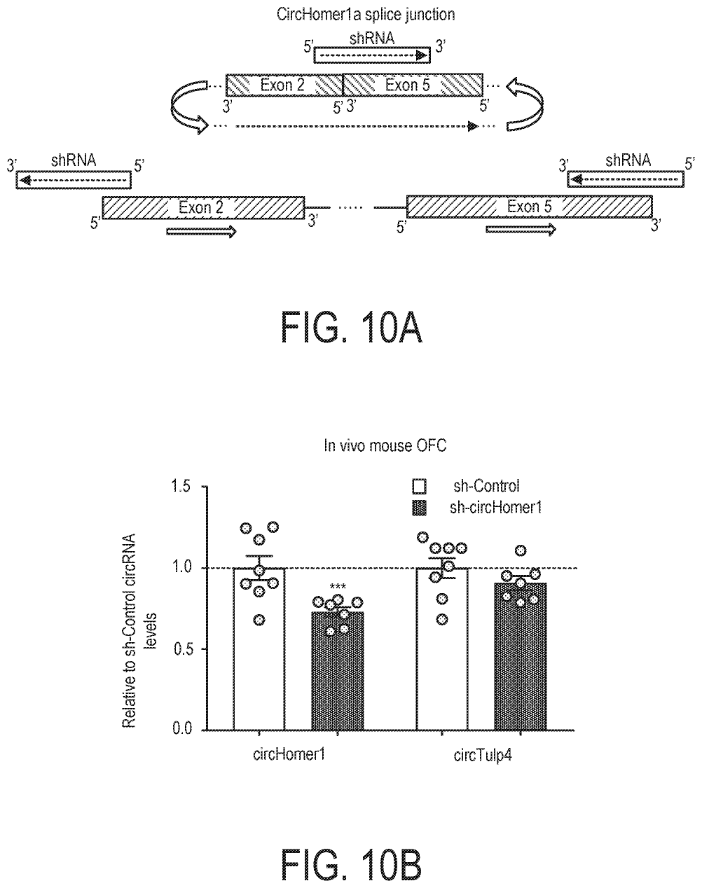

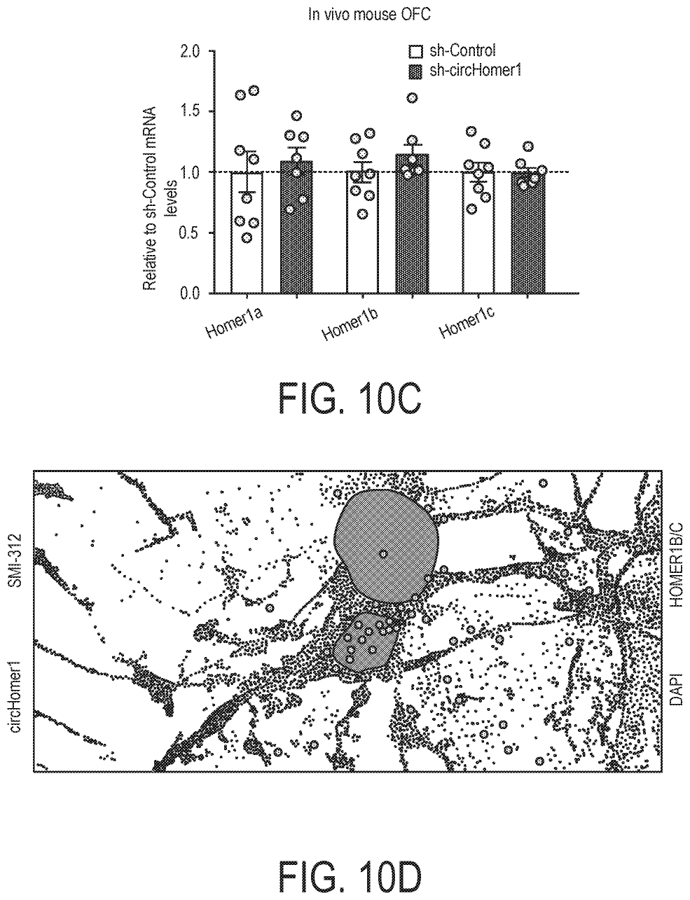

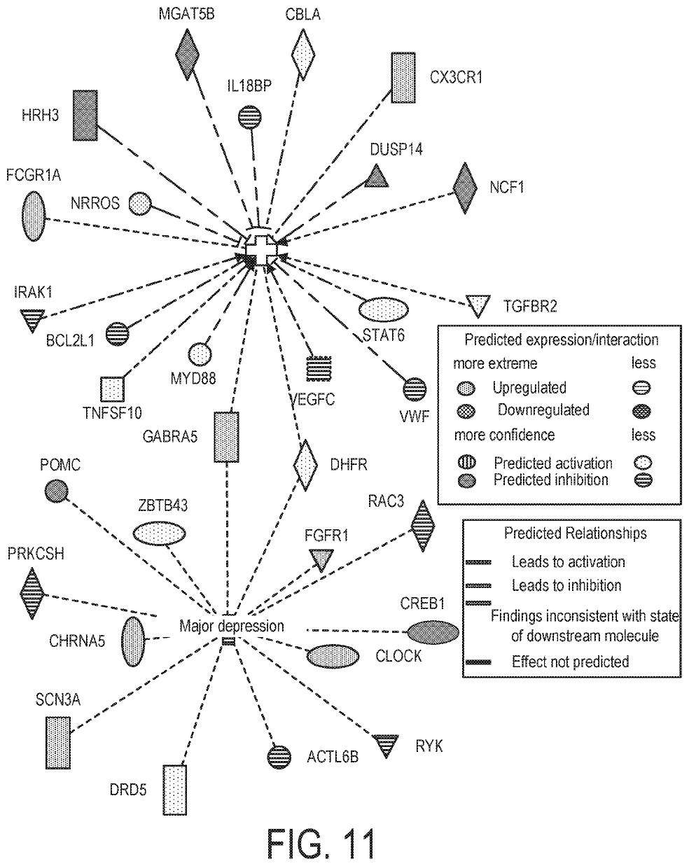

In addition to its reduced expression in multiple postmortem brain regions, circHomer1a was also found to be reduced in stem cell-derived (iPSC) neuronal cultures from patients with SCZ and BD (FIG. 9A-B). Using a uniquely-designed circRNA-specific shRNA approach that targets the splice junction in an asymmetric way between the two exons so as to avoid any inhibition of the expression of the linear mRNAs stemming from mouse Homer1 (same approach was used for human circHomer1a knockdown) and incorporating the shRNA in a lentiviral vector we managed to knockdown expression of circHomer1a in vivo in mouse OFC via lentiviral injection (FIGS. 10A, 11). This approach resulted in significant and specific reductions in circHomer1a (FIGS. 10B-D, 11).

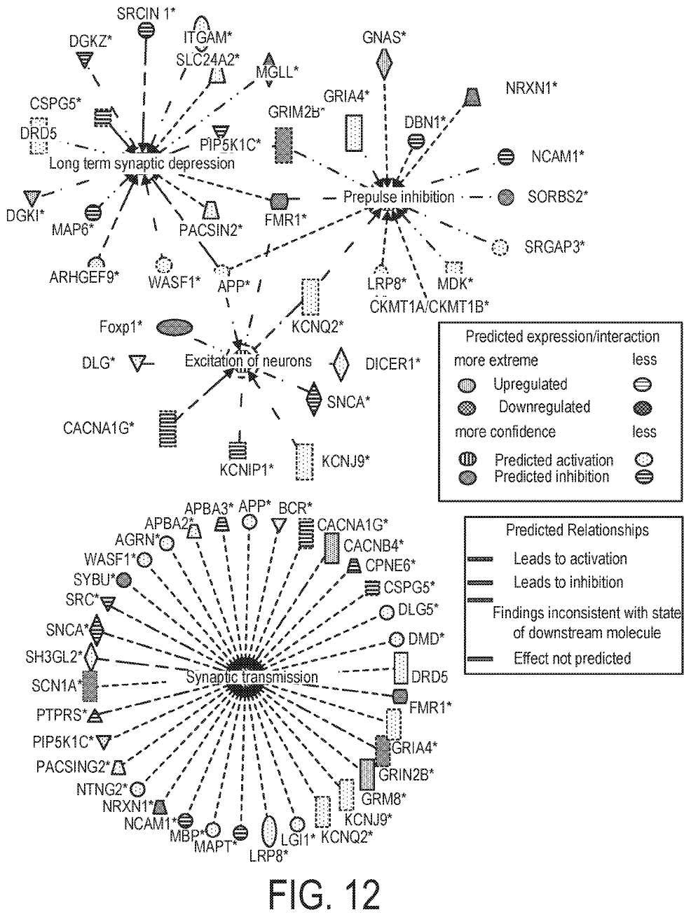

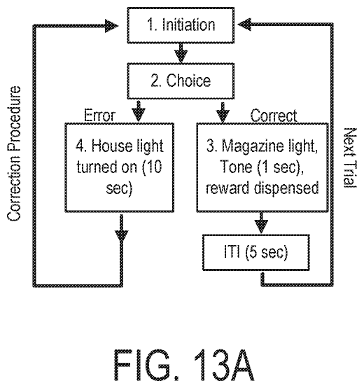

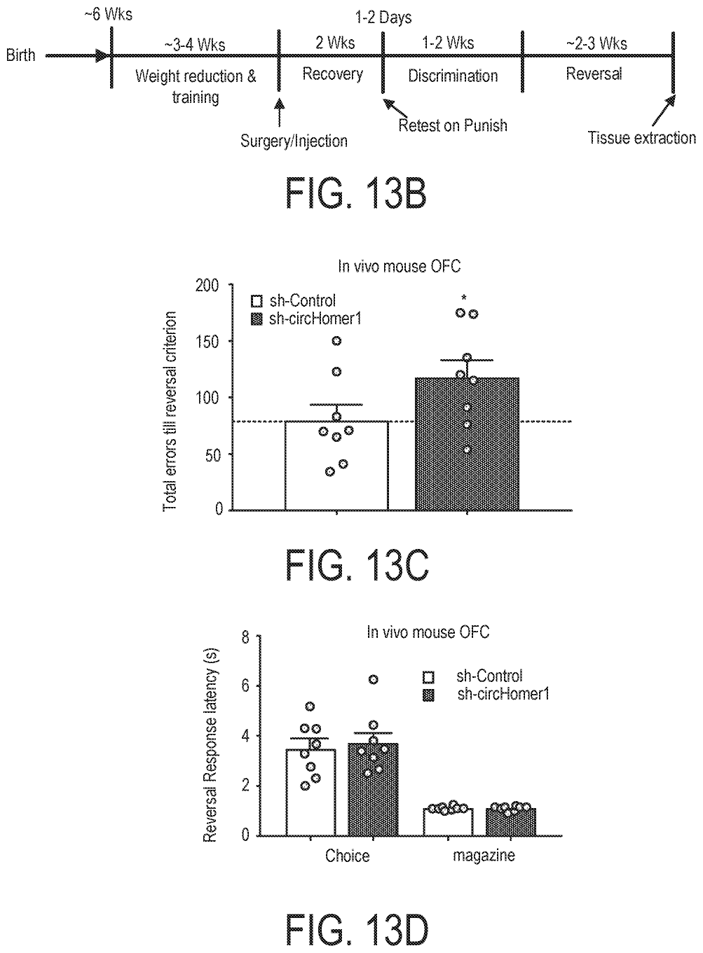

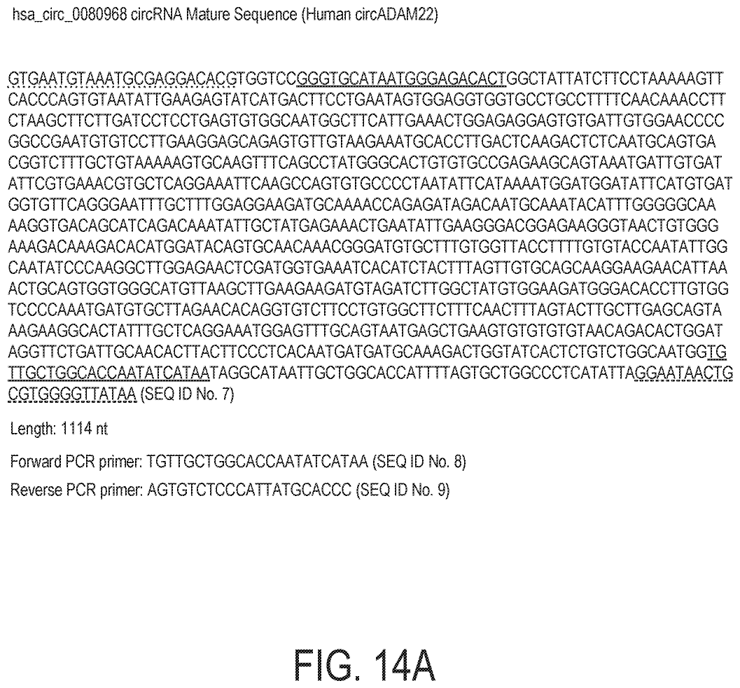

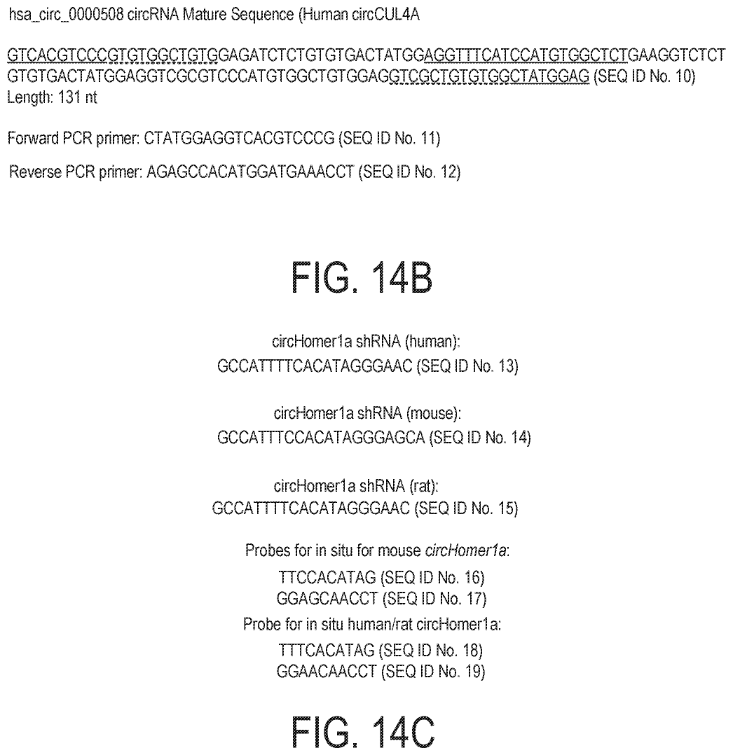

We also designed probes that span the circHomer1a splice junction for in situ hybridization of mouse/human/rat circHomer1a (sequences shown in FIG. 14). In vivo shRNA-mediated knockdown of circHomer1a in mouse OFC also resulted in the alterations in overall expression of mRNA involved in major depression and inflammation (FIG. 12) and on robust changes in the expression of alterative mRNA isoforms involved in neuronal excitation, synaptic transmission, long-term memory, and prepulse inhibition (FIG. 12). Lastly, circHomer1a revealed that it is necessary for OFC-mediated reversal learning, as demonstrated by in vivo knockdown of circHomer1 in mouse OFC (FIG. 13A-D).

According to yet another specific embodiment, the present disclosure provides circADAM22 (circRNA alias via circbase and circinteractome databases: hsa_circ_0080968), a circRNA derived from the epilepsy-related gene ADAM metallopeptidase domain 22 (ADAM22) as a biomarker for brain disorders including, but not necessarily limited to BD. The sequence for human circADAM22 is shown in FIGS. 14(A-C) (See SEQ ID. NO 7). circADAM22 was found to have decreased expression in the OFC of BD patients compared to unaffected individuals, while expression was unchanged for SCZ patients (FIG. 4E).

According to another specific embodiment, the present disclosure provides circCUL4A(circRNA alias via circbase and circinteractome databases: hsa_circ_0000508), as a biomarker for brain disorders including, but not necessarily limited to SCZ. The sequence for human circCUL4A is shown in FIG. 14 (See SEQ ID. NO. 10). circCUL4A was found to have increased expression in the OFC of SCZ patients compared to unaffected individuals, while expression was unchanged for BD patients (FIG. 4B).

Sequences of both circADAM22 and circCUL4A and primers used for their detection are shown in FIG. 14) (See SEQ ID. NOs 8, 9, 11, and 12).

It will be understood that while the brain disorder-associated circRNA biomarkers were identified by studying human subjects with SCZ and BD, it is well known that many psychiatric and neurodevelopmental disorders share risk genes and biomarkers. Accordingly, it is believed that while the brain disorder-associated circRNA biomarkers provided herein are clearly useful for the diagnosis and monitoring of SCZ and BD, they would be similarly useful for the diagnosis and monitoring of other brain disorders.

According to an embodiment, the present disclosure provides a composition comprising a panel of at least 1 circRNA, wherein the circRNA is a brain disorder-associated circRNA biomarker. According to a further embodiment, the present disclosure provides a composition comprising a panel of at least 5 circRNAs, where the 5 circRNAs are brain disorder-associated circRNA biomarkers. According to a still further embodiment, the present disclosure provides a composition comprising a panel of at least 10, 25, 50, 100, 200, 500, 1000, 2000, or 4000 circRNAs, where the circRNAs are brain disorder-associated circRNA biomarkers. According to an embodiment, at least one of the circRNAs in the composition may be circHomer1a.

According to an embodiment, the present disclosure provides an assay for the identification, diagnosis, screening, treatment and/or monitoring of a patient who has been or is being diagnosed, treated for, or who is suspected of having a brain disorder. For the purposes of the present disclosure, the terms “patient” or “subject” refer to any animal (e.g., mammal), including, but not limited to, humans, non-human primates, equines, canines, felines, rodents, and the like.

In general, the assay may include obtaining or providing a sample from a patient or an individual with an increased risk for a brain disorder (family history of brain disease and/or genetic predisposition, or a person with developmental delay or other clinical symptoms that could be associated with increased risk for developing a brain disorder) and measuring the expression of one or more of the brain disorder-associated circRNA biomarkers disclosed herein to produce an expression profile. According to a specific embodiment, at least one of the circRNAs in the expression profile may be circHomer1a. According to another specific embodiment, at least one of the circRNAs in the expression profile may be circCUL4A. According to yet another specific embodiment, at least one of the circRNAs in the expression profile may be circADAM22. The assay may further include comparing the patient's expression profile to a baseline. For identification, diagnostic, screening, or risk assessment purposes, the baseline may be an expression profile derived from one or more unaffected individuals (i.e. “a control profile”) or from one or more affected individuals (i.e. a “disease-state profile”). For treatment or monitoring purposes the baseline may be a control profile, disease state profile, or a previously obtained expression profile from the same individual. Because brain disorders tend to be highly inherited, there may circumstances wherein it is useful or informative for the baseline to be an expression profile derived from one or more individuals who are blood relatives of the patient.

According to some embodiments the assay may include or provide access to a database of expression profiles including, for example, control profiles, disease-state profiles, and the like. The database may include expression profiles from individuals or consolidated expression profiles from groups of individuals. The database may be curated such that all identifying information from any specific individual is scrubbed. The database may enable review of individual or consolidated expression profiles over time in order to, for example, monitor and/or evaluate disease progression, drug-response, therapy response, etc.

According to an embodiment, the sample may be, or may be produced from, a bodily fluid such as blood (including blood products such as serum, whole blood, plasma, and blood components and cells) cerebral fluid, saliva, urine, or blood from umbilical cords. According to another embodiment, the sample may be, or may produced from, bodily tissue, such as a skin biopsy, umbilical cord tissue or deciduous teeth.

According to a specific example, the sample may be neurons taken from the patient (such as those in the gut's enteric system) or derived from the patient from any cells from the aforementioned bodily fluids and tissues. For example, the neurons could be derived from induced pluripotent stem cells (iPSCs), embryonic stem cells, mesenchymal stem cells, or engineered somatic cells, which can in turn be derived from cells of bodily fluids and tissues See also, Kastenberg Z. J., et al, (2008) Alternative sources of pluripotency: science, ethics, and stem cells. Transplant Rev (Orlando) 22, 215-222, which is hereby incorporated by reference for all purposes.

According to a specific embodiment, the neurons are derived from iPSCs. iPSCs are similar to embryonic stem cells (ESC) in that iPSCs can be expanded indefinitely at the pluripotent stage and are able to differentiate into all three primary germ layers and, therefore, potentially into all the cell types of the body. iPSCs are derived from somatic cells and the process does not involve the use of embryonic cells, removing ethnical concerns.

Moreover, iPSC cells can be derived from patient samples that are easily and even non-invasively obtained such as skin, saliva, blood, or urine samples. Specific methods for generating iPSC cells are provided in Xia, G, et al. (2013). Generation of neural cells from DM1 induced pluripotent stem cells as cellular model for the study of central nervous system neuropathogenesis. Cell Reprogram 15: 166-177; and Zhou Y Y et al., Integration-free methods for generating induced pluripotent stem cells. Genomics Proteomics Bioinformatics. 2013 October; 11(5):284-7, each of which is hereby incorporated by reference for all purposes.

According to various embodiments, the assay may include any number of sample preparation techniques and compositions including, for example, sample isolation and/or culturing and suitable reagents therefore, the use of suitable buffering solutions, etc.

According to another embodiment, the assay may include amplification of one or more brain disorder-associated circRNA biomarkers in a patient sample. Suitable amplification techniques include, for example, polymerase chain reaction (PCR), including Quantitative Real-Time PCR (qRT-PCR), reverse transcription PCR (RT-PCR), quantitative reverse transcription RT-PCR (QRT-PCR), Multiplex PCR, Nested PCR, Quantitative PCR, Hot-start PCR, Touchdown PCR, Assembly PCR, and droplet PCR.

In general, PCRs and qRT-PCRs require the use of primers which are specifically designed to hybridize to and amplify a sequence of interest using multiple cycles of denaturation, annealing of primer pairs to opposite strands, and primer extension to exponentially produce copies of target nucleic acid sequence. Such PCRs can also be done following reverse transcription of RNA, including RNase-R-treated RNA. Accordingly, the present disclosure includes PCR and qRT-PCR primers that amplify at least a portion of one or more brain disorder-associated circRNA biomarkers to facilitate detection and/or quantification thereof.

According to an embodiment, at least one of the circRNAs being amplified may be circHomer1a, circHomer1b, circCUL4A or circADAM22. According to a specific embodiment, at least one of the primers used to amplify at least one of the circRNAs may comprise a sequence selected from the group consisting of SEQ ID NOS. 13, 14, 15, 16, 17, 18 and 19.

According to an embodiment, the assay may be or include a hybridization assay wherein a sample from a patient is obtained and interrogated using one or more probes designed to hybridize to one or more of the brain disorder-associated circRNA biomarkers or an amplicon thereof. Those of skill in the art will be familiar with a variety of different hybridization assays for detecting RNA including, but not limited to, microarrays, Northern blotting, PCR arrays. Accordingly, the present disclosure provides probes that hybridize to one or more of the brain disorder-associated circRNA biomarkers disclosed herein or an amplicon thereof and shRNA sequences aimed at specifically knocking down the expression of specific circRNAs.

According to an embodiment, at least one of the circRNAs being interrogated may be circHomer1a, circHomer1b, circCUL4A or circADAM22. According to a specific embodiment, at least one of the probes used to interrogate at least one of the circRNAs may comprise a sequence selected from the group consisting of SEQ ID NOS. 13, 14, 15, 16, 17, 18 and 19.

Those of skill in the art will be familiar with the process for designing appropriate circRNA splice junction probes and primers. However according to a specific embodiment of shRNA design disclosed herein, the circRNA splice junction probes and primers have the unique attribute of using shRNA sequences that span the splice junction in an asymmetric-manner. This design prevents any notable knockdown of linear mRNAs (FIGS. 10A-D). Depending on the particular assay begin designed, it will be understood that the probes may be appropriately labeled. Examples of commonly used labels for hybridization assay probes include, for example, radiolabels, chemiluminescent labels, intercalating dyes, fluorochromes, etc.

According to an embodiment, the assay may be or include DNA or RNA sequencing techniques. Those of skill in the art will be familiar with a variety of sequencing techniques including, but not necessarily limited to, chain terminator (Sanger) sequencing, dye terminator sequencing, pyrosequencing and single-molecule sequencing. In some cases, RNA sequences may be reverse transcribed into DNA sequencing prior to sequencing.

According to various embodiments, the present disclosure provides a kit comprising various reagents to enable the performance of one or more assays, wherein the assay comprises obtaining or providing a sample from a patient and measuring the expression of one or more of the brain disorder-associated circRNA biomarkers disclosed herein to produce an expression profile. The kit may include, for example, buffers, primers, and/or probes as needed in order to perform the assay. The kit may include only some or all of the reagents required to complete the assay. The kit may further include a database or access to a database to which the expression profile obtained from the assay may be compared.

According to various embodiments, the present disclosure provides methods for the identification, diagnosis, screening, treatment, risk assessment, and/or monitoring of brain disorders. In general, the method comprises obtaining, providing, or receiving a patient sample; assaying the sample to determine the expression levels of one or more of the brain disorder-associated circRNA biomarkers disclosed herein; and comparing the expression level to a baseline profile. The baseline profile may be, for example, a control profile, a disease-state profile, or a previously obtained expression profile from the same individual or one or more blood relatives of the patient. Comparison of the expression level of one or more of the brain disorder-associated circRNA biomarkers in the patient sample to the baseline can then be used to identify, diagnose, screen, treat, assess risk, or monitor one or more brain disorders in the patient. For example, a specific expression profile compared to a baseline profile may indicate that the patient should be classified as having a certain brain disorder such as schizophrenia or bipolar disorder or ASD, or that the patient could have increased chances for developing brain disorders. Alternatively or additionally, a specific expression profile compared to a baseline profile may indicate that a patient is more or less likely to respond to a particular medication, or that a particular medication is or is not providing a benefit or has adverse effects. Such information can be used by the patient, a caretaker, or a medical provider to make both medical and non-medical decisions including, for example, starting, stopping, or changing medication(s), changing dosage(s), examining the patient's compliance, etc.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

All patents and publications referenced below and/or mentioned herein are indicative of the levels of skill of those skilled in the art to which the invention pertains, and each such referenced patent or publication is hereby incorporated by reference to the same extent as if it had been incorporated by reference in its entirety individually or set forth herein in its entirety. Applicants reserve the right to physically incorporate into this specification any and all materials and information from any such cited patents or publications.

TABLE 1

| | | | | circRNA | | Fold | p-value | |
|---|---|---|---|---|---|---|---|---|
| Tx Start | Tx End | Chrom | Strand | type | circRNA Alias | SCZ iNPCs | SCZ iNPCs | GeneSymbol |
| 141285756 | 141301177 | chr8 | − | exo | hsa_circ_0085694 | 0.6801 | 0.000011 | TRAPPC9 |
| 121491550 | 121563485 | chr6 | − | exo | hsa_circ_0077745 | 0.5248 | 0.000016 | TBC1D32 |
| 211092981 | 211093411 | chr1 | − | exo | hsa_circ_0006123 | 0.5644 | 0.000084 | KCNH1 |
| 44424435 | 44448984 | chr21 | + | exo | hsa_circ_0061854 | 0.6541 | 0.000124 | PKNOX1 |
| 74655974 | 74656069 | chr2 | − | exo | hsa_circ_0055323 | 1.2405 | 0.000140 | RTKN |
| 86064658 | 86087186 | chr15 | + | exo | hsa_circ_0008454 | 0.7315 | 0.000164 | AKAP13 |
| 9758948 | 9769947 | chr12 | + | s/o | | 0.6531 | 0.000175 | LOC374443 |
| 33271149 | 33278223 | chr9 | + | exo | hsa_circ_0003575 | 0.6709 | 0.000205 | CHMP5 |
| 150634289 | 150636239 | chr1 | − | exo | | 0.6395 | 0.000274 | GOLPH3L |
| 532468 | 534580 | chr6 | − | s/o | | 0.7636 | 0.000296 | EXOC2 |
| 129396968 | 129411699 | chr12 | + | exo | hsa_circ_0029403 | 0.6079 | 0.000308 | GLT1D1 |
| 22151196 | 22199917 | chr1 | − | exo | hsa_circ_0010553 | 0.6301 | 0.000377 | HSPG2 |
| 232153372 | 232156156 | chr2 | + | exo | hsa_circ_0006604 | 1.3041 | 0.000388 | ARMC9 |
| 47512341 | 47522502 | chr6 | + | exo | | 0.6085 | 0.000392 | CD2AP |
| 42481721 | 42524149 | chr13 | − | exo | hsa_circ_0030123 | 0.8583 | 0.000496 | VWA8 |
| 130915557 | 130916831 | chr8 | − | exo | hsa_circ_0085567 | 0.7053 | 0.000509 | FAM49B |
| 72165697 | 72176381 | chr14 | + | exo | hsa_circ_0032475 | 0.7136 | 0.000514 | SIPA1L1 |
| 126519981 | 126554909 | chr9 | − | exo | hsa_circ_0088475 | 1.1620 | 0.000518 | DENND1A |
| 47842375 | 47844511 | chr1 | + | exo | hsa_circ_0012384 | 0.6849 | 0.000541 | CMPK1 |
| 58610366 | 58617086 | chr16 | − | exo | hsa_circ_0006275 | 0.8107 | 0.000556 | CNOT1 |
| 35936438 | 35945538 | chr17 | − | exo | hsa_circ_0043297 | 0.8022 | 0.000607 | SYNRG |
| 79523912 | 79564342 | chr17 | − | exo | hsa_circ_0046157 | 1.1774 | 0.000691 | NPLOC4 |
| 11289020 | 11289396 | chr19 | − | exo | hsa_circ_0008554 | 0.8117 | 0.000694 | KANK2 |
| 27458872 | 27460012 | chr10 | + | exo | hsa_circ_0018037 | 0.7468 | 0.000727 | MASTL |
| 5077135 | 5078186 | chr16 | − | exo | hsa_circ_0007146 | 0.5487 | 0.000770 | NAGPA |
| 57056124 | 57069951 | chr18 | − | s/o | hsa_circ_0007105 | 0.7657 | 0.000807 | XLOC_012858 |
| 101149911 | 101152632 | chr15 | + | exo | hsa_circ_0007550 | 1.1425 | 0.000835 | ASB7 |
| 67602967 | 67614669 | chr18 | − | int | hsa_circ_0008721 | 0.7388 | 0.000840 | CD226 |
| 141622461 | 141640905 | chr3 | + | exo | | 1.3890 | 0.000859 | ATP1B3 |
| 46342260 | 46342972 | chr11 | + | exo | hsa_circ_0021832 | 0.6700 | 0.000871 | CREB3L1 |
| 246490502 | 246498776 | chr1 | − | exo | hsa_circ_0017306 | 0.8727 | 0.000908 | SMYD3 |
| 172013152 | 172028671 | chr3 | + | exo | hsa_circ_0067997 | 1.2012 | 0.000939 | FNDC3B |
| 792249 | 792543 | chr16 | + | ing | hsa_circ_0006173 | 0.6746 | 0.000945 | |
| 20939730 | 20939965 | chr2 | − | exo | hsa_circ_0052877 | 0.8591 | 0.000974 | C2orf43 |
| 75704190 | 75705103 | chr15 | − | int | | 1.2553 | 0.001057 | SIN3A |
| 155891165 | 155891709 | chr1 | − | exo | hsa_circ_0008998 | 0.7843 | 0.001082 | KIAA0907 |
| 25830104 | 25830751 | chr2 | − | int | | 1.2270 | 0.001111 | DTNB |
| 82917099 | 82947551 | chr11 | + | exo | hsa_circ_0023858 | 1.2410 | 0.001239 | ANKRD42 |
| 116879948 | 116925405 | chr10 | + | exo | hsa_circ_0020094 | 0.7552 | 0.001318 | ATRNL1 |
| 26518046 | 26519239 | chr17 | + | exo | | 1.3912 | 0.001326 | NLK |
| 15907144 | 15907576 | chr17 | + | exo | hsa_circ_0042138 | 0.6495 | 0.001420 | TTC19 |
| 8615158 | 8615503 | chr19 | + | as | hsa_circ_0000887 | 0.7806 | 0.001430 | MYO1F |
| 55948718 | 55949857 | chr20 | + | exo | hsa_circ_0060969 | 0.6147 | 0.001458 | RAE1 |
| 122797766 | 122800683 | chr2 | + | ing | | 0.7803 | 0.001525 | |
| 121335202 | 121340050 | chr10 | − | s/o | hsa_circ_0004498 | 0.7622 | 0.001560 | TIAL1 |
| 28282081 | 28282196 | chr1 | + | s/o | hsa_circ_0000037 | 0.8851 | 0.001569 | SMPDL3B |
| 35644044 | 36220485 | chr13 | + | exo | hsa_circ_0029987 | 1.2237 | 0.001644 | NBEA |
| 217536827 | 217541605 | chr2 | − | exo | hsa_circ_0058196 | 2.0570 | 0.001651 | IGFBP5 |
| 155448623 | 155451551 | chr6 | + | exo | hsa_circ_0008289 | 0.6258 | 0.001714 | TIAM2 |
| 131271154 | 131271376 | chr9 | + | exo | hsa_circ_0088807 | 0.7886 | 0.001803 | GLE1 |
| 218480772 | 218511325 | chr1 | + | exo | hsa_circ_0016476 | 0.8139 | 0.001847 | RRP15 |
| 100847246 | 100848083 | chr14 | + | exo | hsa_circ_0004310 | 0.6627 | 0.001870 | WDR25 |
| 121756744 | 121770873 | chr6 | + | exo | hsa_circ_0077753 | 0.6072 | 0.001881 | GJA1 |
| 37246951 | 37247979 | chr2 | − | exo | | 1.1426 | 0.001894 | HEATR5B |
| 66378927 | 66403383 | chr1 | + | s/o | hsa_circ_0005822 | 0.8344 | 0.001970 | PDE4B |
| 16199310 | 16203173 | chr1 | + | exo | hsa_circ_0002237 | 1.2876 | 0.001977 | SPEN |
| 14562975 | 14572514 | chr10 | − | s/o | | 0.8148 | 0.002008 | FAM107B |
| 232596632 | 232607274 | chr1 | − | exo | | 0.7431 | 0.002018 | SIPA1L2 |
| 43239233 | 43241193 | chr1 | + | s/o | | 1.1905 | 0.002026 | C1orf50 |
| 24840803 | 24841057 | chr1 | + | exo | hsa_circ_0003553 | 1.1468 | 0.002077 | RCAN3 |
| 6659106 | 6659512 | chr1 | − | exo | hsa_circ_0004885 | 0.7811 | 0.002131 | KLHL21 |
| 44714009 | 44715748 | chr7 | + | exo | hsa_circ_0001701 | 1.1999 | 0.002166 | OGDH |
| 3647544 | 3648604 | chr19 | − | int | hsa_circ_0092295 | 0.6434 | 0.002191 | PIP5K1C |
| 39830696 | 39833706 | chr20 | − | exo | hsa_circ_0060394 | 1.4514 | 0.002197 | ZHX3 |
| 2369581 | 2369841 | chr16 | − | exo | hsa_circ_0037516 | 1.2267 | 0.002207 | ABCA3 |
| 37301538 | 37303516 | chr5 | − | exo | hsa_circ_0003227 | 0.6868 | 0.002264 | NUP155 |
| 50902104 | 50902741 | chr19 | + | exo | hsa_circ_0052011 | 0.7240 | 0.002288 | POLD1 |
| 48508372 | 48511758 | chr8 | + | exo | hsa_circ_0004511 | 0.6952 | 0.002295 | SPIDR |
| 64637472 | 64641629 | chr17 | + | exo | hsa_circ_0045372 | 1.4348 | 0.002316 | PRKCA |
| 71799391 | 71803602 | chr16 | − | exo | hsa_circ_0007770 | 1.2837 | 0.002330 | AP1G1 |
| 91409421 | 91444868 | chr14 | − | exo | | 0.8438 | 0.002350 | RPS6KA5 |
| 73034214 | 73039802 | chr10 | + | s/o | | 0.7566 | 0.002382 | UNC5B |
| 241492330 | 241494472 | chr2 | − | exo | hsa_circ_0005059 | 1.1794 | 0.002396 | ANKMY1 |
| 133491152 | 133491392 | chr9 | + | int | hsa_circ_0092350 | 0.7942 | 0.002489 | FUBP3 |
| 32576002 | 32578596 | chr3 | − | exo | hsa_circ_0003211 | 0.8406 | 0.002530 | DYNC1LI1 |
| 148590288 | 148612863 | chr5 | + | exo | hsa_circ_0006270 | 1.1157 | 0.002628 | ABLIM3 |
| 30366964 | 30375041 | chr22 | + | exo | hsa_circ_0006303 | 1.4897 | 0.002636 | MTMR3 |
| 88044886 | 88047860 | chr5 | − | exo | hsa_circ_0073271 | 0.7689 | 0.002740 | MEF2C |
| 197872529 | 197954756 | chr2 | − | exo | hsa_circ_0057619 | 1.1476 | 0.002748 | ANKRD44 |
| 89390065 | 89391240 | chr3 | + | exo | hsa_circ_0066598 | 2.1194 | 0.002765 | EPHA3 |
| 102442048 | 102507010 | chr14 | + | exo | hsa_circ_0033211 | 1.2363 | 0.002777 | DYNC1H1 |
| 84267098 | 84303596 | chr9 | − | exo | hsa_circ_0087333 | 1.7924 | 0.002808 | TLE1 |
| 99700297 | 99700583 | chr7 | + | exo | hsa_circ_0081423 | 0.9229 | 0.002897 | AP4M1 |
| 17796781 | 17799020 | chr8 | + | int | hsa_circ_0001780 | 0.9091 | 0.002958 | PCM1 |
| 122669065 | 122670847 | chr12 | + | exo | | 0.7786 | 0.003021 | LRRC43 |
| 151167656 | 151195266 | chr7 | − | exo | | 0.7799 | 0.003045 | RHEB |
| 122125405 | 122135069 | chr2 | − | s/o | | 0.8367 | 0.003078 | CLASP1 |
| 34957782 | 34963668 | chr19 | + | s/o | | 1.1301 | 0.003146 | UBA2 |
| 93118615 | 93118705 | chr14 | + | s/o | hsa_circ_0000561 | 0.8034 | 0.003157 | RIN3 |
| 74696029 | 74706928 | chr5 | − | exo | hsa_circ_0007350 | 0.8720 | 0.003158 | COL4A3BP |
| 80837263 | 80881107 | chr6 | + | exo | | 1.2416 | 0.003164 | BCKDHB |
| 29972857 | 30006301 | chr18 | − | s/o | | 1.4644 | 0.003168 | GAREM |
| 32399131 | 32409407 | chr2 | + | exo | hsa_circ_0005695 | 0.6935 | 0.003169 | SLC30A6 |
| 92798947 | 92811471 | chr1 | + | exo | hsa_circ_0004463 | 0.8245 | 0.003175 | RPAP2 |
| 123199572 | 123210404 | chr9 | − | exo | hsa_circ_0088281 | 1.2594 | 0.003191 | CDK5RAP2 |
| 73854236 | 73866136 | chr15 | − | exo | hsa_circ_0007390 | 0.8175 | 0.003193 | NPTN |
| 70245970 | 70246161 | chr14 | + | as | hsa_circ_0000550 | 0.9089 | 0.003203 | SLC10A1 |
| 5821117 | 5827280 | chrX | − | exo | | 1.2783 | 0.003232 | NLGN4X |
| 31947298 | 31981684 | chr15 | − | exo | hsa_circ_0034326 | 0.8388 | 0.003298 | OTUD7A |
| 64878082 | 64879797 | chr12 | + | exo | hsa_circ_0000413 | 1.0976 | 0.003301 | TBK1 |
| 127294245 | 127294652 | chr3 | − | exo | hsa_circ_0067159 | 0.8118 | 0.003320 | TPRA1 |
| 7586 | 15888 | chrM | − | exo | hsa_circ_0089761 | 0.5663 | 0.003366 | JA760602 |
| 45515734 | 45518119 | chr3 | + | exo | hsa_circ_0065078 | 1.2598 | 0.003369 | LARS2 |
| 36064839 | 36104804 | chr14 | − | exo | hsa_circ_0031668 | 1.1920 | 0.003394 | RALGAPA1 |
| 170013698 | 170015181 | chr3 | + | exo | hsa_circ_0067934 | 0.7784 | 0.003489 | PRKCI |
| 88542064 | 88570096 | chr12 | + | exo | hsa_circ_0027689 | 1.2382 | 0.003509 | TMTC3 |
| 133607388 | 133632466 | chrX | + | exo | | 0.8140 | 0.003546 | HPRT1 |
| 51501542 | 51509135 | chr13 | + | exo | hsa_circ_0030286 | 0.6154 | 0.003554 | RNASEH2B |
| 47540449 | 47541488 | chr11 | − | int | hsa_circ_0000305 | 0.8451 | 0.003589 | CELF1 |
| 41199659 | 41215968 | chr17 | − | exo | hsa_circ_0043947 | 0.8536 | 0.003633 | BRCA1 |
| 11988810 | 11991892 | chr16 | − | exo | hsa_circ_0037911 | 1.3187 | 0.003648 | GSPT1 |
| 9117835 | 9122679 | chr18 | + | exo | | 0.8355 | 0.003669 | NDUFV2 |
| 108483817 | 108510471 | chr9 | + | exo | | 0.6675 | 0.003671 | TMEM38B |
| 43552465 | 43555513 | chr17 | − | exo | hsa_circ_0003434 | 1.3178 | 0.003701 | PLEKHM1 |
| 35586872 | 35604935 | chr6 | − | exo | hsa_circ_0076155 | 0.8744 | 0.003758 | FKBP5 |
| 133291443 | 133294706 | chr12 | + | exo | hsa_circ_0029545 | 1.3748 | 0.003792 | PGAM5 |
| 58785259 | 58796887 | chr14 | + | exo | hsa_circ_0000542 | 0.8345 | 0.003804 | ARID4A |
| 51664287 | 51664947 | chr3 | + | exo | hsa_circ_0065926 | 1.1684 | 0.003827 | RAD54L2 |
| 100006372 | 100009557 | chr6 | − | exo | hsa_circ_0003326 | 0.8060 | 0.003897 | CCNC |
| 118997644 | 119003929 | chr11 | + | exo | hsa_circ_0024553 | 0.7522 | 0.003940 | HINFP |
| 61488746 | 61490878 | chr20 | − | exo | hsa_circ_0006458 | 0.7143 | 0.003951 | TCFL5 |
| 128824688 | 128825122 | chr3 | − | int | hsa_circ_0001338 | 0.7970 | 0.003972 | RAB43 |
| 77085372 | 77103586 | chr11 | − | exo | hsa_circ_0023685 | 0.7293 | 0.004014 | PAK1IP1 |
| 208017318 | 208017404 | chr1 | − | int | hsa_circ_0000178 | 0.7668 | 0.004015 | C1orf132 |
| 31474264 | 31484852 | chr11 | − | int | hsa_circ_0003712 | 0.7615 | 0.004022 | IMMP1L |
| 33442333 | 33442726 | chr20 | − | exo | hsa_circ_0060004 | 1.2779 | 0.004035 | GGT7 |
| 618892 | 635941 | chr7 | − | exo | hsa_circ_0079039 | 0.8679 | 0.004045 | PRKAR1B |
| 38641940 | 38644025 | chr22 | − | exo | hsa_circ_0005243 | 1.1865 | 0.004096 | TMEM184B |
| 88883054 | 88904247 | chr14 | + | exo | | 1.2489 | 0.004178 | SPATA7 |
| 12257738 | 12259466 | chr10 | + | exo | | 1.1889 | 0.004249 | CDC123 |
| 32987845 | 32995095 | chr11 | + | exo | | 1.3522 | 0.004279 | QSER1 |
| 139818045 | 139828890 | chr5 | + | exo | hsa_circ_0007402 | 1.1649 | 0.004286 | ANKHD1 |
| 36437624 | 36439112 | chr1 | + | exo | hsa_circ_0011631 | 0.8330 | 0.004310 | AGO3 |
| 102432244 | 102433485 | chr5 | − | exo | hsa_circ_0006499 | 0.8015 | 0.004360 | GIN1 |
| 24133942 | 24135875 | chr22 | + | exo | hsa_circ_0004208 | 0.7830 | 0.004396 | SMARCB1 |
| 107236366 | 107254190 | chr12 | + | exo | hsa_circ_0028021 | 0.8171 | 0.004411 | RIC8B |
| 43712372 | 43735919 | chr2 | − | exo | | 1.2483 | 0.004422 | THADA |
| 86433228 | 86434450 | chr2 | + | exo | hsa_circ_0055521 | 0.7360 | 0.004478 | MRPL35 |
| 189455528 | 189456563 | chr3 | + | exo | hsa_circ_0068514 | 0.8212 | 0.004496 | TP63 |
| 57827025 | 57843886 | chr3 | + | exo | hsa_circ_0001975 | 1.1095 | 0.004496 | SLMAP |
| 81536885 | 81568718 | chr12 | + | exo | | 0.6874 | 0.004536 | ACSS3 |
| 107673726 | 107673881 | chr11 | − | exo | hsa_circ_0024162 | 0.8492 | 0.004556 | SLC35F2 |
| 41474465 | 41475261 | chr1 | + | exo | hsa_circ_0005157 | 0.7879 | 0.004571 | CTPS1 |
| 64066892 | 64067848 | chr15 | − | exo | hsa_circ_0007112 | 1.2262 | 0.004617 | HERC1 |
| 47734629 | 47735461 | chr21 | − | exo | hsa_circ_0002792 | 0.8518 | 0.004649 | C21orf58 |
| 9444552 | 9452550 | chr11 | + | exo | | 1.1482 | 0.004658 | IPO7 |
| 46945730 | 46957922 | chr21 | − | exo | hsa_circ_0062022 | 0.7049 | 0.004749 | SLC19A1 |
| 30500849 | 30503232 | chr17 | + | exo | hsa_circ_0005397 | 0.7187 | 0.004787 | RHOT1 |
| 17411664 | 17412433 | chr19 | − | exo | | 1.1753 | 0.004805 | ABHD8 |
| 64737776 | 64738878 | chr17 | + | exo | hsa_circ_0006736 | 1.5755 | 0.004826 | PRKCA |
| 63951934 | 63952135 | chr15 | − | exo | hsa_circ_0035729 | 1.3073 | 0.004865 | HERC1 |
| 27087346 | 27089776 | chr1 | + | exo | hsa_circ_0006913 | 1.2572 | 0.004877 | ARID1A |
| 27668989 | 27672064 | chr7 | − | exo | hsa_circ_0003958 | 0.7883 | 0.004898 | HIBADH |
| 22856779 | 22862386 | chr10 | − | exo | hsa_circ_0017969 | 0.8253 | 0.004924 | PIP4K2A |
| 157431605 | 157435966 | chr6 | + | int | hsa_circ_0001659 | 0.7953 | 0.004945 | ARID1B |
| 57258696 | 57259335 | chr11 | − | exo | hsa_circ_0005204 | 0.7503 | 0.005047 | SLC43A1 |
| 45898718 | 45959242 | chr22 | + | exo | hsa_circ_0063739 | 0.8103 | 0.005110 | FBLN1 |
| 40720487 | 40722201 | chr17 | + | exo | hsa_circ_0006823 | 0.7442 | 0.005217 | MLX |
| 59426338 | 59426942 | chr11 | − | exo | hsa_circ_0004162 | 1.2651 | 0.005220 | PATL1 |
| 109186061 | 109186605 | chr12 | − | exo | hsa_circ_0002957 | 1.3186 | 0.005224 | SSH1 |
| 68211443 | 68214701 | chr8 | − | exo | hsa_circ_0001808 | 1.2916 | 0.005244 | ARFGEF1 |
| 104847269 | 104847527 | chr14 | − | ing | hsa_circ_0000573 | 0.8285 | 0.005284 | |
| 115636279 | 115644139 | chr10 | + | exo | | 0.8371 | 0.005329 | NHLRC2 |
| 156711898 | 156713247 | chr1 | − | exo | hsa_circ_0014784 | 0.7078 | 0.005331 | HDGF |
| 25156459 | 25159970 | chr8 | + | exo | hsa_circ_0083694 | 0.7841 | 0.005376 | DOCK5 |
| 37049238 | 37063962 | chr20 | − | exo | hsa_circ_0060332 | 0.8187 | 0.005443 | SNHG17 |
| 51868106 | 51874004 | chr1 | − | exo | hsa_circ_0005567 | 0.7869 | 0.005466 | EPS15 |
| 33133825 | 33138502 | chr1 | + | exo | hsa_circ_0008672 | 0.8495 | 0.005591 | RBBP4 |
| 12183625 | 12183966 | chr11 | + | exo | | 0.8035 | 0.005610 | MICAL2 |
| 152650864 | 152653025 | chr6 | − | exo | hsa_circ_0078328 | 0.7543 | 0.005648 | SYNE1 |
| 2804256 | 2812362 | chr9 | − | s/o | hsa_circ_0007850 | 0.8089 | 0.005692 | KIAA0020 |
| 48259219 | 48259759 | chr19 | + | int | hsa_circ_0092320 | 0.8754 | 0.005713 | GLTSCR2 |
| 111057235 | 111067404 | chr6 | − | int | | 0.8622 | 0.005716 | CDK19 |
| 58255047 | 58256791 | chr3 | + | exo | hsa_circ_0066380 | 0.7204 | 0.005732 | ABHD6 |
| 126275990 | 126283543 | chr11 | + | exo | hsa_circ_0024825 | 1.3019 | 0.005786 | ST3GAL4 |
| 31376659 | 31381401 | chr20 | + | exo | | 0.5853 | 0.005919 | DNMT3B |
| 68874847 | 68875691 | chr5 | + | exo | hsa_circ_0004914 | 0.8501 | 0.005946 | GTF2H2C_2 |
| 71736500 | 71748704 | chr16 | − | exo | hsa_circ_0003681 | 0.7460 | 0.005969 | PHLPP2 |
| 147581750 | 147612308 | chr6 | + | exo | hsa_circ_0078162 | 1.2278 | 0.006007 | STXBP5 |
| 46529041 | 46541970 | chrX | − | exo | hsa_circ_0090360 | 0.7941 | 0.006053 | SLC9A7 |
| 115005725 | 115007010 | chr1 | − | exo | hsa_circ_0004127 | 1.1912 | 0.006074 | TRIM33 |
| 137654907 | 137665336 | chr5 | − | exo | | 1.3247 | 0.006107 | CDC25C |
| 42604156 | 42604482 | chr12 | − | exo | hsa_circ_0000392 | 0.9104 | 0.006208 | YAF2 |
| 151630710 | 151665959 | chr1 | + | exo | | 0.8654 | 0.006240 | SNX27 |
| 19116002 | 19146167 | chr18 | − | exo | hsa_circ_0047073 | 0.6897 | 0.006278 | ESCO1 |
| 113487898 | 113488149 | chr12 | − | ing | hsa_circ_0000440 | 0.7582 | 0.006292 | |
| 48744511 | 48747484 | chr20 | − | exo | hsa_circ_0002168 | 1.1583 | 0.006326 | TMEM189 |
| 114095571 | 114120265 | chr4 | + | exo | hsa_circ_0070700 | 1.5762 | 0.006376 | ANK2 |
| 104376576 | 104379506 | chr12 | + | exo | hsa_circ_0008166 | 0.8400 | 0.006435 | TDG |
| 101580519 | 101582342 | chr2 | + | int | | 0.8089 | 0.006456 | NPAS2 |
| 13233298 | 13234568 | chr10 | + | exo | hsa_circ_0004477 | 0.8428 | 0.006478 | MCM10 |
| 48587449 | 48596355 | chr16 | − | exo | | 1.0749 | 0.006503 | N4BP1 |
| 149921131 | 149922533 | chr5 | + | exo | hsa_circ_0007551 | 1.6472 | 0.006522 | NDST1 |
| 73072005 | 73072154 | chrX | + | s/o | hsa_circ_0001929 | 1.4202 | 0.006551 | XLOC_008015 |
| 19144163 | 19148055 | chr18 | − | exo | hsa_circ_0009110 | 0.7706 | 0.006587 | ESCO1 |
| 117013217 | 117047785 | chr6 | + | exo | hsa_circ_0006146 | 1.2055 | 0.006591 | KPNA5 |
| 76168527 | 76171529 | chr15 | + | exo | hsa_circ_0036380 | 1.2055 | 0.006635 | UBE2Q2 |
| 128867400 | 128903244 | chr8 | + | int | hsa_circ_0009143 | 0.8216 | 0.006667 | PVT1 |
| 43334706 | 43342194 | chr21 | − | exo | hsa_circ_0061817 | 0.8540 | 0.006683 | C2CD2 |
| 151960100 | 152012423 | chr7 | − | exo | hsa_circ_0006784 | 0.7452 | 0.006686 | KMT2C |
| 25315871 | 25326656 | chr2 | + | exo | hsa_circ_0053015 | 1.3369 | 0.006763 | EFR3B |
| 24985656 | 24989530 | chr12 | − | exo | hsa_circ_0004426 | 0.7027 | 0.006844 | BCAT1 |
| 6925099 | 7002978 | chr4 | + | exo | hsa_circ_0069104 | 0.7578 | 0.006878 | TBC1D14 |
| 120907659 | 120936298 | chr12 | + | exo | hsa_circ_0028882 | 1.1820 | 0.006894 | DYNLL1 |
| 55621921 | 55634000 | chr15 | + | exo | hsa_circ_0035381 | 1.3976 | 0.006956 | PIGB |
| 240910625 | 240910719 | chr2 | + | as | hsa_circ_0001117 | 0.7428 | 0.006983 | NDUFA10 |
| 206036934 | 206050587 | chr2 | + | exo | hsa_circ_0007756 | 0.7834 | 0.007061 | PARD3B |
| 116091160 | 116094330 | chr9 | − | exo | | 0.8570 | 0.007115 | WDR31 |
| 15616097 | 15616181 | chr5 | + | s/o | | 1.7119 | 0.007143 | FBXL7 |
| 77201159 | 77202117 | chr7 | + | int | | 0.7804 | 0.007181 | PTPN12 |
| 73790216 | 73795193 | chr7 | + | exo | hsa_circ_0080576 | 1.7441 | 0.007182 | CLIP2 |
| 26512204 | 26512291 | chr17 | + | exo | hsa_circ_0042521 | 0.8365 | 0.007216 | NLK |
| 37291188 | 37315092 | chr3 | + | s/o | hsa_circ_0001282 | 0.8968 | 0.007381 | GOLGA4 |
| 132952745 | 132971910 | chr8 | + | exo | hsa_circ_0006522 | 0.6521 | 0.007391 | EFR3A |
| 34157357 | 34159791 | chr22 | − | int | | 0.8601 | 0.007466 | LARGE |
| 54786787 | 54786942 | chr5 | − | exo | | 0.7363 | 0.007514 | PPAP2A |
| 23997527 | 24001306 | chr3 | + | exo | | 1.1313 | 0.007539 | NR1D2 |
| 73739331 | 73745323 | chr11 | − | exo | hsa_circ_0007169 | 1.4533 | 0.007590 | C2CD3 |
| 66303083 | 66397591 | chr17 | + | exo | | 1.3385 | 0.007593 | ARSG |
| 25877593 | 25885213 | chr17 | + | exo | | 1.3000 | 0.007631 | KSR1 |
| 46321391 | 46321685 | chr12 | + | as | hsa_circ_0000393 | 0.7951 | 0.007640 | SCAF11 |
| 112704965 | 112705661 | chr9 | + | exo | | 0.8421 | 0.007681 | PALM2-AKAP2 |
| 44085363 | 44085855 | chr19 | + | exo | hsa_circ_0051348 | 0.7329 | 0.007708 | PINLYP |
| 159851413 | 159867952 | chr2 | + | s/o | | 0.7194 | 0.007728 | TANC1 |
| 102603979 | 102669865 | chr7 | − | exo | | 0.6534 | 0.007761 | FBXL13 |
| 50369762 | 50370182 | chr19 | − | int | hsa_circ_0092285 | 0.7050 | 0.007769 | PNKP |
| 105305512 | 105305735 | chr7 | − | exo | hsa_circ_0007856 | 1.1749 | 0.007798 | ATXN7L1 |
| 2639040 | 2640231 | chr20 | − | exo | hsa_circ_0059204 | 1.2553 | 0.007880 | IDH3B |
| 75654163 | 75656362 | chr16 | − | exo | hsa_circ_0000716 | 0.8901 | 0.007922 | ADAT1 |
| 47920060 | 47920607 | chr19 | − | exo | hsa_circ_0051685 | 1.5172 | 0.007930 | MEIS3 |
| 15601324 | 15618413 | chr2 | − | exo | hsa_circ_0008083 | 0.7416 | 0.007954 | NBAS |
| 223084858 | 223097002 | chr2 | − | exo | hsa_circ_0007333 | 0.5596 | 0.007990 | PAX3 |
| 54248444 | 54250043 | chr4 | + | exo | hsa_circ_0069738 | 1.2476 | 0.008030 | FIP1L1 |
| 47856010 | 47856992 | chr19 | + | exo | hsa_circ_0051672 | 0.7186 | 0.008062 | DHX34 |
| 20107645 | 20109225 | chr17 | + | exo | hsa_circ_0000745 | 2.0869 | 0.008135 | SPECC1 |
| 134862377 | 134862465 | chr5 | − | ing | hsa_circ_0001532 | 0.8731 | 0.008138 | |
| 55457930 | 55467710 | chr14 | − | exo | | 0.7880 | 0.008197 | WDHD1 |
| 99584817 | 99585905 | chr3 | + | int | | 0.6720 | 0.008222 | CMSS1 |
| 35797838 | 35804870 | chr17 | + | exo | hsa_circ_0043280 | 0.8648 | 0.008322 | TADA2A |
| 69112132 | 69117159 | chr3 | − | exo | | 1.3804 | 0.008349 | UBA3 |
| 95498782 | 95535315 | chr12 | − | exo | | 0.8299 | 0.008373 | FGD6 |
| 62579578 | 62582288 | chr17 | − | exo | hsa_circ_0045322 | 0.8040 | 0.008415 | SMURF2 |
| 122928045 | 122978436 | chr3 | + | exo | | 0.7949 | 0.008417 | SEC22A |
| 135624111 | 135626611 | chr2 | − | s/o | | 1.3620 | 0.008460 | CCNT2-AS1 |
| 82821160 | 82906047 | chr15 | − | exo | hsa_circ_0036567 | 0.7872 | 0.008568 | RPS17L |
| 30332294 | 30361953 | chr8 | + | exo | hsa_circ_0006539 | 0.8139 | 0.008602 | RBPMS |
| 27106071 | 27106460 | chr6 | − | exo | hsa_circ_0075900 | 0.7803 | 0.008603 | HIST1H2BK |
| 25630392 | 25638642 | chr17 | + | exo | hsa_circ_0005336 | 0.7679 | 0.008632 | WSB1 |
| 126395204 | 126395456 | chr10 | − | exo | hsa_circ_0005734 | 1.3409 | 0.008645 | FAM53B |
| 51402258 | 51404549 | chr12 | − | exo | hsa_circ_0008802 | 1.3779 | 0.008645 | SLC11A2 |
| 25101189 | 25132957 | chr8 | + | exo | hsa_circ_0007190 | 0.7919 | 0.008653 | DOCK5 |
| 75101156 | 75118768 | chr10 | − | s/o | | 1.1697 | 0.008680 | CFAP70 |
| 26993605 | 26994323 | chr10 | + | exo | hsa_circ_0018000 | 0.8349 | 0.008681 | PDSS1 |
| 22403053 | 22440043 | chr4 | − | exo | hsa_circ_0069323 | 1.3945 | 0.008812 | GPR125 |
| 46855947 | 46906128 | chr18 | − | int | | 0.6857 | 0.008817 | DYM |
| 174309663 | 174325105 | chr4 | + | as | | 0.6765 | 0.008818 | SCRG1 |
| 77423853 | 77461496 | chr5 | − | exo | hsa_circ_0001501 | 0.6539 | 0.008821 | AP3B1 |
| 32207322 | 32228337 | chr20 | + | exo | hsa_circ_0059864 | 1.4404 | 0.008824 | CBFA2T2 |
| 122181534 | 122199644 | chr12 | + | exo | hsa_circ_0029000 | 1.3534 | 0.008853 | TMEM120B |
| 77885259 | 77885898 | chr7 | − | exo | | 1.4543 | 0.008863 | MAGI2 |
| 175996707 | 175996824 | chr1 | − | exo | hsa_circ_0015358 | 0.8194 | 0.008889 | RFWD2 |
| 134072602 | 134077104 | chr9 | + | exo | hsa_circ_0089183 | 1.2196 | 0.008995 | NUP214 |
| 68290258 | 68292294 | chr14 | + | exo | hsa_circ_0032304 | 0.9358 | 0.009002 | RAD51B |
| 3515899 | 3520982 | chr20 | + | exo | | 1.1887 | 0.009186 | ATRN |
| 91842508 | 91855996 | chr7 | − | exo | hsa_circ_0081006 | 0.7383 | 0.009186 | KRIT1 |
| 97999787 | 97999925 | chr10 | − | int | hsa_circ_0000253 | 0.7749 | 0.009208 | BLNK |
| 6237962 | 6312055 | chr18 | − | exo | hsa_circ_0046760 | 0.6089 | 0.009244 | L3MBTL4 |
| 10246411 | 10246963 | chr19 | − | exo | hsa_circ_0049192 | 0.7341 | 0.009327 | DNMT1 |
| 5249777 | 5249948 | chr3 | + | exo | hsa_circ_0064090 | 1.1093 | 0.009334 | EDEM1 |
| 5021962 | 5044520 | chr9 | + | exo | hsa_circ_0002824 | 0.6890 | 0.009361 | JAK2 |
| 32584305 | 32594208 | chr7 | + | exo | hsa_circ_0001694 | 1.3596 | 0.009366 | AVL9 |
| 102744013 | 102744164 | chr9 | − | s/o | hsa_circ_0001877 | 0.8546 | 0.009402 | ERP44 |
| 4455174 | 4455921 | chr17 | − | exo | hsa_circ_0041565 | 1.2335 | 0.009445 | MYBBP1A |
| 161002504 | 161003029 | chr3 | + | ing | | 1.1085 | 0.009523 | |
| 66337415 | 66352945 | chr17 | + | s/o | | 0.7229 | 0.009528 | ARSG |
| 71209067 | 71209308 | chr14 | − | exo | hsa_circ_0032377 | 0.8258 | 0.009627 | MAP3K9 |
| 89484691 | 89497734 | chr16 | − | exo | hsa_circ_0000727 | 0.7781 | 0.009654 | ANKRD11 |
| 120138549 | 120148425 | chr12 | − | exo | hsa_circ_0028681 | 0.8436 | 0.009858 | CIT |
| 18258252 | 18258434 | chr19 | + | exo | | 1.2512 | 0.009910 | MAST3 |
| 4763457 | 4794491 | chr12 | + | exo | | 0.8456 | 0.009991 | NDUFA9 |
| 96692646 | 96694138 | chr12 | − | exo | hsa_circ_0007881 | 0.7988 | 0.009992 | CDK17 |
| 27283886 | 27284084 | chr17 | + | int | hsa_circ_0000748 | 1.1743 | 0.010011 | PIPOX |
| 67524151 | 67529158 | chr15 | − | exo | hsa_circ_0000620 | 0.6543 | 0.010093 | AAGAB |
| 121275020 | 121286936 | chr10 | − | exo | hsa_circ_0005516 | 0.8079 | 0.010119 | RGS10 |
| 224599128 | 224607317 | chr1 | − | exo | hsa_circ_0002577 | 1.2496 | 0.010132 | WDR26 |
| 133493397 | 133509713 | chr5 | − | s/o | hsa_circ_0004145 | 0.6371 | 0.010160 | SKP1 |
| 13375816 | 13387028 | chr10 | − | exo | hsa_circ_0003136 | 0.8183 | 0.010229 | SEPHS1 |
| 11835008 | 11836142 | chr19 | − | exo | hsa_circ_0002534 | 0.7440 | 0.010256 | ZNF823 |
| 65140077 | 65169748 | chr10 | − | s/o | hsa_circ_0005144 | 0.7518 | 0.010389 | JMJD1C |
| 81837331 | 81837529 | chr14 | − | exo | hsa_circ_0032832 | 0.7927 | 0.010417 | STON2 |
| 76198579 | 76198832 | chr17 | + | exo | hsa_circ_0045881 | 0.7184 | 0.010475 | AFMID |
| 236706402 | 236715975 | chr2 | + | exo | hsa_circ_0058806 | 1.6104 | 0.010502 | AGAP1 |
| 134508189 | 134509862 | chr5 | + | s/o | hsa_circ_0006652 | 0.6080 | 0.010600 | C5orf66 |
| 104445572 | 104465246 | chr10 | − | exo | | 0.8683 | 0.010637 | ARL3 |
| 13035452 | 13039661 | chr19 | − | exo | hsa_circ_0049627 | 1.3113 | 0.010690 | FARSA |
| 32453345 | 32474403 | chr8 | + | exo | hsa_circ_0007279 | 0.6725 | 0.010716 | NRG1 |
| 229422232 | 229449507 | chr1 | + | s/o | | 0.9031 | 0.010719 | RAB4A |
| 99166819 | 99194903 | chr12 | − | exo | | 0.7086 | 0.010740 | ANKS1B |
| 26772806 | 26774151 | chr1 | + | exo | hsa_circ_0000034 | 0.8235 | 0.010763 | DHDDS |
| 42980912 | 42981094 | chr6 | − | s/o | hsa_circ_0001606 | 0.7632 | 0.010809 | MEA1 |
| 23224688 | 23224917 | chr7 | + | exo | hsa_circ_0005096 | 0.7610 | 0.010848 | NUPL2 |
| 53410256 | 53416411 | chr12 | + | exo | hsa_circ_0026497 | 1.2352 | 0.010897 | EIF4B |
| 19850719 | 19861884 | chr17 | − | exo | | 0.7693 | 0.010932 | AKAP10 |
| 86281854 | 86282092 | chr6 | − | exo | hsa_circ_0077248 | 0.7875 | 0.010962 | SNX14 |
| 113734352 | 113800365 | chr9 | − | exo | hsa_circ_0087962 | 0.5306 | 0.010975 | LPAR1 |
| 5413685 | 5417655 | chr7 | − | exo | hsa_circ_0006824 | 1.3350 | 0.010983 | TNRC18 |
| 38225401 | 38248504 | chr13 | − | exo | | 0.8334 | 0.011021 | TRPC4 |
| 177069283 | 177073118 | chr4 | + | exo | | 0.8891 | 0.011028 | WDR17 |
| 105106994 | 105108756 | chr10 | − | exo | hsa_circ_0005052 | 0.8077 | 0.011084 | PCGF6 |
| 77147164 | 77147491 | chr3 | + | exo | hsa_circ_0066556 | 1.4072 | 0.011212 | ROBO2 |
| 158850767 | 158873300 | chr6 | + | exo | hsa_circ_0002987 | 1.1481 | 0.011231 | TULP4 |
| 76349027 | 76368567 | chr14 | + | exo | hsa_circ_0032731 | 0.8173 | 0.011370 | TTLL5 |
| 94798450 | 94803547 | chr8 | + | exo | hsa_circ_0084889 | 1.1794 | 0.011416 | TMEM67 |
| 91491883 | 91497323 | chr15 | + | exo | hsa_circ_0036906 | 1.2147 | 0.011424 | UNC45A |
| 80914570 | 80923622 | chr17 | − | exo | hsa_circ_0046598 | 0.7741 | 0.011491 | B3GNTL1 |
| 28654060 | 28671129 | chr8 | − | exo | | 0.7402 | 0.011614 | INTS9 |
| 44640275 | 44640841 | chr20 | + | s/o | hsa_circ_0001161 | 0.6068 | 0.011633 | MMP9 |
| 32681480 | 32686439 | chr20 | − | exo | | 0.6985 | 0.011665 | EIF2S2 |
| 124243539 | 124251384 | chr8 | − | exo | hsa_circ_0002663 | 0.7732 | 0.011666 | C8orf76 |
| 29346047 | 29347038 | chr15 | + | exo | hsa_circ_0004695 | 1.3291 | 0.011727 | APBA2 |
| 64038182 | 64041145 | chr12 | − | exo | hsa_circ_0007104 | 0.8092 | 0.011776 | DPY19L2 |
| 73999275 | 73999479 | chr17 | + | exo | hsa_circ_0045751 | 0.8133 | 0.011808 | CDK3 |
| 149784242 | 149784743 | chr5 | − | exo | hsa_circ_0074530 | 0.8395 | 0.011821 | CD74 |
| 25191214 | 25194811 | chr7 | − | exo | | 0.8877 | 0.011902 | C7orf31 |
| 38045815 | 38050233 | chr6 | + | int | | 1.1099 | 0.011917 | ZFAND3 |
| 91924202 | 91981956 | chr7 | + | exo | hsa_circ_0002744 | 0.8227 | 0.011971 | ANKIB1 |
| 78298829 | 78311797 | chr17 | + | exo | hsa_circ_0000811 | 1.3058 | 0.011987 | RNF213 |
| 54348615 | 54348949 | chr1 | − | exo | hsa_circ_0007074 | 0.8907 | 0.011989 | YIPF1 |
| 50209130 | 50226379 | chr15 | − | exo | | 0.8658 | 0.012038 | ATP8B4 |
| 62462593 | 62463260 | chr14 | + | exo | | 0.7869 | 0.012062 | SYT16 |
| 17543067 | 17551835 | chr6 | + | exo | | 1.1346 | 0.012068 | CAP2 |
| 101775286 | 101775782 | chr15 | − | exo | hsa_circ_0005019 | 1.2666 | 0.012113 | CHSY1 |
| 124832370 | 124835283 | chr12 | − | exo | hsa_circ_0029301 | 1.3908 | 0.012191 | NCOR2 |
| 48734062 | 48741472 | chr17 | + | exo | hsa_circ_0044603 | 1.1923 | 0.012191 | ABCC3 |
| 33735522 | 33735618 | chr21 | − | s/o | hsa_circ_0001182 | 1.2157 | 0.012202 | URB1 |
| 138994281 | 139003046 | chr5 | + | exo | hsa_circ_0007452 | 1.1605 | 0.012423 | UBE2D2 |
| 51065351 | 51077601 | chr10 | − | exo | hsa_circ_0018340 | 0.6738 | 0.012512 | PARG |
| 33467081 | 33481417 | chr3 | − | exo | hsa_circ_0064743 | 1.3175 | 0.012532 | UBP1 |
| 150838348 | 150859050 | chr5 | + | exo | hsa_circ_0074623 | 0.8900 | 0.012586 | SLC36A1 |
| 147425000 | 147431201 | chr4 | − | exo | hsa_circ_0071095 | 1.1813 | 0.012614 | SLC10A7 |
| 33114035 | 33118729 | chr3 | − | exo | | 0.8572 | 0.012692 | GLB1 |
| 146112224 | 146115788 | chr8 | − | exo | | 0.8744 | 0.012714 | ZNF250 |
| 132841908 | 132848755 | chr9 | + | exo | hsa_circ_0002625 | 1.2226 | 0.012745 | GPR107 |
| 41758353 | 41758724 | chr19 | + | int | | 0.7701 | 0.012792 | AXL |
| 149563797 | 149589941 | chr3 | + | exo | hsa_circ_0067716 | 0.8644 | 0.012810 | RNF13 |
| 122181534 | 122190129 | chr12 | + | exo | hsa_circ_0006030 | 1.3463 | 0.012855 | TMEM120B |
| 42187824 | 42262894 | chr7 | − | exo | | 0.6729 | 0.012857 | GLI3 |
| 36181656 | 36185787 | chr6 | + | exo | hsa_circ_0009139 | 1.1518 | 0.012861 | BRPF3 |
| 113734352 | 113773970 | chr9 | − | exo | hsa_circ_0087961 | 0.6192 | 0.012890 | LPAR1 |
| 205072984 | 205074296 | chr1 | − | exo | hsa_circ_0003527 | 1.3023 | 0.012915 | RBBP5 |
| 79168800 | 79169629 | chr17 | − | int | | 0.6353 | 0.012937 | AZI1 |
| 34090650 | 34209207 | chr1 | − | exo | hsa_circ_0011491 | 0.7033 | 0.012942 | CSMD2 |
| 79249768 | 79257302 | chr17 | − | exo | hsa_circ_0046131 | 1.2978 | 0.013056 | SLC38A10 |
| 1746096 | 1756483 | chr17 | + | exo | hsa_circ_0000734 | 0.8567 | 0.013063 | RPA1 |
| 20972052 | 20972473 | chr1 | + | s/o | | 1.1146 | 0.013075 | PINK1 |
| 51138484 | 51151225 | chr12 | − | as | | 0.8472 | 0.013120 | DIP2B |
| 69575302 | 69577315 | chr2 | − | exo | hsa_circ_0055054 | 1.2673 | 0.013170 | GFPT1 |
| 140675368 | 140678382 | chr3 | + | exo | hsa_circ_0067567 | 0.7758 | 0.013220 | SLC25A36 |
| 162036124 | 162061304 | chr2 | + | exo | hsa_circ_0005227 | 1.2268 | 0.013301 | TANK |
| 31382950 | 31424289 | chr16 | + | s/o | hsa_circ_0000694 | 0.7712 | 0.013313 | ITGAX |
| 27222466 | 27226329 | chr16 | + | int | hsa_circ_0005642 | 0.8773 | 0.013340 | KDM8 |
| 41721567 | 41721922 | chr22 | + | exo | hsa_circ_0007704 | 1.4229 | 0.013424 | ZC3H7B |
| 79534442 | 79539114 | chr17 | − | exo | hsa_circ_0046178 | 1.2426 | 0.013443 | NPLOC4 |
| 89202707 | 89221015 | chr14 | − | exo | hsa_circ_0032880 | 0.8027 | 0.013443 | EML5 |
| 52400580 | 52441862 | chr6 | − | exo | hsa_circ_0076769 | 1.2351 | 0.013451 | TRAM2 |
| 128627027 | 128631846 | chr2 | − | exo | hsa_circ_0056370 | 1.2205 | 0.013489 | AMMECR1L |
| 10534462 | 10550251 | chr18 | + | s/o | hsa_circ_0004922 | 0.8088 | 0.013510 | NAPG |
| 111150126 | 111152952 | chr12 | − | ing | | 0.8279 | 0.013514 | |
| 108219096 | 108295048 | chr5 | + | exo | hsa_circ_0006425 | 0.8456 | 0.013534 | FER |
| 82935193 | 82941559 | chr6 | − | exo | hsa_circ_0004905 | 0.8399 | 0.013545 | IBTK |
| 38083916 | 38084999 | chr22 | + | exo | hsa_circ_0004543 | 0.8525 | 0.013576 | NOL12 |
| 40859224 | 40948371 | chr22 | − | exo | hsa_circ_0063453 | 1.2310 | 0.013628 | MKL1 |
| 82302569 | 82302742 | chr1 | + | exo | | 0.6906 | 0.013662 | LPHN2 |
| 183558357 | 183562114 | chr3 | − | exo | hsa_circ_0068252 | 1.2418 | 0.013735 | PARL |
| 72181974 | 72229924 | chr8 | − | exo | | 1.3533 | 0.013746 | EYA1 |
| 24663284 | 24681487 | chr7 | + | exo | hsa_circ_0079619 | 1.5120 | 0.013767 | MPP6 |
| 20107645 | 20163607 | chr17 | + | exo | hsa_circ_0042428 | 1.8766 | 0.013778 | SPECC1 |
| 8866406 | 8866637 | chr12 | + | exo | hsa_circ_0025372 | 1.4463 | 0.013789 | RIMKLB |
| 158850767 | 158852695 | chr6 | + | s/o | hsa_circ_0001660 | 1.1591 | 0.013844 | TULP4 |
| 194169185 | 194175121 | chr3 | − | exo | hsa_circ_0001376 | 1.3965 | 0.013870 | ATP13A3 |
| 3916742 | 3936296 | chr17 | − | exo | | 1.2042 | 0.013978 | ZZEF1 |
| 36579939 | 36580208 | chr19 | + | exo | hsa_circ_0003139 | 1.2954 | 0.013978 | WDR62 |
| 133420612 | 133425310 | chr12 | − | exo | hsa_circ_0029584 | 1.2915 | 0.014060 | CHFR |
| 85223943 | 85234875 | chr15 | − | exo | hsa_circ_0000647 | 0.8300 | 0.014097 | SEC11A |
| 78022381 | 78032779 | chr17 | + | exo | | 1.4087 | 0.014110 | CCDC40 |
| 34024320 | 34101636 | chr6 | − | exo | hsa_circ_0076039 | 0.8394 | 0.014126 | GRM4 |
| 128103455 | 128109220 | chr9 | + | exo | hsa_circ_0005249 | 0.8563 | 0.014163 | GAPVD1 |
| 106739425 | 106746234 | chr2 | − | exo | hsa_circ_0055940 | 0.6878 | 0.014163 | UXS1 |
| 128584535 | 128609022 | chr4 | + | exo | | 0.8236 | 0.014218 | INTU |
| 30374430 | 30387659 | chr22 | + | exo | hsa_circ_0002954 | 1.1963 | 0.014278 | MTMR3 |
| 25666964 | 25678184 | chr1 | + | exo | hsa_circ_0007054 | 0.8326 | 0.014334 | TMEM50A |
| 2595269 | 2622150 | chr12 | + | exo | hsa_circ_0003792 | 0.7802 | 0.014336 | CACNA1C |
| 137716445 | 137717750 | chr9 | + | exo | hsa_circ_0003596 | 0.7232 | 0.014347 | COL5A1 |
| 65040617 | 65043837 | chr15 | − | exo | | 0.7537 | 0.014391 | RBPMS2 |
| 109288780 | 109289485 | chr2 | + | exo | hsa_circ_0008348 | 1.4118 | 0.014412 | LIMS1 |
| 1696500 | 1705427 | chr4 | − | exo | hsa_circ_0068851 | 1.3147 | 0.014444 | SLBP |
| 50689402 | 50689520 | chr22 | − | int | hsa_circ_0001256 | 1.0929 | 0.014466 | HDAC10 |
| 74489529 | 74489999 | chr9 | − | exo | hsa_circ_0002921 | 0.7129 | 0.014512 | ABHD17B |
| 113771427 | 113773970 | chr9 | − | int | hsa_circ_0005752 | 0.6689 | 0.014543 | LPAR1 |
| 120830753 | 120836157 | chr2 | + | exo | hsa_circ_0001064 | 0.7822 | 0.014591 | EPB41L5 |
| 19760861 | 19761115 | chr19 | − | int | hsa_circ_0000919 | 0.7068 | 0.014678 | ATP13A1 |
| 41347444 | 41348904 | chr15 | − | exo | hsa_circ_0005632 | 1.2369 | 0.014746 | INO80 |
| 118830673 | 118849440 | chr8 | − | exo | hsa_circ_0008035 | 1.4236 | 0.014833 | EXT1 |
| 33331154 | 33347029 | chr21 | + | exo | | 1.3806 | 0.014887 | HUNK |
| 75703832 | 75706652 | chr15 | − | exo | hsa_circ_0000636 | 1.1013 | 0.014897 | SIN3A |
| 40852275 | 40854198 | chr5 | + | int | hsa_circ_0005895 | 0.8638 | 0.014942 | CARD6 |
| 109687788 | 109694041 | chr12 | + | exo | | 1.1566 | 0.014998 | ACACB |
| 16526335 | 16528616 | chr10 | + | exo | hsa_circ_0017854 | 0.7992 | 0.015009 | PTER |
| 23419522 | 23421892 | chr14 | − | exo | hsa_circ_0005663 | 0.7620 | 0.015096 | HAUS4 |
| 118940943 | 118944661 | chr11 | + | exo | hsa_circ_0009166 | 1.2212 | 0.015108 | VPS11 |
| 164514761 | 164515093 | chr4 | + | as | hsa_circ_0001455 | 0.8339 | 0.015153 | MARCH1 |
| 172182350 | 172188377 | chr2 | − | exo | hsa_circ_0004384 | 1.2467 | 0.015207 | METTL8 |
| 65218264 | 65223783 | chr15 | + | exo | | 0.8102 | 0.015207 | ANKDD1A |
| 19883137 | 19884168 | chr16 | − | exo | hsa_circ_0038381 | 1.1259 | 0.015286 | GPRC5B |
| 15651543 | 15660752 | chr6 | − | exo | | 1.2071 | 0.015323 | DTNBP1 |
| 3925823 | 3944671 | chr20 | − | exo | hsa_circ_0059342 | 0.8766 | 0.015332 | RNF24 |
| 133734285 | 133769540 | chr8 | − | exo | hsa_circ_0008205 | 0.7447 | 0.015344 | TMEM71 |
| 17910716 | 17974508 | chr4 | − | exo | | 0.7595 | 0.015362 | LCORL |
| 52139686 | 52145377 | chr12 | + | exo | hsa_circ_0006763 | 0.8737 | 0.015463 | SCN8A |
| 152487289 | 152510078 | chr4 | + | exo | | 0.6738 | 0.015483 | FAM160A1 |
| 110451444 | 110452678 | chr4 | + | exo | hsa_circ_0007184 | 0.8082 | 0.015517 | SEC24B |
| 15480588 | 15531345 | chr8 | + | exo | hsa_circ_0083390 | 0.6210 | 0.015531 | TUSC3 |
| 31596990 | 31602881 | chr14 | − | exo | hsa_circ_0002301 | 1.4030 | 0.015645 | HECTD1 |
| 55832281 | 55832783 | chr2 | − | int | | 1.2098 | 0.015687 | SMEK2 |
| 94291504 | 94297307 | chr10 | − | exo | hsa_circ_0000251 | 1.1251 | 0.015798 | IDE |
| 34100754 | 34101636 | chr6 | − | exo | hsa_circ_0076041 | 0.8489 | 0.015804 | GRM4 |
| 154006958 | 154019343 | chrX | − | exo | hsa_circ_0092148 | 0.9119 | 0.015846 | MPP1 |
| 23424768 | 23430148 | chr16 | − | exo | | 1.2150 | 0.015915 | COG7 |
| 129752887 | 129783380 | chr4 | + | exo | hsa_circ_0070948 | 0.7847 | 0.015919 | JADE1 |
| 136671202 | 136677338 | chr9 | − | exo | hsa_circ_0089392 | 1.2870 | 0.016080 | VAV2 |
| 33921690 | 33973235 | chr9 | − | exo | hsa_circ_0086684 | 0.7737 | 0.016190 | UBAP2 |
| 114170357 | 114175048 | chr13 | + | int | | 0.7632 | 0.016347 | TMCO3 |
| 76576097 | 76588078 | chr15 | − | exo | hsa_circ_0002226 | 0.7625 | 0.016367 | ETFA |
| 103964838 | 103966537 | chr14 | + | exo | | 0.8801 | 0.016413 | MARK3 |
| 159992704 | 160007067 | chr2 | + | exo | hsa_circ_0008010 | 1.3432 | 0.016428 | TANC1 |
| 46189424 | 46202902 | chr22 | + | s/o | | 0.5708 | 0.016497 | ATXN10 |
| 46281750 | 46282780 | chr19 | − | exo | | 1.1870 | 0.016556 | DMPK |
| 40554466 | 40575273 | chr17 | − | exo | hsa_circ_0043821 | 0.7924 | 0.016559 | PTRF |
| 90069143 | 90074398 | chr10 | − | exo | | 0.8458 | 0.016591 | RNLS |
| 154315413 | 154318485 | chr4 | + | exo | hsa_circ_0006225 | 0.7841 | 0.016653 | MND1 |
| 6263945 | 6312055 | chr18 | − | exo | hsa_circ_0000824 | 1.2419 | 0.016672 | L3MBTL4 |
| 154231452 | 154232505 | chr1 | + | exo | hsa_circ_0014405 | 0.6684 | 0.016765 | UBAP2L |
| 37026330 | 37027514 | chr5 | + | exo | hsa_circ_0001473 | 1.2021 | 0.016821 | NIPBL |
| 52941283 | 52945896 | chr6 | + | exo | hsa_circ_0076780 | 1.0978 | 0.016889 | FBXO9 |
| 104702610 | 104707254 | chr7 | + | exo | hsa_circ_0081813 | 1.2737 | 0.016891 | KMT2E |
| 124091978 | 124101150 | chr12 | + | exo | hsa_circ_0000459 | 0.8660 | 0.016891 | DDX55 |
| 76115051 | 76115473 | chr17 | − | exo | | 0.8259 | 0.016993 | TMC6 |
| 29140758 | 29152576 | chr2 | + | exo | | 0.7803 | 0.017024 | WDR43 |
| 35279644 | 35281515 | chr21 | − | exo | hsa_circ_0061577 | 0.7858 | 0.017063 | ATP5O |
| 25156459 | 25158173 | chr8 | + | exo | hsa_circ_0004219 | 0.6890 | 0.017070 | DOCK5 |
| 118619175 | 118619412 | chr12 | − | exo | hsa_circ_0000446 | 1.2247 | 0.017116 | TAOK3 |
| 51208299 | 51211083 | chr14 | − | exo | hsa_circ_0008945 | 0.8074 | 0.017191 | NIN |
| 58208194 | 58208649 | chr19 | − | s/o | | 0.7593 | 0.017211 | ZNF154 |
| 50768734 | 50769735 | chr14 | − | exo | hsa_circ_0031845 | 1.1874 | 0.017305 | L2HGDH |
| 119147944 | 119177708 | chr6 | − | exo | | 1.1253 | 0.017449 | MCM9 |
| 73748522 | 73850873 | chr11 | − | exo | hsa_circ_0023535 | 1.1799 | 0.017471 | C2CD3 |
| 131113791 | 131113904 | chr7 | + | exo | hsa_circ_0082413 | 0.7619 | 0.017548 | MKLN1 |
| 105260646 | 105264714 | chr7 | − | exo | hsa_circ_0005755 | 1.2888 | 0.017615 | ATXN7L1 |
| 34489906 | 34493056 | chr11 | − | as | hsa_circ_0000289 | 0.8420 | 0.017630 | CAT |
| 3789810 | 3797251 | chr11 | − | exo | hsa_circ_0007401 | 1.3498 | 0.017696 | NUP98 |
| 160472466 | 160472794 | chr1 | + | as | hsa_circ_0000144 | 0.9023 | 0.017791 | SLAMF6 |
| 48561918 | 48565892 | chr20 | + | exo | hsa_circ_0008212 | 1.3261 | 0.017816 | RNF114 |
| 67861087 | 67868100 | chr1 | − | as | | 0.7939 | 0.017892 | IL12RB2 |
| 6518679 | 6520169 | chr11 | − | as | | 0.9139 | 0.017932 | DNHD1 |
| 88655653 | 88667506 | chr9 | − | exo | hsa_circ_0087400 | 1.7770 | 0.018039 | GOLM1 |
| 73315177 | 73316443 | chr2 | − | exo | hsa_circ_0007273 | 0.8675 | 0.018071 | RAB11FIP5 |
| 36773370 | 36773564 | chr1 | + | exo | hsa_circ_0011685 | 0.7533 | 0.018074 | SH3D21 |
| 9002122 | 9025596 | chr11 | − | exo | hsa_circ_0021120 | 0.8972 | 0.018085 | NRIP3 |
| 25932459 | 25938655 | chr17 | + | exo | | 1.4515 | 0.018088 | KSR1 |
| 71193260 | 71229161 | chr15 | + | exo | | 0.8046 | 0.018120 | LRRC49 |
| 39766030 | 39766488 | chr17 | − | exo | hsa_circ_0043626 | 0.8149 | 0.018153 | KRT16 |
| 20067587 | 20077417 | chr13 | − | exo | hsa_circ_0029601 | 0.8347 | 0.018262 | TPTE2 |
| 118890859 | 118892596 | chr11 | + | exo | | 0.8460 | 0.018342 | TRAPPC4 |
| 40619626 | 40630598 | chr21 | − | exo | hsa_circ_0008452 | 0.8318 | 0.018371 | BRWD1 |
| 46931559 | 46934702 | chr16 | + | exo | hsa_circ_0039216 | 2.0954 | 0.018385 | GPT2 |
| 70368695 | 70387650 | chrX | + | exo | hsa_circ_0090986 | 1.6174 | 0.018394 | NLGN3 |
| 50473795 | 50474137 | chr14 | − | s/o | hsa_circ_0000536 | 0.8234 | 0.018395 | LOC100506499 |
| 79573716 | 79596831 | chr17 | − | exo | hsa_circ_0046204 | 1.4478 | 0.018416 | NPLOC4 |
| 26965269 | 26972177 | chr17 | − | exo | hsa_circ_0042645 | 1.2864 | 0.018487 | KIAA0100 |
| 23998916 | 24048958 | chr12 | − | exo | | 0.6975 | 0.018500 | SOX5 |
| 162620 | 180590 | chr16 | − | exo | hsa_circ_0037134 | 1.1994 | 0.018507 | NPRL3 |
| 57416991 | 57417600 | chr20 | − | exo | | 0.8900 | 0.018526 | GNAS-AS1 |
| 62651928 | 62651997 | chr11 | + | exo | hsa_circ_0022589 | 1.2542 | 0.018531 | SLC3A2 |
| 12999419 | 13042333 | chr18 | + | exo | hsa_circ_0046999 | 0.9003 | 0.018565 | CEP192 |
| 24155604 | 24156680 | chr12 | − | int | | 0.8008 | 0.018589 | SOX5 |
| 126212971 | 126641300 | chr9 | − | exo | hsa_circ_0088439 | 1.1298 | 0.018601 | DENND1A |
| 398169 | 398574 | chr20 | + | exo | hsa_circ_0004525 | 1.1907 | 0.018641 | RBCK1 |
| 10784445 | 10808849 | chr2 | − | exo | hsa_circ_0000977 | 0.8136 | 0.018664 | NOL10 |
| 87760370 | 87764250 | chr16 | − | exo | hsa_circ_0040784 | 1.2338 | 0.018689 | KLHDC4 |
| 3270774 | 3275275 | chr20 | − | exo | hsa_circ_0059283 | 1.2971 | 0.018715 | C20orf194 |
| 1437392 | 1461853 | chr17 | − | exo | hsa_circ_0041252 | 1.3278 | 0.018769 | PITPNA |
| 14034145 | 14034624 | chr19 | + | exo | hsa_circ_0049700 | 0.8115 | 0.018844 | CC2D1A |
| 31596990 | 31641328 | chr14 | − | exo | hsa_circ_0031482 | 1.2457 | 0.018844 | HECTD1 |
| 67578201 | 67578733 | chr16 | + | exo | | 1.0685 | 0.018865 | FAM65A |
| 65924470 | 65928135 | chr17 | + | exo | hsa_circ_0045462 | 1.2912 | 0.018910 | BPTF |
| 755878 | 760253 | chr11 | + | exo | hsa_circ_0003307 | 1.0935 | 0.018928 | TALDO1 |
| 151749331 | 151753128 | chr4 | − | exo | hsa_circ_0071210 | 0.7385 | 0.018945 | LRBA |
| 150997086 | 150999803 | chr1 | + | exo | hsa_circ_0000126 | 0.8563 | 0.018999 | PRUNE |
| 147733519 | 147744289 | chrX | + | exo | hsa_circ_0091669 | 1.3769 | 0.019007 | AFF2 |
| 35426709 | 35441308 | chr10 | + | int | hsa_circ_0008851 | 0.6783 | 0.019023 | CREM |
| 44877652 | 44878394 | chr1 | + | exo | hsa_circ_0012152 | 5.1653 | 0.019063 | RNF220 |
| 97318671 | 97321850 | chr8 | + | exo | hsa_circ_0084984 | 1.2244 | 0.019131 | PTDSS1 |
| 85012794 | 85015600 | chr16 | − | exo | hsa_circ_0040719 | 1.2975 | 0.019165 | ZDHHC7 |
| 66364462 | 66369027 | chr18 | − | exo | hsa_circ_0002567 | 0.7451 | 0.019185 | TMX3 |
| 145197456 | 145205763 | chr5 | − | exo | hsa_circ_0006528 | 0.7209 | 0.019265 | PRELID2 |
| 71571951 | 71572157 | chr14 | + | exo | hsa_circ_0032457 | 1.1211 | 0.019286 | PCNX |
| 99721116 | 99735230 | chr9 | − | exo | | 1.4845 | 0.019307 | HIATL2 |
| 178735010 | 178743013 | chr3 | − | exo | hsa_circ_0068067 | 1.1969 | 0.019348 | ZMAT3 |
| 25348845 | 25348942 | chr12 | − | as | hsa_circ_0000382 | 0.8511 | 0.019379 | LYRM5 |
| 41292569 | 41292883 | chr19 | + | exo | hsa_circ_0051147 | 1.1676 | 0.019379 | RAB4B |
| 50902107 | 50902741 | chr19 | + | exo | hsa_circ_0052012 | 0.7589 | 0.019380 | POLD1 |
| 212977661 | 212981190 | chr1 | + | exo | hsa_circ_0000181 | 0.6892 | 0.019386 | TATDN3 |
| 43107459 | 43112367 | chr17 | − | exo | hsa_circ_0044136 | 1.4512 | 0.019569 | DCAKD |
| 11778020 | 11830089 | chr16 | − | exo | hsa_circ_0037866 | 0.7099 | 0.019666 | TXNDC11 |
| 19750893 | 19751380 | chr19 | − | exo | hsa_circ_0000918 | 1.2842 | 0.019751 | GMIP |
| 5289579 | 5290906 | chr11 | − | exo | hsa_circ_0020967 | 0.7656 | 0.019769 | HBE1 |
| 20126716 | 20131279 | chr22 | + | exo | hsa_circ_0004100 | 0.8102 | 0.019798 | ZDHHC8 |
| 556530 | 565135 | chr17 | − | exo | hsa_circ_0002879 | 1.2689 | 0.019828 | VPS53 |
| 154736558 | 154743926 | chrX | − | exo | | 0.8814 | 0.019832 | TMLHE |
| 79872718 | 79873138 | chr17 | − | int | hsa_circ_0092305 | 0.8028 | 0.019839 | SIRT7 |
| 424004 | 427848 | chr16 | − | exo | hsa_circ_0000663 | 1.2093 | 0.019878 | TMEM8A |
| 116734383 | 116744352 | chr11 | − | exo | hsa_circ_0000357 | 1.3066 | 0.019962 | SIK3 |
| 1735857 | 1770677 | chr1 | − | exo | hsa_circ_0005907 | 1.8178 | 0.019980 | GNB1 |
| 23395341 | 23396869 | chr14 | − | exo | hsa_circ_0002928 | 0.7996 | 0.020071 | PRMT5 |
| 75167801 | 75173934 | chr11 | − | exo | hsa_circ_0023624 | 1.0792 | 0.020087 | GDPD5 |
| 140244417 | 140302342 | chr7 | − | exo | hsa_circ_0082720 | 1.3349 | 0.020106 | DENND2A |
| 127500782 | 127505094 | chr10 | − | exo | hsa_circ_0008369 | 0.8395 | 0.020107 | UROS |
| 45688268 | 45701813 | chr7 | + | exo | hsa_circ_0080121 | 1.3565 | 0.020190 | ADCY1 |
| 53699213 | 53701307 | chr1 | − | exo | | 1.1760 | 0.020270 | MAGOH |
| 102027080 | 102038934 | chr2 | − | exo | hsa_circ_0004121 | 0.8339 | 0.020409 | RFX8 |
| 11727842 | 11727982 | chr19 | − | as | hsa_circ_0000891 | 0.7760 | 0.020485 | ZNF627 |
| 231079551 | 231097049 | chr1 | − | exo | hsa_circ_0004531 | 0.8392 | 0.020513 | TTC13 |
| 229599289 | 229601266 | chr1 | − | exo | | 0.8546 | 0.020545 | NUP133 |
| 15858833 | 15889942 | chr10 | − | exo | hsa_circ_0000219 | 0.7454 | 0.020553 | FAM188A |
| 148947249 | 148949886 | chr7 | + | exo | | 0.7760 | 0.020736 | ZNF212 |
| 27224905 | 27226329 | chr16 | + | exo | hsa_circ_0038704 | 0.7723 | 0.020746 | KDM8 |
| 10702601 | 10702872 | chr6 | + | exo | hsa_circ_0005917 | 0.8571 | 0.020782 | PAK1IP1 |
| 36375930 | 36376124 | chr9 | − | exo | hsa_circ_0001855 | 1.4344 | 0.020812 | RNF38 |
| 4421355 | 4421489 | chr19 | + | int | | 0.7149 | 0.020816 | CHAF1A |
| 149563797 | 149629870 | chr3 | + | exo | hsa_circ_0003502 | 0.7312 | 0.020864 | RNF13 |
| 236839408 | 236877267 | chr2 | + | exo | hsa_circ_0003272 | 1.4184 | 0.020983 | AGAP1 |
| 29910348 | 29941246 | chr3 | + | exo | hsa_circ_0064644 | 0.7427 | 0.021010 | RBMS3 |
| 61871449 | 61871624 | chr11 | − | ing | | 0.7747 | 0.021079 | |
| 103070781 | 103072661 | chr9 | − | exo | hsa_circ_0087765 | 0.9130 | 0.021120 | TEX10 |
| 9215040 | 9229179 | chr11 | − | exo | hsa_circ_0003546 | 1.3907 | 0.021170 | DENND5A |
| 126799558 | 126799662 | chr10 | − | exo | hsa_circ_0020354 | 0.6760 | 0.021175 | CTBP2 |
| 139316347 | 139416814 | chr7 | − | exo | hsa_circ_0082680 | 1.4604 | 0.021221 | HIPK2 |
| 15280896 | 15281636 | chr19 | − | exo | | 1.8190 | 0.021233 | NOTCH3 |
| 90414706 | 90432372 | chr15 | − | exo | hsa_circ_0009156 | 0.8586 | 0.021281 | AP3S2 |
| 134353337 | 134353833 | chr9 | + | int | | 0.7968 | 0.021311 | PRRC2B |
| 98660130 | 98691146 | chr9 | + | exo | hsa_circ_0087605 | 0.8117 | 0.021377 | ERCC6L2 |
| 14405956 | 14420130 | chr5 | + | exo | hsa_circ_0008697 | 1.9240 | 0.021389 | TRIO |
| 44010870 | 44015718 | chr19 | − | exo | hsa_circ_0051334 | 0.7702 | 0.021458 | ETHE1 |
| 32749557 | 32764217 | chr12 | + | s/o | hsa_circ_0000389 | 1.6364 | 0.021498 | FGD4 |
| 124515234 | 124567405 | chr3 | − | exo | | 1.8393 | 0.021695 | ITGB5 |
| 61975298 | 62063932 | chr3 | + | exo | | 1.5238 | 0.021731 | PTPRG |
| 132337477 | 132350323 | chr3 | − | exo | hsa_circ_0005875 | 0.7970 | 0.021838 | ACAD11 |
| 102542007 | 102559661 | chr12 | + | exo | hsa_circ_0000432 | 0.7780 | 0.021884 | PARPBP |
| 34376033 | 34409158 | chr18 | − | s/o | hsa_circ_0047467 | 0.8017 | 0.021893 | TPGS2 |
| 1747194 | 1770677 | chr1 | − | exo | hsa_circ_0008702 | 1.7498 | 0.022074 | GNB1 |
| 3505743 | 3505854 | chr9 | − | int | hsa_circ_0001836 | 0.8645 | 0.022107 | RFX3 |
| 172477945 | 172482290 | chr3 | + | exo | | 1.3340 | 0.022115 | ECT2 |
| 42909540 | 42924974 | chr2 | + | exo | hsa_circ_0054272 | 1.1158 | 0.022135 | MTA3 |
| 243542022 | 243589860 | chr1 | + | exo | | 0.8509 | 0.022165 | SDCCAG8 |
| 29750638 | 29752605 | chr22 | − | exo | hsa_circ_0062762 | 1.1324 | 0.022293 | AP1B1 |
| 85692787 | 85737409 | chr11 | − | exo | hsa_circ_0023908 | 1.3674 | 0.022306 | PICALM |
| 51860052 | 51866625 | chr1 | − | exo | hsa_circ_0012421 | 0.8374 | 0.022328 | EPS15 |
| 65860865 | 65864782 | chr7 | − | exo | | 0.8562 | 0.022447 | LINC00174 |
| 16727794 | 16727995 | chr9 | − | exo | hsa_circ_0086418 | 0.7849 | 0.022465 | BNC2 |
| 156936308 | 156940513 | chr5 | − | exo | hsa_circ_0003868 | 1.4467 | 0.022491 | ADAM19 |
| 77199096 | 77203637 | chr12 | + | exo | hsa_circ_0027622 | 0.8660 | 0.022539 | ZDHHC17 |
| 70497071 | 70497677 | chr16 | + | exo | hsa_circ_0002599 | 0.9152 | 0.022570 | FUK |
| 12788710 | 12788908 | chr12 | + | exo | hsa_circ_0002873 | 0.8483 | 0.022593 | CREBL2 |
| 1664640 | 1682366 | chr16 | + | exo | hsa_circ_0037318 | 1.3423 | 0.022642 | CRAMP1L |
| 42941740 | 42942776 | chr6 | − | exo | hsa_circ_0005038 | 1.2621 | 0.022643 | PEX6 |
| 6243671 | 6245937 | chr11 | − | exo | hsa_circ_0020990 | 0.7979 | 0.022649 | FAM160A2 |
| 1721833 | 1724750 | chr1 | − | exo | hsa_circ_0009344 | 1.4860 | 0.022683 | GNB1 |
| 36369715 | 36376124 | chr9 | − | exo | hsa_circ_0087023 | 1.4477 | 0.022737 | RNF38 |
| 129008502 | 129008714 | chr3 | + | int | | 0.8730 | 0.022737 | HMCES |
| 173423435 | 173435552 | chr2 | + | exo | hsa_circ_0057090 | 0.7087 | 0.022758 | PDK1 |
| 65041198 | 65043837 | chr15 | − | exo | | 0.7676 | 0.022760 | RBPMS2 |
| 18303496 | 18308257 | chr11 | − | exo | | 0.8843 | 0.022798 | HPS5 |
| 242606059 | 242608100 | chr2 | + | exo | hsa_circ_0006464 | 1.2099 | 0.022921 | ATG4B |
| 59805629 | 59812070 | chr1 | + | exo | hsa_circ_0000076 | 0.6102 | 0.022999 | FGGY |
| 33572585 | 33573787 | chr11 | + | exo | hsa_circ_0021609 | 1.3941 | 0.023032 | KIAA1549L |
| 78398080 | 78398276 | chr15 | − | exo | hsa_circ_0036444 | 0.7850 | 0.023097 | CIB2 |
| 62500850 | 62500949 | chr17 | + | as | hsa_circ_0000797 | 0.6924 | 0.023206 | DDX5 |
| 52215816 | 52231653 | chr1 | + | exo | hsa_circ_0008022 | 0.8310 | 0.023243 | OSBPL9 |
| 242265407 | 242275513 | chr2 | + | exo | hsa_circ_0005154 | 1.4072 | 0.023246 | SEPT2 |
| 139865173 | 139866645 | chr5 | + | exo | hsa_circ_0074264 | 0.6787 | 0.023287 | ANKHD1 |
| 71808337 | 71814433 | chr11 | − | exo | hsa_circ_0023424 | 1.1759 | 0.023295 | LAMTOR1 |
| 6880011 | 6893232 | chr9 | + | exo | hsa_circ_0001839 | 0.8515 | 0.023309 | KDM4C |
| 12632755 | 12639440 | chr1 | − | exo | hsa_circ_0010020 | 0.8580 | 0.023319 | DHRS3 |
| 110321942 | 110343415 | chr2 | − | exo | hsa_circ_0003065 | 0.7140 | 0.023356 | SEPT10 |
| 89319314 | 89327676 | chr14 | + | exo | hsa_circ_0006603 | 0.8436 | 0.023359 | TTC8 |
| 54867533 | 54870603 | chr1 | − | exo | | 1.4008 | 0.023390 | SSBP3 |
| 47782533 | 47782822 | chr20 | − | exo | hsa_circ_0004400 | 1.2260 | 0.023392 | STAU1 |
| 73768209 | 73774608 | chr7 | + | exo | | 1.1641 | 0.023553 | CLIP2 |
| 48917223 | 48918803 | chr1 | − | exo | hsa_circ_0012389 | 1.1937 | 0.023579 | SPATA6 |
| 5154168 | 5155925 | chr20 | + | exo | hsa_circ_0059379 | 1.2782 | 0.023579 | CDS2 |
| 155504370 | 155504634 | chr6 | + | exo | | 0.7401 | 0.023606 | TIAM2 |
| 7837219 | 7841492 | chr1 | + | exo | hsa_circ_0009548 | 0.7450 | 0.023698 | VAMP3 |
| 203382173 | 203383775 | chr2 | + | s/o | | 1.2289 | 0.023772 | BMPR2 |
| 52156394 | 52172318 | chr3 | − | exo | hsa_circ_0004884 | 0.8072 | 0.023829 | POC1A |
| 131102948 | 131104197 | chr2 | + | exo | hsa_circ_0056459 | 1.0770 | 0.023896 | IMP4 |
| 12783660 | 12784130 | chr19 | + | exo | hsa_circ_0049540 | 1.1676 | 0.023937 | WDR83 |
| 15297688 | 15303330 | chr19 | − | exo | hsa_circ_0049831 | 1.8079 | 0.023939 | NOTCH3 |
| 68434283 | 68439038 | chr15 | + | exo | hsa_circ_0008378 | 1.0763 | 0.023960 | PIAS1 |
| 219520872 | 219521186 | chr2 | − | exo | hsa_circ_0007648 | 1.1195 | 0.024065 | ZNF142 |
| 19538077 | 19538212 | chr2 | + | as | hsa_circ_0000980 | 0.9057 | 0.024065 | AC092594.1 |
| 86590527 | 86611866 | chr1 | − | exo | | 0.8069 | 0.024109 | COL24A1 |
| 35936463 | 35944813 | chr1 | − | exo | hsa_circ_0011571 | 1.3178 | 0.024160 | KIAA0319L |
| 91974291 | 92000921 | chr7 | + | exo | | 0.8491 | 0.024197 | ANKIB1 |
| 46739158 | 46742441 | chr22 | + | exo | | 0.8300 | 0.024253 | TRMU |
| 29906579 | 29907181 | chr16 | − | exo | hsa_circ_0008535 | 1.2044 | 0.024256 | SEZ6L2 |
| 3976053 | 3985461 | chr19 | − | exo | hsa_circ_0048545 | 0.8690 | 0.024268 | EEF2 |
| 75210552 | 75211467 | chr7 | − | exo | hsa_circ_0080731 | 2.0076 | 0.024336 | HIP1 |
| 112898406 | 112900819 | chr9 | + | exo | hsa_circ_0008706 | 0.8151 | 0.024364 | PALM2-AKAP2 |
| 85810160 | 85814886 | chr16 | − | s/o | | 0.7630 | 0.024400 | EMC8 |
| 112968568 | 112969680 | chr3 | + | exo | hsa_circ_0007897 | 0.5240 | 0.024402 | BOC |
| 145640307 | 145643194 | chr5 | + | exo | hsa_circ_0074441 | 0.8640 | 0.024430 | RBM27 |
| 52937061 | 52946887 | chr18 | − | exo | | 1.6965 | 0.024489 | TCF4 |
| 44826336 | 44835918 | chr3 | + | exo | hsa_circ_0006084 | 0.7749 | 0.024538 | KIF15 |
| 8499646 | 8525035 | chr9 | − | exo | hsa_circ_0086359 | 1.5429 | 0.024600 | PTPRD |
| 149563797 | 149677923 | chr3 | + | exo | hsa_circ_0067718 | 0.7688 | 0.024615 | RNF13 |
| 9225206 | 9229179 | chr11 | − | exo | hsa_circ_0004099 | 1.4935 | 0.024625 | DENND5A |
| 118949432 | 118950495 | chr9 | + | exo | hsa_circ_0005456 | 0.7693 | 0.024657 | PAPPA |
| 109318233 | 109318497 | chr13 | + | exo | hsa_circ_0030830 | 0.4955 | 0.024699 | MYO16 |
| 91030185 | 91035943 | chr15 | + | exo | hsa_circ_0007201 | 1.1686 | 0.024723 | IQGAP1 |
| 69687138 | 69693802 | chr16 | + | exo | | 0.7228 | 0.024735 | NFAT5 |
| 94428283 | 94428763 | chr10 | − | s/o | hsa_circ_0007472 | 1.1257 | 0.024805 | EIF2S2P3 |
| 58340777 | 58347472 | chr12 | + | exo | hsa_circ_0000412 | 0.9277 | 0.024841 | XRCC6BP1 |
| 51957464 | 51963580 | chr13 | − | exo | hsa_circ_0030296 | 0.7029 | 0.024900 | INTS6 |
| 35846859 | 35865177 | chr1 | + | exo | hsa_circ_0011545 | 0.7750 | 0.024987 | ZMYM4 |
| 231081134 | 231097049 | chr1 | − | exo | hsa_circ_0000194 | 0.8492 | 0.025072 | TTC13 |
| 56977647 | 56990226 | chr1 | − | exo | hsa_circ_0002861 | 1.5070 | 0.025086 | PPAP2B |
| 27351506 | 27353580 | chr16 | + | exo | hsa_circ_0038718 | 0.7246 | 0.025126 | IL4R |
| 12332743 | 12356334 | chr12 | − | exo | hsa_circ_0025489 | 1.5967 | 0.025138 | LRP6 |
| 31885078 | 31928627 | chr16 | + | s/o | hsa_circ_0039183 | 1.1308 | 0.025178 | ZNF267 |
| 37866065 | 37872192 | chr17 | + | exo | | 0.7938 | 0.025225 | ERBB2 |
| 154244751 | 154252211 | chr5 | + | exo | hsa_circ_0074719 | 0.7904 | 0.025299 | CNOT8 |
| 54491883 | 54492285 | chrX | − | exo | hsa_circ_0090830 | 1.3395 | 0.025306 | FGD1 |
| 9931806 | 9950565 | chr18 | + | exo | hsa_circ_0046909 | 1.1540 | 0.025373 | VAPA |
| 178370233 | 178388533 | chr2 | + | exo | hsa_circ_0057183 | 1.0657 | 0.025407 | AGPS |
| 220077108 | 220079804 | chr2 | − | exo | hsa_circ_0058346 | 1.3347 | 0.025518 | ABCB6 |
| 45472160 | 45483666 | chr21 | + | exo | hsa_circ_0061920 | 0.8072 | 0.025569 | TRAPPC10 |
| 89799889 | 89804313 | chr16 | + | exo | hsa_circ_0040987 | 1.2931 | 0.025573 | ZNF276 |
| 8043537 | 8099878 | chr7 | + | exo | hsa_circ_0001678 | 1.1984 | 0.025592 | GLCCI1 |
| 170501992 | 170511960 | chr4 | − | exo | hsa_circ_0071434 | 0.8433 | 0.025593 | NEK1 |
| 98865473 | 98865667 | chr13 | + | exo | hsa_circ_0002557 | 1.4394 | 0.025690 | FARP1 |
| 148963425 | 148964313 | chr7 | + | exo | hsa_circ_0082864 | 1.1733 | 0.025735 | ZNF783 |
| 34974539 | 34985912 | chr21 | − | exo | | 0.8505 | 0.025744 | CRYZL1 |
| 112645677 | 112657293 | chr12 | − | exo | hsa_circ_0028381 | 1.2182 | 0.025751 | HECTD4 |
| 77631183 | 77632364 | chr9 | − | exo | hsa_circ_0004189 | 0.7954 | 0.025813 | C9orf41 |
| 167217940 | 167254788 | chr3 | − | exo | hsa_circ_0001357 | 0.7844 | 0.025901 | WDR49 |
| 37061800 | 37067498 | chr3 | + | exo | | 1.2205 | 0.025907 | MLH1 |
| 118629482 | 118634015 | chr11 | − | exo | hsa_circ_0000360 | 1.5954 | 0.025938 | DDX6 |
| 3205916 | 3209234 | chr10 | − | exo | hsa_circ_0003022 | 0.9023 | 0.025963 | PITRM1 |
| 74926381 | 74937998 | chr16 | − | exo | hsa_circ_0040507 | 0.6659 | 0.025966 | WDR59 |
| 54717288 | 54722859 | chr1 | − | exo | hsa_circ_0012651 | 1.2809 | 0.026135 | SSBP3 |
| 57048187 | 57069951 | chr18 | − | s/o | | 0.8538 | 0.026165 | XLOC_012858 |
| 31849672 | 31852977 | chr14 | − | exo | hsa_circ_0031570 | 1.1141 | 0.026188 | HEATR5A |
| 20156817 | 20163607 | chr17 | + | exo | hsa_circ_0042437 | 1.6550 | 0.026219 | SPECC1 |
| 85657103 | 85661070 | chr15 | + | exo | hsa_circ_0005889 | 0.8205 | 0.026222 | PDE8A |
| 138244331 | 138256189 | chr3 | − | exo | | 0.8301 | 0.026240 | CEP70 |
| 36036501 | 36038429 | chr19 | + | exo | hsa_circ_0050607 | 0.6939 | 0.026349 | TMEM147 |
| 116730126 | 116734144 | chr10 | + | exo | hsa_circ_0020092 | 0.7923 | 0.026365 | TRUB1 |
| 18974246 | 18975060 | chr19 | + | exo | hsa_circ_0005706 | 1.3982 | 0.026440 | UPF1 |
| 33493152 | 33493906 | chr19 | − | exo | hsa_circ_0050436 | 1.2157 | 0.026454 | RHPN2 |
| 64171161 | 64179425 | chr17 | − | exo | | 0.8885 | 0.026510 | CEP112 |
| 97535283 | 97563284 | chr9 | + | exo | hsa_circ_0002191 | 0.7625 | 0.026567 | C9orf3 |
| 148771477 | 148777809 | chr7 | − | exo | hsa_circ_0082854 | 1.2993 | 0.026584 | ZNF786 |
| 114214355 | 114216087 | chr1 | + | exo | hsa_circ_0006583 | 0.8445 | 0.026600 | MAGI3 |
| 136162128 | 136221621 | chr3 | − | exo | | 1.2133 | 0.026699 | STAG1 |
| 33932559 | 33933626 | chr9 | − | exo | hsa_circ_0086694 | 0.7187 | 0.026712 | UBAP2 |
| 28601335 | 28605752 | chr1 | + | exo | | 1.2650 | 0.026738 | SESN2 |
| 213403839 | 213405598 | chr1 | + | exo | hsa_circ_0016430 | 1.1576 | 0.026739 | RPS6KC1 |
| 1343311 | 1360219 | chr4 | + | exo | | 0.8903 | 0.026891 | UVSSA |
| 14042035 | 14075982 | chr1 | + | exo | | 0.7354 | 0.026922 | PRDM2 |
| 15073885 | 15076316 | chr3 | + | exo | hsa_circ_0064460 | 1.3070 | 0.026934 | NR2C2 |
| 71369656 | 71370884 | chr11 | + | ing | | 0.7986 | 0.027147 | |
| 1737913 | 1749314 | chr1 | − | exo | hsa_circ_0009357 | 2.0525 | 0.027308 | GNB1 |
| 80299959 | 80338802 | chr5 | + | s/o | hsa_circ_0001991 | 0.8889 | 0.027399 | RASGRF2 |
| 38967262 | 38968132 | chr5 | − | exo | hsa_circ_0072329 | 1.1685 | 0.027400 | RICTOR |
| 66413497 | 66413944 | chr11 | + | exo | hsa_circ_0023016 | 0.8609 | 0.027429 | RBM4 |
| 29483000 | 29497015 | chr17 | + | exo | hsa_circ_0007542 | 0.7438 | 0.027455 | NF1 |
| 66021409 | 66031213 | chr15 | − | exo | hsa_circ_0035944 | 0.8268 | 0.027552 | DENND4A |
| 72589152 | 72601994 | chr18 | + | exo | hsa_circ_0047964 | 0.8000 | 0.027659 | ZNF407 |
| 4650703 | 4651269 | chr4 | + | exo | hsa_circ_0009031 | 0.6655 | 0.027719 | STX18-AS1 |
| 147743428 | 147744289 | chrX | + | exo | hsa_circ_0001947 | 1.4730 | 0.027808 | AFF2 |
| 92462409 | 92463134 | chr7 | − | s/o | hsa_circ_0001724 | 1.2612 | 0.027855 | CDK6 |
| 233022758 | 233075115 | chr2 | + | s/o | | 0.7728 | 0.027948 | DIS3L2 |
| 51032748 | 51061888 | chr1 | − | exo | hsa_circ_0008197 | 0.8164 | 0.027994 | FAF1 |
| 150244686 | 150246573 | chr1 | + | exo | | 0.7469 | 0.028040 | C1orf54 |
| 172276997 | 172277280 | chr5 | + | int | hsa_circ_0001556 | 1.2739 | 0.028071 | ERGIC1 |
| 71242862 | 71248104 | chr6 | + | exo | | 0.7579 | 0.028303 | FAM135A |
| 88813098 | 88822592 | chr10 | − | exo | hsa_circ_0019032 | 1.2130 | 0.028309 | GLUD1 |
| 49609654 | 49618211 | chr10 | + | exo | hsa_circ_0002968 | 1.1661 | 0.028356 | MAPK8 |
| 64096074 | 64100213 | chr5 | + | exo | | 1.1228 | 0.028406 | CWC27 |
| 64456696 | 64509673 | chr12 | + | exo | hsa_circ_0008594 | 0.7304 | 0.028466 | SRGAP1 |
| 710803 | 713464 | chr9 | + | exo | hsa_circ_0005062 | 1.3132 | 0.028545 | KANK1 |
| 75296026 | 75299351 | chr10 | − | exo | hsa_circ_0004568 | 1.6570 | 0.028561 | USP54 |
| 124904502 | 124915333 | chr12 | − | exo | hsa_circ_0029308 | 1.4554 | 0.028577 | NCOR2 |
| 60962120 | 60963576 | chr20 | + | exo | hsa_circ_0061107 | 0.7589 | 0.028594 | RPS21 |
| 226054811 | 226067110 | chr1 | − | exo | hsa_circ_0016682 | 1.3378 | 0.028656 | TMEM63A |
| 231090078 | 231097049 | chr1 | − | exo | hsa_circ_0000195 | 0.8524 | 0.028839 | TTC13 |
| 185307901 | 185318643 | chr3 | + | exo | hsa_circ_0068400 | 0.7169 | 0.028866 | SENP2 |
| 134049441 | 134053797 | chr9 | + | exo | hsa_circ_0089172 | 0.8011 | 0.028933 | NUP214 |
| 82443805 | 82444764 | chr15 | − | exo | hsa_circ_0005745 | 0.7946 | 0.028937 | EFTUD1 |
| 30034641 | 30038146 | chr8 | + | exo | hsa_circ_0001786 | 0.8457 | 0.029022 | DCTN6 |
| 69708332 | 69728600 | chr15 | + | exo | | 1.1277 | 0.029042 | KIF23 |
| 27445667 | 27449008 | chr16 | + | exo | hsa_circ_0038728 | 0.8244 | 0.029053 | IL21R |
| 32154531 | 32161046 | chr22 | + | exo | hsa_circ_0062938 | 0.8421 | 0.029106 | DEPDC5 |
| 137218315 | 137300965 | chr9 | + | exo | hsa_circ_0089414 | 1.3032 | 0.029158 | RXRA |
| 64404771 | 64415745 | chr15 | + | exo | hsa_circ_0002178 | 1.2209 | 0.029174 | SNX1 |
| 34059659 | 34101636 | chr6 | − | exo | hsa_circ_0076040 | 0.8714 | 0.029191 | GRM4 |
| 15508205 | 15531345 | chr8 | + | exo | hsa_circ_0006410 | 0.7551 | 0.029253 | TUSC3 |
| 25630392 | 25636298 | chr17 | + | exo | hsa_circ_0007716 | 0.9035 | 0.029254 | WSB1 |
| 156758963 | 156759786 | chr7 | + | exo | hsa_circ_0004210 | 0.8210 | 0.029305 | NOM1 |
| 172096787 | 172097333 | chr5 | + | exo | | 1.2636 | 0.029315 | NEURL1B |
| 17933230 | 17934761 | chr20 | − | exo | hsa_circ_0006704 | 0.6914 | 0.029326 | SNX5 |
| 28187218 | 28192352 | chr16 | − | exo | hsa_circ_0038822 | 0.7251 | 0.029336 | XPO6 |
| 66044716 | 66053776 | chr15 | − | exo | hsa_circ_0035957 | 0.8849 | 0.029350 | DENND4A |
| 51076920 | 51090958 | chr12 | + | exo | hsa_circ_0007730 | 1.4428 | 0.029459 | DIP2B |
| 31947382 | 31971378 | chr22 | + | s/o | hsa_circ_0008323 | 0.6407 | 0.029572 | SFI1 |
| 240158288 | 240274613 | chr2 | − | exo | hsa_circ_0058912 | 1.3478 | 0.029582 | HDAC4 |
| 70299035 | 70299440 | chr16 | − | int | | 0.6859 | 0.029586 | AARS |
| 35280078 | 35280549 | chr6 | + | exo | hsa_circ_0076125 | 1.0645 | 0.029685 | DEF6 |
| 46750273 | 46753788 | chr10 | − | s/o | hsa_circ_0005575 | 0.8297 | 0.029720 | BMS1P5 |
| 51474985 | 51477919 | chr3 | − | exo | hsa_circ_0007485 | 1.1011 | 0.029795 | VPRBP |
| 44470542 | 44471017 | chr18 | − | exo | hsa_circ_0047596 | 1.1114 | 0.029873 | PIAS2 |
| 12159936 | 12183966 | chr11 | + | exo | hsa_circ_0007192 | 0.6585 | 0.029921 | MICAL2 |
| 11348416 | 11383753 | chr3 | + | exo | hsa_circ_0064292 | 1.1145 | 0.029982 | ATG7 |
| 37002644 | 37020798 | chr9 | − | exo | hsa_circ_0001857 | 1.3615 | 0.030014 | PAX5 |
| 24663284 | 24690331 | chr7 | + | exo | hsa_circ_0001685 | 1.5149 | 0.030023 | MPP6 |
| 11992073 | 12022903 | chr12 | + | exo | | 1.3105 | 0.030115 | ETV6 |
| 66288740 | 66291353 | chr11 | + | exo | hsa_circ_0022997 | 0.8072 | 0.030237 | BBS1 |
| 47466974 | 47484581 | chr3 | − | exo | hsa_circ_0065215 | 0.7481 | 0.030279 | SCAP |
| 20819794 | 20874806 | chr9 | + | exo | hsa_circ_0003782 | 0.7872 | 0.030354 | FOCAD |
| 201303844 | 201305507 | chr2 | + | exo | hsa_circ_0057703 | 1.4670 | 0.030463 | SPATS2L |
| 128927861 | 128928858 | chr2 | + | exo | hsa_circ_0056433 | 1.2670 | 0.030502 | UGGT1 |
| 58311223 | 58316858 | chr2 | + | exo | | 0.8120 | 0.030518 | VRK2 |
| 112116954 | 112121111 | chr12 | − | exo | hsa_circ_0006396 | 0.9059 | 0.030527 | BRAP |
| 37427243 | 37431350 | chr8 | − | ing | | 1.1621 | 0.030549 | |
| 119203985 | 119259469 | chr4 | − | exo | hsa_circ_0070727 | 1.3270 | 0.030641 | PRSS12 |
| 40872407 | 40882768 | chr1 | + | exo | hsa_circ_0011898 | 1.1870 | 0.030659 | SMAP2 |
| 238933982 | 238944581 | chr2 | + | exo | hsa_circ_0007713 | 0.8165 | 0.030683 | UBE2F |
| 113661854 | 113662145 | chr1 | + | exo | hsa_circ_0013587 | 0.8939 | 0.030694 | LRIG2 |
| 84163152 | 84164932 | chr16 | − | exo | hsa_circ_0040680 | 1.2170 | 0.030697 | HSDL1 |
| 4760668 | 4766974 | chr20 | − | exo | hsa_circ_0059354 | 1.4733 | 0.030720 | RASSF2 |
| 44859621 | 44861703 | chr15 | − | exo | hsa_circ_0035072 | 0.8591 | 0.030838 | SPG11 |
| 3792977 | 3797251 | chr11 | − | exo | hsa_circ_0004637 | 1.2082 | 0.030954 | NUP98 |
| 40825609 | 40831558 | chr22 | − | exo | hsa_circ_0063452 | 1.1460 | 0.031006 | MKL1 |
| 45516745 | 45553909 | chr1 | − | exo | | 1.3714 | 0.031022 | ZSWIM5 |
| 179007947 | 179020646 | chr5 | + | exo | | 1.1085 | 0.031061 | RUFY1 |
| 89724655 | 89734790 | chr16 | + | int | | 1.2540 | 0.031072 | C16orf55 |
| 31815277 | 31867987 | chr7 | − | exo | | 0.7964 | 0.031157 | PDE1C |
| 4776460 | 4781708 | chr20 | − | exo | hsa_circ_0059359 | 0.8347 | 0.031172 | RASSF2 |
| 62587201 | 62594608 | chr17 | − | exo | hsa_circ_0004789 | 0.8448 | 0.031193 | SMURF2 |
| 17924436 | 17931973 | chr17 | − | exo | | 0.7488 | 0.031287 | ATPAF2 |
| 45203311 | 45204674 | chr11 | + | exo | | 0.8230 | 0.031328 | PRDM11 |
| 47474879 | 47481653 | chr7 | − | exo | hsa_circ_0008729 | 0.8072 | 0.031330 | TNS3 |
| 78334098 | 78444927 | chr12 | + | exo | hsa_circ_0027631 | 1.2722 | 0.031422 | NAV3 |
| 62587201 | 62602758 | chr17 | − | exo | hsa_circ_0002862 | 0.8734 | 0.031469 | SMURF2 |
| 48701821 | 48718309 | chr2 | + | exo | hsa_circ_0008358 | 0.8867 | 0.031479 | PPP1R21 |
| 111057235 | 111095140 | chr6 | − | int | | 0.8623 | 0.031547 | CDK19 |
| 3620270 | 3620690 | chr19 | − | int | hsa_circ_0092303 | 0.8261 | 0.031552 | CACTIN |
| 57051718 | 57103316 | chr14 | + | exo | hsa_circ_0032040 | 1.2460 | 0.031555 | TMEM260 |
| 101296023 | 101296583 | chr6 | − | exo | hsa_circ_0077497 | 0.9096 | 0.031563 | ASCC3 |
| 65568074 | 65568341 | chr14 | − | int | | 0.8631 | 0.031660 | MAX |
| 67004005 | 67008836 | chr15 | + | exo | hsa_circ_0036005 | 0.8574 | 0.031705 | SMAD6 |
| 44429476 | 44434152 | chr19 | − | s/o | | 0.9124 | 0.031751 | ZNF45 |
| 20581548 | 20586395 | chr18 | + | exo | | 0.9058 | 0.031864 | RBBP8 |
| 117849251 | 117856275 | chr10 | − | exo | hsa_circ_0005239 | 0.6932 | 0.031870 | GFRA1 |
| 74524751 | 74528329 | chr7 | + | exo | hsa_circ_0080649 | 1.1911 | 0.032045 | GTF2IRD2 |
| 180256410 | 180257652 | chr1 | − | s/o | | 0.8754 | 0.032101 | ACBD6 |
| 124035952 | 124042956 | chr8 | − | exo | hsa_circ_0085434 | 0.7924 | 0.032173 | DERL1 |
| 33794470 | 33815499 | chr1 | − | exo | hsa_circ_0011477 | 1.4629 | 0.032220 | PHC2 |
| 33429698 | 33429839 | chr1 | + | as | hsa_circ_0000049 | 0.7283 | 0.032277 | RNF19B |
| 73897062 | 73898235 | chr17 | − | exo | hsa_circ_0045737 | 1.1643 | 0.032299 | MRPL38 |
| 36872779 | 36872903 | chr22 | − | exo | hsa_circ_0063158 | 1.1304 | 0.032300 | TXN2 |
| 74273404 | 74275538 | chr2 | + | exo | hsa_circ_0006251 | 1.2583 | 0.032424 | TET3 |
| 50655754 | 50656296 | chr13 | − | int | hsa_circ_0000487 | 0.8573 | 0.032430 | DLEU2 |
| 64526630 | 64527428 | chr7 | + | exo | hsa_circ_0080302 | 0.9026 | 0.032476 | CCT6P3 |
| 51926762 | 51930998 | chr1 | − | exo | hsa_circ_0007330 | 0.9206 | 0.032527 | EPS15 |
| 65266939 | 65275931 | chr15 | − | exo | hsa_circ_0003526 | 1.1968 | 0.032550 | SPG21 |
| 74431765 | 74434317 | chr7 | + | exo | hsa_circ_0080635 | 1.5079 | 0.032567 | GATSL1 |
| 15970016 | 15976318 | chr1 | + | exo | hsa_circ_0010058 | 0.8688 | 0.032669 | DDI2 |
| 47857202 | 47862140 | chr11 | − | exo | | 1.1796 | 0.032685 | NUP160 |
| 35236117 | 35236221 | chr20 | + | exo | hsa_circ_0001151 | 1.1174 | 0.032689 | C20orf24 |
| 65055186 | 65063461 | chr11 | + | exo | hsa_circ_0022812 | 0.7728 | 0.032689 | POLA2 |
| 47388673 | 47395345 | chr17 | − | exo | hsa_circ_0044413 | 0.8208 | 0.032694 | ZNF652 |
| 104037959 | 104053701 | chr14 | + | exo | | 0.8024 | 0.032707 | APOPT1 |
| 75264607 | 75265184 | chr10 | − | exo | hsa_circ_0018818 | 0.7908 | 0.032708 | USP54 |
| 201283972 | 201284219 | chr2 | + | exo | hsa_circ_0003071 | 1.3359 | 0.032750 | SPATS2L |
| 59445788 | 59450578 | chr15 | − | exo | hsa_circ_0035505 | 1.4838 | 0.032799 | MYO1E |
| 180171387 | 180171482 | chr1 | + | s/o | hsa_circ_0000163 | 0.9143 | 0.032823 | QSOX1 |
| 145806229 | 145806661 | chr8 | − | exo | hsa_circ_0086154 | 1.0909 | 0.032843 | ARHGAP39 |
| 238896604 | 238903451 | chr2 | + | exo | hsa_circ_0058850 | 1.1756 | 0.032860 | UBE2F |
| 90431752 | 90432372 | chr15 | − | exo | hsa_circ_0002080 | 0.8790 | 0.032871 | AP3S2 |
| 29087976 | 29096597 | chr16 | + | exo | | 0.8955 | 0.032937 | RRN3P2 |
| 55420603 | 55420882 | chr19 | + | exo | hsa_circ_0052165 | 0.6802 | 0.032939 | NCR1 |
| 7980924 | 7981203 | chr20 | − | int | hsa_circ_0001131 | 1.1720 | 0.032940 | TMX4 |
| 33351557 | 33354890 | chr9 | + | exo | hsa_circ_0006728 | 0.8031 | 0.033056 | NFX1 |
| 25450125 | 25472749 | chr6 | + | exo | | 0.8312 | 0.033077 | LRRC16A |
| 72167171 | 72168707 | chr18 | + | exo | hsa_circ_0000855 | 0.8741 | 0.033180 | CNDP2 |
| 117054494 | 117054855 | chr11 | + | exo | hsa_circ_0024379 | 1.2132 | 0.033195 | SIDT2 |
| 152456984 | 152457139 | chr6 | − | int | hsa_circ_0001653 | 1.1963 | 0.033248 | SYNE1 |
| 82242288 | 82268990 | chr9 | + | exo | hsa_circ_0087288 | 0.7615 | 0.033338 | TLE4 |
| 53288349 | 53308214 | chr16 | + | exo | hsa_circ_0000702 | 1.4329 | 0.033372 | CHD9 |
| 51107467 | 51107611 | chr14 | − | exo | | 0.7795 | 0.033398 | SAV1 |
| 134058382 | 134059459 | chr10 | − | s/o | | 1.1999 | 0.033424 | STK32C |
| 32761408 | 32807433 | chr10 | + | exo | | 1.1006 | 0.033427 | CCDC7 |
| 31025753 | 31040421 | chr19 | + | exo | | 0.5505 | 0.033464 | ZNF536 |
| 71668272 | 71671937 | chr11 | + | exo | hsa_circ_0023404 | 0.7055 | 0.033476 | RNF121 |
| 201681944 | 201687883 | chr1 | + | exo | hsa_circ_0002869 | 1.3659 | 0.033528 | NAV1 |
| 91940462 | 91949645 | chr9 | + | exo | hsa_circ_0001871 | 0.8315 | 0.033531 | SECISBP2 |
| 34525901 | 34526015 | chr15 | − | s/o | hsa_circ_0000590 | 0.7308 | 0.033668 | SLC12A6 |
| 205138290 | 205156934 | chr1 | − | exo | hsa_circ_0016199 | 1.0717 | 0.033708 | DSTYK |
| 34969052 | 34991833 | chr11 | + | exo | hsa_circ_0006254 | 1.0844 | 0.033723 | PDHX |
| 26853824 | 26854543 | chr22 | − | exo | hsa_circ_0005175 | 1.2538 | 0.033849 | HPS4 |
| 21968636 | 21970045 | chr14 | − | exo | hsa_circ_0005957 | 0.8788 | 0.033859 | METTL3 |
| 44595024 | 44596428 | chr1 | + | exo | | 0.8631 | 0.033980 | KLF17 |
| 36953719 | 36976504 | chr5 | + | exo | hsa_circ_0001472 | 1.1074 | 0.034032 | NIPBL |
| 53289511 | 53302038 | chr16 | + | exo | hsa_circ_0039365 | 1.6191 | 0.034060 | CHD9 |
| 66982810 | 66983420 | chr11 | + | exo | hsa_circ_0023060 | 1.2860 | 0.034103 | KDM2A |
| 151682934 | 151729550 | chr4 | − | exo | hsa_circ_0071185 | 0.6438 | 0.034152 | LRBA |
| 46604837 | 46624651 | chr21 | + | exo | hsa_circ_0062011 | 1.3316 | 0.034163 | ADARB1 |
| 129410903 | 129469509 | chr12 | + | exo | hsa_circ_0029409 | 1.1410 | 0.034261 | GLT1D1 |
| 91808723 | 91810001 | chr14 | − | exo | hsa_circ_0032959 | 1.2430 | 0.034322 | CCDC88C |
| 122215262 | 122216551 | chr2 | − | exo | hsa_circ_0056310 | 1.4682 | 0.034373 | CLASP1 |
| 95494501 | 95507595 | chr4 | + | exo | hsa_circ_0070467 | 0.7193 | 0.034375 | PDLIM5 |
| 149563797 | 149619949 | chr3 | + | exo | hsa_circ_0004565 | 0.8621 | 0.034386 | RNF13 |
| 137313521 | 137326053 | chr9 | + | exo | | 1.7505 | 0.034417 | RXRA |
| 30428647 | 30446010 | chr21 | − | exo | hsa_circ_0061376 | 0.7927 | 0.034417 | CCT8 |
| 42294506 | 42295095 | chr8 | − | exo | hsa_circ_0084125 | 0.8599 | 0.034510 | SLC20A2 |
| 95110953 | 95111328 | chr10 | − | exo | | 0.8756 | 0.034563 | MYOF |
| 45050202 | 45053302 | chr21 | − | exo | hsa_circ_0061879 | 0.9120 | 0.034572 | HSF2BP |
| 33540210 | 33540336 | chr2 | + | exo | hsa_circ_0053936 | 0.6625 | 0.034615 | LTBP1 |
| 112743867 | 112757546 | chr12 | − | exo | hsa_circ_0028411 | 1.6140 | 0.034643 | HECTD4 |
| 79536041 | 79539114 | chr17 | − | exo | hsa_circ_0002279 | 1.2970 | 0.034720 | NPLOC4 |
| 66021409 | 66048810 | chr15 | − | exo | hsa_circ_0035946 | 0.8783 | 0.034808 | DENND4A |
| 108690900 | 108703915 | chr1 | − | exo | hsa_circ_0004270 | 0.8903 | 0.034820 | SLC25A24 |
| 74140611 | 74143892 | chr2 | + | exo | hsa_circ_0055264 | 0.7743 | 0.034984 | ACTG2 |
| 38695899 | 38696957 | chr22 | − | exo | hsa_circ_0063300 | 1.4664 | 0.034996 | CSNK1E |
| 169018296 | 169038600 | chr2 | − | exo | hsa_circ_0001079 | 0.7227 | 0.035034 | STK39 |
| 236192849 | 236201553 | chr1 | − | exo | hsa_circ_0017109 | 1.3808 | 0.035107 | NID1 |
| 169982931 | 170034621 | chr6 | − | exo | hsa_circ_0078724 | 0.7345 | 0.035111 | WDR27 |
| 46832463 | 46834530 | chr19 | + | exo | | 0.6940 | 0.035269 | HIF3A |
| 17117929 | 17128675 | chr22 | + | exo | hsa_circ_0062142 | 0.7036 | 0.035282 | TPTEP1 |
| 92564787 | 92577898 | chr11 | + | exo | | 1.1632 | 0.035287 | FAT3 |
| 10685092 | 10685705 | chr19 | − | exo | hsa_circ_0049279 | 0.8786 | 0.035417 | AP1M2 |
| 156376894 | 156377214 | chr7 | − | int | | 0.8155 | 0.035484 | LINC01006 |
| 47073938 | 47078385 | chr11 | + | int | | 1.3197 | 0.035533 | C11orf49 |
| 137569740 | 137570248 | chr7 | − | exo | hsa_circ_0082564 | 0.6443 | 0.035578 | CREB3L2 |
| 75625762 | 75634722 | chr7 | − | s/o | hsa_circ_0001718 | 1.1219 | 0.035615 | STYXL1 |
| 191523883 | 191537878 | chr2 | + | exo | hsa_circ_0002024 | 1.5086 | 0.035646 | NAB1 |
| 59996760 | 60002460 | chr17 | − | exo | hsa_circ_0007064 | 0.8299 | 0.035647 | INTS2 |
| 104352338 | 104359301 | chr10 | + | exo | hsa_circ_0003779 | 0.8721 | 0.035660 | SUFU |
| 48216600 | 48246391 | chr19 | + | exo | hsa_circ_0051695 | 0.8155 | 0.035794 | EHD2 |
| 20200273 | 20209395 | chr17 | + | exo | hsa_circ_0042438 | 1.5907 | 0.035848 | SPECC1 |
| 102022404 | 102038934 | chr2 | − | exo | hsa_circ_0005536 | 0.8278 | 0.035848 | RFX8 |
| 18419661 | 18462483 | chr3 | − | exo | hsa_circ_0064555 | 1.2270 | 0.035922 | SATB1 |
| 25477659 | 25477979 | chr20 | − | int | hsa_circ_0092338 | 0.7912 | 0.035924 | NINL |
| 139285162 | 139416814 | chr7 | − | exo | hsa_circ_0082672 | 1.6648 | 0.036005 | HIPK2 |
| 88029300 | 88045722 | chr11 | − | exo | hsa_circ_0023977 | 0.7759 | 0.036010 | CTSC |
| 138784244 | 138842320 | chr5 | − | exo | hsa_circ_0074216 | 0.7689 | 0.036021 | ECSCR |
| 32730071 | 32730272 | chr2 | + | exo | hsa_circ_0053816 | 1.1182 | 0.036087 | BIRC6 |
| 49329942 | 49332031 | chr3 | − | exo | hsa_circ_0065623 | 1.2687 | 0.036118 | USP4 |
| 104247906 | 104250370 | chr10 | − | exo | hsa_circ_0019753 | 1.3321 | 0.036147 | ACTR1A |
| 202013725 | 202019130 | chr2 | + | s/o | | 0.7399 | 0.036175 | CFLAR |
| 120995348 | 120995485 | chr12 | + | exo | hsa_circ_0028904 | 1.2076 | 0.036188 | RNF10 |
| 89958600 | 89962491 | chr16 | + | exo | hsa_circ_0002507 | 1.4732 | 0.036190 | TCF25 |
| 20235837 | 20244503 | chr13 | + | exo | hsa_circ_0029605 | 1.1771 | 0.036207 | MPHOSPH8 |
| 8474368 | 8486369 | chr11 | − | exo | | 1.2965 | 0.036219 | STK33 |
| 154317537 | 154319114 | chrX | + | exo | hsa_circ_0092163 | 1.2111 | 0.036250 | BRCC3 |
| 51706885 | 51716483 | chr14 | + | exo | hsa_circ_0031896 | 0.8068 | 0.036271 | TMX1 |
| 56167366 | 56167744 | chr19 | + | int | hsa_circ_0000961 | 0.9255 | 0.036361 | U2AF2 |
| 138552720 | 138554507 | chr7 | − | exo | | 1.1792 | 0.036373 | KIAA1549 |
| 31645077 | 31645307 | chr15 | − | as | hsa_circ_0000589 | 0.8759 | 0.036491 | KLF13 |
| 76166897 | 76167135 | chr17 | + | exo | hsa_circ_0045871 | 0.7043 | 0.036577 | SYNGR2 |
| 9075228 | 9083523 | chr12 | + | exo | hsa_circ_0025402 | 1.3379 | 0.036578 | PHC1 |
| 4186092 | 4192703 | chr17 | − | exo | hsa_circ_0002368 | 0.8290 | 0.036619 | UBE2G1 |
| 24366276 | 24368731 | chr12 | − | int | | 0.8277 | 0.036703 | SOX5 |
| 47647225 | 47648679 | chr11 | − | exo | hsa_circ_0022025 | 0.7205 | 0.036710 | MTCH2 |
| 117399269 | 117401006 | chr9 | + | exo | hsa_circ_0002318 | 1.0819 | 0.036739 | C9orf91 |
| 77052382 | 77065626 | chr4 | − | exo | hsa_circ_0070037 | 1.1083 | 0.036753 | NUP54 |
| 48922490 | 48923071 | chr19 | + | exo | hsa_circ_0051749 | 1.4951 | 0.036762 | GRIN2D |
| 17604597 | 17646046 | chr10 | − | s/o | | 0.8721 | 0.036792 | HACD1 |
| 42641806 | 42641893 | chr20 | + | int | hsa_circ_0001160 | 1.2044 | 0.036794 | TOX2 |
| 6629914 | 6641771 | chr7 | + | exo | hsa_circ_0079387 | 0.8474 | 0.036826 | C7orf26 |
| 143486218 | 143605362 | chr6 | + | exo | hsa_circ_0078051 | 0.9361 | 0.036903 | AIG1 |
| 151400298 | 151403317 | chr1 | − | exo | hsa_circ_0008704 | 1.2914 | 0.036913 | POGZ |
| 48585964 | 48587667 | chr3 | − | exo | | 0.6681 | 0.036970 | PFKFB4 |
| 95426872 | 95428726 | chr1 | + | s/o | | 0.7621 | 0.037046 | LOC729970 |
| 27995214 | 28019595 | chr8 | + | exo | hsa_circ_0083789 | 0.7808 | 0.037192 | ELP3 |
| 186180001 | 186188309 | chr4 | + | exo | hsa_circ_0005066 | 0.6851 | 0.037224 | SNX25 |
| 37763625 | 37770736 | chr9 | + | exo | | 1.1259 | 0.037297 | TRMT10B |
| 240492375 | 240519208 | chr1 | + | exo | hsa_circ_0002636 | 0.7592 | 0.037319 | FMN2 |
| 19076880 | 19077003 | chr22 | − | exo | hsa_circ_0062204 | 1.2901 | 0.037350 | DGCR2 |
| 49227059 | 49228280 | chr12 | − | exo | | 1.2939 | 0.037352 | DDX23 |
| 158870027 | 158882761 | chr6 | + | exo | hsa_circ_0078467 | 1.3110 | 0.037370 | TULP4 |
| 6825911 | 6826411 | chr4 | + | exo | hsa_circ_0069096 | 1.1300 | 0.037483 | KIAA0232 |
| 146757974 | 146759412 | chr1 | + | exo | hsa_circ_0006296 | 0.9011 | 0.037494 | CHD1L |
| 37006469 | 37020798 | chr9 | − | exo | hsa_circ_0001858 | 1.1763 | 0.037599 | PAX5 |
| 38208893 | 38209616 | chr22 | + | exo | hsa_circ_0007513 | 0.8109 | 0.037602 | GCAT |
| 93543741 | 93558139 | chr15 | + | exo | hsa_circ_0037002 | 0.8000 | 0.037735 | CHD2 |
| 9868680 | 9871079 | chr3 | + | exo | | 1.1899 | 0.037774 | TTLL3 |
| 1754959 | 1756341 | chr12 | + | s/o | hsa_circ_0008349 | 0.7993 | 0.037865 | WNT5B |
| 125042721 | 125054119 | chr9 | + | exo | hsa_circ_0003250 | 0.8357 | 0.037895 | MRRF |
| 217363519 | 217366188 | chr2 | + | exo | hsa_circ_0058191 | 0.8249 | 0.037899 | RPL37A |
| 51749067 | 51764480 | chr10 | + | exo | hsa_circ_0018379 | 1.3440 | 0.037969 | AGAP6 |
| 51123962 | 51364771 | chr10 | − | exo | hsa_circ_0018343 | 1.5551 | 0.037983 | PARG |
| 74734363 | 74750169 | chr17 | + | exo | | 0.8694 | 0.037987 | MFSD11 |
| 140920348 | 140920479 | chr4 | − | int | hsa_circ_0001443 | 0.6488 | 0.038022 | MAML3 |
| 25334804 | 25335610 | chr4 | + | exo | hsa_circ_0001398 | 0.7711 | 0.038110 | ZCCHC4 |
| 48239625 | 48239790 | chr19 | + | exo | hsa_circ_0051700 | 2.0635 | 0.038198 | EHD2 |
| 156619298 | 156619438 | chr7 | − | exo | hsa_circ_0083126 | 0.7674 | 0.038469 | LMBR1 |
| 1417498 | 1432689 | chr19 | + | exo | hsa_circ_0048234 | 0.8667 | 0.038532 | DAZAP1 |
| 36246027 | 36246479 | chr9 | − | exo | hsa_circ_0087008 | 1.2551 | 0.038544 | GNE |
| 41191341 | 41196173 | chr15 | + | exo | hsa_circ_0034642 | 0.8248 | 0.038554 | VPS18 |
| 15421611 | 15421841 | chr19 | − | int | | 0.8872 | 0.038578 | BRD4 |
| 160107793 | 160131735 | chr4 | + | exo | | 0.7524 | 0.038620 | RAPGEF2 |
| 158994451 | 159050851 | chr6 | + | exo | hsa_circ_0078488 | 1.3653 | 0.038714 | TMEM181 |
| 96322568 | 96333944 | chr10 | + | exo | | 0.8793 | 0.038732 | HELLS |
| 140646904 | 140812121 | chr4 | − | exo | hsa_circ_0071000 | 1.2243 | 0.038755 | MAML3 |
| 20758088 | 20770237 | chr9 | + | exo | hsa_circ_0005572 | 0.8744 | 0.038804 | FOCAD |
| 97177439 | 97200810 | chr9 | + | exo | hsa_circ_0003092 | 1.1639 | 0.038866 | HIATL1 |
| 116879948 | 116889297 | chr10 | + | exo | hsa_circ_0020093 | 0.8038 | 0.038874 | ATRNL1 |
| 100146859 | 100182391 | chr8 | + | exo | hsa_circ_0085061 | 0.7498 | 0.038978 | VPS13B |
| 178287348 | 178287568 | chr5 | + | int | hsa_circ_0092298 | 0.8693 | 0.038992 | ZNF354B |
| 44713610 | 44717214 | chr1 | − | int | hsa_circ_0000065 | 0.9121 | 0.039032 | ERI3 |
| 99896085 | 99896878 | chr13 | + | exo | hsa_circ_0030722 | 0.8598 | 0.039037 | UBAC2 |
| 8866406 | 8906689 | chr12 | + | exo | hsa_circ_0025373 | 1.1262 | 0.039073 | RIMKLB |
| 21461899 | 21470081 | chr18 | + | exo | hsa_circ_0047232 | 0.8961 | 0.039080 | LAMA3 |
| 67956934 | 67995746 | chr15 | + | exo | hsa_circ_0003440 | 0.8681 | 0.039081 | MAP2K5 |
| 125366356 | 125379252 | chr6 | + | exo | hsa_circ_0008536 | 0.7409 | 0.039323 | RNF217 |
| 112139965 | 112150461 | chr12 | + | exo | | 1.1504 | 0.039330 | ACAD10 |
| 48215773 | 48219478 | chr3 | − | exo | hsa_circ_0002023 | 0.8778 | 0.039330 | CDC25A |
| 30694790 | 30700472 | chr17 | + | exo | hsa_circ_0043031 | 1.2457 | 0.039353 | ZNF207 |
| 77270158 | 77275895 | chrX | + | exo | hsa_circ_0001934 | 0.7899 | 0.039433 | ATP7A |
| 28146536 | 28181230 | chr16 | − | exo | hsa_circ_0038815 | 1.2191 | 0.039449 | XPO6 |
| 136561072 | 136565428 | chr3 | − | s/o | | 1.2629 | 0.039543 | NCK1-AS1 |
| 88694029 | 88695262 | chr16 | + | exo | hsa_circ_0040831 | 0.8146 | 0.039545 | ZC3H18 |
| 180962498 | 180974599 | chr1 | − | exo | hsa_circ_0000165 | 0.8834 | 0.039716 | STX6 |
| 124560229 | 124578293 | chr3 | − | exo | | 1.4383 | 0.039764 | ITGB5 |
| 45865069 | 45985474 | chr20 | − | exo | hsa_circ_0060604 | 0.8503 | 0.039791 | ZMYND8 |
| 20101609 | 20104725 | chr11 | + | exo | hsa_circ_0004819 | 1.1564 | 0.039829 | NAV2 |
| 234054804 | 234091145 | chr2 | + | exo | hsa_circ_0058741 | 0.8195 | 0.039863 | INPP5D |
| 76598440 | 76603236 | chr10 | + | exo | hsa_circ_0005164 | 0.6146 | 0.039893 | KAT6B |
| 34921480 | 34957919 | chr19 | + | exo | hsa_circ_0050512 | 0.6839 | 0.040102 | UBA2 |
| 26825465 | 26866681 | chr15 | − | exo | | 0.7812 | 0.040107 | GABRB3 |
| 28827559 | 28828036 | chr8 | + | exo | hsa_circ_0083824 | 1.1942 | 0.040177 | HMBOX1 |
| 16685846 | 16690028 | chr5 | − | exo | hsa_circ_0071991 | 2.0236 | 0.040345 | MYO10 |
| 62567915 | 62587267 | chr17 | − | exo | hsa_circ_0006644 | 0.8759 | 0.040348 | SMURF2 |
| 38591573 | 38617010 | chr15 | + | exo | hsa_circ_0004229 | 0.8300 | 0.040412 | SPRED1 |
| 131124339 | 131374017 | chr8 | − | exo | hsa_circ_0085578 | 0.8476 | 0.040516 | ASAP1 |
| 145215872 | 145218751 | chr8 | + | exo | hsa_circ_0085976 | 1.1602 | 0.040582 | MROH1 |
| 240421244 | 240555862 | chr1 | + | exo | hsa_circ_0007019 | 0.5454 | 0.040779 | FMN2 |
| 30029633 | 30032700 | chr3 | + | exo | hsa_circ_0064651 | 0.7996 | 0.040835 | RBMS3 |
| 33311103 | 33319125 | chr9 | + | exo | hsa_circ_0086645 | 0.9198 | 0.040962 | NFX1 |
| 151719232 | 151729550 | chr4 | − | exo | hsa_circ_0004636 | 0.6796 | 0.040980 | LRBA |
| 48630533 | 48631277 | chr19 | − | exo | hsa_circ_0051718 | 1.3501 | 0.041026 | LIG1 |
| 116202261 | 116206889 | chr1 | + | exo | hsa_circ_0002623 | 1.1939 | 0.041028 | VANGL1 |
| 101876363 | 101877994 | chr2 | − | as | | 0.8596 | 0.041039 | CNOT11 |
| 92301363 | 92330608 | chr8 | + | exo | | 0.7446 | 0.041163 | SLC26A7 |
| 113795789 | 113798724 | chr3 | + | int | | 0.8427 | 0.041210 | QTRTD1 |
| 149912506 | 149915447 | chr5 | + | exo | hsa_circ_0074543 | 1.4336 | 0.041285 | NDST1 |
| 151727422 | 151729550 | chr4 | − | exo | hsa_circ_0071196 | 0.5736 | 0.041297 | LRBA |
| 44838346 | 44839479 | chr7 | + | int | | 0.8071 | 0.041306 | PPIA |
| 69565029 | 69565697 | chr2 | − | exo | hsa_circ_0001025 | 0.8618 | 0.041386 | GFPT1 |
| 52192265 | 52199707 | chr20 | − | exo | hsa_circ_0060918 | 0.8067 | 0.041393 | ZNF217 |
| 64485055 | 64509673 | chr12 | + | exo | | 0.7304 | 0.041439 | SRGAP1 |
| 131796425 | 131799517 | chr2 | + | exo | hsa_circ_0056472 | 1.7408 | 0.041467 | ARHGEF4 |
| 40506473 | 40506637 | chr1 | + | int | hsa_circ_0000059 | 0.8407 | 0.041721 | CAP1 |
| 33525517 | 33525640 | chr2 | + | exo | hsa_circ_0053932 | 0.6171 | 0.041728 | LTBP1 |
| 150814181 | 150815719 | chr7 | + | exo | hsa_circ_0003027 | 1.3647 | 0.041759 | AGAP3 |
| 128561179 | 128643455 | chr6 | − | exo | hsa_circ_0077816 | 1.1229 | 0.041801 | PTPRK |
| 78199635 | 78201759 | chr17 | + | exo | hsa_circ_0045999 | 0.8280 | 0.041827 | SLC26A11 |
| 142416760 | 142437312 | chr5 | + | exo | hsa_circ_0074368 | 0.8813 | 0.041868 | ARHGAP26 |
| 186946056 | 186964557 | chr2 | − | s/o | | 0.8729 | 0.041885 | LINC01473 |
| 16836696 | 16838378 | chrX | + | exo | hsa_circ_0006020 | 0.9221 | 0.041896 | TXLNG |
| 77333544 | 77340944 | chr11 | − | exo | hsa_circ_0023696 | 0.7975 | 0.041946 | CLNS1A |
| 94899365 | 94945248 | chr15 | + | exo | hsa_circ_0000660 | 0.9230 | 0.041988 | MCTP2 |
| 79249768 | 79249951 | chr17 | − | exo | hsa_circ_0046127 | 1.2272 | 0.042090 | SLC38A10 |
| 123078822 | 123109235 | chr12 | + | exo | | 0.7294 | 0.042109 | KNTC1 |
| 108186549 | 108206688 | chr11 | + | exo | hsa_circ_0024231 | 1.1670 | 0.042138 | ATM |
| 219366471 | 219414650 | chr1 | + | s/o | hsa_circ_0006963 | 0.6537 | 0.042224 | LYPLAL1 |
| 345853 | 348841 | chr6 | + | exo | | 1.2634 | 0.042328 | DUSP22 |
| 145613012 | 145616995 | chr5 | + | exo | hsa_circ_0074428 | 0.8030 | 0.042335 | RBM27 |
| 24613084 | 24708279 | chr7 | + | exo | hsa_circ_0079614 | 1.7275 | 0.042431 | MPP6 |
| 113143415 | 113153625 | chr1 | − | exo | hsa_circ_0000108 | 0.8840 | 0.042467 | ST7L |
| 76301164 | 76415337 | chr13 | + | exo | hsa_circ_0030448 | 1.1545 | 0.042574 | LMO7 |
| 6880240 | 6885270 | chr1 | + | exo | hsa_circ_0009541 | 0.7838 | 0.042621 | CAMTA1 |
| 70193988 | 70195501 | chr12 | + | exo | hsa_circ_0000419 | 0.7474 | 0.042721 | RAB3IP |
| 86017286 | 86017524 | chr11 | + | exo | hsa_circ_0023956 | 1.1124 | 0.042771 | C11orf73 |
| 45697517 | 45702728 | chr15 | + | exo | | 0.8827 | 0.042849 | SPATA5L1 |
| 156848913 | 156849949 | chr1 | + | exo | hsa_circ_0014798 | 0.8427 | 0.042928 | NTRK1 |
| 36230203 | 36231267 | chr4 | − | exo | hsa_circ_0069399 | 1.0555 | 0.042940 | ARAP2 |
| 81929411 | 81942196 | chr16 | + | exo | hsa_circ_0000721 | 0.8739 | 0.043008 | PLCG2 |
| 151509199 | 151520283 | chr4 | − | exo | hsa_circ_0004780 | 1.1362 | 0.043053 | LRBA |
| 33632307 | 33637797 | chr20 | − | exo | | 1.3521 | 0.043138 | TRPC4AP |
| 135172273 | 135187243 | chr9 | − | exo | hsa_circ_0089282 | 0.8497 | 0.043182 | SETX |
| 18685866 | 18688270 | chr19 | + | exo | hsa_circ_0050139 | 1.4238 | 0.043186 | UBA52 |
| 29068914 | 29070498 | chr1 | + | exo | hsa_circ_0000039 | 0.8495 | 0.043292 | YTHDF2 |
| 289226 | 289944 | chr4 | − | exo | | 0.8145 | 0.043303 | ZNF732 |
| 87593948 | 87607727 | chr7 | + | exo | hsa_circ_0004674 | 1.2155 | 0.043339 | ADAM22 |
| 123974474 | 123980354 | chr5 | − | exo | hsa_circ_0073713 | 0.8702 | 0.043467 | ZNF608 |
| 49458943 | 49464519 | chr19 | + | exo | hsa_circ_0051799 | 0.7911 | 0.043549 | BAX |
| 3955264 | 3968123 | chr17 | − | exo | hsa_circ_0005856 | 1.1008 | 0.043573 | ZZEF1 |
| 73418464 | 73431944 | chr11 | − | exo | hsa_circ_0003908 | 0.7861 | 0.043587 | RAB6A |
| 36823839 | 36824423 | chr1 | − | exo | hsa_circ_0011692 | 0.6609 | 0.043709 | STK40 |
| 149275007 | 149285699 | chr6 | + | exo | | 0.8654 | 0.043748 | UST |
| 40596333 | 40601362 | chr21 | − | exo | hsa_circ_0001194 | 1.2032 | 0.043820 | BRWD1 |
| 31205314 | 31230683 | chr1 | − | exo | hsa_circ_0011206 | 1.3649 | 0.043833 | LAPTM5 |
| 76240770 | 76247617 | chr7 | − | exo | hsa_circ_0080813 | 0.9326 | 0.043940 | POMZP3 |
| 95813442 | 95840796 | chr13 | − | exo | hsa_circ_0030586 | 0.9203 | 0.044129 | ABCC4 |
| 53478829 | 53481229 | chr17 | − | exo | hsa_circ_0002015 | 0.8175 | 0.044164 | MMD |
| 1721833 | 1722035 | chr1 | − | exo | hsa_circ_0009343 | 1.9088 | 0.044204 | GNB1 |
| 82181402 | 82185119 | chr16 | − | exo | | 0.8309 | 0.044386 | MPHOSPH6 |
| 67471916 | 67477081 | chr16 | − | exo | hsa_circ_0039830 | 0.7854 | 0.044572 | ATP6V0D1 |
| 110397651 | 110399490 | chr12 | − | exo | hsa_circ_0028152 | 0.7391 | 0.044589 | GIT2 |
| 27132716 | 27143560 | chr12 | − | exo | hsa_circ_0025705 | 1.2194 | 0.044619 | TM7SF3 |
| 101550654 | 101551042 | chr15 | + | exo | hsa_circ_0037078 | 0.8948 | 0.044749 | LRRK1 |
| 78177431 | 78178966 | chr1 | − | exo | hsa_circ_0012989 | 0.8899 | 0.044759 | USP33 |
| 197621364 | 197627499 | chr1 | − | exo | hsa_circ_0015796 | 1.3162 | 0.044765 | DENND1B |
| 46998521 | 47000299 | chr17 | + | exo | | 1.2481 | 0.044902 | UBE2Z |
| 40555081 | 40558179 | chr1 | − | exo | hsa_circ_0011885 | 1.1445 | 0.044980 | PPT1 |
| 81813151 | 81816161 | chr10 | − | exo | hsa_circ_0018974 | 0.9121 | 0.045033 | TMEM254-AS1 |
| 68359043 | 68367962 | chr11 | + | exo | hsa_circ_0001968 | 0.7570 | 0.045033 | PPP6R3 |
| 77237532 | 77239554 | chr14 | + | exo | | 2.6587 | 0.045097 | VASH1 |
| 53459074 | 53459374 | chr1 | − | s/o | hsa_circ_0006906 | 1.1584 | 0.045112 | RP11-334A14.2 |
| 206603511 | 206632293 | chr1 | + | exo | hsa_circ_0016229 | 1.3701 | 0.045127 | SRGAP2 |
| 118378243 | 118382740 | chr11 | + | exo | | 1.2720 | 0.045143 | KMT2A |
| 97178494 | 97222866 | chr12 | + | exo | | 0.8322 | 0.045188 | C12orf55 |
| 114265310 | 114277601 | chr13 | + | exo | hsa_circ_0031038 | 1.2829 | 0.045258 | TFDP1 |
| 20671447 | 20671623 | chr15 | − | exo | hsa_circ_0004934 | 0.8219 | 0.045449 | HERC2P3 |
| 465742 | 489604 | chr17 | − | exo | | 1.6179 | 0.045453 | VPS53 |
| 59934576 | 59943360 | chr5 | − | exo | hsa_circ_0002512 | 0.6956 | 0.045468 | DEPDC1B |
| 80412669 | 80423635 | chr15 | + | exo | | 0.9039 | 0.045489 | ZFAND6 |
| 78771724 | 78787649 | chr10 | − | exo | hsa_circ_0005967 | 0.8488 | 0.045490 | KCNMA1 |
| 3150990 | 3151271 | chr10 | + | int | | 0.8734 | 0.045498 | PFKP |
| 46889530 | 46906128 | chr18 | − | exo | hsa_circ_0000849 | 0.6479 | 0.045574 | DYM |
| 9450336 | 9450536 | chr11 | + | int | hsa_circ_0092372 | 1.1063 | 0.045591 | IPO7 |
| 144817634 | 144819722 | chr5 | − | s/o | | 0.8723 | 0.045638 | XLOC_005041 |
| 93788384 | 93789309 | chr12 | + | exo | hsa_circ_0008916 | 1.3891 | 0.045648 | NUDT4 |
| 57276883 | 57301820 | chr3 | + | exo | hsa_circ_0066251 | 1.2734 | 0.045671 | APPL1 |
| 151277130 | 151281551 | chr6 | + | exo | hsa_circ_0078292 | 0.8466 | 0.045681 | MTHFD1L |
| 27867712 | 27878600 | chr12 | + | exo | | 0.6384 | 0.045723 | MRPS35 |
| 51107467 | 51111732 | chr14 | − | exo | hsa_circ_0007101 | 0.7896 | 0.045885 | SAV1 |
| 151198769 | 151209117 | chr6 | + | exo | | 0.8668 | 0.046081 | MTHFD1L |
| 18851019 | 18851268 | chr16 | − | exo | hsa_circ_0006976 | 1.2504 | 0.046290 | SMG1 |
| 80714040 | 80726442 | chr17 | + | exo | hsa_circ_0046447 | 1.5253 | 0.046434 | TBCD |
| 1255698 | 1256967 | chr19 | + | int | | 0.9576 | 0.046435 | MIDN |
| 101957689 | 101957837 | chr7 | − | as | hsa_circ_0001734 | 1.0760 | 0.046509 | SH2B2 |
| 142146607 | 142148236 | chr8 | + | exo | | 0.8050 | 0.046514 | DENND3 |
| 33406276 | 33407007 | chr8 | − | s/o | hsa_circ_0001787 | 1.1333 | 0.046626 | RNF122 |
| 125832628 | 125839041 | chr9 | + | exo | hsa_circ_0007661 | 0.8365 | 0.046633 | RABGAP1 |
| 47285639 | 47291842 | chr19 | − | exo | hsa_circ_0051638 | 0.7379 | 0.046642 | SLC1A5 |
| 62567915 | 62582288 | chr17 | − | exo | hsa_circ_0005637 | 0.8219 | 0.046712 | SMURF2 |
| 28800065 | 28800663 | chr1 | + | exo | hsa_circ_0002909 | 0.7404 | 0.046803 | PHACTR4 |
| 45008891 | 45012133 | chr20 | − | exo | hsa_circ_0060580 | 1.5109 | 0.046841 | ELMO2 |
| 76566752 | 76578822 | chr15 | − | exo | hsa_circ_0036398 | 1.0918 | 0.046900 | ETFA |
| 116668337 | 116675510 | chr12 | − | s/o | hsa_circ_0000443 | 1.2970 | 0.046906 | MED13L |
| 198190128 | 198222310 | chr1 | + | s/o | hsa_circ_0008593 | 0.7791 | 0.046923 | NEK7 |
| 50059614 | 50062337 | chr12 | − | exo | hsa_circ_0006802 | 1.2044 | 0.046939 | FMNL3 |
| 32695598 | 32718742 | chr7 | − | exo | | 0.8935 | 0.046960 | DPY19L1P1 |
| 51797729 | 51800460 | chr18 | + | exo | hsa_circ_0007180 | 0.7851 | 0.047037 | POLI |
| 51749067 | 51767836 | chr10 | + | exo | hsa_circ_0018380 | 1.3275 | 0.047075 | AGAP6 |
| 67522117 | 67522837 | chr5 | + | exo | hsa_circ_0006411 | 1.3010 | 0.047081 | PIK3R1 |
| 19358063 | 19359646 | chr18 | + | exo | hsa_circ_0008190 | 0.8180 | 0.047115 | MIB1 |
| 27267947 | 27268309 | chr1 | + | exo | hsa_circ_0003940 | 1.5024 | 0.047117 | NUDC |
| 114440461 | 114444507 | chr1 | − | exo | hsa_circ_0000112 | 0.8892 | 0.047201 | AP4B1 |
| 16362557 | 16497935 | chr11 | − | exo | hsa_circ_0021352 | 1.1034 | 0.047210 | SOX6 |
| 74733078 | 74733379 | chr2 | − | exo | hsa_circ_0055339 | 1.2029 | 0.047306 | PCGF1 |
| 97541420 | 97554192 | chr8 | + | int | hsa_circ_0002977 | 0.8763 | 0.047322 | SDC2 |
| 35163017 | 35163328 | chr11 | + | int | hsa_circ_0000291 | 0.7333 | 0.047328 | CD44 |
| 66455621 | 66512933 | chr3 | − | exo | hsa_circ_0001318 | 3.6383 | 0.047399 | LRIG1 |
| 45011271 | 45016256 | chr7 | − | exo | hsa_circ_0080099 | 1.0717 | 0.047428 | MYO1G |
| 147131761 | 147131890 | chr1 | − | exo | hsa_circ_0013959 | 0.6452 | 0.047436 | ACP6 |
| 3017117 | 3017219 | chr6 | + | exo | hsa_circ_0075494 | 1.2362 | 0.047437 | NQO2 |
| 15939756 | 15946230 | chr19 | + | exo | hsa_circ_0049851 | 0.8017 | 0.047441 | XLOC_012981 |
| 43655941 | 43671480 | chr10 | + | exo | hsa_circ_0018255 | 1.1762 | 0.047594 | CSGALNACT2 |
| 612404 | 620376 | chr5 | + | exo | hsa_circ_0071649 | 0.8293 | 0.047714 | CEP72 |
| 46334641 | 46334846 | chr19 | − | exo | hsa_circ_0008001 | 0.7813 | 0.047762 | SYMPK |
| 33399937 | 33400583 | chr6 | + | exo | hsa_circ_0075968 | 1.5439 | 0.047782 | SYNGAP1 |
| 4192524 | 4200109 | chr17 | − | exo | hsa_circ_0041555 | 0.8476 | 0.047802 | UBE2G1 |
| 54245398 | 54251644 | chr19 | + | s/o | | 0.6695 | 0.047809 | MIR520H |
| 84792321 | 84793881 | chr16 | + | exo | hsa_circ_0040704 | 1.1886 | 0.047996 | USP10 |
| 77798585 | 77807398 | chr13 | − | exo | hsa_circ_0030514 | 0.8131 | 0.048048 | MYCBP2 |
| 196793574 | 196846401 | chr3 | − | exo | hsa_circ_0068684 | 0.7162 | 0.048100 | DLG1 |
| 53322669 | 53329829 | chr1 | + | exo | hsa_circ_0002361 | 0.9247 | 0.048181 | ZYG11A |
| 20907514 | 20910300 | chr17 | − | exo | hsa_circ_0042451 | 1.5882 | 0.048215 | USP22 |
| 16979023 | 16981390 | chr17 | + | exo | hsa_circ_0006600 | 1.7139 | 0.048342 | MPRIP |
| 8093409 | 8095466 | chr6 | − | int | | 1.0946 | 0.048376 | EEF1E1 |
| 154645235 | 154649223 | chrX | − | s/o | hsa_circ_0004666 | 0.8938 | 0.048742 | XLOC_008269 |
| 5274420 | 5275744 | chr11 | − | exo | hsa_circ_0020964 | 0.7663 | 0.048816 | HBG2 |
| 78055344 | 78055857 | chr17 | + | exo | | 1.2246 | 0.048916 | CCDC40 |
| 183817132 | 183818059 | chr2 | − | exo | hsa_circ_0002092 | 1.1458 | 0.048945 | NCKAP1 |
| 88648196 | 88655783 | chr9 | − | exo | | 1.4652 | 0.049075 | GOLM1 |
| 12332743 | 12340053 | chr12 | − | exo | hsa_circ_0008932 | 1.2991 | 0.049112 | LRP6 |
| 38656908 | 38666209 | chr15 | + | ing | | 0.6024 | 0.049181 | |
| 118451960 | 118454073 | chr11 | + | exo | | 1.4133 | 0.049185 | ARCN1 |
| 129760588 | 129762042 | chr7 | + | exo | hsa_circ_0002190 | 0.8238 | 0.049214 | KLHDC10 |
| 33442618 | 33447218 | chr2 | + | exo | hsa_circ_0053907 | 0.5297 | 0.049274 | LTBP1 |
| 87275682 | 87292020 | chr4 | − | exo | | 1.3370 | 0.049281 | MAPK10 |
| 71912018 | 71921687 | chr10 | − | s/o | hsa_circ_0005624 | 0.8290 | 0.049407 | SAR1A |
| 48381417 | 48382201 | chr16 | + | exo | hsa_circ_0005813 | 0.8468 | 0.049450 | LONP2 |
| 97024782 | 97033111 | chr2 | + | exo | hsa_circ_0001046 | 0.8868 | 0.049484 | NCAPH |
| 136660789 | 136662931 | chr9 | − | exo | hsa_circ_0089386 | 1.5695 | 0.049486 | VAV2 |
| 44128265 | 44131942 | chr19 | − | exo | | 1.6440 | 0.049562 | CADM4 |
| 217329319 | 217340174 | chr2 | + | exo | hsa_circ_0058189 | 0.8543 | 0.049564 | SMARCAL1 |
| 158288578 | 158296208 | chr6 | + | exo | hsa_circ_0002971 | 1.1185 | 0.049718 | SNX9 |
| 44492094 | 44493451 | chr21 | − | exo | | 0.7123 | 0.049740 | CBS |
| 43242459 | 43258484 | chr15 | − | exo | hsa_circ_0008546 | 0.8782 | 0.049741 | UBR1 |
| 104577350 | 104579560 | chr4 | − | exo | | 0.8217 | 0.049830 | TACR3 |
| 71761715 | 71762097 | chr15 | + | int | | 0.9057 | 0.049899 | THSD4 |
| 12825860 | 12825983 | chr19 | − | exo | hsa_circ_0049586 | 1.8325 | 0.049965 | TNPO2 |
| 65592690 | 65599361 | chr7 | + | exo | hsa_circ_0004604 | 1.1545 | 0.049994 | CRCP |

TABLE 2

| | | | | circRNA | | Fold | p-value | |
|---|---|---|---|---|---|---|---|---|
| Tx Start | Tx End | Chrom | Strand | type | circRNA Alias | SCZ iNs | SCZ iNs | GeneSymbol |
| 94721980 | 94735290 | chr12 | − | s/o | hsa_circ_0000429 | 1.6276 | 0.000002 | CEP83 |
| 44713610 | 44785416 | chr1 | − | exo | | 1.4124 | 0.000002 | ERI3 |
| 32617574 | 32619410 | chr20 | + | s/o | | 1.5239 | 0.000002 | RALY |
| 18160113 | 18166609 | chr6 | + | exo | hsa_circ_0001580 | 0.6003 | 0.000004 | KDM1B |
| 131862419 | 131897848 | chr2 | + | exo | hsa_circ_0056485 | 1.9094 | 0.000005 | PLEKHB2 |
| 8946801 | 8949183 | chr16 | − | s/o | hsa_circ_0037798 | 0.6667 | 0.000023 | CARHSP1 |
| 26772806 | 26786635 | chr1 | + | exo | hsa_circ_0011000 | 1.2000 | 0.000024 | DHDDS |
| 8728631 | 8734282 | chr11 | − | exo | hsa_circ_0021102 | 1.5681 | 0.000033 | ST5 |
| 7225183 | 7225329 | chr17 | − | exo | hsa_circ_0041821 | 1.1988 | 0.000041 | NEURL4 |
| 64113474 | 64118696 | chr2 | + | exo | hsa_circ_0054947 | 1.1721 | 0.000051 | UGP2 |
| 47905209 | 47907363 | chr4 | − | exo | hsa_circ_0008828 | 1.3847 | 0.000052 | NFXL1 |
| 75104453 | 76279715 | chr7 | − | s/o | hsa_circ_0006766 | 0.6370 | 0.000056 | CCL24 |
| 3983107 | 3983289 | chr19 | − | exo | hsa_circ_0048574 | 0.5166 | 0.000061 | EEF2 |
| 20113099 | 20113439 | chr22 | + | int | hsa_circ_0092314 | 0.5291 | 0.000063 | RANBP1 |
| 227729319 | 227779067 | chr2 | + | exo | hsa_circ_0058495 | 0.4401 | 0.000064 | RHBDD1 |
| 86198267 | 86237420 | chr10 | + | exo | hsa_circ_0018998 | 0.2050 | 0.000067 | CCSER2 |
| 66254010 | 66255478 | chr11 | + | exo | hsa_circ_0022989 | 1.1880 | 0.000068 | DPP3 |
| 99952765 | 99954506 | chr7 | + | exo | hsa_circ_0081467 | 1.4946 | 0.000077 | STAG3L5P- |
| | | | | | | | | PVRIG2P-PILRB |
| 176964873 | 176966148 | chr5 | − | exo | hsa_circ_0075236 | 1.3945 | 0.000079 | FAM193B |
| 957580 | 970704 | chr1 | + | exo | hsa_circ_0009213 | 1.3438 | 0.000084 | AGRN |
| 27267947 | 27268309 | chr1 | + | exo | hsa_circ_0003940 | 1.8185 | 0.000085 | NUDC |
| 20235837 | 20244503 | chr13 | + | exo | hsa_circ_0029605 | 0.8427 | 0.000086 | MPHOSPH8 |
| 109336203 | 109339348 | chr1 | + | exo | hsa_circ_0000100 | 1.9389 | 0.000090 | STXBP3 |
| 3925823 | 3996216 | chr20 | − | exo | hsa_circ_0059343 | 1.4257 | 0.000090 | RNF24 |
| 20107645 | 20163607 | chr17 | + | exo | hsa_circ_0042428 | 1.7001 | 0.000101 | SPECC1 |
| 48664628 | 48668880 | chr19 | − | exo | | 1.1451 | 0.000102 | LIG1 |
| 20149238 | 20163607 | chr17 | + | exo | hsa_circ_0003545 | 1.5735 | 0.000109 | SPECC1 |
| 34981280 | 34986676 | chr19 | + | exo | hsa_circ_0008354 | 1.5184 | 0.000114 | WTIP |
| 118461218 | 118463047 | chr1 | − | exo | hsa_circ_0013755 | 0.6413 | 0.000119 | GDAP2 |
| 113114596 | 113135474 | chr3 | − | exo | | 0.3107 | 0.000124 | CFAP44 |
| 112723882 | 112745523 | chr10 | + | exo | hsa_circ_0020028 | 0.7068 | 0.000125 | SHOC2 |
| 110048311 | 110064975 | chr6 | + | exo | | 0.3089 | 0.000127 | FIG4 |
| 465246 | 465357 | chr5 | − | as | hsa_circ_0001462 | 1.6057 | 0.000130 | EXOC3 |
| 66055536 | 66084622 | chr5 | + | exo | hsa_circ_0072741 | 2.6752 | 0.000131 | MAST4 |
| 65228581 | 65237897 | chr2 | + | exo | hsa_circ_0054971 | 1.6399 | 0.000138 | SLC1A4 |
| 42163939 | 42165064 | chr17 | − | exo | hsa_circ_0000773 | 1.6720 | 0.000144 | HDAC5 |
| 73584563 | 73584911 | chr17 | − | as | hsa_circ_0000802 | 1.5273 | 0.000157 | MYO15B |
| 207200838 | 207201024 | chr1 | − | exo | | 2.3610 | 0.000158 | C1orf116 |
| 3822518 | 3824406 | chr10 | − | exo | hsa_circ_0017516 | 3.0951 | 0.000170 | KLF6 |
| 4941539 | 4941943 | chr19 | + | exo | hsa_circ_0005837 | 1.5038 | 0.000173 | UHRF1 |
| 97399255 | 97403804 | chr2 | − | exo | | 1.2595 | 0.000175 | LMAN2L |
| 37868480 | 37870715 | chr22 | − | exo | hsa_circ_0001228 | 1.5436 | 0.000177 | MFNG |
| 6646474 | 6646556 | chr12 | + | exo | hsa_circ_0025201 | 2.2661 | 0.000182 | GAPDH |
| 158870027 | 158873300 | chr6 | + | exo | hsa_circ_0002073 | 0.3577 | 0.000200 | TULP4 |
| 46931559 | 46934702 | chr16 | + | exo | hsa_circ_0039216 | 2.5205 | 0.000201 | GPT2 |
| 77208781 | 77211816 | chr18 | + | exo | hsa_circ_0000859 | 1.4535 | 0.000204 | NFATC1 |
| 52237741 | 52238395 | chr1 | + | exo | hsa_circ_0002039 | 1.0864 | 0.000204 | OSBPL9 |
| 101149911 | 101152632 | chr15 | + | exo | hsa_circ_0007550 | 1.2740 | 0.000207 | ASB7 |
| 28595035 | 28600731 | chr8 | + | exo | hsa_circ_0005411 | 0.5565 | 0.000207 | EXTL3 |
| 9073580 | 9074346 | chr12 | + | exo | hsa_circ_0025395 | 1.3118 | 0.000220 | PHC1 |
| 85554223 | 85554349 | chr2 | − | s/o | hsa_circ_0001036 | 1.6906 | 0.000229 | TGOLN2 |
| 213302869 | 213349835 | chr1 | + | exo | hsa_circ_0007772 | 0.6647 | 0.000229 | RPS6KC1 |
| 1125950 | 1151181 | chr10 | + | exo | hsa_circ_0017449 | 2.4778 | 0.000239 | WDR37 |
| 69575302 | 69577315 | chr2 | − | exo | hsa_circ_0055054 | 0.8167 | 0.000240 | GFPT1 |
| 10602252 | 10602938 | chr19 | − | exo | hsa_circ_0006149 | 1.6013 | 0.000242 | KEAP1 |
| 41916541 | 41929074 | chr19 | + | exo | hsa_circ_0000937 | 2.0692 | 0.000243 | BCKDHA |
| 59323002 | 59323901 | chr15 | + | exo | hsa_circ_0001982 | 1.7981 | 0.000246 | RNF111 |
| 29682911 | 29683123 | chr22 | + | exo | hsa_circ_0008044 | 2.0562 | 0.000247 | EWSR1 |
| 90446447 | 90454051 | chr15 | − | exo | hsa_circ_0036750 | 1.6549 | 0.000261 | ARPIN |
| 47745912 | 47748131 | chr1 | − | exo | hsa_circ_0000069 | 0.6809 | 0.000266 | STIL |
| 62579578 | 62582288 | chr17 | − | exo | hsa_circ_0045322 | 0.6068 | 0.000277 | SMURF2 |
| 44750506 | 44785416 | chr1 | − | exo | hsa_circ_0005716 | 1.5195 | 0.000278 | ERI3 |
| 78268954 | 78279552 | chr1 | + | exo | | 0.6459 | 0.000279 | FAM73A |
| 46808366 | 46906128 | chr18 | − | exo | hsa_circ_0005209 | 0.8018 | 0.000288 | DYM |
| 206902379 | 206907630 | chr1 | + | exo | hsa_circ_0016263 | 1.1668 | 0.000288 | MAPKAPK2 |
| 14369008 | 14369452 | chr5 | + | int | | 2.6141 | 0.000290 | TRIO |
| 102076623 | 102080295 | chr11 | + | exo | hsa_circ_0006286 | 1.4774 | 0.000293 | YAP1 |
| 170033042 | 170058454 | chr6 | − | exo | hsa_circ_0078738 | 2.4952 | 0.000294 | WDR27 |
| 90451511 | 90454051 | chr15 | − | exo | hsa_circ_0036751 | 1.8109 | 0.000296 | ARPIN |
| 85554139 | 85554349 | chr2 | − | s/o | hsa_circ_0001035 | 1.5385 | 0.000297 | TGOLN2 |
| 128768965 | 129202693 | chr10 | + | exo | hsa_circ_0020407 | 1.2756 | 0.000299 | DOCK1 |
| 32497124 | 32504220 | chr1 | + | exo | | 1.1858 | 0.000303 | KHDRBS1 |
| 52180325 | 52188425 | chr12 | + | exo | hsa_circ_0026337 | 2.3075 | 0.000310 | SCN8A |
| 20156817 | 20163607 | chr17 | + | exo | hsa_circ_0042437 | 1.7312 | 0.000311 | SPECC1 |
| 159389691 | 159459616 | chr2 | + | exo | hsa_circ_0006870 | 1.2269 | 0.000312 | PKP4 |
| 58538023 | 58539422 | chr17 | − | exo | hsa_circ_0002496 | 0.9019 | 0.000316 | APPBP2 |
| 1746096 | 1798389 | chr17 | + | exo | hsa_circ_0041362 | 1.1939 | 0.000316 | RPA1 |
| 87376482 | 87380856 | chr16 | − | exo | hsa_circ_0040766 | 1.3635 | 0.000326 | FBXO31 |
| 1599765 | 1608285 | chr1 | − | exo | hsa_circ_0009306 | 0.2104 | 0.000326 | SLC35E2B |
| 101775286 | 101775782 | chr15 | − | exo | hsa_circ_0005019 | 1.1769 | 0.000328 | CHSY1 |
| 194877177 | 194947585 | chr3 | − | exo | hsa_circ_0008219 | 1.1482 | 0.000342 | XXYLT1 |
| 91808723 | 91810001 | chr14 | − | exo | hsa_circ_0032959 | 1.3405 | 0.000344 | CCDC88C |
| 42482087 | 42482950 | chr19 | − | exo | hsa_circ_0051258 | 1.3595 | 0.000346 | ATP1A3 |
| 80136349 | 80151706 | chr17 | − | exo | | 1.1966 | 0.000346 | CCDC57 |
| 18609120 | 18609801 | chr22 | + | exo | hsa_circ_0062191 | 1.3928 | 0.000347 | TUBA8 |
| 39743554 | 39745851 | chr2 | + | exo | hsa_circ_0054220 | 2.3704 | 0.000349 | LOC728730 |
| 1191424 | 1203372 | chr1 | − | exo | hsa_circ_0009246 | 1.1120 | 0.000351 | UBE2J2 |
| 26784281 | 26786635 | chr1 | + | exo | hsa_circ_0007537 | 1.1769 | 0.000359 | DHDDS |
| 183454505 | 183480067 | chr3 | + | exo | hsa_circ_0009131 | 1.3714 | 0.000363 | YEATS2 |
| 15000663 | 15003999 | chr3 | + | int | | 1.1428 | 0.000364 | NR2C2 |
| 141595217 | 141616013 | chr8 | − | int | hsa_circ_0007313 | 1.2339 | 0.000374 | AGO2 |
| 34854918 | 34864974 | chr17 | − | exo | hsa_circ_0043151 | 1.3079 | 0.000378 | MYO19 |
| 51454240 | 51456330 | chr3 | − | exo | hsa_circ_0065898 | 1.3719 | 0.000379 | VPRBP |
| 89598311 | 89599044 | chr16 | + | exo | hsa_circ_0040943 | 1.4207 | 0.000385 | SPG7 |
| 47767859 | 47768203 | chr19 | + | exo | hsa_circ_0000944 | 1.5160 | 0.000388 | CCDC9 |
| 52941283 | 52945896 | chr6 | + | exo | hsa_circ_0076780 | 1.1405 | 0.000393 | FBXO9 |
| 19076880 | 19077003 | chr22 | − | exo | hsa_circ_0062204 | 1.2863 | 0.000396 | DGCR2 |
| 36681397 | 36681703 | chr22 | − | int | | 0.5386 | 0.000407 | MYH9 |
| 47187818 | 47189802 | chr11 | − | exo | hsa_circ_0000298 | 0.6358 | 0.000410 | ARFGAP2 |
| 14263120 | 14271109 | chr19 | − | exo | hsa_circ_0049729 | 1.4880 | 0.000413 | LPHN1 |
| 6646474 | 6647162 | chr12 | + | exo | hsa_circ_0025202 | 2.2824 | 0.000425 | GAPDH |
| 114840817 | 114842445 | chr9 | − | exo | hsa_circ_0088036 | 0.4047 | 0.000441 | SUSD1 |
| 20811282 | 20811436 | chr14 | − | s/o | hsa_circ_0000512 | 3.4321 | 0.000443 | RPPH1 |
| 23595985 | 23615961 | chr22 | + | exo | hsa_circ_0062522 | 1.2335 | 0.000443 | BCR |
| 117037381 | 117045538 | chr6 | + | exo | | 1.2659 | 0.000445 | KPNA5 |
| 129799616 | 129813722 | chrX | − | exo | | 0.3486 | 0.000447 | ENOX2 |
| 29994794 | 30008684 | chr7 | − | exo | | 1.6017 | 0.000456 | SCRN1 |
| 112321384 | 112321572 | chr12 | + | exo | hsa_circ_0002488 | 1.1274 | 0.000462 | MAPKAPK5 |
| 10395790 | 10397291 | chr19 | + | exo | hsa_circ_0049241 | 2.3321 | 0.000464 | ICAM1 |
| 18846213 | 18856973 | chr16 | − | exo | hsa_circ_0038258 | 0.7939 | 0.000466 | SMG1 |
| 44773981 | 44785416 | chr1 | − | exo | hsa_circ_0012144 | 1.2976 | 0.000471 | ERI3 |
| 13925328 | 13925679 | chr19 | + | exo | | 1.0909 | 0.000482 | ZSWIM4 |
| 30856464 | 30857207 | chr6 | + | exo | hsa_circ_0005133 | 2.4648 | 0.000485 | DDR1 |
| 68501974 | 68503656 | chr15 | − | exo | hsa_circ_0036052 | 1.1859 | 0.000496 | CLN6 |
| 80085567 | 80086473 | chr17 | − | exo | hsa_circ_0046377 | 1.4679 | 0.000499 | CCDC57 |
| 99220660 | 99233376 | chr9 | + | exo | hsa_circ_0087631 | 1.3804 | 0.000508 | HABP4 |
| 76886266 | 77067234 | chr18 | + | exo | hsa_circ_0048009 | 0.5867 | 0.000518 | ATP9B |
| 115636279 | 115644139 | chr10 | + | exo | | 0.7899 | 0.000518 | NHLRC2 |
| 11258493 | 11258789 | chr19 | − | s/o | hsa_circ_0007223 | 1.2275 | 0.000520 | SPC24 |
| 41650311 | 41677086 | chr22 | − | exo | hsa_circ_0063526 | 1.6944 | 0.000521 | RANGAP1 |
| 24056373 | 24057381 | chr22 | − | exo | hsa_circ_0001212 | 3.0590 | 0.000542 | GUSBP11 |
| 47920060 | 47920607 | chr19 | − | exo | hsa_circ_0051685 | 1.7058 | 0.000555 | MEIS3 |
| 9792628 | 9793633 | chr3 | + | exo | hsa_circ_0008027 | 1.4640 | 0.000567 | OGG1 |
| 13532533 | 13581264 | chr20 | − | int | | 0.3384 | 0.000573 | TASP1 |
| 121220457 | 121222396 | chr12 | − | exo | hsa_circ_0003472 | 0.7490 | 0.000575 | SPPL3 |
| 67283695 | 67293141 | chrX | − | exo | | 1.6142 | 0.000576 | OPHN1 |
| 67942582 | 67943609 | chr16 | + | exo | | 1.4808 | 0.000580 | PSKH1 |
| 18805920 | 18806903 | chr16 | − | exo | hsa_circ_0038230 | 1.6317 | 0.000596 | ARL6IP1 |
| 151719232 | 151738409 | chr4 | − | exo | hsa_circ_0008618 | 0.4593 | 0.000601 | LRBA |
| 37583953 | 37584043 | chr17 | − | exo | hsa_circ_0043438 | 0.3472 | 0.000609 | MED1 |
| 152480271 | 152498816 | chr7 | + | exo | hsa_circ_0006890 | 0.6492 | 0.000609 | ACTR3B |
| 46225531 | 46234019 | chr21 | − | exo | hsa_circ_0061976 | 1.3765 | 0.000612 | SUMO3 |
| 78267015 | 78280916 | chr1 | + | exo | hsa_circ_0002570 | 0.5871 | 0.000615 | FAM73A |
| 30801548 | 30857928 | chr13 | − | exo | hsa_circ_0008068 | 0.4199 | 0.000620 | KATNAL1 |
| 76060802 | 76071359 | chr17 | + | exo | | 1.3041 | 0.000632 | TNRC6C |
| 37283582 | 37284623 | chr2 | − | exo | hsa_circ_0054086 | 0.7205 | 0.000636 | HEATR5B |
| 125639749 | 125659855 | chr9 | − | exo | hsa_circ_0088390 | 0.6258 | 0.000664 | RC3H2 |
| 15219972 | 15220075 | chr19 | + | s/o | hsa_circ_0000900 | 1.1689 | 0.000671 | SYDE1 |
| 40409395 | 40486735 | chr12 | − | s/o | hsa_circ_0000391 | 0.8019 | 0.000675 | SLC2A13 |
| 15480588 | 15531345 | chr8 | + | exo | hsa_circ_0083390 | 0.6281 | 0.000676 | TUSC3 |
| 180962498 | 180974599 | chr1 | − | exo | hsa_circ_0000165 | 0.8529 | 0.000693 | STX6 |
| 5700799 | 5706003 | chr19 | − | exo | hsa_circ_0048775 | 1.3992 | 0.000693 | LONP1 |
| 42331129 | 42333998 | chr20 | + | exo | hsa_circ_0006332 | 1.4701 | 0.000703 | MYBL2 |
| 41036244 | 41037457 | chr15 | − | exo | hsa_circ_0004942 | 1.6307 | 0.000703 | RMDN3 |
| 66642211 | 66643906 | chr16 | + | exo | hsa_circ_0008450 | 1.3999 | 0.000707 | CMTM3 |
| 98513345 | 98515302 | chr7 | + | exo | hsa_circ_0081258 | 1.2232 | 0.000720 | TRRAP |
| 15456270 | 15456465 | chr21 | + | exo | hsa_circ_0061265 | 1.2511 | 0.000722 | BC048201 |
| 151960100 | 152012423 | chr7 | − | exo | hsa_circ_0006784 | 0.6990 | 0.000730 | KMT2C |
| 860660 | 860741 | chr19 | + | s/o | hsa_circ_0000864 | 2.4602 | 0.000738 | CFD |
| 36248634 | 36248980 | chr11 | + | exo | hsa_circ_0006988 | 1.9736 | 0.000742 | LDLRAD3 |
| 34389412 | 34459751 | chr20 | + | exo | hsa_circ_0060144 | 0.4169 | 0.000749 | PHF20 |
| 126519981 | 126531842 | chr9 | − | exo | hsa_circ_0088474 | 1.3275 | 0.000760 | DENND1A |
| 72341954 | 72425114 | chr16 | − | s/o | | 0.8223 | 0.000762 | LINC01572 |
| 20715320 | 20789607 | chr9 | + | exo | | 0.7665 | 0.000765 | FOCAD |
| 125032151 | 125042288 | chr3 | − | exo | hsa_circ_0007557 | 0.7301 | 0.000779 | ZNF148 |
| 76173360 | 76187046 | chr14 | + | exo | hsa_circ_0032704 | 0.2592 | 0.000792 | TTLL5 |
| 99695763 | 99695845 | chr7 | − | int | hsa_circ_0001728 | 1.2837 | 0.000799 | MCM7 |
| 54926056 | 54933986 | chr17 | + | exo | hsa_circ_0000786 | 0.8435 | 0.000799 | DGKE |
| 44788577 | 44789336 | chr15 | + | exo | hsa_circ_0007705 | 0.3747 | 0.000804 | CTDSPL2 |
| 39138266 | 39219794 | chr19 | + | exo | hsa_circ_0050898 | 2.5652 | 0.000805 | ACTN4 |
| 63719709 | 63720311 | chr11 | + | exo | hsa_circ_0022623 | 1.4156 | 0.000807 | NAA40 |
| 94645539 | 94649052 | chr9 | − | exo | | 0.7435 | 0.000825 | ROR2 |
| 27345636 | 27351594 | chr16 | + | s/o | hsa_circ_0004939 | 0.6130 | 0.000831 | IL4R |
| 31116189 | 31116505 | chr6 | − | s/o | | 1.3096 | 0.000845 | CCHCR1 |
| 8866406 | 8906689 | chr12 | + | exo | hsa_circ_0025373 | 1.2180 | 0.000849 | RIMKLB |
| 8732391 | 8734282 | chr11 | − | exo | hsa_circ_0005944 | 1.4480 | 0.000852 | ST5 |
| 325634 | 336173 | chr19 | − | exo | hsa_circ_0002005 | 1.3128 | 0.000855 | MIER2 |
| 36245469 | 36246795 | chr19 | − | exo | hsa_circ_0050648 | 0.3904 | 0.000870 | HSPB6 |
| 56598731 | 56632649 | chr19 | − | exo | hsa_circ_0052282 | 2.2070 | 0.000878 | ZNF787 |
| 100799874 | 100800766 | chr7 | + | exo | | 1.5786 | 0.000884 | AP1S1 |
| 129304794 | 129305562 | chr3 | − | exo | | 1.2494 | 0.000897 | PLXND1 |
| 114860749 | 114875148 | chr9 | − | exo | hsa_circ_0088046 | 0.5601 | 0.000899 | SUSD1 |
| 125932180 | 125946577 | chr9 | − | exo | hsa_circ_0001888 | 1.3136 | 0.000909 | STRBP |
| 97373450 | 97400263 | chr2 | − | exo | | 1.3080 | 0.000921 | LMAN2L |
| 156220621 | 156220731 | chr1 | − | int | hsa_circ_0000141 | 1.1751 | 0.000931 | SMG5 |
| 56821323 | 56822377 | chr20 | − | int | | 0.7782 | 0.000938 | PPP4R1L |
| 15212782 | 15219664 | chr16 | − | exo | hsa_circ_0038032 | 0.3918 | 0.000947 | LOC399491 |
| 131102948 | 131104197 | chr2 | + | exo | hsa_circ_0056459 | 1.1344 | 0.000947 | IMP4 |
| 44684925 | 44687358 | chr7 | + | exo | hsa_circ_0003340 | 1.5092 | 0.000977 | OGDH |
| 90103642 | 90103807 | chr16 | + | s/o | | 1.1720 | 0.000982 | GAS8 |
| 39328342 | 39329036 | chr19 | − | int | | 1.0945 | 0.000983 | HNRNPL |
| 93182480 | 93183806 | chr14 | − | exo | hsa_circ_0033010 | 1.2355 | 0.000984 | LGMN |
| 42331129 | 42331543 | chr20 | + | exo | hsa_circ_0060461 | 1.4027 | 0.000987 | MYBL2 |
| 79977183 | 79977257 | chr17 | − | exo | hsa_circ_0046298 | 1.2675 | 0.000987 | STRA13 |
| 113869721 | 113869956 | chr13 | + | int | hsa_circ_0000507 | 1.1525 | 0.000987 | CUL4A |
| 62123796 | 62124616 | chr11 | + | exo | | 1.1779 | 0.000990 | ASRGL1 |
| 83372195 | 83378191 | chr4 | + | exo | | 1.2607 | 0.001010 | ENOPH1 |
| 47155365 | 47168153 | chr3 | − | exo | hsa_circ_0065173 | 1.1359 | 0.001016 | SETD2 |
| 118544152 | 118544325 | chrX | + | exo | hsa_circ_0006789 | 0.5457 | 0.001021 | SLC25A43 |
| 992502 | 994245 | chr11 | + | exo | hsa_circ_0020748 | 1.2722 | 0.001021 | AP2A2 |
| 27453132 | 27465643 | chr3 | − | exo | hsa_circ_0002901 | 0.4728 | 0.001023 | SLC4A7 |
| 90800369 | 90815443 | chr15 | + | exo | hsa_circ_0036783 | 2.0161 | 0.001053 | TTLL13 |
| 53331118 | 53331571 | chr14 | − | exo | hsa_circ_0031968 | 0.3778 | 0.001063 | FERMT2 |
| 28181070 | 28181230 | chr16 | − | exo | hsa_circ_0038821 | 1.2384 | 0.001076 | XPO6 |
| 49713126 | 49720934 | chr3 | + | exo | hsa_circ_0065673 | 1.2618 | 0.001079 | APEH |
| 99166819 | 99194903 | chr12 | − | exo | | 0.3990 | 0.001103 | ANKS1B |
| 139756570 | 139757900 | chr9 | − | exo | hsa_circ_0089592 | 1.1183 | 0.001108 | EDF1 |
| 19570107 | 19570509 | chr1 | − | exo | hsa_circ_0010389 | 1.2023 | 0.001110 | EMC1 |
| 11394061 | 11400811 | chr11 | − | exo | | 1.2456 | 0.001113 | GALNT18 |
| 153791266 | 153792211 | chr1 | − | exo | | 1.3051 | 0.001123 | GATAD2B |
| 20811305 | 20811436 | chr14 | − | s/o | hsa_circ_0000514 | 2.8163 | 0.001124 | RPPH1 |
| 103352525 | 103363738 | chr14 | + | exo | hsa_circ_0000570 | 1.2811 | 0.001130 | TRAF3 |
| 111746088 | 111746330 | chr12 | + | exo | | 1.8383 | 0.001132 | CUX2 |
| 24679553 | 24681035 | chr14 | − | exo | hsa_circ_0031357 | 1.3692 | 0.001147 | CHMP4A |
| 196129822 | 196134264 | chr3 | − | exo | hsa_circ_0068655 | 2.0199 | 0.001147 | UBXN7 |
| 46275124 | 46285362 | chr21 | − | exo | hsa_circ_0061985 | 1.3230 | 0.001150 | PTTG1IP |
| 42470415 | 42470768 | chr15 | − | exo | hsa_circ_0034786 | 0.4130 | 0.001159 | VPS39 |
| 50285026 | 50299637 | chr13 | − | exo | hsa_circ_0000486 | 1.1126 | 0.001170 | KPNA3 |
| 64495280 | 64508912 | chr15 | − | exo | hsa_circ_0001955 | 0.2896 | 0.001171 | CSNK1G1 |
| 85595808 | 85596953 | chr2 | + | exo | hsa_circ_0008059 | 1.2439 | 0.001180 | ELMOD3 |
| 88553890 | 88554628 | chr12 | + | exo | hsa_circ_0027691 | 0.7895 | 0.001191 | TMTC3 |
| 23735156 | 23745632 | chr1 | − | exo | | 1.5937 | 0.001195 | TCEA3 |
| 54867533 | 54870603 | chr1 | − | exo | | 2.0906 | 0.001198 | SSBP3 |
| 22696395 | 22777499 | chr11 | + | exo | | 0.2204 | 0.001201 | GAS2 |
| 353844 | 376831 | chr5 | + | exo | hsa_circ_0005694 | 1.2721 | 0.001201 | AHRR |
| 27440257 | 27444215 | chr2 | + | exo | hsa_circ_0053173 | 1.1955 | 0.001205 | CAD |
| 120285606 | 120286575 | chr1 | − | as | hsa_circ_0000121 | 0.5072 | 0.001211 | PHGDH |
| 36681395 | 36681695 | chr22 | − | int | hsa_circ_0092283 | 0.5657 | 0.001211 | MYH9 |
| 17387303 | 17387718 | chr19 | + | exo | hsa_circ_0003253 | 1.5413 | 0.001213 | BABAM1 |
| 78115117 | 78115647 | chr17 | − | exo | hsa_circ_0045989 | 1.2950 | 0.001215 | EIF4A3 |
| 34918468 | 34923339 | chr5 | + | exo | hsa_circ_0006970 | 1.1397 | 0.001221 | BRIX1 |
| 155547476 | 155551830 | chr3 | − | exo | hsa_circ_0001350 | 0.7999 | 0.001228 | SLC33A1 |
| 55282872 | 55287883 | chr18 | − | exo | hsa_circ_0047771 | 0.2548 | 0.001233 | NARS |
| 17907047 | 17945940 | chr1 | + | exo | hsa_circ_0010206 | 1.4329 | 0.001234 | ARHGEF10L |
| 25328542 | 25339121 | chr15 | + | exo | | 1.6875 | 0.001236 | SNURF-SNRPN |
| 4947116 | 4951008 | chr19 | + | exo | | 1.6594 | 0.001237 | UHRF1 |
| 27151596 | 27151827 | chr8 | − | exo | hsa_circ_0083756 | 1.4317 | 0.001238 | TRIM35 |
| 138979956 | 139003046 | chr5 | + | exo | hsa_circ_0004503 | 1.3886 | 0.001239 | UBE2D2 |
| 108565957 | 108622441 | chr4 | − | exo | hsa_circ_0070610 | 0.5808 | 0.001240 | PAPSS1 |
| 155547476 | 155560408 | chr3 | − | exo | hsa_circ_0067772 | 0.7097 | 0.001248 | SLC33A1 |
| 45781180 | 45781873 | chr19 | + | exo | hsa_circ_0000941 | 1.2729 | 0.001259 | MARK4 |
| 149246283 | 149250107 | chr7 | − | exo | | 0.5205 | 0.001266 | ZNF767P |
| 34864900 | 34867294 | chr17 | − | exo | hsa_circ_0003507 | 1.4555 | 0.001279 | MYO19 |
| 15860731 | 15873366 | chr1 | + | exo | | 0.6604 | 0.001294 | DNAJC16 |
| 23615267 | 23615961 | chr22 | + | exo | hsa_circ_0001211 | 1.2657 | 0.001309 | BCR |
| 1158623 | 1164326 | chr1 | − | exo | hsa_circ_0009244 | 1.3982 | 0.001310 | SDF4 |
| 33272082 | 33276658 | chr1 | − | exo | hsa_circ_0011448 | 1.5130 | 0.001312 | YARS |
| 115024714 | 115060196 | chr9 | − | exo | hsa_circ_0008192 | 0.3948 | 0.001326 | PTBP3 |
| 235747039 | 235758811 | chr1 | − | exo | hsa_circ_0017076 | 1.1358 | 0.001335 | GNG4 |
| 810107 | 814799 | chr7 | + | exo | hsa_circ_0001667 | 1.4178 | 0.001337 | DNAAF5 |
| 24989150 | 24989715 | chr1 | + | exo | hsa_circ_0010921 | 1.8258 | 0.001338 | SRRM1 |
| 6657590 | 6657991 | chr12 | − | exo | hsa_circ_0000375 | 1.4580 | 0.001339 | IFFO1 |
| 34978930 | 35006275 | chr11 | + | exo | hsa_circ_0003768 | 0.6064 | 0.001357 | PDHX |
| 115837905 | 115840678 | chr5 | − | exo | hsa_circ_0073647 | 0.2040 | 0.001367 | SEMA6A |
| 76134888 | 76180068 | chr13 | + | s/o | hsa_circ_0030433 | 0.8431 | 0.001368 | UCHL3 |
| 72769034 | 72769178 | chr17 | − | exo | hsa_circ_0045564 | 1.2221 | 0.001383 | NAT9 |
| 29669729 | 29674205 | chr22 | + | exo | hsa_circ_0062746 | 2.1539 | 0.001388 | EWSR1 |
| 19358063 | 19359646 | chr18 | + | exo | hsa_circ_0008190 | 0.7287 | 0.001390 | MIB1 |
| 88061088 | 88071617 | chr16 | + | exo | hsa_circ_0040823 | 1.8961 | 0.001393 | BANP |
| 3726429 | 3733958 | chr11 | − | exo | | 0.3499 | 0.001417 | NUP98 |
| 10694281 | 10694746 | chr19 | − | exo | hsa_circ_0049282 | 0.4496 | 0.001424 | AP1M2 |
| 5998341 | 6002530 | chr10 | − | exo | | 0.6241 | 0.001449 | IL15RA |
| 38341277 | 38351497 | chr1 | − | exo | | 1.1325 | 0.001449 | INPP5B |
| 110820647 | 110834257 | chr12 | − | exo | hsa_circ_0028190 | 1.1124 | 0.001472 | ANAPC7 |
| 118544152 | 118560317 | chrX | + | exo | hsa_circ_0006727 | 0.4736 | 0.001483 | SLC25A43 |
| 15616097 | 15616181 | chr5 | + | s/o | | 1.9196 | 0.001488 | FBXL7 |
| 33825619 | 33840156 | chr21 | + | exo | hsa_circ_0061481 | 1.1512 | 0.001497 | EVA1C |
| 11958205 | 12016681 | chr17 | + | s/o | | 0.7927 | 0.001508 | MAP2K4 |
| 20811282 | 20811431 | chr14 | − | s/o | hsa_circ_0000511 | 3.0392 | 0.001508 | RPPH1 |
| 15766546 | 15771806 | chr16 | + | exo | | 1.1734 | 0.001515 | NDE1 |
| 36282482 | 36292436 | chr1 | + | exo | hsa_circ_0003986 | 0.6691 | 0.001528 | AGO4 |
| 3977580 | 3978020 | chr19 | − | int | hsa_circ_0000874 | 1.4046 | 0.001541 | EEF2 |
| 8093409 | 8095466 | chr6 | − | int | | 1.1028 | 0.001560 | EEF1E1 |
| 95549330 | 95550574 | chr8 | − | exo | hsa_circ_0006896 | 1.5944 | 0.001563 | KIAA1429 |
| 5047486 | 5047680 | chr19 | + | exo | hsa_circ_0048695 | 1.3424 | 0.001565 | KDM4B |
| 48209335 | 48219478 | chr3 | − | exo | hsa_circ_0005467 | 1.1811 | 0.001571 | CDC25A |
| 26364067 | 26388013 | chr4 | + | exo | | 2.1420 | 0.001572 | RBPJ |
| 41961025 | 41962156 | chr15 | + | exo | hsa_circ_0000591 | 0.6266 | 0.001574 | MGA |
| 91229394 | 91234198 | chr4 | + | exo | | 0.7086 | 0.001576 | CCSER1 |
| 13035452 | 13039661 | chr19 | − | exo | hsa_circ_0049627 | 1.3496 | 0.001586 | FARSA |
| 27794162 | 27809306 | chr17 | + | exo | | 0.6238 | 0.001590 | TAOK1 |
| 75425277 | 75448593 | chr16 | − | s/o | | 1.3140 | 0.001597 | CFDP1 |
| 25402743 | 25402983 | chr12 | + | as | hsa_circ_0000383 | 1.7081 | 0.001604 | KRAS |
| 41738532 | 41739580 | chr22 | + | exo | hsa_circ_0001236 | 1.4704 | 0.001607 | ZC3H7B |
| 79813017 | 79813462 | chr17 | − | exo | hsa_circ_0046263 | 2.0533 | 0.001621 | P4HB |
| 152497615 | 152498816 | chr7 | + | exo | hsa_circ_0083061 | 0.7451 | 0.001622 | ACTR3B |
| 15601324 | 15651474 | chr2 | − | exo | hsa_circ_0052767 | 0.4849 | 0.001637 | NBAS |
| 74927307 | 74929309 | chr15 | − | s/o | hsa_circ_0000632 | 1.1102 | 0.001647 | EDC3 |
| 125557491 | 125558715 | chr10 | − | exo | hsa_circ_0020302 | 1.1386 | 0.001653 | CPXM2 |
| 45241972 | 45242292 | chr1 | + | int | hsa_circ_0092275 | 0.7468 | 0.001654 | RPS8 |
| 38894089 | 38894578 | chr22 | − | exo | hsa_circ_0063329 | 1.2615 | 0.001663 | DDX17 |
| 175740693 | 175751760 | chr5 | + | exo | hsa_circ_0075094 | 1.1590 | 0.001668 | SIMC1 |
| 115013208 | 115015068 | chr9 | − | exo | hsa_circ_0088072 | 0.7012 | 0.001670 | PTBP3 |
| 85657103 | 85664245 | chr15 | + | exo | hsa_circ_0036629 | 0.7404 | 0.001670 | PDE8A |
| 5568791 | 5570232 | chr7 | − | exo | hsa_circ_0079276 | 2.3387 | 0.001674 | ACTB |
| 152913372 | 152916781 | chrX | + | exo | hsa_circ_0091751 | 1.1000 | 0.001675 | DUSP9 |
| 43226421 | 43227255 | chr20 | + | int | | 1.8499 | 0.001689 | PKIG |
| 24164288 | 24200931 | chr13 | + | exo | hsa_circ_0008784 | 1.9023 | 0.001689 | TNFRSF19 |
| 140669560 | 140676849 | chr9 | + | exo | hsa_circ_0004073 | 1.1802 | 0.001694 | EHMT1 |
| 2191265 | 2192802 | chr9 | + | s/o | hsa_circ_0006082 | 1.2691 | 0.001727 | SMARCA2 |
| 81155190 | 81157389 | chr16 | − | exo | | 1.8357 | 0.001731 | PKD1L2 |
| 120592773 | 120613015 | chr12 | − | exo | hsa_circ_0028799 | 1.5454 | 0.001740 | GCN1L1 |
| 43139929 | 43142681 | chr20 | − | exo | hsa_circ_0060491 | 0.8057 | 0.001751 | SERINC3 |
| 35207238 | 35219339 | chr15 | − | exo | | 0.8761 | 0.001751 | AQR |
| 99416041 | 99424741 | chr10 | + | exo | hsa_circ_0019376 | 1.1247 | 0.001759 | PI4K2A |
| 33163212 | 33163532 | chr11 | − | exo | | 1.2502 | 0.001759 | CSTF3 |
| 29904598 | 29911710 | chr12 | − | exo | hsa_circ_0025767 | 1.2661 | 0.001764 | TMTC1 |
| 20758088 | 20770237 | chr9 | + | exo | hsa_circ_0005572 | 0.5883 | 0.001776 | FOCAD |
| 34952950 | 35006275 | chr11 | + | exo | hsa_circ_0021708 | 0.5767 | 0.001791 | PDHX |
| 19823348 | 19839743 | chr17 | − | exo | | 0.3552 | 0.001795 | AKAP10 |
| 43294157 | 43299077 | chr5 | − | exo | hsa_circ_0072389 | 1.9573 | 0.001799 | HMGCS1 |
| 70301561 | 70302282 | chr16 | − | exo | hsa_circ_0040203 | 1.2737 | 0.001819 | AARS |
| 4700732 | 4701790 | chr17 | + | exo | hsa_circ_0041597 | 1.9523 | 0.001821 | PSMB6 |
| 1089470 | 1089961 | chr19 | − | exo | hsa_circ_0048164 | 1.4808 | 0.001828 | POLR2E |
| 58436990 | 58441922 | chr19 | − | exo | | 1.1464 | 0.001831 | ZNF418 |
| 123632934 | 123636251 | chr9 | − | int | | 1.1492 | 0.001833 | PHF19 |
| 42915719 | 42976365 | chr22 | − | s/o | hsa_circ_0001243 | 1.8209 | 0.001853 | RRP7A |
| 13228047 | 13230425 | chr6 | + | exo | hsa_circ_0002268 | 1.3532 | 0.001873 | PHACTR1 |
| 131071878 | 131084192 | chr7 | + | exo | hsa_circ_0001747 | 0.6969 | 0.001875 | MKLN1 |
| 70255936 | 70260758 | chr11 | + | exo | hsa_circ_0023336 | 1.2436 | 0.001878 | CTTN |
| 71693806 | 71701763 | chr11 | + | exo | hsa_circ_0023409 | 1.1856 | 0.001910 | RNF121 |
| 77193578 | 77211816 | chr18 | + | exo | | 2.2192 | 0.001936 | NFATC1 |
| 18768781 | 18769265 | chr17 | + | exo | hsa_circ_0002278 | 0.7626 | 0.001936 | PRPSAP2 |
| 74988220 | 74988302 | chr15 | − | int | hsa_circ_0000634 | 1.4498 | 0.001941 | EDC3 |
| 114860749 | 114864565 | chr9 | − | exo | hsa_circ_0088045 | 0.5646 | 0.001942 | SUSD1 |
| 43591211 | 43607219 | chr3 | − | exo | hsa_circ_0009084 | 0.5839 | 0.001956 | ANO10 |
| 105131084 | 105131167 | chr14 | − | s/o | hsa_circ_0000574 | 0.5558 | 0.001957 | RP11-982M15.8 |
| 110764194 | 110780253 | chr12 | + | exo | hsa_circ_0028173 | 1.5278 | 0.001969 | ATP2A2 |
| 1090906 | 1091906 | chr19 | − | exo | hsa_circ_0048171 | 1.3102 | 0.001974 | POLR2E |
| 77830227 | 77835260 | chr11 | − | exo | hsa_circ_0023787 | 0.8238 | 0.001975 | ALG8 |
| 47466974 | 47476627 | chr3 | − | exo | hsa_circ_0065214 | 1.8386 | 0.001981 | SCAP |
| 30601792 | 30602041 | chr11 | − | exo | hsa_circ_0021554 | 1.3569 | 0.001989 | MPPED2 |
| 42284534 | 42284717 | chr2 | + | int | | 1.4206 | 0.001995 | PKDCC |
| 7409610 | 7423911 | chr10 | − | exo | hsa_circ_0017648 | 0.6042 | 0.001996 | SFMBT2 |
| 56676203 | 56680429 | chr12 | − | exo | hsa_circ_0026983 | 1.4527 | 0.002003 | CS |
| 155644800 | 155649303 | chr1 | − | exo | hsa_circ_0003608 | 1.5824 | 0.002011 | YY1AP1 |
| 89202707 | 89212627 | chr14 | − | exo | hsa_circ_0032879 | 0.8348 | 0.002018 | EML5 |
| 19627435 | 19663412 | chr16 | + | exo | hsa_circ_0005699 | 0.3901 | 0.002027 | C16orf62 |
| 70245970 | 70246161 | chr14 | + | as | hsa_circ_0000550 | 1.1747 | 0.002043 | SLC10A1 |
| 29643162 | 29643836 | chr6 | − | exo | | 0.4464 | 0.002044 | ZFP57 |
| 69206201 | 69206389 | chr5 | + | exo | hsa_circ_0072805 | 2.5120 | 0.002051 | SMA4 |
| 154736558 | 154754293 | chrX | − | exo | hsa_circ_0007183 | 0.8244 | 0.002060 | TMLHE |
| 16044387 | 16047883 | chr1 | + | exo | hsa_circ_0000021 | 1.2951 | 0.002061 | PLEKHM2 |
| 73038275 | 73038754 | chr17 | − | exo | hsa_circ_0006942 | 1.2307 | 0.002066 | ATP5H |
| 1872300 | 1873038 | chr16 | − | exo | hsa_circ_0037353 | 1.2165 | 0.002067 | HAGH |
| 11548696 | 11548945 | chr19 | + | exo | hsa_circ_0049462 | 1.5716 | 0.002068 | PRKCSH |
| 20924072 | 20925019 | chr14 | + | exo | hsa_circ_0000521 | 1.3598 | 0.002070 | APEX1 |
| 36914809 | 36915584 | chr22 | − | exo | hsa_circ_0006385 | 1.3328 | 0.002078 | EIF3D |
| 44750998 | 44751217 | chr20 | + | int | | 1.2648 | 0.002078 | CD40 |
| 154548756 | 154553990 | chr4 | + | exo | hsa_circ_0071312 | 0.3074 | 0.002110 | KIAA0922 |
| 151174418 | 151188100 | chr7 | − | exo | hsa_circ_0082950 | 1.5286 | 0.002130 | RHEB |
| 128768965 | 128788867 | chr10 | + | exo | hsa_circ_0007142 | 0.7800 | 0.002136 | DOCK1 |
| 41211000 | 41211352 | chr19 | − | exo | | 1.3090 | 0.002139 | ADCK4 |
| 72020039 | 72022817 | chr12 | − | exo | hsa_circ_0008912 | 0.8443 | 0.002141 | ZFC3H1 |
| 79652634 | 79655857 | chr17 | + | exo | hsa_circ_0046215 | 1.1068 | 0.002147 | HGS |
| 89628179 | 89628539 | chr16 | + | int | hsa_circ_0092337 | 2.9248 | 0.002161 | RPL13 |
| 134032815 | 134039622 | chr5 | + | exo | hsa_circ_0073993 | 0.7776 | 0.002174 | SEC24A |
| 53672262 | 53707139 | chrX | − | exo | hsa_circ_0002724 | 1.2739 | 0.002195 | HUWE1 |
| 66761590 | 66763002 | chr16 | − | exo | hsa_circ_0008222 | 1.3343 | 0.002199 | DYNC1LI2 |
| 18805920 | 18810156 | chr16 | − | exo | | 2.2273 | 0.002207 | ARL6IP1 |
| 11047480 | 11049621 | chr21 | − | exo | hsa_circ_0061260 | 0.7820 | 0.002221 | BAGE4 |
| 50341265 | 50351184 | chrX | − | exo | hsa_circ_0090585 | 1.1476 | 0.002225 | SHROOM4 |
| 79186178 | 79188586 | chr4 | + | exo | hsa_circ_0070113 | 1.3659 | 0.002239 | FRAS1 |
| 76134888 | 76143643 | chr13 | + | exo | hsa_circ_0000494 | 0.7893 | 0.002240 | UCHL3 |
| 241463301 | 241468926 | chr2 | − | exo | hsa_circ_0004474 | 1.1725 | 0.002242 | ANKMY1 |
| 81385187 | 81411307 | chr16 | + | s/o | hsa_circ_0003857 | 0.5563 | 0.002244 | GAN |
| 88598621 | 88635842 | chr10 | + | exo | hsa_circ_0006675 | 0.6521 | 0.002245 | BMPR1A |
| 117423010 | 117426674 | chr12 | + | exo | hsa_circ_0006150 | 1.4274 | 0.002266 | FBXW8 |
| 72102292 | 72121116 | chr4 | + | exo | hsa_circ_0069922 | 1.1601 | 0.002276 | SLC4A4 |
| 100457506 | 100459533 | chr7 | + | exo | hsa_circ_0081536 | 1.3049 | 0.002278 | SLC12A9 |
| 35578644 | 35583373 | chr17 | − | exo | hsa_circ_0043244 | 0.8379 | 0.002281 | ACACA |
| 9814016 | 9845688 | chr18 | + | exo | hsa_circ_0046900 | 1.2565 | 0.002287 | RAB31 |
| 58616696 | 58617086 | chr16 | − | exo | hsa_circ_0039697 | 0.7321 | 0.002289 | CNOT1 |
| 140074632 | 140074836 | chr9 | − | exo | hsa_circ_0003129 | 1.1931 | 0.002291 | ANAPC2 |
| 101389973 | 101391057 | chr3 | − | exo | hsa_circ_0003179 | 0.8764 | 0.002308 | ZBTB11 |
| 17972481 | 17972901 | chr19 | + | int | hsa_circ_0092327 | 0.4379 | 0.002319 | RPL18A |
| 22154945 | 22155327 | chr1 | − | int | | 0.5183 | 0.002339 | HSPG2 |
| 89484691 | 89492069 | chr16 | − | s/o | hsa_circ_0000726 | 1.7259 | 0.002356 | ANKRD11 |
| 74100546 | 74100928 | chr10 | − | exo | hsa_circ_0008662 | 1.4526 | 0.002368 | DNAJB12 |
| 53585106 | 53590604 | chr12 | − | exo | hsa_circ_0026568 | 2.4711 | 0.002371 | ITGB7 |
| 6995260 | 7003512 | chrX | − | int | | 0.7002 | 0.002374 | HDHD1 |
| 65307715 | 65307853 | chr11 | − | exo | hsa_circ_0000327 | 1.2300 | 0.002391 | LTBP3 |
| 110826316 | 110834257 | chr12 | − | exo | hsa_circ_0028196 | 0.2801 | 0.002430 | ANAPC7 |
| 11230767 | 11231198 | chr19 | + | exo | hsa_circ_0006877 | 1.6372 | 0.002445 | LDLR |
| 70534860 | 70546335 | chr16 | − | exo | | 1.1998 | 0.002454 | COG4 |
| 20907148 | 20933102 | chr9 | + | exo | hsa_circ_0086564 | 0.7043 | 0.002459 | FOCAD |
| 89638703 | 89650899 | chr6 | − | exo | hsa_circ_0077296 | 0.6018 | 0.002461 | RNGTT |
| 54717288 | 54717533 | chr1 | − | exo | hsa_circ_0012650 | 1.2583 | 0.002462 | SSBP3 |
| 35065247 | 35068359 | chr9 | − | exo | hsa_circ_0086779 | 1.4741 | 0.002465 | VCP |
| 36867201 | 36870210 | chr6 | + | exo | hsa_circ_0004764 | 1.1868 | 0.002469 | C6orf89 |
| 6350782 | 6351078 | chr17 | + | exo | hsa_circ_0041732 | 1.0741 | 0.002486 | FAM64A |
| 2137009 | 2138713 | chr19 | − | exo | hsa_circ_0048344 | 1.3715 | 0.002490 | AP3D1 |
| 2404006 | 2404164 | chr7 | + | exo | hsa_circ_0079166 | 1.4170 | 0.002502 | EIF3B |
| 91947919 | 91957146 | chr14 | − | exo | hsa_circ_0032965 | 0.8089 | 0.002531 | SMEK1 |
| 68018139 | 68028357 | chr8 | + | exo | hsa_circ_0001806 | 0.4256 | 0.002537 | CSPP1 |
| 3623685 | 3624160 | chr19 | − | exo | hsa_circ_0002661 | 1.5687 | 0.002539 | CACTIN |
| 202724398 | 202729701 | chr1 | − | exo | hsa_circ_0016040 | 0.7855 | 0.002543 | KDM5B |
| 25282854 | 25290211 | chr20 | − | exo | hsa_circ_0059660 | 1.1358 | 0.002551 | ABHD12 |
| 25644553 | 25645709 | chr1 | − | int | | 0.3967 | 0.002552 | C1orf63 |
| 45883610 | 45893784 | chr11 | + | exo | hsa_circ_0000295 | 1.2547 | 0.002557 | CRY2 |
| 127337917 | 127341124 | chr3 | − | as | hsa_circ_0001334 | 1.7019 | 0.002566 | MCM2 |
| 63042932 | 63052295 | chr1 | − | exo | hsa_circ_0002032 | 0.7327 | 0.002569 | DOCK7 |
| 18809246 | 18810156 | chr16 | − | exo | hsa_circ_0004833 | 1.9745 | 0.002586 | ARL6IP1 |
| 24190831 | 24197887 | chrX | + | exo | hsa_circ_0007108 | 0.6828 | 0.002591 | ZFX |
| 63951934 | 63952135 | chr15 | − | exo | hsa_circ_0035729 | 0.8970 | 0.002597 | HERC1 |
| 71712657 | 71715808 | chr16 | − | exo | hsa_circ_0000715 | 0.7258 | 0.002603 | PHLPP2 |
| 74095537 | 74100928 | chr10 | − | exo | hsa_circ_0018746 | 1.2648 | 0.002611 | DNAJB12 |
| 68115314 | 68133170 | chr11 | + | exo | hsa_circ_0023182 | 1.1263 | 0.002638 | LRP5 |
| 114904599 | 114919893 | chr9 | − | exo | hsa_circ_0088058 | 0.7687 | 0.002655 | SUSD1 |
| 231506307 | 231509845 | chr1 | − | exo | hsa_circ_0000196 | 0.9060 | 0.002672 | EGLN1 |
| 153735203 | 153737539 | chr1 | + | exo | hsa_circ_0006939 | 1.1893 | 0.002672 | INTS3 |
| 23530292 | 23530831 | chr14 | − | exo | | 1.1995 | 0.002679 | ACIN1 |
| 110036280 | 110064975 | chr6 | + | exo | hsa_circ_0077607 | 0.5112 | 0.002710 | FIG4 |
| 44467984 | 44468657 | chr1 | − | exo | hsa_circ_0012133 | 1.7922 | 0.002717 | SLC6A9 |
| 108136973 | 108140201 | chr12 | − | exo | hsa_circ_0007468 | 0.9067 | 0.002722 | PRDM4 |
| 27977671 | 28030080 | chr17 | − | exo | hsa_circ_0042806 | 0.6762 | 0.002724 | SSH2 |
| 25622036 | 25622213 | chr3 | + | exo | hsa_circ_0064584 | 1.1493 | 0.002738 | RARB |
| 44128265 | 44131942 | chr19 | − | exo | | 1.7911 | 0.002747 | CADM4 |
| 63881857 | 63888812 | chr14 | − | exo | hsa_circ_0032154 | 0.2653 | 0.002755 | PPP2R5E |
| 13214375 | 13214765 | chr10 | + | exo | hsa_circ_0002109 | 1.1981 | 0.002762 | MCM10 |
| 129662158 | 129679387 | chr7 | − | exo | hsa_circ_0082319 | 1.2061 | 0.002762 | ZC3HC1 |
| 99589361 | 99607298 | chr9 | − | exo | | 0.8622 | 0.002765 | ZNF782 |
| 22142545 | 22162135 | chr22 | − | exo | hsa_circ_0062439 | 0.8066 | 0.002770 | MAPK1 |
| 29756705 | 29756841 | chr16 | + | s/o | hsa_circ_0000687 | 1.2344 | 0.002779 | BC041466 |
| 87764157 | 87788898 | chr16 | − | exo | hsa_circ_0040787 | 1.2478 | 0.002801 | KLHDC4 |
| 28141776 | 28155940 | chr13 | − | exo | hsa_circ_0000468 | 1.2879 | 0.002805 | LNX2 |
| 4516153 | 4519466 | chr16 | − | exo | hsa_circ_0007788 | 0.7115 | 0.002807 | NMRAL1 |
| 50473795 | 50474137 | chr14 | − | s/o | hsa_circ_0000536 | 1.4968 | 0.002820 | LOC100506499 |
| 100451351 | 100455560 | chr3 | + | exo | hsa_circ_0008839 | 1.1630 | 0.002821 | TFG |
| 239184383 | 239186596 | chr2 | − | exo | hsa_circ_0003854 | 1.1404 | 0.002831 | PER2 |
| 19296842 | 19297814 | chr19 | − | exo | hsa_circ_0050211 | 1.1981 | 0.002843 | MEF2BNB- |
| | | | | | | | | MEF2B |
| 19288593 | 19300329 | chr9 | + | exo | | 0.7503 | 0.002844 | DENND4C |
| 29191568 | 29192081 | chr22 | − | s/o | hsa_circ_0001216 | 0.4379 | 0.002868 | XBP1 |
| 89961445 | 89970613 | chr16 | + | exo | hsa_circ_0007027 | 1.6379 | 0.002871 | TCF25 |
| 188202379 | 188242575 | chr3 | + | exo | hsa_circ_0006517 | 2.2507 | 0.002883 | LPP |
| 20907148 | 20929595 | chr9 | + | exo | hsa_circ_0008508 | 0.7864 | 0.002898 | FOCAD |
| 82836502 | 82836622 | chr5 | + | int | hsa_circ_0001510 | 0.8262 | 0.002899 | VCAN |
| 7402357 | 7402812 | chr17 | + | s/o | | 1.4459 | 0.002907 | POLR2A |
| 430683 | 440932 | chr19 | − | exo | | 1.4508 | 0.002919 | SHC2 |
| 77214859 | 77230123 | chr7 | + | exo | hsa_circ_0003764 | 0.7876 | 0.002927 | PTPN12 |
| 19615443 | 19653155 | chr12 | + | exo | hsa_circ_0006128 | 0.6039 | 0.002947 | AEBP2 |
| 100976372 | 101023156 | chr3 | − | exo | | 0.8733 | 0.002950 | IMPG2 |
| 20811305 | 20811534 | chr14 | − | s/o | hsa_circ_0000515 | 3.2964 | 0.002957 | RPPH1 |
| 4944143 | 4947223 | chr19 | + | exo | hsa_circ_0009107 | 1.8038 | 0.002957 | UHRF1 |
| 80898774 | 80906017 | chr4 | − | exo | hsa_circ_0070216 | 1.1677 | 0.002977 | ANTXR2 |
| 156638555 | 156647189 | chr1 | − | exo | hsa_circ_0014772 | 1.4874 | 0.003008 | NES |
| 30267304 | 30300250 | chr17 | + | exo | hsa_circ_0005804 | 0.5662 | 0.003024 | SUZ12 |
| 102038066 | 102045130 | chr7 | + | exo | hsa_circ_0001735 | 1.3795 | 0.003032 | PRKRIP1 |
| 33932559 | 33933626 | chr9 | − | exo | hsa_circ_0086694 | 0.8013 | 0.003042 | UBAP2 |
| 37880978 | 37882106 | chr17 | + | exo | hsa_circ_0043469 | 1.4474 | 0.003043 | ERBB2 |
| 20819794 | 20874806 | chr9 | + | exo | hsa_circ_0003782 | 0.5529 | 0.003061 | FOCAD |
| 929569 | 961079 | chr16 | − | exo | hsa_circ_0005483 | 1.1362 | 0.003063 | LMF1 |
| 149823791 | 149829319 | chr5 | − | exo | hsa_circ_0074537 | 2.0943 | 0.003070 | RPS14 |
| 110321942 | 110323436 | chr2 | − | exo | hsa_circ_0009020 | 0.6822 | 0.003071 | SEPT10 |
| 43650344 | 43671480 | chr10 | + | exo | | 0.6552 | 0.003084 | CSGALNACT2 |
| 1352719 | 1356247 | chr20 | − | exo | hsa_circ_0059181 | 1.2304 | 0.003095 | FKBP1A |
| 186287578 | 186287960 | chr1 | − | exo | | 1.1613 | 0.003099 | TPR |
| 43292575 | 43299077 | chr5 | − | exo | hsa_circ_0072386 | 1.8758 | 0.003120 | HMGCS1 |
| 71142242 | 71146174 | chr10 | + | exo | | 1.2505 | 0.003120 | HK1 |
| 50131152 | 50131308 | chr3 | + | exo | hsa_circ_0065793 | 1.1521 | 0.003124 | RBM5 |
| 10368897 | 10369361 | chr19 | + | exo | hsa_circ_0008762 | 1.3818 | 0.003139 | MRPL4 |
| 152163070 | 152165562 | chr3 | + | exo | hsa_circ_0007630 | 0.4955 | 0.003155 | MBNL1 |
| 33095509 | 33111164 | chr13 | − | exo | hsa_circ_0029937 | 0.6220 | 0.003158 | N4BP2L2 |
| 61608136 | 61617668 | chr3 | + | int | hsa_circ_0002848 | 1.1637 | 0.003170 | PTPRG |
| 16093870 | 16101291 | chr1 | + | exo | hsa_circ_0010096 | 2.0339 | 0.003174 | FBLIM1 |
| 35207143 | 35207369 | chr20 | + | exo | hsa_circ_0060191 | 1.2848 | 0.003196 | TGIF2 |
| 45545508 | 45545708 | chr21 | + | int | hsa_circ_0092315 | 1.9925 | 0.003206 | PWP2 |
| 25630392 | 25636298 | chr17 | + | exo | hsa_circ_0007716 | 0.8492 | 0.003221 | WSB1 |
| 34082350 | 34086545 | chr20 | + | exo | hsa_circ_0060078 | 1.2037 | 0.003232 | CEP250 |
| 303021 | 303397 | chr20 | − | s/o | | 1.1735 | 0.003279 | NRSN2-AS1 |
| 70096955 | 70101437 | chr10 | + | exo | hsa_circ_0018499 | 1.5924 | 0.003285 | HNRNPH3 |
| 24495906 | 24507344 | chr1 | − | exo | hsa_circ_0010906 | 2.2234 | 0.003288 | IFNLR1 |
| 246490502 | 246498776 | chr1 | − | exo | hsa_circ_0017306 | 0.8160 | 0.003300 | SMYD3 |
| 129180070 | 129188260 | chr3 | + | exo | | 1.3086 | 0.003335 | IFT122 |
| 89801934 | 89817535 | chr15 | + | exo | | 1.1018 | 0.003380 | FANCI |
| 109907319 | 109907533 | chr12 | − | exo | hsa_circ_0028132 | 1.0902 | 0.003388 | KCTD10 |
| 140954867 | 140955044 | chr3 | − | as | hsa_circ_0001343 | 0.3468 | 0.003405 | PXYLP1 |
| 140458885 | 140459606 | chr9 | − | exo | hsa_circ_0006502 | 1.2366 | 0.003517 | DPH7 |
| 86293355 | 86301070 | chr9 | − | exo | hsa_circ_0087357 | 0.5240 | 0.003527 | UBQLN1 |
| 44127898 | 44172067 | chr17 | − | exo | hsa_circ_0005940 | 0.8317 | 0.003528 | KANSL1 |
| 93760203 | 93762503 | chr14 | − | exo | hsa_circ_0000563 | 0.5541 | 0.003536 | BTBD7 |
| 45802132 | 45802362 | chr7 | − | int | hsa_circ_0001703 | 1.1734 | 0.003538 | SEPT7P2 |
| 10471474 | 10473308 | chr1 | + | exo | hsa_circ_0009732 | 2.6186 | 0.003538 | PGD |
| 97462748 | 97483239 | chr2 | + | s/o | | 1.2244 | 0.003543 | CNNM3 |
| 29063135 | 29096287 | chr1 | + | exo | hsa_circ_0011164 | 2.0085 | 0.003556 | YTHDF2 |
| 241991885 | 241992093 | chr2 | + | int | hsa_circ_0001120 | 1.1346 | 0.003560 | SNED1 |
| 1499235 | 1499964 | chr12 | + | int | | 0.5840 | 0.003566 | ERC1 |
| 29917108 | 29917448 | chr16 | + | exo | | 0.4761 | 0.003605 | ASPHD1 |
| 89853414 | 89866052 | chr12 | − | exo | | 0.6894 | 0.003625 | POC1B |
| 40632684 | 40646491 | chr17 | + | exo | hsa_circ_0043829 | 0.8981 | 0.003636 | ATP6V0A1 |
| 242079935 | 242082489 | chr2 | − | exo | hsa_circ_0058988 | 1.3276 | 0.003672 | PASK |
| 64876751 | 64879180 | chr11 | + | s/o | hsa_circ_0022764 | 1.2350 | 0.003705 | VPS51 |
| 109186061 | 109186605 | chr12 | − | exo | hsa_circ_0002957 | 1.2430 | 0.003726 | SSH1 |
| 7262372 | 7269899 | chr10 | − | exo | hsa_circ_0017625 | 0.8894 | 0.003745 | SFMBT2 |
| 44714009 | 44714867 | chr7 | + | exo | hsa_circ_0005004 | 1.2886 | 0.003751 | OGDH |
| 44054401 | 44054671 | chr1 | + | exo | hsa_circ_0012077 | 2.6591 | 0.003755 | PTPRF |
| 48248829 | 48248993 | chr19 | + | s/o | hsa_circ_0000945 | 1.4895 | 0.003755 | GLTSCR2 |
| 133438052 | 133447369 | chr12 | − | exo | | 1.1649 | 0.003767 | CHFR |
| 42521847 | 42523653 | chr12 | − | int | hsa_circ_0003402 | 0.5472 | 0.003775 | GXYLT1 |
| 109094899 | 109095099 | chr12 | − | exo | hsa_circ_0007830 | 1.2456 | 0.003790 | CORO1C |
| 41926690 | 41936799 | chr22 | − | exo | hsa_circ_0063575 | 1.5126 | 0.003798 | POLR3H |
| 73481508 | 73482507 | chr17 | + | exo | | 1.2347 | 0.003804 | KIAA0195 |
| 61655830 | 61662690 | chr17 | + | exo | hsa_circ_0045203 | 1.1465 | 0.003804 | DCAF7 |
| 50701162 | 50714058 | chr10 | − | exo | hsa_circ_0006243 | 1.0588 | 0.003814 | ERCC6 |
| 201970503 | 201970895 | chr1 | + | exo | hsa_circ_0015966 | 1.3615 | 0.003820 | RNPEP |
| 73614502 | 73690399 | chr14 | + | exo | hsa_circ_0032505 | 0.8685 | 0.003828 | PSEN1 |
| 3425103 | 3449137 | chr19 | + | exo | hsa_circ_0048467 | 1.3988 | 0.003854 | NFIC |
| 24572276 | 24578301 | chr4 | − | exo | hsa_circ_0002518 | 1.1813 | 0.003874 | DHX15 |
| 136969725 | 136975666 | chr5 | − | exo | hsa_circ_0074063 | 1.3320 | 0.003880 | KLHL3 |
| 95511601 | 95550574 | chr8 | − | exo | hsa_circ_0084903 | 0.3295 | 0.003941 | KIAA1429 |
| 56824664 | 56826308 | chr12 | − | exo | hsa_circ_0000408 | 1.2541 | 0.003957 | TIMELESS |
| 108243000 | 108250718 | chr6 | − | exo | hsa_circ_0001635 | 0.6971 | 0.003975 | SEC63 |
| 45715378 | 45780869 | chr2 | − | exo | | 0.6166 | 0.003980 | SRBD1 |
| 24551661 | 24570263 | chr6 | − | exo | | 2.4762 | 0.003986 | KIAA0319 |
| 34941169 | 34957919 | chr19 | + | exo | hsa_circ_0050547 | 2.0113 | 0.004028 | UBA2 |
| 29691716 | 29691882 | chr18 | + | exo | hsa_circ_0047376 | 0.5422 | 0.004029 | RNF138 |
| 65622881 | 65623563 | chr11 | − | int | hsa_circ_0000328 | 1.6545 | 0.004033 | CFL1 |
| 53174429 | 53174754 | chr14 | + | int | hsa_circ_0000538 | 1.1342 | 0.004037 | PSMC6 |
| 23610594 | 23615961 | chr22 | + | exo | hsa_circ_0062525 | 1.1700 | 0.004096 | BCR |
| 4848946 | 4850115 | chr17 | − | exo | hsa_circ_0041631 | 1.3418 | 0.004108 | PFN1 |
| 13418 | 13499 | chrM | + | s/o | hsa_circ_0008882 | 2.8151 | 0.004117 | MTND5 |
| 44530905 | 44531243 | chr20 | − | exo | hsa_circ_0060558 | 1.5141 | 0.004127 | PLTP |
| 89962397 | 89967202 | chr16 | + | exo | hsa_circ_0041103 | 1.3645 | 0.004144 | TCF25 |
| 43679047 | 43680248 | chr7 | − | exo | hsa_circ_0001700 | 1.2574 | 0.004156 | COA1 |
| 139620450 | 139620843 | chr9 | − | s/o | hsa_circ_0001903 | 1.1519 | 0.004171 | SNORA43 |
| 34143977 | 34145405 | chr20 | + | exo | hsa_circ_0060102 | 1.3929 | 0.004178 | ERGIC3 |
| 21464367 | 21464870 | chr14 | + | exo | hsa_circ_0031132 | 0.4728 | 0.004183 | METTL17 |
| 139978870 | 140060651 | chr4 | − | exo | hsa_circ_0070982 | 0.8386 | 0.004189 | ELF2 |
| 13213456 | 13213544 | chr19 | + | as | hsa_circ_0000898 | 1.2216 | 0.004213 | LYL1 |
| 84644859 | 84680000 | chr1 | + | exo | hsa_circ_0004083 | 0.6925 | 0.004219 | PRKACB |
| 25830104 | 25830751 | chr2 | − | int | | 0.9171 | 0.004248 | DTNB |
| 134308003 | 134330553 | chr9 | + | exo | hsa_circ_0005708 | 1.1283 | 0.004250 | PRRC2B |
| 143381846 | 143399995 | chr8 | − | exo | hsa_circ_0085800 | 1.1800 | 0.004271 | TSNARE1 |
| 62567915 | 62582288 | chr17 | − | exo | hsa_circ_0005637 | 0.7613 | 0.004275 | SMURF2 |
| 29346047 | 29347038 | chr15 | + | exo | hsa_circ_0004695 | 1.1677 | 0.004317 | APBA2 |
| 71680927 | 71681075 | chr2 | − | as | hsa_circ_0001029 | 2.2372 | 0.004338 | DYSF |
| 56111729 | 56114504 | chr19 | + | exo | hsa_circ_0052254 | 1.1345 | 0.004357 | ZNF524 |
| 99538969 | 99608397 | chr8 | − | exo | hsa_circ_0002798 | 0.8395 | 0.004365 | STK3 |
| 79249768 | 79257302 | chr17 | − | exo | hsa_circ_0046131 | 1.1573 | 0.004369 | SLC38A10 |
| 37425918 | 37426651 | chr9 | + | exo | hsa_circ_0087063 | 2.1397 | 0.004388 | GRHPR |
| 1270926 | 1274439 | chr19 | + | exo | | 1.9970 | 0.004394 | CIRBP |
| 103173821 | 103194718 | chr11 | + | exo | hsa_circ_0024143 | 1.2965 | 0.004396 | DYNC2H1 |
| 174169134 | 174169591 | chr4 | + | s/o | | 1.2124 | 0.004411 | GALNT7 |
| 119002244 | 119003929 | chr11 | + | exo | hsa_circ_0006932 | 1.2292 | 0.004414 | HINFP |
| 114905750 | 114905903 | chr9 | − | exo | hsa_circ_0088059 | 1.8992 | 0.004419 | SUSD1 |
| 176050287 | 176105683 | chr1 | − | exo | hsa_circ_0003572 | 0.7836 | 0.004443 | RFWD2 |
| 74638992 | 74672811 | chr18 | + | exo | hsa_circ_0005776 | 0.8985 | 0.004458 | ZNF236 |
| 46889530 | 46906128 | chr18 | − | exo | hsa_circ_0000849 | 0.4706 | 0.004473 | DYM |
| 19626181 | 19653155 | chr12 | + | exo | hsa_circ_0005257 | 0.6261 | 0.004483 | AEBP2 |
| 118474142 | 118476193 | chr12 | − | exo | hsa_circ_0007681 | 1.5446 | 0.004518 | WSB2 |
| 66050718 | 66050862 | chr11 | + | exo | hsa_circ_0022963 | 0.6450 | 0.004520 | CNIH2 |
| 48063446 | 48064400 | chr21 | + | exo | hsa_circ_0003781 | 2.0614 | 0.004528 | PRMT2 |
| 592468 | 619543 | chr6 | − | exo | hsa_circ_0075437 | 0.8910 | 0.004573 | EXOC2 |
| 118533337 | 118533619 | chr12 | − | exo | hsa_circ_0028644 | 1.1944 | 0.004574 | VSIG10 |
| 141228409 | 141248644 | chr3 | + | s/o | hsa_circ_0001344 | 0.8459 | 0.004578 | RASA2 |
| 12841453 | 12842235 | chr19 | − | exo | hsa_circ_0049592 | 1.5273 | 0.004598 | C19orf43 |
| 95030455 | 95032265 | chr9 | − | exo | hsa_circ_0008367 | 0.8739 | 0.004600 | IARS |
| 50904191 | 50904269 | chr22 | − | exo | hsa_circ_0063981 | 1.4406 | 0.004602 | SBF1 |
| 141889166 | 141917775 | chr3 | − | exo | hsa_circ_0067597 | 0.8739 | 0.004640 | GK5 |
| 36245469 | 36247930 | chr19 | − | exo | hsa_circ_0050649 | 0.3259 | 0.004643 | HSPB6 |
| 38416786 | 38416919 | chr17 | + | exo | hsa_circ_0043522 | 1.2700 | 0.004645 | WIPF2 |
| 66281876 | 66287219 | chr11 | + | exo | hsa_circ_0000329 | 1.1453 | 0.004658 | BBS1 |
| 43135467 | 43142681 | chr20 | − | exo | hsa_circ_0002615 | 0.7778 | 0.004661 | SERINC3 |
| 105718843 | 105752713 | chr14 | − | exo | hsa_circ_0008538 | 1.1865 | 0.004668 | BRF1 |
| 131967696 | 131969900 | chr10 | + | exo | hsa_circ_0020508 | 0.8594 | 0.004700 | GLRX3 |
| 112073555 | 112151290 | chr5 | + | exo | hsa_circ_0073584 | 1.3083 | 0.004707 | APC |
| 69853296 | 69856305 | chr12 | + | s/o | | 0.5247 | 0.004710 | RP11-956E11.1 |
| 29691716 | 29704808 | chr18 | + | exo | hsa_circ_0047378 | 0.7173 | 0.004712 | RNF138 |
| 947816 | 948781 | chr20 | − | exo | | 1.6379 | 0.004730 | RSPO4 |
| 230795209 | 230807386 | chr1 | + | exo | hsa_circ_0016863 | 0.8469 | 0.004759 | COG2 |
| 19519008 | 19522266 | chr16 | − | exo | | 1.1483 | 0.004764 | GDE1 |
| 30510394 | 30510810 | chr16 | + | exo | hsa_circ_0039053 | 1.4786 | 0.004807 | ITGAL |
| 5690345 | 5691678 | chr19 | + | exo | hsa_circ_0048764 | 0.4174 | 0.004825 | RPL36 |
| 103002341 | 103027210 | chr9 | + | exo | | 1.1841 | 0.004828 | INVS |
| 71502781 | 71518729 | chr14 | + | exo | hsa_circ_0032446 | 0.6943 | 0.004835 | PCNX |
| 28250417 | 28252532 | chr11 | + | int | | 0.6637 | 0.004843 | METTL15 |
| 6646085 | 6647162 | chr12 | + | exo | hsa_circ_0025196 | 1.5955 | 0.004848 | GAPDH |
| 101542470 | 101544855 | chr9 | − | exo | hsa_circ_0006928 | 1.1920 | 0.004855 | ANKS6 |
| 37853894 | 37853978 | chr19 | − | as | hsa_circ_0000930 | 0.4457 | 0.004867 | HKR1 |
| 27206356 | 27216044 | chr17 | − | exo | hsa_circ_0042722 | 1.0914 | 0.004882 | FLOT2 |
| 72338064 | 72338975 | chr15 | − | exo | hsa_circ_0006509 | 0.6096 | 0.004895 | MYO9A |
| 136277418 | 136278041 | chr9 | − | exo | hsa_circ_0089371 | 1.4172 | 0.004904 | REXO4 |
| 4177075 | 4179283 | chr19 | − | exo | hsa_circ_0048584 | 1.4843 | 0.004919 | SIRT6 |
| 46225531 | 46229033 | chr21 | − | exo | hsa_circ_0061975 | 1.3215 | 0.004925 | SUMO3 |
| 183388821 | 183390272 | chr3 | + | exo | | 0.6164 | 0.004928 | KLHL24 |
| 45347283 | 45363116 | chr1 | − | exo | | 1.1093 | 0.004934 | EIF2B3 |
| 6776706 | 6777567 | chr7 | + | exo | hsa_circ_0007607 | 0.7488 | 0.004947 | PMS2CL |
| 143704384 | 143704688 | chr3 | + | exo | hsa_circ_0067680 | 0.8420 | 0.004966 | C3orf58 |
| 54735989 | 54743442 | chr12 | + | exo | | 1.1399 | 0.004980 | COPZ1 |
| 27444085 | 27466654 | chr2 | + | exo | hsa_circ_0053179 | 1.1570 | 0.005006 | CAD |
| 32874967 | 32889268 | chr22 | + | exo | hsa_circ_0062984 | 0.7648 | 0.005009 | FBXO7 |
| 85656607 | 85669605 | chr15 | + | exo | hsa_circ_0036627 | 0.8625 | 0.005015 | PDE8A |
| 137708362 | 137735701 | chr5 | + | exo | hsa_circ_0074104 | 1.4586 | 0.005020 | KDM3B |
| 102106263 | 102106717 | chr7 | + | exo | hsa_circ_0003327 | 1.7105 | 0.005026 | LRWD1 |
| 21875557 | 21875757 | chr13 | + | exo | | 1.0881 | 0.005045 | MIPEPP3 |
| 126370175 | 126370948 | chr10 | − | exo | hsa_circ_0000267 | 1.5602 | 0.005070 | FAM53B |
| 219445283 | 219457430 | chr2 | + | exo | hsa_circ_0006745 | 1.1054 | 0.005090 | RQCD1 |
| 70880874 | 70886091 | chr7 | + | exo | hsa_circ_0080425 | 0.6566 | 0.005092 | WBSCR17 |
| 3205916 | 3209234 | chr10 | − | exo | hsa_circ_0003022 | 1.4267 | 0.005111 | PITRM1 |
| 73770739 | 73771807 | chr7 | + | exo | hsa_circ_0002755 | 1.3794 | 0.005114 | CLIP2 |
| 398169 | 409233 | chr20 | + | exo | hsa_circ_0059151 | 1.1358 | 0.005119 | RBCK1 |
| 70411602 | 70432802 | chr10 | + | exo | | 0.4174 | 0.005124 | TET1 |
| 2017333 | 2017502 | chr9 | + | int | hsa_circ_0001834 | 1.6884 | 0.005129 | SMARCA2 |
| 185655590 | 185655721 | chr3 | − | s/o | hsa_circ_0001373 | 1.5910 | 0.005135 | TRA2B |
| 86992149 | 86993147 | chr2 | + | exo | | 1.5250 | 0.005156 | RMND5A |
| 19345732 | 19383975 | chr18 | + | exo | hsa_circ_0007504 | 0.7838 | 0.005159 | MIB1 |
| 18685866 | 18688270 | chr19 | + | exo | hsa_circ_0050139 | 0.8218 | 0.005159 | UBA52 |
| 89790493 | 89790692 | chr6 | + | s/o | hsa_circ_0001618 | 0.4012 | 0.005177 | PNRC1 |
| 37511920 | 37512718 | chr9 | − | s/o | | 1.2991 | 0.005180 | FBXO10 |
| 70301561 | 70316687 | chr16 | − | exo | | 1.5937 | 0.005190 | AARS |
| 200729966 | 200784772 | chr1 | + | exo | hsa_circ_0015839 | 0.8276 | 0.005214 | CAMSAP2 |
| 114195512 | 114206774 | chr9 | − | exo | hsa_circ_0003697 | 0.8189 | 0.005216 | KIAA0368 |
| 45725821 | 45781640 | chr13 | + | exo | hsa_circ_0007259 | 0.7072 | 0.005224 | GTF2F2 |
| 233231500 | 233363117 | chr1 | − | exo | hsa_circ_0016950 | 0.7271 | 0.005232 | PCNXL2 |
| 28163979 | 28167848 | chr16 | − | exo | hsa_circ_0007807 | 0.8559 | 0.005254 | XPO6 |
| 135887564 | 135888291 | chr2 | + | exo | | 0.8345 | 0.005268 | RAB3GAP1 |
| 7478436 | 7478576 | chr17 | + | exo | hsa_circ_0041915 | 1.3782 | 0.005270 | EIF4A1 |
| 17503341 | 17506098 | chr4 | − | exo | hsa_circ_0069248 | 1.5418 | 0.005273 | QDPR |
| 179131199 | 179144030 | chr3 | − | exo | hsa_circ_0005009 | 0.5169 | 0.005294 | GNB4 |
| 45773870 | 45789895 | chr2 | − | exo | hsa_circ_0000997 | 0.6762 | 0.005301 | SRBD1 |
| 158580694 | 158591763 | chr7 | − | exo | hsa_circ_0001777 | 0.7427 | 0.005306 | ESYT2 |
| 74911537 | 74912566 | chr15 | + | exo | hsa_circ_0036287 | 1.3479 | 0.005308 | CLK3 |
| 57554979 | 57560039 | chr16 | − | exo | | 1.1133 | 0.005310 | CCDC102A |
| 5269501 | 5276395 | chr11 | − | exo | hsa_circ_0020962 | 1.1927 | 0.005318 | HBG2 |
| 54717288 | 54722859 | chr1 | − | exo | hsa_circ_0012651 | 1.3843 | 0.005335 | SSBP3 |
| 131558 | 177370 | chr17 | − | exo | hsa_circ_0041150 | 1.9879 | 0.005344 | RPH3AL |
| 6998051 | 7006651 | chr4 | + | exo | hsa_circ_0003201 | 0.4733 | 0.005347 | TBC1D14 |
| 65373231 | 65375919 | chr11 | − | exo | hsa_circ_0022847 | 0.6147 | 0.005361 | MAP3K11 |
| 217536827 | 217541605 | chr2 | − | exo | hsa_circ_0058196 | 1.1903 | 0.005383 | IGFBP5 |
| 128655032 | 128658211 | chr7 | − | exo | hsa_circ_0001741 | 0.6293 | 0.005412 | TNPO3 |
| 42641806 | 42641893 | chr20 | + | int | hsa_circ_0001160 | 1.1564 | 0.005420 | TOX2 |
| 45773870 | 45780869 | chr2 | − | exo | hsa_circ_0005542 | 0.6463 | 0.005431 | SRBD1 |
| 38698865 | 38710174 | chr22 | − | exo | hsa_circ_0063306 | 1.6424 | 0.005463 | CSNK1E |
| 36865777 | 36868169 | chr17 | + | s/o | hsa_circ_0000760 | 1.8184 | 0.005482 | MLLT6 |
| 151226785 | 151293194 | chr6 | + | exo | hsa_circ_0078279 | 0.8749 | 0.005492 | MTHFD1L |
| 48839753 | 48842572 | chr8 | − | exo | hsa_circ_0084443 | 0.8573 | 0.005500 | PRKDC |
| 68290258 | 68301913 | chr14 | + | exo | hsa_circ_0032305 | 1.1593 | 0.005502 | RAD51B |
| 15280896 | 15281636 | chr19 | − | exo | | 1.4109 | 0.005505 | NOTCH3 |
| 48154337 | 48157739 | chr17 | + | exo | hsa_circ_0044480 | 1.1485 | 0.005525 | ITGA3 |
| 31376659 | 31381401 | chr20 | + | exo | | 1.1321 | 0.005528 | DNMT3B |
| 42373768 | 42375484 | chr19 | + | exo | hsa_circ_0051246 | 1.1372 | 0.005532 | RPS19 |
| 6676816 | 6680397 | chr1 | + | exo | hsa_circ_0000011 | 1.1875 | 0.005542 | PHF13 |
| 236175220 | 236212289 | chr1 | − | exo | | 1.1261 | 0.005560 | NID1 |
| 114541671 | 114541925 | chr9 | + | as | | 0.3769 | 0.005565 | C9orf84 |
| 20448120 | 20456784 | chr9 | − | exo | hsa_circ_0002606 | 1.2040 | 0.005571 | MLLT3 |
| 84188738 | 84194770 | chr4 | − | exo | hsa_circ_0070271 | 1.2239 | 0.005583 | COQ2 |
| 35842162 | 35854205 | chr20 | + | exo | | 1.1315 | 0.005587 | RPN2 |
| 73685715 | 73687787 | chr11 | − | exo | hsa_circ_0023525 | 0.4652 | 0.005591 | UCP2 |
| 105905008 | 105920647 | chr14 | + | exo | | 1.3497 | 0.005628 | MTA1 |
| 51926762 | 51929470 | chr1 | − | exo | hsa_circ_0012448 | 0.7567 | 0.005631 | EPS15 |
| 2931959 | 2933418 | chr12 | + | exo | hsa_circ_0002009 | 1.9323 | 0.005638 | ITFG2 |
| 67351934 | 67352712 | chr11 | + | exo | hsa_circ_0023115 | 0.2458 | 0.005647 | GSTP1 |
| 115005725 | 115006178 | chr1 | − | exo | hsa_circ_0013637 | 0.8707 | 0.005655 | TRIM33 |
| 49658864 | 49667113 | chr12 | + | exo | hsa_circ_0026134 | 0.4850 | 0.005678 | TUBA1C |
| 7478510 | 7478576 | chr17 | + | int | hsa_circ_0007008 | 1.5952 | 0.005691 | EIF4A1 |
| 22940727 | 22947086 | chr15 | + | exo | hsa_circ_0034072 | 1.1110 | 0.005713 | CYFIP1 |
| 64863339 | 64868113 | chr5 | + | exo | hsa_circ_0072697 | 0.7393 | 0.005716 | PPWD1 |
| 9792628 | 9796569 | chr3 | + | exo | hsa_circ_0064155 | 1.4555 | 0.005721 | OGG1 |
| 32703702 | 32704685 | chr2 | + | exo | hsa_circ_0002408 | 0.6379 | 0.005730 | BIRC6 |
| 4702038 | 4705982 | chr19 | − | exo | | 1.1095 | 0.005734 | DPP9 |
| 12359258 | 12364785 | chr1 | + | exo | hsa_circ_0007677 | 1.3981 | 0.005748 | VPS13D |
| 9208654 | 9239554 | chr18 | + | int | hsa_circ_0005715 | 0.7226 | 0.005749 | RP11-21J18.1 |
| 128638012 | 128651918 | chr11 | + | exo | hsa_circ_0024834 | 1.1158 | 0.005751 | FLI1 |
| 11020812 | 11029717 | chr21 | − | exo | hsa_circ_0061251 | 0.6662 | 0.005778 | TPTE |
| 20811282 | 20811360 | chr14 | − | s/o | hsa_circ_0006853 | 2.7409 | 0.005784 | RPPH1 |
| 19345732 | 19399607 | chr18 | + | exo | hsa_circ_0000836 | 0.7522 | 0.005800 | MIB1 |
| 153593274 | 153593598 | chrX | + | as | hsa_circ_0001951 | 1.5063 | 0.005819 | FLNA |
| 102180052 | 102180538 | chr7 | − | exo | hsa_circ_0081682 | 1.4219 | 0.005823 | POLR2J3 |
| 31586088 | 31595859 | chr12 | − | exo | hsa_circ_0025821 | 0.7934 | 0.005840 | DENND5B |
| 49372459 | 49373029 | chr3 | − | int | | 1.1465 | 0.005840 | USP4 |
| 131113791 | 131113904 | chr7 | + | exo | hsa_circ_0082413 | 0.7121 | 0.005846 | MKLN1 |
| 117528017 | 117538407 | chrX | + | exo | | 0.7954 | 0.005864 | WDR44 |
| 124997967 | 125032500 | chr3 | − | exo | hsa_circ_0067100 | 0.7541 | 0.005908 | ZNF148 |
| 6094176 | 6094335 | chr7 | − | exo | hsa_circ_0079330 | 1.1792 | 0.005912 | EIF2AK1 |
| 299338 | 308428 | chrX | − | exo | hsa_circ_0002172 | 1.3911 | 0.005913 | PPP2R3B |
| 69703659 | 69785930 | chr6 | + | exo | hsa_circ_0076943 | 0.7606 | 0.005922 | BAI3 |
| 66532271 | 66548526 | chr7 | + | exo | hsa_circ_0007252 | 1.4277 | 0.005923 | TYW1 |
| 109455640 | 109473426 | chr2 | + | exo | hsa_circ_0001061 | 0.8366 | 0.005923 | CCDC138 |
| 43465618 | 43471444 | chr6 | + | exo | | 1.1833 | 0.005927 | TJAP1 |
| 49227059 | 49228280 | chr12 | − | exo | | 1.0969 | 0.005934 | DDX23 |
| 87788799 | 87795646 | chr16 | − | exo | hsa_circ_0040792 | 1.2500 | 0.005937 | KLHDC4 |
| 83799882 | 83803090 | chr4 | − | exo | hsa_circ_0001421 | 0.6553 | 0.005938 | SEC31A |
| 56167366 | 56167744 | chr19 | + | int | hsa_circ_0000961 | 1.0986 | 0.005948 | U2AF2 |
| 57842331 | 57889147 | chr17 | + | exo | hsa_circ_0044879 | 0.7223 | 0.005980 | VMP1 |
| 32602655 | 32626691 | chr2 | + | exo | hsa_circ_0007337 | 0.7372 | 0.005997 | BIRC6 |
| 2664564 | 2674099 | chr4 | + | exo | hsa_circ_0003869 | 0.2705 | 0.005998 | FAM193A |
| 51065351 | 51077601 | chr10 | − | exo | hsa_circ_0018340 | 0.6187 | 0.006051 | PARG |
| 62559687 | 62562375 | chr20 | + | exo | hsa_circ_0007609 | 1.3620 | 0.006053 | DNAJC5 |
| 19746674 | 19775434 | chr16 | + | exo | hsa_circ_0000680 | 0.5063 | 0.006066 | IQCK |
| 10186276 | 10188722 | chr2 | + | exo | | 1.2766 | 0.006069 | KLF11 |
| 21178613 | 21193019 | chr22 | − | exo | hsa_circ_0062397 | 1.2044 | 0.006070 | PI4KA |
| 5941287 | 5949747 | chr7 | + | exo | hsa_circ_0004703 | 0.7096 | 0.006080 | CCZ1 |
| 35704665 | 35706108 | chr9 | − | exo | hsa_circ_0086905 | 1.2259 | 0.006090 | TLN1 |
| 52773707 | 52788209 | chr20 | − | exo | hsa_circ_0060927 | 1.1733 | 0.006166 | CYP24A1 |
| 44839300 | 44842716 | chr7 | + | exo | hsa_circ_0080091 | 1.2463 | 0.006200 | PPIA |
| 71495398 | 71508556 | chr8 | − | exo | | 0.8234 | 0.006232 | TRAM1 |
| 25477659 | 25477979 | chr20 | − | int | hsa_circ_0092338 | 0.6823 | 0.006239 | NINL |
| 30366964 | 30375041 | chr22 | + | exo | hsa_circ_0006303 | 1.1757 | 0.006285 | MTMR3 |
| 80963811 | 80997230 | chr14 | − | exo | hsa_circ_0007332 | 1.2619 | 0.006301 | CEP128 |
| 100506410 | 100506635 | chr2 | − | int | hsa_circ_0001054 | 1.4996 | 0.006307 | AFF3 |
| 21039946 | 21042132 | chr1 | − | exo | hsa_circ_0010433 | 1.0957 | 0.006314 | KIF17 |
| 1192371 | 1203372 | chr1 | − | exo | hsa_circ_0009250 | 1.2125 | 0.006342 | UBE2J2 |
| 165859440 | 165860559 | chr1 | + | exo | hsa_circ_0006758 | 0.4065 | 0.006353 | UCK2 |
| 19656207 | 19663412 | chr16 | + | exo | hsa_circ_0003645 | 0.6304 | 0.006381 | C16orf62 |
| 151927007 | 151933018 | chr7 | − | exo | hsa_circ_0083026 | 0.8340 | 0.006405 | KMT2C |
| 52911373 | 52912944 | chr12 | − | exo | hsa_circ_0026457 | 1.3654 | 0.006426 | KRT5 |
| 88061088 | 88069000 | chr16 | + | exo | hsa_circ_0040822 | 1.6405 | 0.006429 | BANP |
| 1828213 | 1830915 | chr8 | + | exo | | 1.2443 | 0.006431 | ARHGEF10 |
| 45911303 | 45914319 | chr13 | − | exo | hsa_circ_0030162 | 1.8395 | 0.006432 | TPT1 |
| 41089303 | 41097305 | chr19 | + | exo | hsa_circ_0051110 | 0.5319 | 0.006433 | SHKBP1 |
| 32117060 | 32157204 | chr2 | − | exo | hsa_circ_0003001 | 0.7066 | 0.006437 | MEMO1 |
| 7754370 | 7755918 | chr18 | + | int | hsa_circ_0008584 | 0.2571 | 0.006488 | PTPRM |
| 62325639 | 62336454 | chr15 | − | exo | hsa_circ_0005761 | 0.9040 | 0.006506 | VPS13C |
| 56419830 | 56423287 | chr16 | − | exo | hsa_circ_0000704 | 1.1252 | 0.006563 | AMFR |
| 34868500 | 34871833 | chr17 | − | exo | | 1.1572 | 0.006573 | MYO19 |
| 153555574 | 153562137 | chr7 | + | ing | | 1.7949 | 0.006606 | |
| 145001577 | 145009097 | chr8 | − | exo | hsa_circ_0085912 | 1.2123 | 0.006614 | PLEC |
| 30601792 | 30607930 | chr11 | − | exo | hsa_circ_0021555 | 1.5031 | 0.006623 | MPPED2 |
| 151169883 | 151175136 | chr5 | + | exo | | 1.2182 | 0.006628 | G3BP1 |
| 48701711 | 48805817 | chr8 | − | exo | hsa_circ_0084275 | 1.1921 | 0.006693 | PRKDC |
| 50929382 | 50930851 | chr14 | − | exo | hsa_circ_0031859 | 0.7241 | 0.006711 | MAP4K5 |
| 4354945 | 4355170 | chr19 | + | exo | hsa_circ_0003999 | 1.0826 | 0.006718 | MPND |
| 9023225 | 9024254 | chr16 | − | exo | hsa_circ_0004344 | 1.3196 | 0.006740 | USP7 |
| 42071996 | 42072196 | chr6 | − | int | hsa_circ_0092341 | 1.1726 | 0.006740 | C6orf132 |
| 9919687 | 9919910 | chr18 | + | int | hsa_circ_0000828 | 1.1025 | 0.006745 | VAPA |
| 6475390 | 6477808 | chr9 | + | exo | hsa_circ_0086306 | 0.9254 | 0.006749 | UHRF2 |
| 959436 | 972255 | chr11 | + | exo | | 1.2449 | 0.006761 | AP2A2 |
| 79158663 | 79176529 | chr4 | + | exo | hsa_circ_0070098 | 1.9171 | 0.006783 | FRAS1 |
| 54974813 | 54978373 | chr8 | − | exo | hsa_circ_0003976 | 1.2460 | 0.006790 | LYPLA1 |
| 33311103 | 33319125 | chr9 | + | exo | hsa_circ_0086645 | 1.1513 | 0.006797 | NFX1 |
| 43702432 | 43703330 | chr18 | + | exo | hsa_circ_0008333 | 0.8240 | 0.006814 | HAUS1 |
| 18839352 | 18845760 | chr16 | − | exo | hsa_circ_0038249 | 0.8400 | 0.006865 | SMG1 |
| 150076352 | 150078193 | chr5 | − | exo | | 1.0924 | 0.006882 | RBM22 |
| 155448623 | 155451551 | chr6 | + | exo | hsa_circ_0008289 | 1.4722 | 0.006891 | TIAM2 |
| 56669779 | 56676775 | chr12 | − | exo | hsa_circ_0026978 | 1.4287 | 0.006912 | CS |
| 42909540 | 42924974 | chr2 | + | exo | hsa_circ_0054272 | 0.8557 | 0.006978 | MTA3 |
| 4145550 | 4145743 | chr17 | − | exo | hsa_circ_0041551 | 1.4338 | 0.007023 | ANKFY1 |
| 8952206 | 8953192 | chr16 | − | exo | hsa_circ_0000669 | 1.6850 | 0.007075 | CARHSP1 |
| 138741982 | 138773640 | chr9 | − | exo | hsa_circ_0089490 | 0.8411 | 0.007108 | CAMSAP1 |
| 51402258 | 51404549 | chr12 | − | exo | hsa_circ_0008802 | 1.0851 | 0.007112 | SLC11A2 |
| 29691716 | 29706763 | chr18 | + | exo | hsa_circ_0005122 | 0.7288 | 0.007119 | RNF138 |
| 42605656 | 42611347 | chr22 | − | exo | hsa_circ_0008561 | 1.3194 | 0.007138 | TCF20 |
| 13054580 | 13054850 | chr19 | + | s/o | hsa_circ_0000897 | 1.4831 | 0.007191 | CALR |
| 56792449 | 56839544 | chr16 | + | exo | | 0.7961 | 0.007223 | NUP93 |
| 43616300 | 43621964 | chr3 | − | exo | hsa_circ_0003636 | 0.8441 | 0.007238 | ANO10 |
| 89724655 | 89734790 | chr16 | + | int | | 1.4512 | 0.007296 | C16orf55 |
| 85610244 | 85610435 | chr15 | + | exo | hsa_circ_0036617 | 0.8256 | 0.007321 | PDE8A |
| 97409853 | 97411719 | chr14 | + | s/o | | 1.2666 | 0.007332 | LINC00618 |
| 112399680 | 112421007 | chr5 | − | exo | hsa_circ_0009054 | 1.5745 | 0.007337 | MCC |
| 94023872 | 94027070 | chr7 | + | exo | hsa_circ_0081055 | 1.4098 | 0.007366 | COL1A2 |
| 128628009 | 128638167 | chr11 | + | exo | hsa_circ_0000368 | 1.1065 | 0.007373 | FLI1 |
| 64083439 | 64085070 | chr2 | + | exo | hsa_circ_0001020 | 1.1606 | 0.007401 | UGP2 |
| 137320945 | 137324004 | chr5 | − | exo | hsa_circ_0001535 | 0.5432 | 0.007407 | FAM13B |
| 56552044 | 56555366 | chr12 | + | exo | hsa_circ_0026885 | 1.3239 | 0.007410 | MYL6 |
| 32557275 | 32566163 | chr1 | + | exo | hsa_circ_0006349 | 0.5320 | 0.007428 | TMEM39B |
| 16133348 | 16208501 | chr11 | − | exo | | 1.3961 | 0.007465 | SOX6 |
| 37971709 | 37978667 | chr8 | + | exo | hsa_circ_0006302 | 0.7464 | 0.007468 | ASH2L |
| 6230580 | 6270770 | chr19 | − | exo | hsa_circ_0004594 | 1.4568 | 0.007472 | MLLT1 |
| 2972488 | 3000467 | chr11 | − | exo | hsa_circ_0020816 | 1.7461 | 0.007490 | NAP1L4 |
| 48726970 | 48728915 | chr3 | − | exo | hsa_circ_0003748 | 0.8465 | 0.007504 | IP6K2 |
| 131761180 | 131761386 | chr5 | + | int | hsa_circ_0001525 | 1.3567 | 0.007531 | C5orf56 |
| 48491442 | 48495818 | chr3 | + | exo | hsa_circ_0065384 | 0.6958 | 0.007535 | ATRIP |
| 97025880 | 97031796 | chr2 | + | exo | hsa_circ_0001047 | 1.2539 | 0.007544 | NCAPH |
| 20811404 | 20811492 | chr14 | − | s/o | hsa_circ_0000517 | 3.7786 | 0.007559 | RPPH1 |
| 136273891 | 136278041 | chr9 | − | exo | | 1.1385 | 0.007572 | REXO4 |
| 195605123 | 195615477 | chr3 | − | exo | hsa_circ_0001377 | 1.3261 | 0.007587 | TNK2 |
| 41620952 | 41622347 | chr22 | + | s/o | hsa_circ_0001234 | 0.7174 | 0.007643 | L3MBTL2 |
| 22999874 | 23030758 | chr7 | − | exo | hsa_circ_0008951 | 0.4987 | 0.007647 | FAM126A |
| 17199439 | 17210916 | chr10 | − | exo | | 0.7877 | 0.007670 | TRDMT1 |
| 86292641 | 86301070 | chr9 | − | exo | hsa_circ_0003715 | 0.7848 | 0.007706 | UBQLN1 |
| 49329991 | 49351252 | chr12 | − | exo | hsa_circ_0026074 | 2.0865 | 0.007737 | ARF3 |
| 54764057 | 54764137 | chr12 | + | int | hsa_circ_0000403 | 1.1807 | 0.007747 | RP11-753H16.3 |
| 47746290 | 47754682 | chr21 | + | exo | hsa_circ_0062084 | 1.2562 | 0.007752 | PCNT |
| 29090019 | 29095925 | chr22 | − | exo | hsa_circ_0062712 | 1.1134 | 0.007767 | CHEK2 |
| 75425277 | 75429107 | chr16 | − | s/o | | 1.2330 | 0.007781 | CFDP1 |
| 30611319 | 30630569 | chr10 | − | exo | | 1.6370 | 0.007794 | MTPAP |
| 1264385 | 1265302 | chr17 | − | exo | hsa_circ_0007643 | 0.8796 | 0.007808 | YWHAE |
| 3201105 | 3202522 | chr10 | − | exo | hsa_circ_0017510 | 0.7644 | 0.007815 | PITRM1 |
| 154186368 | 154187050 | chr1 | − | exo | | 1.3883 | 0.007829 | C1orf43 |
| 16110352 | 16127027 | chr16 | + | exo | hsa_circ_0038090 | 1.2109 | 0.007837 | ABCC1 |
| 89484691 | 89488713 | chr16 | − | s/o | hsa_circ_0004566 | 1.6985 | 0.007850 | ANKRD11 |
| 5039846 | 5047680 | chr19 | + | exo | hsa_circ_0004720 | 1.2210 | 0.007860 | KDM4B |
| 47652106 | 47652639 | chr11 | − | exo | hsa_circ_0022033 | 1.1877 | 0.007864 | MTCH2 |
| 11940357 | 11940700 | chr16 | − | exo | hsa_circ_0000673 | 0.2783 | 0.007865 | RSL1D1 |
| 69785302 | 69804320 | chr10 | − | exo | hsa_circ_0007113 | 0.7189 | 0.007883 | HERC4 |
| 131060182 | 131130662 | chr7 | + | exo | hsa_circ_0003047 | 0.6966 | 0.007909 | MKLN1 |
| 70246900 | 70266475 | chr10 | − | exo | hsa_circ_0007778 | 0.8229 | 0.007975 | SLC25A16 |
| 134305476 | 134308181 | chr9 | + | exo | hsa_circ_0001895 | 1.5336 | 0.008058 | PRRC2B |
| 58256381 | 58256569 | chr6 | − | exo | hsa_circ_0076917 | 2.2053 | 0.008117 | GUSBP4 |
| 88542064 | 88570096 | chr12 | + | exo | hsa_circ_0027689 | 0.8723 | 0.008153 | TMTC3 |
| 16685846 | 16690028 | chr5 | − | exo | hsa_circ_0071991 | 1.4957 | 0.008154 | MYO10 |
| 49699696 | 49700131 | chr19 | + | exo | hsa_circ_0005317 | 0.5282 | 0.008162 | TRPM4 |
| 3802927 | 3808661 | chr17 | − | exo | hsa_circ_0000735 | 0.2407 | 0.008220 | P2RX1 |
| 17421785 | 17514185 | chr6 | + | exo | hsa_circ_0075697 | 1.0898 | 0.008232 | CAP2 |
| 133734285 | 133769540 | chr8 | − | exo | hsa_circ_0008205 | 0.6558 | 0.008253 | TMEM71 |
| 151611363 | 151641111 | chr1 | + | exo | hsa_circ_0000131 | 0.7418 | 0.008267 | SNX27 |
| 124517260 | 124518071 | chr11 | − | exo | hsa_circ_0000367 | 1.2564 | 0.008272 | SIAE |
| 21976935 | 21992425 | chr13 | − | s/o | | 0.7608 | 0.008273 | ZDHHC20 |
| 41938372 | 41944319 | chr19 | − | exo | hsa_circ_0051238 | 1.9454 | 0.008280 | ATP5SL |
| 57814813 | 57816308 | chr17 | + | exo | hsa_circ_0044875 | 0.4563 | 0.008292 | VMP1 |
| 145011146 | 145011239 | chr8 | − | exo | hsa_circ_0085923 | 2.1573 | 0.008294 | PLEC |
| 149563797 | 149629870 | chr3 | + | exo | hsa_circ_0003502 | 0.6867 | 0.008309 | RNF13 |
| 44706334 | 44721447 | chr7 | + | exo | hsa_circ_0080067 | 1.2245 | 0.008324 | OGDH |
| 141595217 | 141595410 | chr8 | − | exo | hsa_circ_0008113 | 1.2050 | 0.008331 | AGO2 |
| 59535778 | 59555632 | chr8 | − | exo | hsa_circ_0005722 | 0.7545 | 0.008347 | NSMAF |
| 137218315 | 137300965 | chr9 | + | exo | hsa_circ_0089414 | 1.3509 | 0.008368 | RXRA |
| 120995084 | 120995485 | chr12 | + | exo | hsa_circ_0028899 | 1.2391 | 0.008375 | RNF10 |
| 102279160 | 102279646 | chr7 | − | exo | hsa_circ_0081710 | 1.3823 | 0.008400 | POLR2J2 |
| 35229039 | 35239635 | chr21 | + | exo | hsa_circ_0061570 | 0.6559 | 0.008426 | ITSN1 |
| 50904191 | 50904503 | chr22 | − | exo | hsa_circ_0063982 | 1.4024 | 0.008431 | SBF1 |
| 6426842 | 6431672 | chr7 | + | exo | hsa_circ_0004349 | 1.1155 | 0.008438 | RAC1 |
| 3777940 | 3778768 | chr11 | − | int | | 0.2537 | 0.008448 | NUP98 |
| 134072602 | 134077104 | chr9 | + | exo | hsa_circ_0089183 | 0.9287 | 0.008491 | NUP214 |
| 122031337 | 122031452 | chr12 | + | s/o | | 1.5469 | 0.008512 | RP13-941N14.1 |
| 96267023 | 96271411 | chr10 | + | exo | hsa_circ_0005691 | 0.6551 | 0.008535 | TBC1D12 |
| 25348845 | 25348942 | chr12 | − | as | hsa_circ_0000382 | 1.1470 | 0.008538 | LYRM5 |
| 45153949 | 45163688 | chr21 | + | exo | hsa_circ_0061891 | 1.3328 | 0.008564 | PDXK |
| 73170846 | 73177283 | chr17 | − | exo | | 1.2823 | 0.008582 | SUMO2 |
| 99924615 | 99925522 | chr14 | − | exo | hsa_circ_0033149 | 0.6723 | 0.008623 | SETD3 |
| 4029116 | 4033441 | chr16 | − | exo | hsa_circ_0007586 | 0.4187 | 0.008633 | ADCY9 |
| 17273159 | 17273932 | chr19 | + | exo | hsa_circ_0000909 | 0.8269 | 0.008635 | MYO9B |
| 155471301 | 155473602 | chr7 | + | exo | hsa_circ_0083092 | 0.6613 | 0.008656 | RBM33 |
| 156012056 | 156012564 | chr1 | − | int | | 1.1187 | 0.008657 | UBQLN4 |
| 36668836 | 36694658 | chr20 | + | exo | hsa_circ_0008006 | 1.1044 | 0.008675 | RPRD1B |
| 24761443 | 24765288 | chr22 | + | exo | hsa_circ_0062610 | 1.2215 | 0.008693 | SPECC1L |
| 59058742 | 59058878 | chr19 | + | exo | hsa_circ_0052372 | 1.2659 | 0.008728 | TRIM28 |
| 140648622 | 140652463 | chr9 | + | exo | hsa_circ_0089727 | 1.3907 | 0.008745 | EHMT1 |
| 88957371 | 88973310 | chr4 | + | exo | hsa_circ_0070400 | 0.7011 | 0.008752 | PKD2 |
| 73215386 | 73285725 | chr2 | − | exo | hsa_circ_0055199 | 1.2498 | 0.008759 | SFXN5 |
| 134076752 | 134079742 | chr5 | + | exo | hsa_circ_0003154 | 1.3900 | 0.008772 | CAMLG |
| 65702515 | 65703708 | chr15 | − | exo | | 1.3065 | 0.008776 | IGDCC4 |
| 24698162 | 24765288 | chr22 | + | exo | hsa_circ_0062603 | 1.2488 | 0.008808 | SPECC1L |
| 53854798 | 53858636 | chr12 | + | exo | hsa_circ_0026646 | 1.4361 | 0.008818 | PCBP2 |
| 114886536 | 114919893 | chr9 | − | exo | hsa_circ_0004286 | 0.5006 | 0.008819 | SUSD1 |
| 18650180 | 18654383 | chr19 | − | exo | hsa_circ_0050125 | 1.9197 | 0.008826 | FKBP8 |
| 219204505 | 219206867 | chr2 | + | exo | hsa_circ_0058230 | 0.9190 | 0.008845 | PNKD |
| 104678572 | 104681470 | chr7 | + | exo | hsa_circ_0007395 | 1.4989 | 0.008857 | KMT2E |
| 13049413 | 13055304 | chr19 | + | exo | hsa_circ_0049637 | 2.4866 | 0.008884 | CALR |
| 38226804 | 38231095 | chr19 | − | s/o | hsa_circ_0000931 | 0.5846 | 0.008886 | ZNF573 |
| 64743439 | 64752510 | chrX | − | exo | hsa_circ_0090879 | 1.2553 | 0.008900 | LAS1L |
| 10099334 | 10105610 | chr4 | − | exo | hsa_circ_0003550 | 1.6344 | 0.008954 | WDR1 |
| 62549365 | 62550297 | chr11 | + | exo | hsa_circ_0006855 | 1.3276 | 0.008985 | TAF6L |
| 180953812 | 180957481 | chr1 | − | exo | hsa_circ_0015524 | 1.3963 | 0.008995 | STX6 |
| 107031202 | 107050797 | chr6 | − | exo | hsa_circ_0008514 | 1.4933 | 0.008999 | RTN4IP1 |
| 118832460 | 118887479 | chr6 | − | exo | hsa_circ_0077736 | 0.6333 | 0.009001 | CEP85L |
| 51926762 | 51938620 | chr1 | − | exo | hsa_circ_0012451 | 0.7018 | 0.009065 | EPS15 |
| 39775691 | 39778763 | chr17 | − | exo | hsa_circ_0043637 | 0.7997 | 0.009067 | KRT17 |
| 20811404 | 20811554 | chr14 | − | s/o | hsa_circ_0000518 | 3.6437 | 0.009080 | RPPH1 |
| 61603333 | 61609229 | chr3 | + | int | hsa_circ_0007038 | 1.1360 | 0.009092 | PTPRG |
| 72509749 | 72511451 | chr15 | − | exo | hsa_circ_0036200 | 1.8993 | 0.009097 | PKM |
| 219114086 | 219114644 | chr2 | + | exo | hsa_circ_0058218 | 1.1785 | 0.009125 | ARPC2 |
| 17907047 | 17914974 | chr1 | + | exo | hsa_circ_0005562 | 1.3479 | 0.009131 | ARHGEF10L |
| 18650180 | 18653782 | chr19 | − | exo | hsa_circ_0050124 | 1.8290 | 0.009135 | FKBP8 |
| 34687538 | 34712643 | chr19 | + | exo | hsa_circ_0000926 | 1.8010 | 0.009142 | LSM14A |
| 18650180 | 18650530 | chr19 | − | exo | hsa_circ_0000915 | 1.7782 | 0.009194 | FKBP8 |
| 47361142 | 47364198 | chr3 | + | exo | hsa_circ_0065182 | 1.1568 | 0.009234 | KLHL18 |
| 105148561 | 105148991 | chr7 | − | exo | | 1.1295 | 0.009296 | PUS7 |
| 107824860 | 107827631 | chr6 | + | exo | hsa_circ_0001633 | 2.0707 | 0.009305 | SOBP |
| 85839338 | 85840607 | chr16 | + | exo | hsa_circ_0040749 | 1.5688 | 0.009323 | COX4I1 |
| 9028147 | 9098771 | chr2 | − | exo | hsa_circ_0008847 | 0.8633 | 0.009335 | MBOAT2 |
| 92798947 | 92811471 | chr1 | + | exo | hsa_circ_0004463 | 0.7668 | 0.009342 | RPAP2 |
| 122260742 | 122287901 | chr2 | − | exo | hsa_circ_0002374 | 0.7119 | 0.009348 | CLASP1 |
| 722678 | 729318 | chr17 | − | exo | hsa_circ_0004931 | 1.2839 | 0.009360 | NXN |
| 168233450 | 168250336 | chr5 | − | exo | hsa_circ_0074930 | 1.4884 | 0.009383 | SLIT3 |
| 157357968 | 157406039 | chr6 | + | exo | hsa_circ_0001658 | 0.5504 | 0.009397 | ARID1B |
| 84112887 | 84132553 | chr14 | + | ing | | 0.6425 | 0.009402 | |
| 1036128 | 1036561 | chr19 | + | exo | hsa_circ_0004667 | 1.5024 | 0.009415 | CNN2 |
| 74433139 | 74434317 | chr7 | + | exo | hsa_circ_0080638 | 0.3889 | 0.009432 | GATSL1 |
| 71714045 | 71715118 | chrX | − | int | | 0.8631 | 0.009438 | HDAC8 |
| 47797121 | 47797798 | chr17 | − | exo | hsa_circ_0000783 | 1.1547 | 0.009482 | FAM117A |
| 51868106 | 51874004 | chr1 | − | exo | hsa_circ_0005567 | 0.6113 | 0.009495 | EPS15 |
| 73969503 | 73972052 | chr7 | + | exo | | 1.1599 | 0.009502 | GTF2IRD1 |
| 1735857 | 1737977 | chr1 | − | exo | hsa_circ_0000007 | 1.2669 | 0.009517 | GNB1 |
| 79813328 | 79813462 | chr17 | − | exo | hsa_circ_0046265 | 1.1948 | 0.009547 | P4HB |
| 35797838 | 35800763 | chr17 | + | exo | hsa_circ_0043278 | 1.3984 | 0.009586 | TADA2A |
| 118378243 | 118382740 | chr11 | + | exo | | 1.1225 | 0.009594 | KMT2A |
| 150464886 | 150469384 | chr1 | + | exo | hsa_circ_0003952 | 1.2141 | 0.009615 | TARS2 |
| 153594675 | 153599729 | chrX | − | exo | hsa_circ_0092022 | 2.9096 | 0.009616 | FLNA |
| 147733519 | 147744289 | chrX | + | exo | hsa_circ_0091669 | 0.8114 | 0.009633 | AFF2 |
| 49405860 | 49413024 | chr3 | − | exo | hsa_circ_0065649 | 1.3965 | 0.009647 | RHOA |
| 38890634 | 38897285 | chr22 | − | exo | hsa_circ_0002211 | 0.5494 | 0.009652 | DDX17 |
| 64025122 | 64040057 | chr15 | − | exo | | 0.8184 | 0.009722 | HERC1 |
| 204354306 | 204356042 | chr2 | − | exo | hsa_circ_0004564 | 0.8481 | 0.009723 | RAPH1 |
| 128103455 | 128109220 | chr9 | + | exo | hsa_circ_0005249 | 0.8841 | 0.009736 | GAPVD1 |
| 12883003 | 12883858 | chr19 | − | exo | hsa_circ_0049600 | 1.0865 | 0.009742 | HOOK2 |
| 139819703 | 139828890 | chr5 | + | exo | hsa_circ_0001541 | 0.7427 | 0.009753 | ANKHD1 |
| 30075049 | 30080299 | chr16 | + | exo | hsa_circ_0038975 | 1.4169 | 0.009779 | ALDOA |
| 23370582 | 23383709 | chr20 | − | exo | hsa_circ_0003853 | 0.8204 | 0.009780 | NAPB |
| 173662 | 173882 | chr5 | + | int | hsa_circ_0092371 | 0.6362 | 0.009791 | PLEKHG4B |
| 22002700 | 22016857 | chr10 | + | exo | hsa_circ_0007084 | 0.7357 | 0.009819 | MLLT10 |
| 93483020 | 93542964 | chr14 | − | exo | hsa_circ_0033024 | 1.2950 | 0.009837 | ITPK1 |
| 27355470 | 27356589 | chr2 | − | exo | hsa_circ_0053141 | 1.3660 | 0.009896 | PREB |
| 9017159 | 9048846 | chr2 | − | exo | hsa_circ_0052507 | 0.8281 | 0.009964 | MBOAT2 |
| 167422629 | 167452651 | chr3 | − | exo | hsa_circ_0067867 | 1.0986 | 0.010042 | PDCD10 |
| 91924202 | 91981956 | chr7 | + | exo | hsa_circ_0002744 | 0.5656 | 0.010066 | ANKIB1 |
| 65043362 | 65049992 | chr11 | + | exo | | 1.2758 | 0.010073 | POLA2 |
| 67524151 | 67529158 | chr15 | − | exo | hsa_circ_0000620 | 0.6311 | 0.010074 | AAGAB |
| 72190367 | 72190535 | chr15 | + | as | hsa_circ_0000625 | 1.4454 | 0.010087 | MYO9A |
| 26596234 | 26598016 | chr2 | + | exo | hsa_circ_0007325 | 0.7986 | 0.010097 | EPT1 |
| 40043851 | 40048700 | chr17 | − | exo | hsa_circ_0002994 | 1.6497 | 0.010108 | ACLY |
| 99696751 | 99697026 | chr7 | − | int | hsa_circ_0001729 | 1.7880 | 0.010114 | MCM7 |
| 58971731 | 59002413 | chr1 | − | exo | hsa_circ_0000072 | 0.7763 | 0.010117 | OMA1 |
| 69555402 | 69573131 | chr2 | − | exo | hsa_circ_0055038 | 0.8500 | 0.010169 | GFPT1 |
| 72797242 | 72804408 | chrX | + | exo | hsa_circ_0091072 | 1.1488 | 0.010195 | CHIC1 |
| 183361267 | 183390272 | chr3 | + | exo | hsa_circ_0001368 | 0.5583 | 0.010222 | KLHL24 |
| 47597226 | 47597337 | chr19 | − | exo | hsa_circ_0051657 | 1.4804 | 0.010302 | ZC3H4 |
| 32377296 | 32385259 | chr1 | − | exo | hsa_circ_0004893 | 0.5734 | 0.010313 | PTP4A2 |
| 68434283 | 68439038 | chr15 | + | exo | hsa_circ_0008378 | 0.8573 | 0.010389 | PIAS1 |
| 64410315 | 64415745 | chr15 | + | exo | hsa_circ_0035818 | 1.4132 | 0.010442 | SNX1 |
| 45103516 | 45104245 | chr7 | + | exo | hsa_circ_0007145 | 1.5786 | 0.010462 | CCM2 |
| 196846280 | 196869639 | chr3 | − | exo | hsa_circ_0068697 | 0.4473 | 0.010467 | DLG1 |
| 99952765 | 99953427 | chr7 | + | int | hsa_circ_0005925 | 1.3549 | 0.010477 | PILRB |
| 66030044 | 66031213 | chr15 | − | exo | hsa_circ_0035949 | 0.7090 | 0.010483 | DENND4A |
| 21788274 | 21795059 | chr12 | − | exo | hsa_circ_0025593 | 1.4355 | 0.010497 | LDHB |
| 45885784 | 45889438 | chr19 | − | exo | | 1.2233 | 0.010509 | PPP1R13L |
| 98231032 | 98232213 | chr9 | − | exo | | 1.1882 | 0.010540 | PTCH1 |
| 196533449 | 196539722 | chr3 | + | exo | hsa_circ_0004950 | 0.8061 | 0.010581 | PAK2 |
| 148963425 | 148964313 | chr7 | + | exo | hsa_circ_0082864 | 1.1824 | 0.010608 | ZNF783 |
| 21971315 | 21972024 | chr14 | − | exo | hsa_circ_0000523 | 1.2852 | 0.010658 | METTL3 |
| 118429198 | 118461358 | chr1 | − | exo | hsa_circ_0013752 | 0.7852 | 0.010675 | GDAP2 |
| 70263118 | 70265962 | chr11 | + | exo | hsa_circ_0023339 | 1.4294 | 0.010682 | CTTN |
| 87760370 | 87795646 | chr16 | − | exo | hsa_circ_0040786 | 1.2153 | 0.010686 | KLHDC4 |
| 133331260 | 133331719 | chr12 | − | exo | hsa_circ_0029554 | 1.1272 | 0.010691 | ANKLE2 |
| 74963795 | 74967483 | chr15 | − | exo | hsa_circ_0000633 | 0.6458 | 0.010704 | EDC3 |
| 84964005 | 84971984 | chr1 | − | exo | hsa_circ_0013055 | 1.5255 | 0.010705 | GNG5 |
| 27248705 | 27251320 | chr17 | − | exo | hsa_circ_0003481 | 1.3302 | 0.010739 | PHF12 |
| 31648743 | 31652605 | chr12 | − | exo | hsa_circ_0007680 | 0.5937 | 0.010742 | DENND5B |
| 32602655 | 32620661 | chr2 | + | exo | hsa_circ_0053441 | 0.6806 | 0.010747 | BIRC6 |
| 13343862 | 13383218 | chr4 | − | s/o | | 0.7267 | 0.010748 | RAB28 |
| 73936249 | 73950301 | chr11 | + | exo | | 1.1379 | 0.010767 | PPME1 |
| 129928347 | 129937061 | chr9 | + | exo | | 0.5111 | 0.010812 | RALGPS1 |
| 10478730 | 10479094 | chr19 | − | exo | hsa_circ_0049249 | 1.3475 | 0.010826 | TYK2 |
| 159583951 | 159609979 | chr3 | + | exo | | 1.1315 | 0.010833 | SCHIP1 |
| 83799882 | 83803093 | chr4 | − | exo | hsa_circ_0006618 | 0.6812 | 0.010862 | SEC31A |
| 61044064 | 61045948 | chr11 | − | exo | hsa_circ_0022249 | 1.2166 | 0.010868 | VWCE |
| 7402357 | 7402810 | chr17 | + | exo | hsa_circ_0000741 | 2.2094 | 0.010869 | POLR2A |
| 33114072 | 33122599 | chr20 | + | exo | | 1.0834 | 0.010870 | DYNLRB1 |
| 1085346 | 1087148 | chr5 | − | exo | hsa_circ_0001464 | 1.5090 | 0.010909 | SLC12A7 |
| 49736908 | 49738142 | chr3 | + | s/o | hsa_circ_0001302 | 1.1934 | 0.010924 | RNF123 |
| 596922 | 598502 | chr11 | + | exo | | 1.7240 | 0.010970 | PHRF1 |
| 19371334 | 19379935 | chr18 | + | exo | | 0.6646 | 0.010971 | MIB1 |
| 31662050 | 31676195 | chr10 | + | int | hsa_circ_0002641 | 0.7562 | 0.010994 | ZEB1 |
| 1756835 | 1770677 | chr1 | − | exo | hsa_circ_0009362 | 1.7546 | 0.011003 | GNB1 |
| 148963425 | 148975618 | chr7 | + | exo | hsa_circ_0082865 | 1.1837 | 0.011011 | ZNF783 |
| 138614015 | 138618873 | chr5 | + | exo | hsa_circ_0074188 | 1.1109 | 0.011024 | SNHG4 |
| 137708362 | 137708530 | chr5 | + | exo | hsa_circ_0074101 | 1.3001 | 0.011030 | KDM3B |
| 73238416 | 73239651 | chr17 | − | exo | hsa_circ_0003684 | 1.2569 | 0.011042 | GGA3 |
| 75211135 | 75216111 | chr15 | − | s/o | | 1.1278 | 0.011051 | COX5A |
| 128750760 | 128754065 | chr2 | − | exo | hsa_circ_0056390 | 1.3396 | 0.011056 | SAP130 |
| 14720962 | 14721193 | chr16 | − | exo | hsa_circ_0002771 | 0.8071 | 0.011060 | PARN |
| 111795586 | 111800397 | chr9 | − | exo | hsa_circ_0087888 | 1.2116 | 0.011072 | TMEM245 |
| 71954641 | 71957283 | chr16 | + | exo | hsa_circ_0005016 | 0.5551 | 0.011106 | IST1 |
| 23495059 | 23495584 | chr14 | − | exo | hsa_circ_0031267 | 1.1742 | 0.011117 | PSMB5 |
| 46515157 | 46570050 | chr11 | − | exo | hsa_circ_0008415 | 0.8861 | 0.011171 | AMBRA1 |
| 172520651 | 172560211 | chr1 | + | exo | hsa_circ_0015261 | 1.1967 | 0.011174 | SUCO |
| 35800609 | 35800763 | chr17 | + | exo | | 1.3288 | 0.011178 | TADA2A |
| 76910271 | 76928416 | chr10 | + | exo | hsa_circ_0018905 | 1.1206 | 0.011184 | SAMD8 |
| 53595651 | 53596787 | chrX | − | exo | hsa_circ_0090750 | 1.3007 | 0.011205 | HUWE1 |
| 165878068 | 165878169 | chr1 | + | s/o | hsa_circ_0000148 | 1.2927 | 0.011244 | UCK2 |
| 119176864 | 119182471 | chr3 | − | s/o | hsa_circ_0066869 | 0.8764 | 0.011256 | TMEM39A |
| 2659529 | 2661781 | chr4 | + | exo | hsa_circ_0068925 | 0.8323 | 0.011272 | FAM193A |
| 36782074 | 36788753 | chr18 | − | s/o | | 1.0637 | 0.011272 | LINC00669 |
| 85781564 | 85781775 | chr4 | − | exo | hsa_circ_0070348 | 0.7712 | 0.011281 | WDFY3 |
| 20625572 | 20641530 | chr13 | + | exo | hsa_circ_0029633 | 0.5357 | 0.011318 | ZMYM2 |
| 51871576 | 51874004 | chr1 | − | exo | hsa_circ_0004877 | 0.7607 | 0.011334 | EPS15 |
| 13632601 | 13644961 | chr6 | − | exo | hsa_circ_0001577 | 0.5947 | 0.011371 | RANBP9 |
| 41652024 | 41657584 | chr22 | − | exo | | 1.3668 | 0.011387 | RANGAP1 |
| 18877942 | 18882804 | chr16 | − | exo | hsa_circ_0038288 | 0.5705 | 0.011397 | SMG1 |
| 14720967 | 14721193 | chr16 | − | int | | 0.7889 | 0.011411 | PARN |
| 46083263 | 46083463 | chr1 | + | int | hsa_circ_0092293 | 1.2304 | 0.011429 | NASP |
| 99672757 | 99675056 | chr7 | − | exo | hsa_circ_0002806 | 1.1830 | 0.011447 | ZNF3 |
| 132984928 | 132988783 | chr9 | + | exo | hsa_circ_0089090 | 1.2383 | 0.011462 | NCS1 |
| 127361340 | 127361454 | chr7 | + | exo | hsa_circ_0082131 | 0.6618 | 0.011521 | SND1 |
| 105818714 | 105821514 | chr14 | + | exo | hsa_circ_0033598 | 1.2270 | 0.011525 | PACS2 |
| 57928838 | 57929628 | chr12 | − | exo | hsa_circ_0004034 | 1.1804 | 0.011565 | DCTN2 |
| 62567915 | 62587267 | chr17 | − | exo | hsa_circ_0006644 | 0.7494 | 0.011580 | SMURF2 |
| 151719232 | 151729550 | chr4 | − | exo | hsa_circ_0004636 | 0.5455 | 0.011591 | LRBA |
| 37976787 | 37978667 | chr8 | + | exo | hsa_circ_0083960 | 0.7803 | 0.011601 | ASH2L |
| 79607545 | 79612827 | chr6 | + | exo | hsa_circ_0077109 | 2.0234 | 0.011607 | IRAK1BP1 |
| 26204839 | 26205043 | chr6 | + | s/o | hsa_circ_0001588 | 2.7898 | 0.011612 | HIST1H4E |
| 105280916 | 105297121 | chr6 | − | exo | hsa_circ_0077520 | 0.8798 | 0.011615 | HACE1 |
| 133907680 | 133914026 | chr3 | − | exo | hsa_circ_0005768 | 0.5832 | 0.011649 | RYK |
| 139415730 | 139416814 | chr7 | − | exo | hsa_circ_0001756 | 0.6065 | 0.011652 | HIPK2 |
| 64298925 | 64302245 | chr17 | + | exo | hsa_circ_0045361 | 1.4344 | 0.011705 | PRKCA |
| 157405795 | 157431695 | chr6 | + | exo | hsa_circ_0004381 | 1.2179 | 0.011706 | ARID1B |
| 201826223 | 201840455 | chr1 | + | exo | hsa_circ_0015936 | 1.2367 | 0.011719 | IPO9 |
| 44290402 | 44303983 | chr1 | + | exo | hsa_circ_0012107 | 1.3922 | 0.011721 | ST3GAL3 |
| 89958600 | 89967202 | chr16 | + | exo | hsa_circ_0003421 | 1.5143 | 0.011738 | TCF25 |
| 36983885 | 37002162 | chr15 | + | exo | hsa_circ_0008295 | 0.8868 | 0.011743 | C15orf41 |
| 19345732 | 19359646 | chr18 | + | exo | hsa_circ_0000835 | 0.7281 | 0.011756 | MIB1 |
| 6599924 | 6613005 | chr4 | + | exo | hsa_circ_0069087 | 2.1733 | 0.011778 | MAN2B2 |
| 3474817 | 3477249 | chr17 | − | exo | | 0.3593 | 0.011802 | TRPV1 |
| 50000450 | 50002944 | chr19 | + | s/o | hsa_circ_0051908 | 1.3069 | 0.011802 | RPS11 |
| 23614293 | 23616004 | chr20 | − | exo | hsa_circ_0059616 | 1.1427 | 0.011807 | CST3 |
| 99196173 | 99197507 | chr10 | − | s/o | hsa_circ_0005887 | 1.2903 | 0.011829 | EXOSC1 |
| 50145502 | 50145737 | chr3 | + | exo | hsa_circ_0001305 | 1.1367 | 0.011866 | RBM5 |
| 2663960 | 2732801 | chr16 | + | s/o | | 1.2070 | 0.011878 | KCTD5 |
| 149905066 | 149905920 | chrX | + | exo | hsa_circ_0006764 | 0.8456 | 0.011962 | MTMR1 |
| 4700365 | 4707364 | chr16 | + | exo | hsa_circ_0007032 | 1.1981 | 0.011963 | MGRN1 |
| 33794470 | 33836186 | chr1 | − | exo | hsa_circ_0011480 | 1.2071 | 0.011964 | PHC2 |
| 100456456 | 100460449 | chr7 | + | exo | | 1.2925 | 0.011976 | SLC12A9 |
| 99347143 | 99375698 | chr6 | − | exo | hsa_circ_0077417 | 0.7582 | 0.011986 | FBXL4 |
| 172477945 | 172482290 | chr3 | + | exo | | 0.8803 | 0.011994 | ECT2 |
| 141712668 | 141716304 | chr8 | − | exo | hsa_circ_0007049 | 1.2384 | 0.012044 | PTK2 |
| 5754784 | 5755476 | chr4 | + | int | | 1.2273 | 0.012088 | EVC |
| 15907201 | 15907487 | chr17 | + | int | | 1.2204 | 0.012136 | TTC19 |
| 47767862 | 47768203 | chr19 | + | s/o | | 1.3356 | 0.012148 | CCDC9 |
| 242644067 | 242651486 | chr2 | + | exo | hsa_circ_0001124 | 1.2339 | 0.012154 | ING5 |
| 50929382 | 50952912 | chr14 | − | exo | | 0.7234 | 0.012190 | MAP4K5 |
| 62291306 | 62292503 | chr11 | − | int | hsa_circ_0000319 | 1.1159 | 0.012191 | AHNAK |
| 87782278 | 87795646 | chr16 | − | exo | hsa_circ_0000724 | 1.2923 | 0.012196 | KLHDC4 |
| 139818045 | 139828890 | chr5 | + | exo | hsa_circ_0007402 | 0.8734 | 0.012200 | ANKHD1 |
| 71428942 | 71479919 | chr14 | + | exo | hsa_circ_0032391 | 1.2891 | 0.012202 | PCNX |
| 170033042 | 170052104 | chr6 | − | exo | hsa_circ_0003130 | 1.2128 | 0.012219 | WDR27 |
| 130782554 | 130782733 | chr7 | + | as | hsa_circ_0001745 | 1.4246 | 0.012246 | LINC-PINT |
| 2521314 | 2521523 | chr1 | − | as | hsa_circ_0000010 | 0.6162 | 0.012298 | FAM213B |
| 122495211 | 122495481 | chr12 | − | as | hsa_circ_0000453 | 0.4478 | 0.012332 | BCL7A |
| 67201209 | 67201305 | chr16 | + | int | | 1.1797 | 0.012341 | HSF4 |
| 34921480 | 34925873 | chr19 | + | exo | hsa_circ_0005325 | 0.8318 | 0.012345 | UBA2 |
| 121002874 | 121009094 | chr12 | + | exo | hsa_circ_0005043 | 0.7230 | 0.012359 | RNF10 |
| 37117031 | 37118448 | chr19 | + | s/o | | 0.8649 | 0.012364 | ZNF382 |
| 116879948 | 116925405 | chr10 | + | exo | hsa_circ_0020094 | 0.7233 | 0.012368 | ATRNL1 |
| 85834810 | 85840607 | chr16 | + | exo | hsa_circ_0040747 | 1.2636 | 0.012378 | COX4I1 |
| 132841908 | 132848755 | chr9 | + | exo | hsa_circ_0002625 | 0.8181 | 0.012411 | GPR107 |
| 39845126 | 39847898 | chr17 | + | exo | hsa_circ_0043650 | 1.7318 | 0.012434 | EIF1 |
| 65473318 | 65492309 | chr2 | + | s/o | hsa_circ_0001022 | 0.6597 | 0.012491 | ACTR2 |
| 11868091 | 11873219 | chr16 | − | exo | hsa_circ_0037886 | 0.7977 | 0.012504 | ZC3H7A |
| 34954930 | 34957919 | chr19 | + | exo | hsa_circ_0006992 | 1.5719 | 0.012584 | UBA2 |
| 33413822 | 33415375 | chr1 | − | exo | hsa_circ_0000048 | 1.1972 | 0.012588 | RNF19B |
| 133291443 | 133294706 | chr12 | + | exo | hsa_circ_0029545 | 1.4302 | 0.012613 | PGAM5 |
| 128099296 | 128099870 | chr9 | + | exo | hsa_circ_0003270 | 1.2368 | 0.012657 | GAPVD1 |
| 36133847 | 36194359 | chr14 | − | exo | hsa_circ_0031677 | 0.6231 | 0.012691 | RALGAPA1 |
| 43102811 | 43164956 | chr15 | − | exo | hsa_circ_0034869 | 0.8938 | 0.012698 | TTBK2 |
| 196118683 | 196120490 | chr3 | − | exo | hsa_circ_0005051 | 1.6335 | 0.012698 | UBXN7 |
| 29706428 | 29706988 | chr22 | + | exo | | 1.2741 | 0.012720 | GAS2L1 |
| 33122307 | 33133051 | chr19 | − | exo | hsa_circ_0050395 | 0.6831 | 0.012732 | ANKRD27 |
| 100410368 | 100410830 | chr7 | − | exo | hsa_circ_0001730 | 1.3752 | 0.012733 | EPHB4 |
| 32207322 | 32211102 | chr20 | + | exo | hsa_circ_0003426 | 1.7039 | 0.012747 | CBFA2T2 |
| 71706495 | 71706597 | chr11 | + | exo | hsa_circ_0023412 | 1.3556 | 0.012748 | RNF121 |
| 37408480 | 37428144 | chr19 | + | exo | | 0.7142 | 0.012776 | ZNF568 |
| 103916775 | 103917971 | chr10 | + | exo | hsa_circ_0000257 | 0.5599 | 0.012793 | NOLC1 |
| 138203933 | 138235925 | chr7 | + | exo | hsa_circ_0082580 | 0.6394 | 0.012794 | TRIM24 |
| 17448965 | 17449496 | chr19 | − | as | hsa_circ_0000911 | 0.7863 | 0.012805 | GTPBP3 |
| 80962990 | 80972395 | chr17 | − | exo | hsa_circ_0046599 | 1.1819 | 0.012805 | B3GNTL1 |
| 47188416 | 47308084 | chr22 | + | exo | hsa_circ_0063846 | 0.8649 | 0.012814 | TBC1D22A |
| 123661905 | 123683844 | chr10 | − | exo | hsa_circ_0002850 | 0.5712 | 0.012844 | ATE1 |
| 5941631 | 5957984 | chr19 | − | exo | hsa_circ_0048801 | 1.4348 | 0.012846 | RANBP3 |
| 73930508 | 73932323 | chr5 | − | exo | hsa_circ_0006859 | 0.5304 | 0.012847 | ENC1 |
| 185643369 | 185644451 | chr3 | − | s/o | hsa_circ_0001372 | 1.6843 | 0.012877 | TRA2B |
| 102114807 | 102116717 | chr7 | − | exo | hsa_circ_0081672 | 1.1419 | 0.012906 | POLR2J |
| 34241449 | 34246936 | chr20 | − | int | hsa_circ_0001146 | 0.8280 | 0.012913 | CPNE1 |
| 35825037 | 35840505 | chr6 | − | exo | hsa_circ_0002165 | 0.9001 | 0.012928 | SRPK1 |
| 50289531 | 50290616 | chr3 | + | exo | hsa_circ_0007083 | 1.7360 | 0.012944 | GNAI2 |
| 12780993 | 12784130 | chr19 | + | exo | hsa_circ_0049539 | 1.1555 | 0.013014 | WDR83 |
| 135106002 | 135116484 | chr10 | − | exo | | 1.1218 | 0.013021 | TUBGCP2 |
| 43655238 | 43657441 | chr2 | − | exo | hsa_circ_0054309 | 0.9231 | 0.013023 | THADA |
| 131365821 | 131367756 | chr9 | + | exo | hsa_circ_0088865 | 1.3051 | 0.013030 | SPTAN1 |
| 20922396 | 20931987 | chr17 | − | exo | hsa_circ_0042474 | 1.6298 | 0.013048 | USP22 |
| 43501643 | 43519190 | chr6 | − | exo | hsa_circ_0076558 | 0.7612 | 0.013131 | XPO5 |
| 125843206 | 125856803 | chr3 | − | exo | hsa_circ_0067127 | 1.2455 | 0.013132 | ALDH1L1 |
| 83667030 | 83754378 | chr6 | − | exo | hsa_circ_0008236 | 0.7553 | 0.013209 | UBE3D |
| 62227835 | 62228117 | chr2 | + | exo | | 1.1972 | 0.013246 | COMMD1 |
| 138589767 | 138597206 | chr7 | − | exo | hsa_circ_0082614 | 0.8692 | 0.013302 | KIAA1549 |
| 15508205 | 15519805 | chr8 | + | exo | hsa_circ_0083391 | 0.7663 | 0.013304 | TUSC3 |
| 78293666 | 78327493 | chr13 | + | exo | hsa_circ_0000497 | 0.6754 | 0.013319 | SLAIN1 |
| 136215068 | 136218280 | chr9 | + | exo | hsa_circ_0089339 | 1.5818 | 0.013342 | RPL7A |
| 196118683 | 196129890 | chr3 | − | exo | hsa_circ_0001380 | 1.3718 | 0.013343 | UBXN7 |
| 134033588 | 134039622 | chr5 | + | exo | hsa_circ_0073997 | 0.8591 | 0.013345 | SEC24A |
| 137371579 | 137372196 | chr5 | − | int | hsa_circ_0001536 | 1.1504 | 0.013352 | FAM13B |
| 81503338 | 81545875 | chr12 | + | exo | | 0.6308 | 0.013363 | ACSS3 |
| 57808781 | 57816308 | chr17 | + | exo | hsa_circ_0006508 | 0.5082 | 0.013365 | VMP1 |
| 131860291 | 131860929 | chr9 | − | exo | hsa_circ_0003362 | 1.2397 | 0.013415 | CRAT |
| 35832287 | 35838568 | chr20 | + | exo | hsa_circ_0060262 | 0.6624 | 0.013457 | RPN2 |
| 4951438 | 4951561 | chr20 | − | exo | hsa_circ_0059369 | 1.3611 | 0.013459 | SLC23A2 |
| 31103152 | 31112611 | chr14 | + | exo | hsa_circ_0008046 | 0.8542 | 0.013516 | SCFD1 |
| 9196840 | 9197116 | chr16 | + | exo | | 1.2794 | 0.013538 | C16orf72 |
| 85667519 | 85695371 | chr16 | + | exo | hsa_circ_0040730 | 1.1669 | 0.013550 | GSE1 |
| 18770594 | 18781183 | chr17 | + | exo | hsa_circ_0042359 | 1.6581 | 0.013566 | PRPSAP2 |
| 78111184 | 78113939 | chr17 | − | exo | hsa_circ_0045986 | 1.3175 | 0.013571 | EIF4A3 |
| 196118683 | 196129888 | chr3 | − | int | | 1.4550 | 0.013576 | UBXN7 |
| 63447819 | 63448720 | chr15 | − | exo | hsa_circ_0035626 | 1.1521 | 0.013584 | RPS27L |
| 95756566 | 95756769 | chr2 | − | int | hsa_circ_0001045 | 1.4760 | 0.013590 | MRPS5 |
| 61655830 | 61657304 | chr17 | + | exo | hsa_circ_0045202 | 1.2731 | 0.013599 | DCAF7 |
| 124243539 | 124251384 | chr8 | − | exo | hsa_circ_0002663 | 0.8217 | 0.013670 | C8orf76 |
| 21006240 | 21013893 | chr13 | − | exo | hsa_circ_0029642 | 1.1389 | 0.013695 | CRYL1 |
| 176050287 | 176055026 | chr1 | − | exo | hsa_circ_0015364 | 0.8356 | 0.013704 | RFWD2 |
| 47441689 | 47446190 | chrX | + | exo | hsa_circ_0090446 | 0.3618 | 0.013709 | TIMP1 |
| 137569740 | 137570248 | chr7 | − | exo | hsa_circ_0082564 | 0.6395 | 0.013744 | CREB3L2 |
| 29927157 | 29931571 | chr8 | − | exo | hsa_circ_0083834 | 1.3444 | 0.013751 | SARAF |
| 46567153 | 46567326 | chr11 | − | exo | hsa_circ_0021866 | 1.1283 | 0.013753 | AMBRA1 |
| 50763829 | 50763992 | chr15 | + | exo | hsa_circ_0035217 | 0.8402 | 0.013789 | USP8 |
| 28233438 | 28233793 | chr1 | − | exo | hsa_circ_0005866 | 1.2861 | 0.013799 | RPA2 |
| 138189034 | 138223548 | chr7 | + | exo | hsa_circ_0082574 | 0.6762 | 0.013801 | TRIM24 |
| 211952259 | 211961069 | chr1 | − | exo | | 0.7884 | 0.013812 | LPGAT1 |
| 46455023 | 46534363 | chr11 | − | exo | hsa_circ_0021845 | 1.2536 | 0.013893 | AMBRA1 |
| 79940776 | 79940857 | chr13 | + | as | hsa_circ_0000499 | 1.1561 | 0.013915 | RBM26 |
| 202104277 | 202104928 | chr1 | − | exo | | 1.2901 | 0.013928 | ARL8A |
| 32874967 | 32881196 | chr22 | + | exo | hsa_circ_0001222 | 0.7014 | 0.013931 | FBXO7 |
| 238997 | 244761 | chr11 | + | exo | | 1.2634 | 0.013936 | PSMD13 |
| 87925620 | 87928449 | chr6 | + | exo | hsa_circ_0004058 | 0.7045 | 0.013968 | ZNF292 |
| 120600873 | 120622046 | chr12 | − | exo | hsa_circ_0028826 | 0.4425 | 0.013978 | GCN1L1 |
| 141004774 | 141009692 | chr5 | − | exo | hsa_circ_0074332 | 1.3237 | 0.013982 | HDAC3 |
| 34450934 | 34459751 | chr20 | + | exo | hsa_circ_0060158 | 0.8189 | 0.013986 | PHF20 |
| 213290658 | 213303232 | chr1 | + | exo | hsa_circ_0005522 | 0.7795 | 0.014008 | RPS6KC1 |
| 61458751 | 61459055 | chr8 | + | int | hsa_circ_0001804 | 1.1780 | 0.014020 | RAB2A |
| 32730071 | 32730272 | chr2 | + | exo | hsa_circ_0053816 | 0.8554 | 0.014023 | BIRC6 |
| 41670603 | 41677086 | chr22 | − | exo | hsa_circ_0001235 | 1.6625 | 0.014035 | RANGAP1 |
| 70096955 | 70098444 | chr10 | + | exo | hsa_circ_0018497 | 1.5635 | 0.014035 | HNRNPH3 |
| 73100965 | 73101425 | chr7 | + | exo | hsa_circ_0005588 | 1.3498 | 0.014043 | WBSCR22 |
| 41923912 | 41940125 | chr22 | − | s/o | hsa_circ_0001237 | 1.1616 | 0.014118 | POLR3H |
| 36297027 | 36301594 | chr1 | + | exo | | 0.6266 | 0.014159 | AGO4 |
| 74489529 | 74489999 | chr9 | − | exo | hsa_circ_0002921 | 0.8690 | 0.014176 | ABHD17B |
| 44386075 | 44386600 | chr1 | + | exo | hsa_circ_0006595 | 1.3189 | 0.014193 | ST3GAL3 |
| 58904342 | 58904854 | chr19 | + | exo | hsa_circ_0005598 | 1.3778 | 0.014228 | RPS5 |
| 87760370 | 87764250 | chr16 | − | exo | hsa_circ_0040784 | 1.1298 | 0.014251 | KLHDC4 |
| 81195758 | 81195954 | chr15 | − | int | hsa_circ_0000644 | 0.7574 | 0.014267 | RP11-351M8.1 |
| 42204878 | 42206295 | chr22 | + | exo | hsa_circ_0001238 | 1.6163 | 0.014304 | CCDC134 |
| 131186934 | 131190123 | chr3 | − | exo | hsa_circ_0004559 | 0.7653 | 0.014313 | MRPL3 |
| 59996760 | 60002460 | chr17 | − | exo | hsa_circ_0007064 | 0.8831 | 0.014324 | INTS2 |
| 17275585 | 17279592 | chr10 | + | exo | hsa_circ_0017873 | 1.8026 | 0.014357 | VIM |
| 133894452 | 133901915 | chr3 | − | exo | hsa_circ_0067434 | 0.5456 | 0.014428 | RYK |
| 126370175 | 126384781 | chr10 | − | exo | hsa_circ_0003570 | 1.4199 | 0.014449 | FAM53B |
| 59963381 | 59964938 | chr15 | − | exo | hsa_circ_0035537 | 0.9253 | 0.014466 | BNIP2 |
| 18648410 | 18649243 | chr19 | − | exo | hsa_circ_0050119 | 1.5874 | 0.014508 | FKBP8 |
| 71479716 | 71479919 | chr14 | + | exo | hsa_circ_0032434 | 1.3009 | 0.014539 | PCNX |
| 153940503 | 153944604 | chrX | − | exo | | 0.3843 | 0.014598 | GAB3 |
| 247039338 | 247040604 | chr1 | − | exo | hsa_circ_0006097 | 0.8425 | 0.014604 | AHCTF1 |
| 141727696 | 141756959 | chr8 | − | exo | hsa_circ_0085743 | 0.8366 | 0.014607 | PTK2 |
| 42204878 | 42204997 | chr22 | + | exo | hsa_circ_0063603 | 0.5593 | 0.014615 | CCDC134 |
| 4848946 | 4849292 | chr17 | − | exo | hsa_circ_0041630 | 1.1671 | 0.014673 | PFN1 |
| 36367548 | 36367938 | chr1 | + | exo | hsa_circ_0000052 | 1.2126 | 0.014674 | AGO1 |
| 15601324 | 15618413 | chr2 | − | exo | hsa_circ_0008083 | 0.6393 | 0.014765 | NBAS |
| 45011271 | 45016256 | chr7 | − | exo | hsa_circ_0080099 | 1.1044 | 0.014779 | MYO1G |
| 75684855 | 75687163 | chr15 | − | s/o | hsa_circ_0000635 | 1.1061 | 0.014780 | SIN3A |
| 69402278 | 69406258 | chr16 | − | exo | | 1.0768 | 0.014785 | TERF2 |
| 8257934 | 8275635 | chr7 | − | exo | hsa_circ_0079422 | 1.7264 | 0.014807 | ICA1 |
| 23395341 | 23396023 | chr14 | − | exo | hsa_circ_0031250 | 1.1688 | 0.014836 | PRMT5 |
| 33222418 | 33233138 | chr20 | − | exo | hsa_circ_0059988 | 0.8788 | 0.014854 | PIGU |
| 127338260 | 127339678 | chr5 | − | int | | 1.1695 | 0.014865 | CTC-228N24.3 |
| 44089823 | 44092540 | chr7 | + | exo | hsa_circ_0080000 | 1.3923 | 0.014886 | DBNL |
| 175996707 | 176015460 | chr1 | − | exo | hsa_circ_0015359 | 0.7912 | 0.014935 | RFWD2 |
| 38792600 | 38798083 | chr21 | + | int | hsa_circ_0004407 | 1.2907 | 0.014938 | DYRK1A |
| 35700187 | 35717783 | chr9 | − | exo | hsa_circ_0086887 | 1.1962 | 0.014985 | TLN1 |
| 148571844 | 148582568 | chrX | − | exo | hsa_circ_0091671 | 0.7860 | 0.014994 | IDS |
| 8942842 | 8948002 | chr20 | + | s/o | | 0.8816 | 0.015027 | PLCB1 |
| 110978278 | 110980107 | chrX | + | exo | | 0.8180 | 0.015049 | ALG13 |
| 73946854 | 73952028 | chr17 | − | exo | hsa_circ_0000804 | 0.9243 | 0.015124 | ACOX1 |
| 90098243 | 90098338 | chr16 | + | int | hsa_circ_0000729 | 1.3693 | 0.015134 | GAS8 |
| 17503341 | 17510986 | chr4 | − | exo | hsa_circ_0069249 | 1.3227 | 0.015139 | QDPR |
| 151043647 | 151049345 | chr5 | − | exo | hsa_circ_0004104 | 1.5340 | 0.015147 | SPARC |
| 49083857 | 49114471 | chr3 | − | exo | | 0.8922 | 0.015159 | QRICH1 |
| 185580493 | 185606838 | chr4 | + | exo | | 0.9257 | 0.015160 | PRIMPOL |
| 180668491 | 180669345 | chr5 | − | exo | hsa_circ_0075402 | 1.2672 | 0.015205 | GNB2L1 |
| 8922945 | 8923402 | chr1 | − | exo | hsa_circ_0009610 | 1.4526 | 0.015238 | ENO1 |
| 42315491 | 42320959 | chr20 | + | exo | hsa_circ_0060455 | 1.1707 | 0.015248 | MYBL2 |
| 18393289 | 18396093 | chr20 | − | exo | | 1.2461 | 0.015256 | DZANK1 |
| 57250018 | 57255327 | chr16 | + | exo | hsa_circ_0039557 | 1.4653 | 0.015263 | RSPRY1 |
| 35846859 | 35865177 | chr1 | + | exo | hsa_circ_0011545 | 1.4694 | 0.015288 | ZMYM4 |
| 129585852 | 129599405 | chr3 | − | int | | 1.2479 | 0.015332 | TMCC1 |
| 70251370 | 70253697 | chr11 | + | exo | hsa_circ_0003376 | 1.3503 | 0.015335 | CTTN |
| 89576897 | 89579445 | chr16 | + | exo | hsa_circ_0040932 | 1.1999 | 0.015343 | SPG7 |
| 124243539 | 124243997 | chr8 | − | exo | hsa_circ_0007779 | 0.8760 | 0.015389 | C8orf76 |
| 25666964 | 25678184 | chr1 | + | exo | hsa_circ_0007054 | 0.7748 | 0.015401 | TMEM50A |
| 49609654 | 49618211 | chr10 | + | exo | hsa_circ_0002968 | 0.7324 | 0.015403 | MAPK8 |
| 41535864 | 41555081 | chr15 | + | exo | hsa_circ_0034682 | 1.1608 | 0.015429 | CHP1 |
| 45745035 | 45745147 | chr21 | + | exo | | 1.4917 | 0.015444 | PFKL |
| 32399131 | 32409407 | chr2 | + | exo | hsa_circ_0005695 | 0.8365 | 0.015457 | SLC30A6 |
| 42191638 | 42202061 | chr15 | − | exo | hsa_circ_0034776 | 1.6340 | 0.015467 | EHD4 |
| 34789429 | 34803241 | chr6 | + | exo | hsa_circ_0076057 | 1.2929 | 0.015481 | UHRF1BP1 |
| 97820925 | 97823884 | chr7 | + | exo | hsa_circ_0081207 | 0.7386 | 0.015487 | LMTK2 |
| 219467402 | 219468493 | chr1 | + | s/o | | 0.5295 | 0.015524 | XLOC_000566 |
| 86335196 | 86335470 | chr15 | − | int | hsa_circ_0000648 | 1.1215 | 0.015553 | KLHL25 |
| 3150990 | 3151271 | chr10 | + | int | | 1.1343 | 0.015559 | PFKP |
| 62651928 | 62651997 | chr11 | + | exo | hsa_circ_0022589 | 1.2827 | 0.015594 | SLC3A2 |
| 10417222 | 10417519 | chr1 | + | int | | 0.8948 | 0.015621 | KIF1B |
| 29237938 | 29246335 | chr18 | − | exo | hsa_circ_0047351 | 0.7722 | 0.015635 | B4GALT6 |
| 37424841 | 37426651 | chr9 | + | exo | hsa_circ_0001861 | 1.3551 | 0.015654 | GRHPR |
| 76388556 | 76411108 | chr17 | + | exo | hsa_circ_0045890 | 1.1171 | 0.015657 | PGS1 |
| 61630443 | 61631258 | chr11 | + | exo | hsa_circ_0022392 | 2.0890 | 0.015679 | FADS2 |
| 1746096 | 1747980 | chr17 | + | exo | hsa_circ_0005946 | 1.2793 | 0.015680 | RPA1 |
| 48561918 | 48565892 | chr20 | + | exo | hsa_circ_0008212 | 1.1417 | 0.015685 | RNF114 |
| 48653019 | 48653467 | chr19 | − | exo | | 1.0902 | 0.015709 | LIG1 |
| 47164293 | 47166311 | chr10 | − | exo | hsa_circ_0018293 | 1.2065 | 0.015736 | ANXA8 |
| 157357968 | 157431695 | chr6 | + | exo | hsa_circ_0004019 | 1.1911 | 0.015779 | ARID1B |
| 2404006 | 2406083 | chr7 | + | exo | hsa_circ_0001671 | 1.1473 | 0.015781 | EIF3B |
| 154330380 | 154346797 | chr5 | + | s/o | | 0.8260 | 0.015796 | MRPL22 |
| 115005725 | 115007010 | chr1 | − | exo | hsa_circ_0004127 | 0.8768 | 0.015800 | TRIM33 |
| 92459222 | 92459688 | chr15 | + | exo | hsa_circ_0003252 | 1.1614 | 0.015809 | SLCO3A1 |
| 15374347 | 15468949 | chr6 | + | exo | | 1.1946 | 0.015830 | JARID2 |
| 47647225 | 47648679 | chr11 | − | exo | hsa_circ_0022025 | 0.8493 | 0.015942 | MTCH2 |
| 155936315 | 155936610 | chr1 | − | int | | 1.1520 | 0.015949 | ARHGEF2 |
| 41936702 | 41936799 | chr22 | − | exo | hsa_circ_0063577 | 1.4459 | 0.015956 | POLR3H |
| 32873245 | 32881962 | chr20 | − | exo | hsa_circ_0059910 | 1.3635 | 0.015964 | AHCY |
| 208017318 | 208017404 | chr1 | − | int | hsa_circ_0000178 | 1.2514 | 0.015965 | C1orf132 |
| 112528529 | 112530940 | chr12 | − | exo | hsa_circ_0028342 | 0.9258 | 0.015995 | NAA25 |
| 55766695 | 55766841 | chr7 | − | exo | hsa_circ_0007536 | 1.2737 | 0.015995 | FKBP9P1 |
| 34240825 | 34240924 | chr20 | − | int | hsa_circ_0001145 | 1.1942 | 0.016035 | CPNE1 |
| 68030482 | 68044315 | chr8 | + | exo | hsa_circ_0084677 | 0.7558 | 0.016062 | CSPP1 |
| 111893832 | 111926584 | chr12 | − | exo | hsa_circ_0028241 | 0.8439 | 0.016070 | ATXN2 |
| 79613111 | 79614128 | chr10 | − | exo | hsa_circ_0006649 | 1.5690 | 0.016098 | DLG5 |
| 21735928 | 21746820 | chr13 | − | exo | hsa_circ_0029696 | 1.5233 | 0.016118 | SKA3 |
| 76483440 | 76483669 | chr3 | + | int | hsa_circ_0008025 | 1.3262 | 0.016126 | ROBO2 |
| 140037802 | 140046483 | chr4 | − | exo | | 0.8354 | 0.016131 | ELF2 |
| 34646852 | 34664195 | chr18 | + | exo | hsa_circ_0047474 | 0.9201 | 0.016143 | KIAA1328 |
| 103204187 | 103204638 | chr9 | + | exo | hsa_circ_0006388 | 0.7468 | 0.016149 | MSANTD3 |
| 76388556 | 76395618 | chr17 | + | exo | hsa_circ_0045888 | 1.2352 | 0.016149 | PGS1 |
| 162546566 | 162567648 | chr1 | + | exo | hsa_circ_0004646 | 0.3939 | 0.016184 | UAP1 |
| 38071612 | 38075809 | chr22 | + | exo | hsa_circ_0063226 | 0.6696 | 0.016192 | LGALS1 |
| 171867882 | 171884915 | chr2 | − | exo | hsa_circ_0057028 | 0.8435 | 0.016195 | TLK1 |
| 26056274 | 26056412 | chr6 | + | as | hsa_circ_0001584 | 1.1395 | 0.016205 | HIST1H1C |
| 39738530 | 39743147 | chr17 | − | exo | hsa_circ_0043614 | 0.7836 | 0.016215 | KRT14 |
| 50655754 | 50656296 | chr13 | − | int | hsa_circ_0000487 | 1.1763 | 0.016219 | DLEU2 |
| 107419754 | 107420500 | chr6 | − | exo | | 1.1157 | 0.016220 | BEND3 |
| 38072992 | 38075809 | chr22 | + | exo | hsa_circ_0063227 | 0.3903 | 0.016274 | LGALS1 |
| 15844604 | 15844890 | chr1 | − | exo | hsa_circ_0010039 | 1.0960 | 0.016277 | CASP9 |
| 27975161 | 27977818 | chr17 | − | exo | hsa_circ_0042799 | 0.8971 | 0.016303 | SSH2 |
| 225933 | 255988 | chr10 | + | exo | hsa_circ_0017359 | 0.8557 | 0.016312 | ZMYND11 |
| 56552467 | 56555366 | chr12 | + | exo | hsa_circ_0026888 | 1.5226 | 0.016344 | MYL6 |
| 154018228 | 154020560 | chrX | − | exo | hsa_circ_0001952 | 0.6646 | 0.016355 | MPP1 |
| 28120137 | 28120955 | chr17 | − | s/o | | 0.7301 | 0.016388 | SSH2 |
| 70200406 | 70202360 | chr11 | + | exo | hsa_circ_0000337 | 0.8684 | 0.016448 | PPFIA1 |
| 20754921 | 20755662 | chr22 | + | exo | hsa_circ_0062317 | 1.2486 | 0.016469 | ZNF74 |
| 33467081 | 33467233 | chr3 | − | exo | hsa_circ_0064742 | 0.7556 | 0.016472 | UBP1 |
| 37463238 | 37465010 | chr2 | + | exo | | 1.1325 | 0.016476 | NDUFAF7 |
| 124515234 | 124567405 | chr3 | − | exo | | 1.5749 | 0.016483 | ITGB5 |
| 9588086 | 9588851 | chr18 | − | exo | hsa_circ_0046881 | 0.6948 | 0.016492 | PPP4R1 |
| 17267718 | 17270294 | chr19 | + | exo | hsa_circ_0003215 | 1.2718 | 0.016524 | MYO9B |
| 123600596 | 123683844 | chr10 | − | exo | hsa_circ_0020246 | 0.5713 | 0.016551 | ATE1 |
| 145044816 | 145083858 | chr5 | − | int | | 0.8200 | 0.016607 | PRELID2 |
| 151630710 | 151641111 | chr1 | + | exo | hsa_circ_0002394 | 0.7708 | 0.016621 | SNX27 |
| 1307231 | 1317024 | chr11 | − | exo | hsa_circ_0008301 | 1.3541 | 0.016634 | TOLLIP |
| 132990021 | 133002144 | chr7 | + | s/o | hsa_circ_0001749 | 0.8878 | 0.016659 | EXOC4 |
| 51381438 | 51387763 | chr10 | + | exo | hsa_circ_0006820 | 1.3525 | 0.016680 | TIMM23B |
| 42604156 | 42604482 | chr12 | − | exo | hsa_circ_0000392 | 1.2070 | 0.016775 | YAF2 |
| 43435781 | 43442579 | chr22 | − | s/o | | 1.4194 | 0.016794 | TTLL1 |
| 42152047 | 42152455 | chr17 | + | exo | hsa_circ_0044013 | 1.3831 | 0.016805 | G6PC3 |
| 52779255 | 52782372 | chr20 | − | exo | hsa_circ_0060937 | 1.1677 | 0.016858 | CYP24A1 |
| 124736287 | 124752049 | chr9 | − | exo | hsa_circ_0088353 | 1.1090 | 0.016878 | TTLL11 |
| 217363519 | 217366188 | chr2 | + | exo | hsa_circ_0058191 | 1.2686 | 0.017006 | RPL37A |
| 117423010 | 117448255 | chr12 | + | exo | hsa_circ_0028613 | 1.0990 | 0.017033 | FBXW8 |
| 128923737 | 128926028 | chr10 | + | exo | hsa_circ_0002456 | 1.1769 | 0.017035 | DOCK1 |
| 11868091 | 11876244 | chr16 | − | exo | hsa_circ_0005394 | 0.8983 | 0.017046 | ZC3H7A |
| 44219735 | 44221614 | chr6 | + | s/o | hsa_circ_0076654 | 1.1238 | 0.017085 | HSP90AB1 |
| 68874847 | 68875691 | chr5 | + | exo | hsa_circ_0004914 | 0.7880 | 0.017097 | GTF2H2C_2 |
| 56350154 | 56351009 | chr17 | + | as | hsa_circ_0000791 | 1.3274 | 0.017113 | MPO |
| 120541629 | 120546271 | chr12 | − | exo | hsa_circ_0028711 | 1.1480 | 0.017136 | RAB35 |
| 69558390 | 69589077 | chr14 | − | exo | hsa_circ_0003485 | 1.1527 | 0.017159 | DCAF5 |
| 78908585 | 78908697 | chr5 | + | int | hsa_circ_0001502 | 1.7057 | 0.017161 | PAPD4 |
| 5117344 | 5118588 | chr17 | + | exo | | 1.1853 | 0.017188 | LOC100130950 |
| 23381682 | 23391205 | chr7 | − | exo | | 1.5646 | 0.017190 | IGF2BP3 |
| 76587931 | 76588078 | chr15 | − | exo | hsa_circ_0036413 | 0.8690 | 0.017208 | ETFA |
| 75197494 | 75197572 | chr15 | − | exo | hsa_circ_0036315 | 1.2532 | 0.017220 | FAM219B |
| 102039994 | 102047945 | chr7 | + | exo | hsa_circ_0007004 | 1.1200 | 0.017221 | PRKRIP1 |
| 14576842 | 14578407 | chr12 | + | exo | | 0.8551 | 0.017254 | ATF7IP |
| 62400036 | 62407203 | chr11 | − | exo | hsa_circ_0022487 | 1.7254 | 0.017269 | GANAB |
| 79158663 | 79258971 | chr4 | + | exo | hsa_circ_0070101 | 1.4822 | 0.017279 | FRAS1 |
| 230798886 | 230807386 | chr1 | + | exo | hsa_circ_0016867 | 0.6408 | 0.017286 | COG2 |
| 129177441 | 129188260 | chr3 | + | exo | | 1.1753 | 0.017300 | IFT122 |
| 141840570 | 141874498 | chr8 | − | exo | hsa_circ_0002908 | 0.6333 | 0.017321 | PTK2 |
| 49051181 | 49051358 | chr3 | − | as | hsa_circ_0001300 | 1.1462 | 0.017326 | WDR6 |
| 132337477 | 132350323 | chr3 | − | exo | hsa_circ_0005875 | 0.6907 | 0.017374 | ACAD11 |
| 1003727 | 1006847 | chr12 | + | exo | hsa_circ_0003251 | 1.7019 | 0.017396 | WNK1 |
| 20632714 | 20633702 | chr13 | + | exo | hsa_circ_0029634 | 0.7371 | 0.017411 | ZMYM2 |
| 161071837 | 161072165 | chr1 | − | exo | hsa_circ_0000145 | 1.3146 | 0.017434 | PFDN2 |
| 74431765 | 74434317 | chr7 | + | exo | hsa_circ_0080635 | 1.4375 | 0.017557 | GATSL1 |
| 45766370 | 45766625 | chr19 | + | exo | hsa_circ_0000940 | 1.4619 | 0.017587 | MARK4 |
| 39623414 | 39628754 | chr14 | − | exo | | 1.1983 | 0.017607 | TRAPPC6B |
| 54398613 | 54426184 | chr18 | + | exo | hsa_circ_0047744 | 0.7895 | 0.017613 | WDR7 |
| 1529263 | 1532681 | chr7 | + | as | hsa_circ_0001668 | 1.0919 | 0.017764 | INTS1 |
| 44785299 | 44804994 | chr1 | − | exo | hsa_circ_0005246 | 1.1736 | 0.017768 | ERI3 |
| 126142863 | 126143349 | chr11 | + | exo | hsa_circ_0002019 | 1.1535 | 0.017775 | FOXRED1 |
| 4860124 | 4860901 | chr9 | + | s/o | hsa_circ_0007592 | 0.7802 | 0.017796 | RCL1 |
| 2641342 | 2641475 | chr20 | − | exo | hsa_circ_0059216 | 1.3015 | 0.017841 | IDH3B |
| 17937576 | 17941969 | chr20 | − | exo | hsa_circ_0009173 | 1.1042 | 0.017850 | SNX5 |
| 204154487 | 204157049 | chr2 | + | exo | hsa_circ_0005307 | 0.7237 | 0.017861 | CYP20A1 |
| 93557925 | 93558139 | chr15 | + | exo | hsa_circ_0037007 | 0.8133 | 0.017866 | CHD2 |
| 104168410 | 104170593 | chr14 | + | as | | 0.4057 | 0.017907 | XRCC3 |
| 89961445 | 89973730 | chr16 | + | exo | hsa_circ_0041098 | 1.2971 | 0.017939 | TCF25 |
| 34574331 | 34574681 | chr6 | − | exo | hsa_circ_0006757 | 1.1987 | 0.017946 | C6orf106 |
| 13375816 | 13378350 | chr10 | − | exo | | 1.2305 | 0.017985 | SEPHS1 |
| 40990747 | 40991397 | chr17 | + | exo | hsa_circ_0007635 | 1.1085 | 0.018016 | PSME3 |
| 46998521 | 47000299 | chr17 | + | exo | | 1.1724 | 0.018024 | UBE2Z |
| 24680620 | 24681035 | chr14 | − | exo | hsa_circ_0031359 | 1.0956 | 0.018048 | CHMP4A |
| 58752099 | 58753159 | chr16 | − | exo | hsa_circ_0039714 | 1.0956 | 0.018086 | GOT2 |
| 33222890 | 33253066 | chr13 | + | exo | hsa_circ_0029941 | 0.7604 | 0.018112 | PDS5B |
| 36668400 | 36706837 | chr2 | + | exo | hsa_circ_0003578 | 1.3906 | 0.018152 | CRIM1 |
| 61833638 | 61835159 | chr20 | − | exo | hsa_circ_0004858 | 1.2632 | 0.018183 | YTHDF1 |
| 99930628 | 99958106 | chr6 | − | exo | | 0.6981 | 0.018195 | USP45 |
| 50824277 | 50829407 | chr12 | + | exo | hsa_circ_0026232 | 0.6971 | 0.018197 | LARP4 |
| 81121205 | 81124712 | chr16 | − | exo | | 0.3559 | 0.018212 | GCSH |
| 57138486 | 57138658 | chr4 | + | exo | hsa_circ_0069821 | 1.5503 | 0.018241 | KIAA1211 |
| 49387188 | 49388817 | chr19 | − | exo | | 1.2312 | 0.018269 | TULP2 |
| 53068519 | 53069224 | chr12 | − | exo | hsa_circ_0026462 | 0.3096 | 0.018342 | KRT1 |
| 7692128 | 7693184 | chr19 | − | exo | | 1.1534 | 0.018347 | XAB2 |
| 20632714 | 20641530 | chr13 | + | exo | hsa_circ_0029636 | 0.5227 | 0.018355 | ZMYM2 |
| 35890583 | 35893796 | chr21 | − | int | | 0.8013 | 0.018384 | RCAN1 |
| 127453811 | 127454077 | chr2 | − | as | hsa_circ_0001065 | 1.6601 | 0.018393 | GYPC |
| 87516379 | 87541609 | chr5 | − | s/o | hsa_circ_0005476 | 0.8544 | 0.018396 | TMEM161B |
| 82302569 | 82372915 | chr1 | + | exo | hsa_circ_0013048 | 0.4893 | 0.018399 | LPHN2 |
| 12923896 | 12924379 | chr19 | + | s/o | | 1.1474 | 0.018432 | RNASEH2A |
| 126370175 | 126395456 | chr10 | − | exo | hsa_circ_0002282 | 1.3883 | 0.018480 | FAM53B |
| 29087976 | 29096597 | chr16 | + | exo | | 0.8885 | 0.018518 | RRN3P2 |
| 35948707 | 35948901 | chr22 | − | as | hsa_circ_0001223 | 1.1953 | 0.018520 | RASD2 |
| 101870646 | 101870949 | chr7 | + | exo | hsa_circ_0006501 | 1.5795 | 0.018525 | CUX1 |
| 131060182 | 131084192 | chr7 | + | exo | hsa_circ_0001746 | 0.7092 | 0.018530 | MKLN1 |
| 76856475 | 76936907 | chr18 | + | exo | hsa_circ_0048000 | 0.8060 | 0.018537 | ATP9B |
| 77200394 | 77221573 | chr7 | + | exo | hsa_circ_0008901 | 0.6886 | 0.018546 | PTPN12 |
| 34951655 | 34957074 | chr21 | − | exo | hsa_circ_0061548 | 0.8611 | 0.018552 | DONSON |
| 44380003 | 44383675 | chr17 | + | exo | hsa_circ_0044195 | 0.7335 | 0.018607 | LRRC37A |
| 69404385 | 69406258 | chr16 | − | exo | | 1.4288 | 0.018666 | TERF2 |
| 47585118 | 47585318 | chr19 | − | int | hsa_circ_0092316 | 0.8843 | 0.018745 | ZC3H4 |
| 197755551 | 197763093 | chr2 | − | exo | hsa_circ_0057607 | 0.8002 | 0.018773 | PGAP1 |
| 58298877 | 58299104 | chr19 | + | int | | 1.0977 | 0.018781 | ZNF587 |
| 204354306 | 204360076 | chr2 | − | exo | hsa_circ_0005613 | 0.8540 | 0.018807 | RAPH1 |
| 47410171 | 47424963 | chr21 | + | exo | hsa_circ_0062035 | 1.2070 | 0.018848 | COL6A1 |
| 175716656 | 175717958 | chr5 | + | exo | hsa_circ_0075092 | 0.7637 | 0.018853 | SIMC1 |
| 40544231 | 40546159 | chr1 | − | exo | hsa_circ_0011883 | 1.2890 | 0.018861 | PPT1 |
| 85701292 | 85742653 | chr11 | − | exo | hsa_circ_0023918 | 0.8448 | 0.018898 | PICALM |
| 77344726 | 77348597 | chr15 | − | exo | hsa_circ_0000641 | 1.1877 | 0.018911 | TSPAN3 |
| 4803230 | 4803902 | chr17 | + | s/o | hsa_circ_0000737 | 1.4445 | 0.018911 | C17orf107 |
| 3029928 | 3031568 | chr12 | + | exo | hsa_circ_0007584 | 1.1403 | 0.019046 | TULP3 |
| 12558109 | 12560696 | chr3 | + | exo | hsa_circ_0005028 | 1.2959 | 0.019099 | TSEN2 |
| 42204878 | 42206004 | chr22 | + | exo | hsa_circ_0063604 | 1.2429 | 0.019130 | CCDC134 |
| 5624475 | 5624965 | chr11 | + | exo | hsa_circ_0002969 | 1.1230 | 0.019135 | TRIM6 |
| 144547886 | 144557755 | chr8 | − | exo | hsa_circ_0008835 | 1.1529 | 0.019140 | ZC3H3 |
| 55756487 | 55757046 | chr19 | − | exo | hsa_circ_0005746 | 1.2654 | 0.019181 | PPP6R1 |
| 38019339 | 38019640 | chr22 | + | s/o | | 1.1421 | 0.019204 | GGA1 |
| 76224429 | 76239510 | chr11 | + | exo | hsa_circ_0008189 | 0.6755 | 0.019266 | C11orf30 |
| 79158663 | 79207693 | chr4 | + | exo | hsa_circ_0070100 | 1.4753 | 0.019272 | FRAS1 |
| 38318005 | 38319154 | chr17 | + | exo | hsa_circ_0043509 | 1.1855 | 0.019293 | CASC3 |
| 5307418 | 5320002 | chr17 | − | exo | hsa_circ_0041700 | 0.8607 | 0.019301 | NUP88 |
| 20811436 | 20811534 | chr14 | − | s/o | hsa_circ_0000519 | 2.5622 | 0.019414 | RPPH1 |
| 129679303 | 129688984 | chr7 | − | exo | hsa_circ_0004364 | 1.1832 | 0.019423 | ZC3HC1 |
| 99196947 | 99197507 | chr10 | − | exo | hsa_circ_0004896 | 1.3360 | 0.019428 | EXOSC1 |
| 1149794 | 1150165 | chr20 | + | int | | 1.1762 | 0.019444 | PSMF1 |
| 30766558 | 30776889 | chr15 | + | s/o | | 0.8045 | 0.019467 | CTD-3092A11.1 |
| 75245155 | 75245386 | chr14 | + | s/o | | 1.2030 | 0.019469 | YLPM1 |
| 172305190 | 172314987 | chr2 | + | exo | hsa_circ_0001081 | 1.1679 | 0.019545 | DCAF17 |
| 123465675 | 123467085 | chr12 | + | exo | hsa_circ_0000457 | 1.4525 | 0.019565 | ARL6IP4 |
| 2761457 | 2763755 | chr19 | − | exo | hsa_circ_0005454 | 1.3277 | 0.019584 | SGTA |
| 58131647 | 58135954 | chr3 | + | exo | hsa_circ_0066367 | 1.2480 | 0.019593 | FLNB |
| 150464886 | 150469104 | chr1 | + | exo | | 1.0881 | 0.019607 | TARS2 |
| 155891165 | 155895634 | chr1 | − | exo | hsa_circ_0000140 | 1.1565 | 0.019619 | KIAA0907 |
| 111372281 | 111585853 | chr7 | − | exo | hsa_circ_0081968 | 0.8673 | 0.019623 | DOCK4 |
| 42365180 | 42375484 | chr19 | + | exo | hsa_circ_0051244 | 1.1967 | 0.019712 | RPS19 |
| 155679563 | 155697529 | chr1 | + | exo | | 1.1394 | 0.019760 | DAP3 |
| 50373934 | 50374070 | chr19 | + | as | hsa_circ_0000950 | 1.1150 | 0.019870 | AKT1S1 |
| 7154584 | 7170346 | chr2 | + | exo | hsa_circ_0052490 | 0.7914 | 0.019896 | RNF144A |
| 44564901 | 44605080 | chr19 | + | s/o | hsa_circ_0000938 | 1.1499 | 0.019926 | ZNF223 |
| 21475023 | 21475314 | cirri6 | − | exo | hsa_circ_0038416 | 1.2480 | 0.019933 | SMG1P3 |
| 52959282 | 52975384 | chr1 | − | exo | hsa_circ_0003632 | 0.6725 | 0.019977 | ZCCHC11 |
| 110778463 | 110780253 | cirri2 | + | exo | hsa_circ_0002628 | 1.3496 | 0.019977 | ATP2A2 |
| 41239064 | 41239269 | chr19 | + | exo | hsa_circ_0051133 | 1.1650 | 0.019990 | ITPKC |
| 5691329 | 5691678 | chr19 | + | exo | hsa_circ_0048766 | 0.7216 | 0.020045 | RPL36 |
| 96437462 | 96438798 | chr5 | + | exo | hsa_circ_0001516 | 1.0972 | 0.020063 | CTD-2215E18.1 |
| 27207184 | 27207404 | chr17 | + | as | hsa_circ_0000746 | 1.1145 | 0.020090 | FLOT2 |
| 112915454 | 112915819 | chr12 | + | exo | hsa_circ_0008666 | 1.0893 | 0.020096 | PTPN11 |
| 100078647 | 100125588 | chr4 | + | s/o | | 1.1299 | 0.020137 | LOC100507053 |
| 120636656 | 120636803 | chr12 | − | exo | hsa_circ_0028861 | 1.7461 | 0.020145 | RPLP0 |
| 44442444 | 44443967 | chr1 | + | exo | hsa_circ_0012125 | 0.5000 | 0.020167 | ATP6V0B |
| 141231004 | 141259451 | chr3 | + | exo | hsa_circ_0067582 | 0.8604 | 0.020173 | RASA2 |
| 29151053 | 29151137 | chr17 | − | int | hsa_circ_0000755 | 1.2158 | 0.020282 | CRLF3 |
| 51032748 | 51061888 | chr1 | − | exo | hsa_circ_0008197 | 0.8276 | 0.020321 | FAF1 |
| 103557736 | 103567658 | chr10 | − | exo | hsa_circ_0019608 | 0.6286 | 0.020346 | MGEA5 |
| 141889569 | 141935848 | chr8 | − | exo | hsa_circ_0006421 | 0.6979 | 0.020372 | PTK2 |
| 16218645 | 16219768 | cirri6 | + | exo | hsa_circ_0000679 | 1.2053 | 0.020405 | ABCC1 |
| 101077886 | 101101559 | chr13 | + | exo | hsa_circ_0030777 | 0.6347 | 0.020412 | PCCA |
| 3316517 | 3344780 | chr4 | + | s/o | hsa_circ_0007215 | 0.4692 | 0.020418 | RGS12 |
| 128902834 | 128951922 | chr8 | + | exo | hsa_circ_0085540 | 1.2894 | 0.020440 | PVT1 |
| 242593960 | 242594758 | chr2 | + | exo | hsa_circ_0003662 | 1.2396 | 0.020479 | ATG4B |
| 13705452 | 13712528 | chr10 | − | exo | hsa_circ_0017819 | 0.7495 | 0.020501 | FRMD4A |
| 15070092 | 15071959 | chr3 | + | exo | hsa_circ_0004046 | 1.3858 | 0.020531 | NR2C2 |
| 109046047 | 109096774 | chr12 | − | exo | hsa_circ_0028067 | 0.6928 | 0.020535 | CORO1C |
| 89961445 | 89967202 | chr16 | + | exo | hsa_circ_0002631 | 1.3642 | 0.020544 | TCF25 |
| 82267504 | 82336803 | chr9 | + | exo | hsa_circ_0087298 | 0.6477 | 0.020551 | TLE4 |
| 170833400 | 170833731 | chr5 | + | exo | hsa_circ_0075021 | 0.8402 | 0.020638 | NPM1 |
| 79293871 | 79385737 | chr4 | + | exo | hsa_circ_0070154 | 1.0739 | 0.020706 | FRAS1 |
| 41754418 | 41754725 | chr19 | + | exo | hsa_circ_0002882 | 0.6458 | 0.020707 | AXL |
| 1032390 | 1036245 | chr19 | + | exo | hsa_circ_0048140 | 1.2471 | 0.020735 | CNN2 |
| 135917473 | 135919313 | chr9 | + | exo | hsa_circ_0089310 | 1.3299 | 0.020737 | GTF3C5 |
| 174241551 | 174247917 | chr1 | + | exo | hsa_circ_0000161 | 0.8823 | 0.020770 | RABGAP1L |
| 69565029 | 69565697 | chr2 | − | exo | hsa_circ_OOOIO25 | 0.8713 | 0.020773 | GFPT1 |
| 126194344 | 126207514 | chr8 | + | s/o | | 0.8727 | 0.020787 | NSMCE2 |
| 120636920 | 120637008 | chr12 | − | exo | hsa_circ_0028864 | 1.8456 | 0.020827 | RPLP0 |
| 10254441 | 10254663 | chr19 | − | exo | hsa_circ_0049201 | 1.2343 | 0.020840 | DNMT1 |
| 737261 | 738476 | chr4 | + | exo | hsa_circ_0001385 | 1.1361 | 0.020843 | PCGF3 |
| 9524591 | 9525849 | chr18 | + | exo | hsa_circ_0005158 | 1.2116 | 0.020901 | RALBP1 |
| 51860052 | 51913807 | chr1 | − | exo | hsa_circ_0012425 | 0.7381 | 0.020922 | EPS15 |
| 20007406 | 20007883 | chr11 | + | int | | 1.0984 | 0.020927 | NAV2 |
| 41641614 | 41654110 | chr22 | − | exo | hsa_circ_0063512 | 1.0790 | 0.020935 | RANGAP1 |
| 81536885 | 81545875 | chr12 | + | exo | hsa_circ_0027679 | 0.7139 | 0.020960 | ACSS3 |
| 134312006 | 134312109 | chr9 | + | exo | hsa_circ_0089202 | 1.3489 | 0.021004 | PRRC2B |
| 71692135 | 71713438 | chr16 | − | exo | hsa_circ_0005638 | 0.7348 | 0.021014 | PHLPP2 |
| 26918715 | 26926848 | chr17 | − | int | | 1.3995 | 0.021016 | RP11-192H23.4 |
| 3076997 | 3078169 | chr6 | + | exo | hsa_circ_0001571 | 1.2091 | 0.021024 | RIPK1 |
| 156169625 | 156170263 | chr1 | + | exo | hsa_circ_0007393 | 1.2359 | 0.021039 | SLC25A44 |
| 31025753 | 31040421 | chr19 | + | exo | | 0.6196 | 0.021041 | ZNF536 |
| 93760203 | 93761283 | chr14 | − | exo | hsa_circ_0006947 | 0.5290 | 0.021053 | BTBD7 |
| 179268906 | 179271227 | chr5 | − | exo | hsa_circ_0007903 | 1.1430 | 0.021189 | C5orf45 |
| 38829400 | 38860865 | chr7 | − | exo | | 1.1586 | 0.021193 | VPS41 |
| 152480271 | 152522207 | chr7 | + | exo | hsa_circ_0005181 | 0.7798 | 0.021234 | ACTR3B |
| 6301901 | 6312055 | chr18 | − | exo | | 1.5685 | 0.021297 | L3MBTL4 |
| 103558598 | 103567658 | chr10 | − | exo | hsa_circ_0019612 | 0.6258 | 0.021301 | MGEA5 |
| 65266845 | 65267149 | chr11 | + | int | hsa_circ_0000325 | 1.2695 | 0.021346 | XLOC_l2_002352 |
| 89958600 | 89962491 | chr16 | + | exo | hsa_circ_0002507 | 1.3083 | 0.021370 | TCF25 |
| 53459000 | 53459207 | chr10 | + | int | hsa_circ_0000238 | 1.3442 | 0.021371 | PRKG1 |
| 68044185 | 68049838 | chr8 | + | exo | hsa_circ_0003388 | 0.8874 | 0.021402 | CSPP1 |
| 20910208 | 20916248 | chr17 | | exo | hsa_circ_0042459 | 1.4704 | 0.021424 | USP22 |
| 68664356 | 68668137 | chr4 | + | int | | 0.5837 | 0.021429 | UBA6-AS1 |
| 228356262 | 228389631 | chr2 | + | exo | hsa_circ_0058514 | 0.8408 | 0.021442 | AGFG1 |
| 37571278 | 37576171 | chr17 | − | exo | hsa_circ_0043415 | 1.2997 | 0.021443 | MED1 |
| 45916934 | 45917292 | chr19 | − | exo | hsa_circ_0051488 | 1.1088 | 0.021520 | ERCC1 |
| 159444736 | 159445499 | chr2 | + | int | | 0.4906 | 0.021522 | PKP4 |
| 10407213 | 10426634 | chr5 | + | exo | hsa_circ_0071862 | 0.8279 | 0.021567 | MARCH6 |
| 41664100 | 41677086 | chr22 | − | exo | hsa_circ_0063534 | 1.3340 | 0.021570 | RANGAP1 |
| 76849058 | 76867088 | chr17 | − | exo | hsa_circ_0045942 | 1.0994 | 0.021571 | TIMP2 |
| 96485180 | 96489456 | chr13 | − | exo | hsa_circ_0008274 | 0.2125 | 0.021608 | UGGT2 |
| 76388556 | 76394432 | chr17 | + | exo | | 1.3537 | 0.021631 | PGS1 |
| 40986531 | 40991029 | chr17 | + | exo | | 1.2420 | 0.021652 | PSME3 |
| 147131761 | 147131890 | chr1 | − | exo | hsa_circ_0013959 | 1.3294 | 0.021696 | ACP6 |
| 69189773 | 69201088 | chr16 | + | exo | hsa_circ_0007018 | 0.7746 | 0.021734 | CIRH1A |
| 57460082 | 57463515 | chr11 | + | exo | hsa_circ_0022168 | 1.2497 | 0.021756 | ZDHHC5 |
| 58267923 | 58292135 | chr17 | − | exo | hsa_circ_0044922 | 0.8529 | 0.021771 | USP32 |
| 36688187 | 36689461 | chr22 | + | as | hsa_circ_0001226 | 1.4102 | 0.021771 | MYH9 |
| 47022647 | 47033857 | chr22 | + | exo | hsa_circ_0001250 | 1.2911 | 0.021804 | GRAMD4 |
| 62743001 | 62749256 | chr12 | + | exo | hsa_circ_0027355 | 1.0902 | 0.021839 | USP15 |
| 41259625 | 41270446 | chr4 | + | exo | hsa_circ_0069574 | 1.1288 | 0.021875 | UCHL1 |
| 34991685 | 35006275 | chr11 | + | exo | hsa_circ_0000290 | 0.7083 | 0.021907 | PDHX |
| 33501898 | 33509669 | chr20 | + | exo | hsa_circ_0060011 | 1.1506 | 0.021931 | ACSS2 |
| 98558883 | 98563518 | chr7 | + | exo | | 0.7953 | 0.021940 | TRRAP |
| 28215844 | 28217612 | chr6 | − | exo | hsa_circ_0075912 | 1.2593 | 0.021941 | ZKSCAN4 |
| 140960310 | 140963182 | chr5 | − | exo | hsa_circ_0074323 | 0.9168 | 0.021944 | DIAPH1 |
| 39943995 | 39944161 | chr19 | + | exo | hsa_circ_0000932 | 1.4956 | 0.021962 | SUPT5H |
| 22236974 | 22237296 | chr16 | + | exo | hsa_circ_0038487 | 1.1509 | 0.022040 | EEF2K |
| 114214355 | 114216087 | chr1 | + | exo | hsa_circ_0006583 | 0.8459 | 0.022042 | MAGI3 |
| 139076432 | 139094456 | chr3 | − | exo | hsa_circ_0067550 | 1.3007 | 0.022049 | COPB2 |
| 126275990 | 126283543 | chr11 | + | exo | hsa_circ_0024825 | 1.1953 | 0.022093 | ST3GAL4 |
| 40655612 | 40657441 | chr2 | − | exo | hsa_circ_0005232 | 0.6457 | 0.022096 | SLC8A1 |
| 73418464 | 73429935 | chr11 | − | exo | hsa_circ_0004596 | 0.8874 | 0.022137 | RAB6A |
| 89611055 | 89617017 | chr16 | + | exo | hsa_circ_0005217 | 1.1581 | 0.022148 | SPG7 |
| 58499574 | 58500089 | chr19 | − | exo | hsa_circ_0052320 | 1.2199 | 0.022163 | ZNF606 |
| 74561481 | 74580819 | chr18 | + | exo | hsa_circ_0002799 | 0.6090 | 0.022200 | ZNF236 |
| 66021409 | 66031213 | chr15 | − | exo | hsa_circ_0035944 | 0.7751 | 0.022263 | DENND4A |
| 203378441 | 203379702 | chr2 | + | exo | hsa_circ_0057805 | 0.7354 | 0.022277 | BMPR2 |
| 157256599 | 157431695 | chr6 | + | exo | hsa_circ_0078402 | 1.2699 | 0.022282 | ARID1B |
| 48660270 | 48660397 | chr19 | − | exo | hsa_circ_0051732 | 1.1868 | 0.022288 | LIG1 |
| 30590251 | 30614497 | chr7 | − | s/o | | 0.6617 | 0.022305 | LOC401320 |
| 48805819 | 48867006 | chr8 | − | exo | hsa_circ_0084402 | 1.3539 | 0.022329 | PRKDC |
| 120335945 | 120338017 | chr11 | + | exo | | 0.6909 | 0.022337 | ARHGEF12 |
| 151139409 | 151139890 | chr1 | + | exo | hsa_circ_0000128 | 0.5841 | 0.022338 | SCNM1 |
| 159020527 | 159020864 | chr1 | + | int | | 1.7059 | 0.022364 | IFI16 |
| 19554859 | 19575269 | chr17 | + | exo | hsa_circ_0008603 | 0.7538 | 0.022373 | ALDH3A2 |
| 73614502 | 73664837 | chr14 | + | exo | hsa_circ_0002289 | 1.0961 | 0.022388 | PSEN1 |
| 25497809 | 25505580 | chr2 | − | exo | hsa_circ_0053028 | 1.3176 | 0.022432 | DNMT3A |
| 150464886 | 150470223 | chr1 | + | exo | hsa_circ_0014022 | 1.2273 | 0.022454 | TARS2 |
| 51459757 | 51459882 | chr3 | − | exo | hsa_circ_0065902 | 0.8793 | 0.022459 | VPRBP |
| 156619298 | 156629579 | chr7 | − | exo | hsa_circ_0005939 | 0.7823 | 0.022470 | LMBR1 |
| 6978421 | 6978937 | chr12 | + | exo | hsa_circ_0025278 | 1.4679 | 0.022481 | TPI1 |
| 4760668 | 4766974 | chr20 | − | exo | hsa_circ_0059354 | 1.0856 | 0.022522 | RASSF2 |
| 77210743 | 77212967 | chr7 | + | exo | hsa_circ_0008139 | 0.7540 | 0.022523 | PTPN12 |
| 46934593 | 46950619 | chr16 | + | exo | hsa_circ_0039219 | 1.4239 | 0.022545 | GPT2 |
| 128627027 | 128628933 | chr2 | − | exo | | 0.9026 | 0.022584 | AMMECR1L |
| 99220665 | 99220823 | chr9 | + | s/o | | 1.3223 | 0.022591 | HABP4 |
| 21117754 | 21120167 | chr19 | + | int | | 1.2696 | 0.022592 | ZNF85 |
| 73985878 | 73991029 | chr4 | − | exo | hsa_circ_0002831 | 0.7250 | 0.022636 | ANKRD17 |
| 77423853 | 77461496 | chr5 | − | exo | hsa_circ_0001501 | 0.7218 | 0.022638 | AP3B1 |
| 145008165 | 145009097 | chr8 | − | exo | hsa_circ_0085918 | 1.5398 | 0.022638 | PLEC |
| 75563802 | 75590926 | chr14 | − | exo | hsa_circ_0032664 | 1.2191 | 0.022707 | NEK9 |
| 43312046 | 43319232 | chr10 | + | exo | hsa_circ_0018242 | 1.3958 | 0.022740 | BMS1 |
| 154213988 | 154221913 | chr1 | + | exo | | 1.1414 | 0.022749 | UBAP2L |
| 36872786 | 36872903 | chr22 | − | s/o | | 1.2318 | 0.022788 | TXN2 |
| 55833019 | 55919286 | chr18 | + | exo | | 1.2316 | 0.022813 | NEDD4L |
| 29810948 | 29811369 | chr16 | + | exo | hsa_circ_0038929 | 0.4226 | 0.022831 | KIF22 |
| 26233194 | 26236101 | chr7 | − | exo | hsa_circ_0001691 | 2.6151 | 0.022834 | HNRNPA2B1 |
| 9931806 | 9950565 | chr18 | + | exo | hsa_circ_0046909 | 0.8656 | 0.022854 | VAPA |
| 8421822 | 8482867 | chr1 | − | exo | | 1.2534 | 0.022870 | RERE |
| 78183551 | 78191447 | chr1 | − | exo | hsa_circ_0000087 | 0.7292 | 0.022926 | USP33 |
| 180933651 | 181019422 | chr1 | + | s/o | | 1.1403 | 0.023044 | MR1 |
| 3659367 | 3659754 | chr1 | − | exo | | 0.8086 | 0.023061 | TP73-AS1 |
| 46529740 | 46534363 | chr11 | − | exo | hsa_circ_0003110 | 1.2477 | 0.023061 | AMBRA1 |
| 18044057 | 18047197 | chr19 | + | int | hsa_circ_0092331 | 1.5433 | 0.023073 | CCDC124 |
| 11958205 | 11984847 | chr17 | + | exo | hsa_circ_0007508 | 0.7951 | 0.023079 | MAP2K4 |
| 11426664 | 11427862 | chr2 | − | exo | hsa_circ_0006526 | 0.8665 | 0.023240 | ROCK2 |
| 68215377 | 68217269 | chr16 | + | exo | hsa_circ_0039934 | 0.5057 | 0.023258 | NFATC3 |
| 57808781 | 57851246 | chr17 | + | exo | hsa_circ_0005077 | 0.6032 | 0.023291 | VMP1 |
| 33307958 | 33350179 | chr11 | + | exo | hsa_circ_0008887 | 0.6092 | 0.023319 | HIPK3 |
| 80721840 | 80772810 | chr17 | + | exo | hsa_circ_0002225 | 0.7544 | 0.023320 | TBCD |
| 3792977 | 3797251 | chr11 | − | exo | hsa_circ_0004637 | 0.8931 | 0.023332 | NUP98 |
| 30354787 | 30358477 | chr17 | + | exo | hsa_circ_0042992 | 0.7303 | 0.023337 | LRRC37B |
| 40655612 | 40657444 | chr2 | − | exo | hsa_circ_0000994 | 0.6032 | 0.023369 | SLC8A1 |
| 41727784 | 41767671 | chr19 | + | exo | hsa_circ_0051167 | 1.1258 | 0.023395 | AXL |
| 17796781 | 17799020 | chr8 | + | int | hsa_circ_0001780 | 1.0898 | 0.023398 | PCM1 |
| 21288066 | 21288532 | chr22 | + | exo | hsa_circ_0001206 | 1.5920 | 0.023412 | CRKL |
| 114384158 | 114385269 | chr12 | − | exo | hsa_circ_0028540 | 1.2128 | 0.023462 | RBM19 |
| 172096787 | 172097333 | chr5 | + | exo | | 1.2661 | 0.023545 | NEURL1B |
| 76240770 | 76247617 | chr7 | − | exo | hsa_circ_0080813 | 1.1510 | 0.023576 | POMZP3 |
| 169982931 | 170052104 | chr6 | − | exo | hsa_circ_0078727 | 1.3842 | 0.023625 | WDR27 |
| 12123470 | 12139995 | chr10 | + | exo | hsa_circ_0017726 | 0.7996 | 0.023650 | DHTKD1 |
| 150157220 | 150174296 | chr6 | − | exo | hsa_circ_0003249 | 1.2231 | 0.023672 | LRP11 |
| 11647388 | 11650591 | chr16 | − | exo | hsa_circ_0037858 | 1.2037 | 0.023705 | LITAF |
| 80521229 | 80545148 | chr17 | + | exo | hsa_circ_0046430 | 1.4376 | 0.023731 | FOXK2 |
| 2790418 | 2811732 | chr19 | + | exo | hsa_circ_0048430 | 1.0897 | 0.023735 | THOP1 |
| 35236117 | 35236221 | chr20 | + | exo | hsa_circ_0001151 | 1.1461 | 0.023743 | C20orf24 |
| 24108020 | 24108462 | chr22 | − | exo | hsa_circ_0062556 | 1.2550 | 0.023800 | CHCHD10 |
| 8926337 | 8927309 | chr1 | − | exo | hsa_circ_0009624 | 1.9642 | 0.023837 | ENO1 |
| 37328806 | 37330036 | chr22 | + | exo | hsa_circ_0063179 | 1.5120 | 0.023874 | CSF2RB |
| 191765289 | 191789319 | chr2 | + | exo | hsa_circ_0001085 | 0.9166 | 0.023891 | GLS |
| 128843224 | 128846428 | chr7 | + | exo | | 0.7754 | 0.023893 | SMO |
| 91947919 | 91952074 | chr14 | − | exo | hsa_circ_0003045 | 0.7610 | 0.023917 | SMEK1 |
| 19992297 | 19992553 | chr3 | + | exo | | 1.2239 | 0.023921 | RAB5A |
| 73215386 | 73228707 | chr2 | − | exo | hsa_circ_0004699 | 1.3153 | 0.023970 | SFXN5 |
| 40293372 | 40301685 | chr13 | + | exo | hsa_circ_0006900 | 0.8425 | 0.024002 | COG6 |
| 65790201 | 65790370 | chr15 | − | exo | hsa_circ_0035907 | 1.1017 | 0.024090 | DPP8 |
| 44424435 | 44448984 | chr21 | + | exo | hsa_circ_0061854 | 0.7695 | 0.024137 | PKNOX1 |
| 23023562 | 23030758 | chr7 | − | exo | hsa_circ_0001682 | 0.6305 | 0.024176 | FAM126A |
| 66759730 | 66763002 | chr16 | − | exo | hsa_circ_0008242 | 1.4557 | 0.024271 | DYNC1LI2 |
| 235634163 | 235658138 | chr1 | − | exo | hsa_circ_0017069 | 1.1059 | 0.024296 | B3GALNT2 |
| 15651543 | 15660752 | chr6 | − | exo | | 1.1570 | 0.024334 | DTNBP1 |
| 32599525 | 32617924 | chr8 | + | exo | | 0.6972 | 0.024348 | NRG1 |
| 128902834 | 128903244 | chr8 | + | exo | hsa_circ_0001821 | 1.3293 | 0.024392 | PVT1 |
| 242606059 | 242608100 | chr2 | + | exo | hsa_circ_0006464 | 1.1331 | 0.024455 | ATG4B |
| 16474935 | 16496309 | chr20 | − | exo | | 0.7061 | 0.024459 | KIF16B |
| 112191581 | 112191719 | chr12 | + | int | | 1.5140 | 0.024496 | ACAD10 |
| 38991050 | 38996984 | chr5 | − | exo | hsa_circ_0072338 | 0.8584 | 0.024509 | RICTOR |
| 96986391 | 96994343 | chr14 | + | exo | hsa_circ_0002793 | 0.7729 | 0.024525 | PAPOLA |
| 1158623 | 1159348 | chr1 | − | exo | hsa_circ_0000002 | 1.2214 | 0.024540 | SDF4 |
| 82319697 | 82324614 | chr9 | + | exo | hsa_circ_0087302 | 1.2192 | 0.024595 | TLE4 |
| 103151076 | 103194718 | chr11 | + | exo | hsa_circ_0024139 | 0.7360 | 0.024731 | DYNC2H1 |
| 156516805 | 156549159 | chr7 | − | exo | | 0.8339 | 0.024741 | LMBR1 |
| 180793888 | 180794480 | chr1 | + | exo | hsa_circ_0005909 | 0.8511 | 0.024783 | XPR1 |
| 904262 | 908576 | chr5 | + | exo | | 1.3044 | 0.024789 | TRIP13 |
| 15601324 | 15601915 | chr2 | − | exo | hsa_circ_0005533 | 0.6736 | 0.024823 | NBAS |
| 25877593 | 25885213 | chr17 | + | exo | | 1.1019 | 0.024837 | KSR1 |
| 7301567 | 7303659 | chr12 | + | exo | | 0.4254 | 0.024900 | CLSTN3 |
| 56062586 | 56065142 | chr7 | + | exo | hsa_circ_0080252 | 1.0679 | 0.024916 | GBAS |
| 153040334 | 153040558 | chrX | + | exo | hsa_circ_0091774 | 0.6887 | 0.024958 | PLXNB3 |
| 88648196 | 88655783 | chr9 | − | exo | | 1.4389 | 0.025009 | GOLM1 |
| 136103119 | 136107785 | chr2 | − | exo | hsa_circ_0009086 | 0.8411 | 0.025014 | ZRANB3 |
| 64846522 | 64847170 | chr11 | + | as | hsa_circ_0000323 | 1.1201 | 0.025085 | CDCA5 |
| 69792863 | 69792948 | chr5 | − | exo | hsa_circ_0072837 | 1.8002 | 0.025109 | SMA5 |
| 77930940 | 77934851 | chr14 | + | exo | hsa_circ_0032777 | 1.3883 | 0.025154 | AHSA1 |
| 10784445 | 10794673 | chr2 | − | exo | hsa_circ_0052612 | 0.7979 | 0.025176 | NOL10 |
| 126445920 | 126452065 | chr3 | + | exo | hsa_circ_0003181 | 1.1906 | 0.025191 | CHCHD6 |
| 44027276 | 44027846 | chr7 | − | exo | hsa_circ_0079990 | 1.6755 | 0.025210 | POLR2J4 |
| 1802434 | 1816167 | chr5 | + | exo | hsa_circ_0071736 | 1.1276 | 0.025249 | NDUFS6 |
| 80962990 | 80992975 | chr17 | − | exo | hsa_circ_0046600 | 1.1956 | 0.025251 | B3GNTL1 |
| 41667909 | 41669502 | chr15 | + | exo | hsa_circ_0002124 | 0.5963 | 0.025274 | NUSAP1 |
| 114289547 | 114296633 | chr9 | + | exo | | 0.6895 | 0.025287 | ZNF483 |
| 44898222 | 44903183 | chr15 | − | exo | hsa_circ_0007457 | 0.8930 | 0.025309 | SPG11 |
| 136610762 | 136610850 | chr6 | − | int | hsa_circ_0001644 | 1.1044 | 0.025334 | BCLAF1 |
| 134032815 | 134044578 | chr5 | + | exo | hsa_circ_0003528 | 0.6606 | 0.025339 | SEC24A |
| 67803928 | 67804114 | chr11 | + | exo | hsa_circ_0023143 | 1.6195 | 0.025375 | NDUFS8 |
| 13064953 | 13065340 | chr19 | − | s/o | | 1.1839 | 0.025376 | GADD45GIP1 |
| 123471177 | 123512691 | chr3 | − | exo | hsa_circ_0002768 | 1.2739 | 0.025446 | MYLK |
| 4344838 | 4346286 | chr3 | − | int | | 1.0969 | 0.025490 | SUMF1 |
| 156468903 | 156489628 | chr6 | − | ing | hsa_circ_0001655 | 0.2962 | 0.025520 | |
| 16506736 | 16509085 | chr20 | − | exo | hsa_circ_0059490 | 1.1458 | 0.025524 | KIF16B |
| 21495206 | 21497305 | chr5 | + | exo | hsa_circ_0072025 | 1.9860 | 0.025538 | GUSBP1 |
| 1491500 | 1492649 | chr11 | − | s/o | hsa_circ_0000272 | 1.1550 | 0.025552 | MOB2 |
| 47551673 | 47552716 | chr3 | − | exo | hsa_circ_0065237 | 1.3161 | 0.025574 | ELP6 |
| 24021007 | 24046439 | chr2 | − | exo | hsa_circ_0052941 | 0.6803 | 0.025582 | ATAD2B |
| 110332157 | 110350696 | chr2 | − | exo | hsa_circ_0007654 | 0.6627 | 0.025651 | SEPT10 |
| 52729602 | 52765544 | chr4 | + | exo | hsa_circ_0069718 | 0.7981 | 0.025664 | DCUN1D4 |
| 12945995 | 12946213 | chr8 | − | exo | hsa_circ_0083377 | 1.1238 | 0.025678 | DLC1 |
| 70633355 | 70635201 | chr14 | − | exo | hsa_circ_0000551 | 0.6425 | 0.025679 | SLC8A3 |
| 108098321 | 108106561 | chr11 | + | exo | hsa_circ_0003641 | 0.8195 | 0.025735 | ATM |
| 19812493 | 19813291 | chr17 | − | exo | hsa_circ_0006256 | 0.8820 | 0.025846 | AKAP10 |
| 80718434 | 80719026 | chr16 | − | exo | hsa_circ_0004087 | 1.0927 | 0.025882 | CDYL2 |
| 105692405 | 105693097 | chr14 | − | exo | hsa_circ_0006153 | 1.3038 | 0.025885 | BRF1 |
| 5857869 | 5862937 | chr4 | − | exo | hsa_circ_0069076 | 1.5391 | 0.025910 | CRMP1 |
| 86297865 | 86301070 | chr9 | − | exo | hsa_circ_0005142 | 0.5488 | 0.025929 | UBQLN1 |
| 1746096 | 1756483 | chr17 | + | exo | hsa_circ_0000734 | 1.1986 | 0.025932 | RPA1 |
| 47466974 | 47470160 | chr3 | − | exo | hsa_circ_0007291 | 1.2285 | 0.025998 | SCAP |
| 133358881 | 133390005 | chr12 | − | exo | hsa_circ_0029562 | 1.2818 | 0.026011 | GOLGA3 |
| 242593960 | 242608100 | chr2 | + | exo | hsa_circ_0007159 | 1.1850 | 0.026025 | ATG4B |
| 31592304 | 31592917 | chr6 | + | int | | 0.4081 | 0.026026 | PRRC2A |
| 75936161 | 75936743 | chr13 | − | exo | hsa_circ_0030431 | 0.5713 | 0.026041 | TBC1D4 |
| 89396991 | 89400658 | chr4 | + | exo | hsa_circ_0070421 | 0.4729 | 0.026105 | HERC5 |
| 95504879 | 95550574 | chr8 | − | exo | hsa_circ_0084900 | 0.7147 | 0.026150 | KIAA1429 |
| 135917473 | 135930351 | chr9 | + | exo | hsa_circ_0089315 | 1.1465 | 0.026261 | GTF3C5 |
| 10159043 | 10177499 | chr8 | + | exo | | 1.3240 | 0.026323 | MSRA |
| 73414865 | 73428368 | chr15 | + | exo | | 0.7094 | 0.026364 | NEO1 |
| 31819817 | 31833504 | chr14 | − | exo | | 0.7565 | 0.026369 | HEATR5A |
| 17378345 | 17379205 | chr19 | + | int | hsa_circ_0092307 | 1.1743 | 0.026379 | BABAM1 |
| 125441236 | 125441681 | chr12 | − | exo | | 1.1403 | 0.026409 | DHX37 |
| 77085372 | 77103586 | chr11 | − | exo | hsa_circ_0023685 | 0.8172 | 0.026418 | PAK1 |
| 22399613 | 22404422 | chr4 | − | int | | 0.2999 | 0.026500 | GPR125 |
| 239169468 | 239186596 | chr2 | − | exo | hsa_circ_0058883 | 1.3817 | 0.026534 | PER2 |
| 16670634 | 16762746 | chr5 | − | exo | hsa_circ_0071981 | 0.8338 | 0.026591 | MYO10 |
| 1307231 | 1311639 | chr11 | − | exo | hsa_circ_0003379 | 1.4118 | 0.026704 | TOLLIP |
| 7209155 | 7239930 | chrY | + | exo | hsa_circ_0006322 | 1.5169 | 0.026771 | PRKY |
| 227075791 | 227076750 | chr1 | + | exo | hsa_circ_0016726 | 1.1323 | 0.026778 | PSEN2 |
| 18568426 | 18588029 | chr11 | − | exo | hsa_circ_0021467 | 0.8104 | 0.026778 | UEVLD |
| 62817884 | 62820990 | chr17 | − | exo | hsa_circ_0004702 | 1.1933 | 0.026829 | PLEKHM1P |
| 1192371 | 1192690 | chr1 | − | exo | hsa_circ_0008063 | 1.1194 | 0.026858 | UBE2J2 |
| 3848207 | 3862213 | chr11 | − | exo | hsa_circ_0020924 | 1.5787 | 0.026946 | RHOG |
| 137654907 | 137664218 | chr5 | − | exo | hsa_circ_0005537 | 1.2336 | 0.026984 | CDC25C |
| 29140758 | 29152576 | chr2 | + | exo | | 0.7076 | 0.027046 | WDR43 |
| 89202707 | 89221015 | chr14 | − | exo | hsa_circ_0032880 | 0.8331 | 0.027046 | EML5 |
| 72338241 | 72356774 | chr9 | − | exo | hsa_circ_0087159 | 0.8746 | 0.027055 | PTAR1 |
| 3550981 | 3551192 | chr19 | − | exo | hsa_circ_0048492 | 1.8490 | 0.027091 | MFSD12 |
| 37126308 | 37126939 | chr9 | + | exo | hsa_circ_0001860 | 0.6277 | 0.027093 | ZCCHC7 |
| 76062243 | 76062964 | chr7 | + | exo | hsa_circ_0080797 | 0.5683 | 0.027108 | ZP3 |
| 34736240 | 34776158 | chr11 | + | s/o | | 0.7331 | 0.027159 | RP4-743O11.1 |
| 39757276 | 39776553 | chr4 | + | exo | hsa_circ_0003381 | 0.7884 | 0.027182 | UBE2K |
| 23939304 | 23945304 | chr13 | − | exo | hsa_circ_0002046 | 0.7265 | 0.027186 | SACS |
| 6007022 | 6010262 | chr19 | − | exo | hsa_circ_0048807 | 1.2121 | 0.027194 | RFX2 |
| 54676862 | 54679030 | chr12 | + | exo | hsa_circ_0026715 | 1.1119 | 0.027201 | HNRNPA1 |
| 231307651 | 231314970 | chr2 | + | exo | hsa_circ_0003922 | 0.7422 | 0.027247 | SP100 |
| 94114814 | 94248681 | chr5 | − | exo | hsa_circ_0073379 | 1.0968 | 0.027251 | MCTP1 |
| 70497071 | 70499079 | chr16 | + | exo | hsa_circ_0040223 | 1.1301 | 0.027288 | FUK |
| 135211938 | 135216277 | chr10 | + | exo | hsa_circ_0020588 | 1.1597 | 0.027349 | MTG1 |
| 50596162 | 50601995 | chr15 | − | exo | hsa_circ_0035209 | 0.8254 | 0.027390 | GABPB1 |
| 126519981 | 126641300 | chr9 | − | exo | hsa_circ_0002544 | 1.2239 | 0.027427 | DENND1A |
| 47635539 | 47672796 | chr2 | + | exo | hsa_circ_0006402 | 0.8962 | 0.027445 | MSH2 |
| 97999787 | 97999925 | chr10 | − | int | hsa_circ_0000253 | 1.0624 | 0.027445 | BLNK |
| 134889723 | 134935760 | chr9 | − | s/o | | 1.1781 | 0.027449 | MED27 |
| 103552595 | 103570071 | chr10 | − | exo | hsa_circ_0019607 | 0.6736 | 0.027543 | MGEA5 |
| 13579682 | 13601181 | chr6 | + | exo | hsa_circ_0075648 | 0.7568 | 0.027676 | SIRT5 |
| 9583114 | 9595151 | chr18 | − | exo | hsa_circ_0007509 | 0.6017 | 0.027678 | PPP4R1 |
| 74371372 | 74372792 | chr16 | − | exo | hsa_circ_0005647 | 1.1054 | 0.027681 | LOC283922 |
| 30740286 | 30740893 | chr16 | + | exo | hsa_circ_0006127 | 1.1600 | 0.027776 | SRCAP |
| 74468040 | 74475660 | chr10 | + | exo | hsa_circ_0000246 | 1.1208 | 0.027834 | MCU |
| 90556280 | 90566918 | chr6 | + | exo | hsa_circ_0001621 | 1.0905 | 0.027900 | CASP8AP2 |
| 82779331 | 82808215 | chr5 | + | exo | | 1.1083 | 0.027906 | VCAN |
| 23502576 | 23502883 | chr14 | − | exo | hsa_circ_0008758 | 1.3609 | 0.027910 | PSMB5 |
| 97535283 | 97563284 | chr9 | + | exo | hsa_circ_0002191 | 1.5512 | 0.027928 | C9orf3 |
| 196612021 | 196613565 | chr3 | + | exo | hsa_circ_0068669 | 0.8646 | 0.027972 | SENP5 |
| 97820039 | 97823884 | chr7 | + | exo | hsa_circ_0001725 | 0.7580 | 0.027988 | LMTK2 |
| 7586 | 15888 | chrM | − | exo | hsa_circ_0089761 | 0.7277 | 0.027990 | JA760602 |
| 185580493 | 185599548 | chr4 | + | exo | hsa_circ_0003788 | 0.7857 | 0.027997 | PRIMPOL |
| 58449076 | 58459247 | chr2 | − | exo | hsa_circ_0001009 | 1.1775 | 0.028025 | FANCL |
| 40648098 | 40649277 | chr21 | − | exo | hsa_circ_0061749 | 0.8674 | 0.028029 | BRWD1 |
| 153507075 | 153507836 | chr1 | − | exo | hsa_circ_0014225 | 1.2946 | 0.028035 | S100A6 |
| 178287348 | 178287568 | chr5 | + | int | hsa_circ_0092298 | 1.1641 | 0.028039 | ZNF354B |
| 117187645 | 117217153 | chr12 | + | exo | | 1.1120 | 0.028113 | RNFT2 |
| 59445788 | 59450578 | chr15 | − | exo | hsa_circ_0035505 | 1.2723 | 0.028128 | MYO1E |
| 31600471 | 31605410 | chr12 | − | exo | hsa_circ_0025825 | 0.7589 | 0.028150 | DENND5B |
| 20635192 | 20657172 | chr13 | + | exo | | 0.4466 | 0.028172 | ZMYM2 |
| 15370487 | 15370655 | chr1 | + | s/o | | 1.3196 | 0.028175 | KAZN |
| 38698865 | 38699253 | chr22 | − | exo | hsa_circ_0063305 | 1.4952 | 0.028176 | CSNK1E |
| 179455865 | 179465394 | chr2 | + | exo | | 0.8579 | 0.028177 | TTN-AS1 |
| 89458787 | 89459087 | chr16 | − | int | | 0.2881 | 0.028210 | ANKRD11 |
| 8076452 | 8143777 | chr18 | + | exo | hsa_circ_0007144 | 0.6120 | 0.028257 | PTPRM |
| 52729602 | 52758017 | chr4 | + | exo | hsa_circ_0007646 | 0.7664 | 0.028265 | DCUN1D4 |
| 110863745 | 110866331 | chr3 | + | exo | hsa_circ_0007315 | 0.5135 | 0.028391 | PVRL3 |
| 176963358 | 176966148 | chr5 | − | exo | hsa_circ_0002972 | 1.2102 | 0.028407 | FAM193B |
| 47370185 | 47433094 | chr22 | + | exo | hsa_circ_0002406 | 1.0868 | 0.028495 | TBC1D22A |
| 3505743 | 3505854 | chr9 | − | int | hsa_circ_0001836 | 1.1428 | 0.028525 | RFX3 |
| 162940560 | 162944680 | chr5 | + | exo | hsa_circ_0074854 | 0.5051 | 0.028551 | MAT2B |
| 41854902 | 41855024 | chr17 | − | int | hsa_circ_0000772 | 1.3473 | 0.028641 | DUSP3 |
| 43552465 | 43553092 | chr17 | − | exo | hsa_circ_0044177 | 1.4638 | 0.028650 | PLEKHM1 |
| 161455290 | 161471011 | chr6 | + | exo | hsa_circ_0078617 | 0.6822 | 0.028687 | MAP3K4 |
| 47380394 | 47397280 | chr11 | − | exo | hsa_circ_0000303 | 1.1940 | 0.028698 | SPI1 |
| 111907996 | 111910121 | chr11 | + | exo | hsa_circ_0024295 | 1.1046 | 0.028709 | DLAT |
| 153377362 | 153414527 | chr5 | − | exo | hsa_circ_0003152 | 0.8702 | 0.028736 | FAM114A2 |
| 47538590 | 47538915 | chr21 | + | int | | 1.4686 | 0.028746 | COL6A2 |
| 30934467 | 30936639 | chr19 | + | exo | | 1.5270 | 0.028770 | ZNF536 |
| 153635690 | 153636950 | chr1 | − | exo | hsa_circ_0014263 | 1.1070 | 0.028785 | ILF2 |
| 75355797 | 75356052 | chr14 | + | exo | hsa_circ_0032627 | 1.2346 | 0.028788 | DLST |
| 67546221 | 67548719 | chr3 | − | exo | hsa_circ_0003060 | 0.7737 | 0.028811 | SUCLG2 |
| 1737913 | 1749314 | chr1 | − | exo | hsa_circ_0009357 | 1.3528 | 0.028813 | GNB1 |
| 74203700 | 74204000 | chr14 | − | exo | hsa_circ_0003121 | 1.1130 | 0.028875 | ELMSAN1 |
| 149940277 | 149984551 | chrX | − | exo | hsa_circ_0091692 | 1.3111 | 0.028915 | CD99L2 |
| 9843344 | 9848789 | chr3 | + | exo | hsa_circ_0064175 | 1.3709 | 0.028936 | ARPC4 |
| 62303908 | 62305446 | chr20 | + | exo | hsa_circ_0061170 | 1.5948 | 0.028940 | RTEL1 |
| 27269150 | 27269556 | chr1 | + | exo | hsa_circ_0005087 | 1.2885 | 0.029003 | NUDC |
| 138340248 | 138341258 | chr3 | + | exo | hsa_circ_0007080 | 1.0888 | 0.029040 | FAIM |
| 87356806 | 87367000 | chr9 | + | exo | hsa_circ_0087378 | 2.0151 | 0.029087 | NTRK2 |
| 191074876 | 191078948 | chr3 | + | exo | hsa_circ_0008124 | 1.1701 | 0.029112 | CCDC50 |
| 11778020 | 11830089 | chr16 | − | exo | hsa_circ_0037866 | 0.7208 | 0.029114 | TXNDC11 |
| 9204473 | 9211782 | chr18 | + | exo | hsa_circ_0000827 | 0.6908 | 0.029172 | ANKRD12 |
| 53856276 | 53862616 | chr12 | + | exo | hsa_circ_0026652 | 1.2935 | 0.029215 | PCBP2 |
| 78183551 | 78184326 | chr1 | − | exo | hsa_circ_0012992 | 0.7739 | 0.029226 | USP33 |
| 141799572 | 141840625 | chr8 | − | exo | hsa_circ_0008305 | 0.7074 | 0.029247 | PTK2 |
| 42602006 | 42635432 | chr20 | + | exo | hsa_circ_0004218 | 1.1837 | 0.029254 | TOX2 |
| 110412482 | 110427668 | chr4 | + | exo | hsa_circ_0070635 | 0.8308 | 0.029267 | SEC24B |
| 32354656 | 32358098 | chr20 | + | exo | hsa_circ_0059880 | 1.2310 | 0.029278 | ZNF341 |
| 51957464 | 51963580 | chr13 | − | exo | hsa_circ_0030296 | 1.2088 | 0.029283 | INTS6 |
| 89165951 | 89185063 | chr11 | − | exo | hsa_circ_0023990 | 0.8453 | 0.029290 | NOX4 |
| 196636 | 204692 | chr18 | + | exo | hsa_circ_0005653 | 0.8092 | 0.029359 | USP14 |
| 20149238 | 20209395 | chr17 | + | exo | hsa_circ_0042435 | 1.4128 | 0.029393 | SPECC1 |
| 154244751 | 154252211 | chr5 | + | exo | hsa_circ_0074719 | 1.2820 | 0.029412 | CNOT8 |
| 74043986 | 74053624 | chr17 | − | exo | | 1.1180 | 0.029412 | SRP68 |
| 33540210 | 33540336 | chr2 | + | exo | hsa_circ_0053936 | 0.7606 | 0.029414 | LTBP1 |
| 197677806 | 197682721 | chr3 | + | exo | hsa_circ_0068767 | 1.1341 | 0.029483 | RPL35A |
| 16935291 | 16948093 | chr8 | + | exo | | 0.8838 | 0.029495 | MICU3 |
| 17212469 | 17213367 | chr19 | + | exo | hsa_circ_0000907 | 6.3801 | 0.029520 | MYO9B |
| 66739280 | 66775151 | chr2 | + | exo | hsa_circ_0055004 | 1.2762 | 0.029556 | MEIS1 |
| 155340294 | 155340774 | chr1 | − | exo | hsa_circ_0007608 | 0.8261 | 0.029570 | ASH1L |
| 2807472 | 2810500 | chr16 | + | exo | hsa_circ_0037563 | 1.1458 | 0.029659 | SRRM2 |
| 111868787 | 111870850 | chr9 | − | exo | hsa_circ_0087938 | 0.8400 | 0.029684 | TMEM245 |
| 105718843 | 105739231 | chr14 | − | exo | hsa_circ_0005309 | 1.0867 | 0.029689 | BRF1 |
| 46275124 | 46281186 | chr21 | − | exo | hsa_circ_0001200 | 1.1910 | 0.029805 | PTTG1IP |
| 9437668 | 9437893 | chr8 | + | exo | hsa_circ_0083294 | 1.1358 | 0.029806 | TNKS |
| 45165959 | 45168961 | chr21 | + | exo | hsa_circ_0008021 | 1.2157 | 0.029814 | PDXK |
| 150070066 | 150070200 | chr7 | + | int | hsa_circ_0001768 | 1.2183 | 0.029929 | ZNF775 |
| 245018859 | 245019265 | chr1 | − | s/o | hsa_circ_0000200 | 1.7438 | 0.029987 | HNRNPU |
| 37864573 | 37866734 | chr17 | + | exo | hsa_circ_0007766 | 1.2216 | 0.030021 | ERBB2 |
| 809935 | 812876 | chr11 | + | exo | hsa_circ_0020704 | 0.5558 | 0.030063 | RPLP2 |
| 12049221 | 12052747 | chr1 | + | exo | hsa_circ_0009910 | 1.5241 | 0.030112 | MFN2 |
| 20436712 | 20466338 | chr10 | + | exo | hsa_circ_0000224 | 0.8275 | 0.030130 | PLXDC2 |
| 152017193 | 152018156 | chr3 | + | exo | hsa_circ_0001348 | 1.2159 | 0.030135 | MBNL1 |
| 40152499 | 40155575 | chr17 | − | exo | | 1.1477 | 0.030148 | DNAJC7 |
| 38128842 | 38137473 | chr21 | − | exo | | 1.4389 | 0.030159 | HLCS |
| 104352338 | 104359301 | chr10 | + | exo | hsa_circ_0003779 | 1.2481 | 0.030165 | SUFU |
| 159664587 | 159664757 | chr5 | − | as | hsa_circ_0001549 | 1.1236 | 0.030168 | FABP6 |
| 100515464 | 100535241 | chr1 | + | exo | hsa_circ_0000096 | 0.8437 | 0.030286 | HIAT1 |
| 56651185 | 56651585 | chr12 | − | int | hsa_circ_0092273 | 2.0133 | 0.030327 | ANKRD52 |
| 4516896 | 4517117 | chr16 | − | int | | 0.6319 | 0.030331 | NMRAL1 |
| 100982814 | 100992513 | chr13 | + | exo | hsa_circ_0008420 | 0.6890 | 0.030332 | PCCA |
| 6679862 | 6680397 | chr1 | + | exo | hsa_circ_0009535 | 1.0979 | 0.030411 | PHF13 |
| 2983142 | 2983691 | chr12 | − | exo | hsa_circ_0025042 | 1.0833 | 0.030426 | FOXM1 |
| 95364926 | 95365046 | chr1 | − | s/o | hsa_circ_0000094 | 1.2192 | 0.030447 | CNN3 |
| 79962925 | 79975155 | chrX | − | exo | hsa_circ_0001936 | 0.8433 | 0.030458 | BRWD3 |
| 114997099 | 115030475 | chr9 | − | exo | hsa_circ_0088070 | 0.6944 | 0.030459 | PTBP3 |
| 50592985 | 50596330 | chr15 | − | exo | hsa_circ_0007983 | 0.9075 | 0.030463 | GABPB1 |
| 32141444 | 32150586 | chr10 | − | exo | hsa_circ_0018100 | 0.7269 | 0.030475 | ARHGAP12 |
| 6599924 | 6602483 | chr4 | + | exo | hsa_circ_0069086 | 1.6282 | 0.030477 | MAN2B2 |
| 30936841 | 30938498 | chr15 | + | exo | | 0.8019 | 0.030494 | ARHGAP11B |
| 3608374 | 3608939 | chr17 | − | s/o | hsa_circ_0006692 | 1.1791 | 0.030545 | XLOC_012358 |
| 34789429 | 34789732 | chr6 | + | exo | hsa_circ_0076054 | 1.2345 | 0.030546 | UHRF1BP1 |
| 24813251 | 24817048 | chr10 | + | exo | hsa_circ_0006580 | 1.1707 | 0.030561 | KIAA1217 |
| 25315667 | 25317022 | chr4 | + | exo | hsa_circ_0001397 | 1.0876 | 0.030648 | ZCCHC4 |
| 61634149 | 61634467 | chr11 | + | s/o | hsa_circ_0000313 | 1.1102 | 0.030653 | FADS2 |
| 140136362 | 140138158 | chr9 | + | exo | hsa_circ_0089660 | 1.0745 | 0.030679 | TUBB4B |
| 125465775 | 125467014 | chr11 | + | exo | hsa_circ_0024758 | 0.9228 | 0.030711 | STT3A |
| 33421409 | 33422241 | chr17 | − | int | | 1.3279 | 0.030778 | RAD51L3-RFFL |
| 84662298 | 84663507 | chr1 | + | exo | | 0.7628 | 0.030826 | PRKACB |
| 138754312 | 138774924 | chr9 | − | exo | hsa_circ_0089494 | 0.8161 | 0.030833 | CAMSAP1 |
| 37967896 | 37968351 | chr8 | + | exo | hsa_circ_0083952 | 1.5282 | 0.030851 | ASH2L |
| 30397715 | 30399760 | cirri0 | + | as | | 1.1143 | 0.030854 | KIAA1462 |
| 50080770 | 50087302 | chr13 | + | exo | | 1.1410 | 0.030972 | PHF11 |
| 47531309 | 47644831 | chr16 | + | exo | hsa_circ_0002820 | 0.7785 | 0.030973 | PHKB |
| 74989492 | 75070408 | chr14 | − | exo | hsa_circ_0032603 | 1.0701 | 0.031011 | LTBP2 |
| 34254200 | 34276465 | chr1 | − | exo | hsa_circ_0011501 | 1.0835 | 0.031055 | CSMD2 |
| 166727312 | 166727530 | chr2 | − | s/o | | 1.4427 | 0.031058 | LOC100506124 |
| 80141649 | 80146295 | chr17 | − | exo | hsa_circ_0003430 | 1.1009 | 0.031073 | CCDC57 |
| 19615443 | 19667718 | chr12 | + | exo | hsa_circ_0025554 | 0.5390 | 0.031074 | AEBP2 |
| 77812691 | 77818094 | chr14 | − | exo | | 1.2090 | 0.031131 | TMED8 |
| 230381808 | 230386306 | chr1 | + | exo | | 1.1898 | 0.031135 | GALNT2 |
| 981641 | 1004666 | chr16 | − | exo | hsa_circ_0037274 | 0.5708 | 0.031137 | LMF1 |
| 5944982 | 5951257 | chr10 | + | exo | hsa_circ_0017594 | 1.1759 | 0.031200 | FBXO18 |
| 55610151 | 55614936 | chr19 | − | exo | hsa_circ_0000958 | 1.2149 | 0.031207 | PPP1R12C |
| 14970014 | 14977563 | chr10 | − | exo | hsa_circ_0017829 | 1.1299 | 0.031232 | DCLRE1C |
| 30693541 | 30724134 | chr21 | + | exo | | 0.6101 | 0.031258 | BACH1 |
| 241492330 | 241494472 | chr2 | − | exo | hsa_circ_0005059 | 1.1694 | 0.031263 | ANKMY1 |
| 242283166 | 242287606 | chr2 | + | exo | | 1.8277 | 0.031269 | SEPT2 |
| 118453893 | 118455359 | chr11 | + | exo | hsa_circ_0007383 | 0.8464 | 0.031310 | ARCN1 |
| 983990 | 1004666 | chr16 | − | exo | | 1.1146 | 0.031326 | LMF1 |
| 74756185 | 74757202 | chr14 | − | exo | hsa_circ_0032547 | 1.2527 | 0.031454 | ABCD4 |
| 93637042 | 93639999 | chr9 | − | as | hsa_circ_0001873 | 0.4739 | 0.031506 | SYK |
| 185153374 | 185200840 | chr1 | + | exo | hsa_circ_0015677 | 0.7323 | 0.031547 | SWT1 |
| 66021409 | 66048810 | chr15 | − | exo | hsa_circ_0035946 | 0.7666 | 0.031561 | DENND4A |
| 66455621 | 66512933 | chr3 | − | exo | hsa_circ_0001318 | 1.9167 | 0.031567 | LRIG1 |
| 59204761 | 59209198 | chr15 | − | exo | hsa_circ_0000605 | 0.7988 | 0.031609 | SLTM |
| 33001547 | 33037285 | chr20 | + | exo | hsa_circ_0001141 | 0.7862 | 0.031665 | ITCH |
| 47858295 | 47858607 | chr19 | + | exo | hsa_circ_0005188 | 1.2050 | 0.031693 | DHX34 |
| 69874028 | 69876023 | chr16 | + | exo | hsa_circ_0040097 | 1.4784 | 0.031715 | WWP2 |
| 38631823 | 38633350 | chr19 | + | exo | hsa_circ_0006670 | 1.2210 | 0.031760 | SIPA1L3 |
| 22477517 | 22496137 | chr16 | + | exo | hsa_circ_0038539 | 0.6650 | 0.031780 | SMG1P1 |
| 44270150 | 44296877 | chr21 | − | exo | hsa_circ_0001196 | 1.0845 | 0.031858 | WDR4 |
| 28250417 | 28255132 | chr11 | + | int | hsa_circ_0000282 | 0.6404 | 0.031866 | METTL15 |
| 37061800 | 37067498 | chr3 | + | exo | | 1.1834 | 0.031868 | MLH1 |
| 36668400 | 36669878 | chr2 | + | exo | hsa_circ_0007386 | 1.7084 | 0.031897 | CRIM1 |
| 39382719 | 39388954 | chr5 | − | exo | hsa_circ_0002649 | 1.1264 | 0.031913 | DAB2 |
| 47285639 | 47287816 | chr19 | − | exo | hsa_circ_0002311 | 0.6287 | 0.031962 | SLC1A5 |
| 107099431 | 107101023 | chr3 | − | s/o | | 0.8485 | 0.032025 | LOC101929579 |
| 43514307 | 43516197 | chr6 | − | exo | hsa_circ_0076560 | 0.8487 | 0.032043 | XPO5 |
| 50929618 | 50941082 | chr15 | − | exo | hsa_circ_0000601 | 0.8023 | 0.032053 | TRPM7 |
| 47865737 | 47865950 | chr19 | + | int | | 1.2275 | 0.032082 | DHX34 |
| 71736500 | 71748704 | chr16 | − | exo | hsa_circ_0003681 | 0.8859 | 0.032140 | PHLPP2 |
| 39422972 | 39423659 | chr19 | + | exo | hsa_circ_0050963 | 1.3209 | 0.032174 | MRPS12 |
| 229599289 | 229601266 | chr1 | − | exo | | 0.7356 | 0.032194 | NUP133 |
| 100296305 | 100297301 | chrX | − | exo | hsa_circ_0001937 | 1.1147 | 0.032249 | TRMT2B |
| 14988854 | 14989971 | chr16 | + | s/o | hsa_circ_0004409 | 1.1749 | 0.032272 | NOMO1 |
| 48949251 | 48953623 | chr19 | + | s/o | hsa_circ_0000947 | 1.2384 | 0.032297 | GRWD1 |
| 46858233 | 46906128 | chr18 | − | exo | hsa_circ_0002501 | 0.7698 | 0.032339 | DYM |
| 242572331 | 242573494 | chr2 | − | exo | hsa_circ_0007647 | 1.4496 | 0.032388 | THAP4 |
| 17198871 | 17206590 | chr8 | − | exo | | 0.7247 | 0.032394 | MTMR7 |
| 39595439 | 39613921 | chr18 | + | exo | hsa_circ_0000843 | 0.8072 | 0.032453 | PIK3C3 |
| 6291961 | 6293703 | chr1 | − | exo | hsa_circ_0007427 | 1.2027 | 0.032465 | ICMT |
| 131669435 | 131671600 | chr9 | + | exo | hsa_circ_0007311 | 1.1240 | 0.032510 | LRRC8A |
| 7074514 | 7076411 | chr12 | − | exo | hsa_circ_0025302 | 1.1467 | 0.032578 | PHB2 |
| 137313521 | 137326053 | chr9 | + | exo | | 1.5719 | 0.032703 | RXRA |
| 196793574 | 196846401 | chr3 | − | exo | hsa_circ_0068684 | 0.8368 | 0.032765 | DLG1 |
| 66444138 | 66444562 | chr11 | − | exo | hsa_circ_0004144 | 1.2197 | 0.032767 | RBM4B |
| 149663820 | 149691235 | chr6 | + | exo | hsa_circ_0005753 | 0.8602 | 0.032776 | TAB2 |
| 3524281 | 3524706 | chr17 | − | s/o | | 1.0843 | 0.032858 | SHPK |
| 100882892 | 100884187 | chr7 | − | exo | hsa_circ_0081594 | 1.0620 | 0.032860 | FIS1 |
| 103427642 | 103436193 | chr10 | − | exo | hsa_circ_0008362 | 0.8296 | 0.033086 | FBXW4 |
| 354303 | 364683 | chr16 | − | exo | hsa_circ_0037158 | 1.1113 | 0.033087 | AXIN1 |
| 47468936 | 47470160 | chr3 | − | exo | hsa_circ_0065220 | 1.2005 | 0.033098 | SCAP |
| 183442206 | 183446639 | chr3 | + | exo | | 1.2405 | 0.033107 | YEATS2 |
| 77860917 | 77921770 | chr6 | − | s/o | hsa_circ_0008029 | 1.2750 | 0.033155 | XLOC_005768 |
| 70510478 | 70521018 | chrX | + | exo | hsa_circ_0090999 | 1.0778 | 0.033165 | NONO |
| 30304419 | 30306012 | chr16 | − | exo | | 0.8628 | 0.033205 | SMG1P5 |
| 20923658 | 20933102 | chr9 | + | exo | hsa_circ_0008678 | 0.7151 | 0.033206 | FOCAD |
| 112707507 | 112717190 | chr12 | − | exo | | 0.7705 | 0.033210 | HECTD4 |
| 173735284 | 173744981 | chr1 | + | exo | hsa_circ_0015279 | 1.2690 | 0.033239 | KLHL20 |
| 77237532 | 77239554 | chr14 | + | exo | | 2.1091 | 0.033313 | VASH1 |
| 126519981 | 126554909 | chr9 | − | exo | hsa_circ_0088475 | 1.2570 | 0.033314 | DENND1A |
| 89999875 | 90002505 | chr16 | + | exo | hsa_circ_0041125 | 1.1430 | 0.033359 | TUBB3 |
| 72050664 | 72054207 | chr12 | − | s/o | hsa_circ_0000420 | 0.8883 | 0.033379 | ZFC3H1 |
| 113243493 | 113278002 | chr2 | + | exo | hsa_circ_0056135 | 1.0812 | 0.033387 | TTL |
| 115961134 | 115968858 | chr9 | − | exo | hsa_circ_0007285 | 0.9547 | 0.033387 | FKBP15 |
| 4490949 | 4494733 | chr3 | − | exo | hsa_circ_0064039 | 1.1711 | 0.033467 | SUMF1 |
| 33608730 | 33610023 | chr19 | + | exo | hsa_circ_0050444 | 1.3957 | 0.033530 | GPATCH1 |
| 13919649 | 13920034 | chr19 | + | exo | hsa_circ_0007836 | 1.2377 | 0.033536 | ZSWIM4 |
| 126310872 | 126311001 | chr10 | − | int | hsa_circ_0000265 | 1.1345 | 0.033548 | RP11-12J10.3 |
| 34789429 | 34791125 | chr6 | + | exo | hsa_circ_0076055 | 1.2282 | 0.033588 | UHRF1BP1 |
| 231222519 | 231226412 | chr2 | + | exo | hsa_circ_0008382 | 0.8212 | 0.033607 | SP140L |
| 53108528 | 53124515 | chr17 | + | exo | | 0.7667 | 0.033618 | STXBP4 |
| 33960823 | 33963789 | chr9 | − | exo | hsa_circ_0001849 | 1.2799 | 0.033686 | UBAP2 |
| 95546095 | 95546753 | chr11 | + | exo | hsa_circ_0024067 | 1.0855 | 0.033691 | CEP57 |
| 85657103 | 85658718 | chr15 | + | exo | hsa_circ_0003856 | 0.9111 | 0.033695 | PDE8A |
| 27424643 | 27493989 | chr3 | − | exo | | 0.7073 | 0.033716 | SLC4A7 |
| 53569403 | 53587377 | chrX | − | exo | hsa_circ_0090682 | 1.6149 | 0.033725 | HUWE1 |
| 51680350 | 51680428 | chr16 | + | s/o | hsa_circ_0005008 | 2.2094 | 0.033829 | HNRNPA1P48 |
| 33711692 | 33714178 | chr20 | − | exo | hsa_circ_0060043 | 1.1096 | 0.033848 | EDEM2 |
| 27223791 | 27224216 | chr1 | − | exo | hsa_circ_0011032 | 1.2819 | 0.033865 | GPATCH3 |
| 153581139 | 153581292 | chrX | − | exo | hsa_circ_0091894 | 2.3366 | 0.033909 | FLNA |
| 16126965 | 16130460 | chr16 | + | exo | hsa_circ_0002078 | 1.1961 | 0.033912 | ABCC1 |
| 71880664 | 71933964 | chr14 | + | int | hsa_circ_0006783 | 0.8130 | 0.033946 | SIPA1L1 |
| 30954186 | 30956926 | chr20 | + | exo | hsa_circ_0001136 | 1.8325 | 0.033986 | ASXL1 |
| 1712336 | 1714425 | chrX | + | exo | hsa_circ_0007352 | 1.1362 | 0.034006 | AKAP17A |
| 46783379 | 46798673 | chr18 | − | exo | hsa_circ_0047641 | 0.4063 | 0.034028 | DYM |
| 45223227 | 45223812 | chr1 | + | exo | hsa_circ_0002563 | 1.2284 | 0.034062 | KIF2C |
| 160206924 | 160231148 | chr1 | − | exo | hsa_circ_0014879 | 1.6411 | 0.034154 | DCAF8 |
| 183522076 | 183575046 | chr4 | + | exo | | 0.6739 | 0.034157 | TENM3 |
| 47287161 | 47308084 | chr22 | + | exo | hsa_circ_0063853 | 0.8603 | 0.034183 | TBC1D22A |
| 17271723 | 17271867 | chr10 | − | s/o | hsa_circ_0000221 | 1.4135 | 0.034208 | VIM-AS1 |
| 1255200 | 1255400 | chr5 | − | int | hsa_circ_0092366 | 1.1413 | 0.034217 | TERT |
| 2182820 | 2224733 | chr3 | + | int | | 1.1741 | 0.034226 | CNTN4 |
| 80063751 | 80088663 | chr5 | + | exo | hsa_circ_0073177 | 0.8624 | 0.034372 | MSH3 |
| 171974248 | 171974367 | chr2 | − | exo | hsa_circ_0057037 | 1.2232 | 0.034443 | TLK1 |
| 94428283 | 94428763 | chr10 | − | s/o | hsa_circ_0007472 | 0.8801 | 0.034525 | EIF2S2P3 |
| 34868407 | 34868786 | chr19 | + | exo | hsa_circ_0050486 | 1.3080 | 0.034530 | GPI |
| 70215873 | 70224020 | chr2 | − | exo | hsa_circ_0055102 | 1.2118 | 0.034533 | PCBP1-AS1 |
| 37840849 | 37842272 | chr17 | − | exo | hsa_circ_0007990 | 0.5606 | 0.034612 | PGAP3 |
| 7414685 | 7414883 | chr5 | + | exo | | 0.5747 | 0.034704 | ADCY2 |
| 27440281 | 27440777 | chr1 | − | int | | 0.7992 | 0.034736 | SLC9A1 |
| 31139461 | 31144271 | chr14 | + | exo | hsa_circ_0009140 | 0.8529 | 0.034738 | SCFD1 |
| 6727768 | 6741097 | chr1 | − | exo | hsa_circ_0008389 | 1.3885 | 0.034739 | DNAJC11 |
| 89220855 | 89221015 | chr14 | − | exo | hsa_circ_0032883 | 0.8217 | 0.034743 | EML5 |
| 232320111 | 232320192 | chr2 | − | s/o | hsa_circ_0001111 | 1.3570 | 0.034771 | NCL |
| 122842182 | 122843190 | chr3 | + | s/o | | 1.1595 | 0.034792 | PDIA5 |
| 69983264 | 69987393 | chr12 | + | exo | hsa_circ_0002940 | 0.8389 | 0.034849 | CCT2 |
| 243708811 | 243736350 | chr1 | − | exo | hsa_circ_0000199 | 0.7026 | 0.034853 | AKT3 |
| 77973094 | 77998530 | chr7 | − | exo | | 0.8167 | 0.034897 | MAGI2 |
| 61719169 | 61724142 | chr2 | − | exo | hsa_circ_0054877 | 0.8590 | 0.034950 | XPO1 |
| 334399 | 336173 | chr19 | − | exo | | 0.7807 | 0.034953 | MIER2 |
| 73646250 | 73659419 | chr2 | + | exo | hsa_circ_0055229 | 0.8742 | 0.034984 | ALMS1 |
| 128488846 | 128489036 | chr7 | + | exo | hsa_circ_0082218 | 1.1567 | 0.035003 | FLNC |
| 82443805 | 82444764 | chr15 | − | exo | hsa_circ_0005745 | 0.8482 | 0.035057 | EFTUD1 |
| 28931883 | 28948677 | chr1 | − | exo | hsa_circ_0011159 | 1.1484 | 0.035170 | TAF12 |
| 20534273 | 20534434 | chr10 | + | exo | | 0.7802 | 0.035173 | PLXDC2 |
| 99966350 | 99970323 | chr13 | + | exo | hsa_circ_0030724 | 1.0789 | 0.035230 | UBAC2 |
| 88655653 | 88667506 | chr9 | − | exo | hsa_circ_0087400 | 1.3000 | 0.035235 | GOLM1 |
| 1475710 | 1489345 | chr17 | − | s/o | hsa_circ_0000731 | 1.1127 | 0.035254 | SLC43A2 |
| 1747194 | 1749314 | chr1 | − | exo | hsa_circ_0009360 | 1.2274 | 0.035334 | GNB1 |
| 138761031 | 138764989 | chr7 | − | exo | hsa_circ_0082628 | 0.7392 | 0.035335 | ZC3HAV1 |
| 48422178 | 48452268 | chr18 | + | exo | hsa_circ_0000850 | 1.2007 | 0.035351 | ME2 |
| 72181471 | 72188374 | chr10 | + | exo | hsa_circ_0018657 | 1.1342 | 0.035395 | EIF4EBP2 |
| 170585337 | 170597207 | chr5 | + | exo | | 0.8605 | 0.035405 | RANBP17 |
| 242079935 | 242080168 | chr2 | − | exo | hsa_circ_0006391 | 1.2724 | 0.035521 | PASK |
| 14066723 | 14067334 | chr19 | + | exo | | 1.1222 | 0.035568 | DCAF15 |
| 134424934 | 134429965 | chrX | − | exo | | 0.9399 | 0.035645 | ZNF75D |
| 60687157 | 60688460 | chr11 | + | exo | hsa_circ_0005701 | 1.1210 | 0.035727 | TMEM109 |
| 99967857 | 99969656 | chr10 | + | exo | hsa_circ_0019390 | 0.9057 | 0.035832 | R3HCC1L |
| 29784412 | 29819177 | chr22 | − | s/o | hsa_circ_0001218 | 1.1832 | 0.035837 | AP1B1 |
| 80180153 | 80183460 | chr12 | − | exo | hsa_circ_0000423 | 1.5443 | 0.035884 | PPP1R12A |
| 71855409 | 71860053 | chr10 | + | exo | | 1.2618 | 0.035905 | H2AFY2 |
| 57466030 | 57467869 | chr11 | + | s/o | hsa_circ_0004997 | 0.8491 | 0.035921 | ZDHHC5 |
| 15735268 | 15735677 | chr2 | + | exo | hsa_circ_0052793 | 1.1192 | 0.035948 | DDX1 |
| 19510304 | 19511780 | chr1 | − | exo | hsa_circ_0003777 | 0.9318 | 0.035967 | UBR4 |
| 47682728 | 47683817 | chr20 | + | exo | hsa_circ_0060733 | 0.8379 | 0.035992 | CSE1L |
| 15452751 | 15457449 | chr3 | − | exo | | 0.7531 | 0.035997 | METTL6 |
| 58999624 | 59000003 | chr1 | − | exo | | 0.7393 | 0.036072 | OMA1 |
| 78458766 | 78466649 | chr16 | + | exo | hsa_circ_0040573 | 0.9101 | 0.036106 | WWOX |
| 20811398 | 20811483 | chr14 | + | as | hsa_circ_0000516 | 1.7375 | 0.036142 | RPPH1 |
| 54308819 | 54310270 | chr4 | + | exo | hsa_circ_0069752 | 0.8108 | 0.036150 | FIP1L1 |
| 29061914 | 29086557 | chr17 | + | exo | | 0.7874 | 0.036164 | SUZ12P1 |
| 15875628 | 15889942 | chr10 | − | exo | hsa_circ_0006665 | 0.7822 | 0.036232 | FAM188A |
| 4445536 | 4447603 | chr19 | + | s/o | hsa_circ_0000877 | 0.6926 | 0.036234 | MIR4746 |
| 6022454 | 6031688 | chr7 | − | exo | hsa_circ_0079311 | 0.8294 | 0.036304 | PMS2 |
| 48175347 | 48177671 | chr11 | + | exo | | 0.8664 | 0.036325 | PTPRJ |
| 111570330 | 111576507 | chr5 | − | exo | | 0.7444 | 0.036361 | EPB41L4A |
| 161293403 | 161310445 | chr1 | + | exo | hsa_circ_0015003 | 1.3479 | 0.036383 | SDHC |
| 26232870 | 26232993 | chr7 | − | exo | hsa_circ_0001689 | 1.3491 | 0.036472 | HNRNPA2B1 |
| 71492833 | 71502874 | chr14 | + | exo | hsa_circ_0007996 | 0.7958 | 0.036487 | PCNX |
| 39499423 | 39505627 | chr2 | − | exo | hsa_circ_0054189 | 0.5639 | 0.036497 | MAP4K3 |
| 76234198 | 76239510 | chr11 | + | exo | | 0.7939 | 0.036544 | C11orf30 |
| 18860538 | 18861789 | chr16 | − | exo | hsa_circ_0038282 | 0.8945 | 0.036575 | SMG1 |
| 65034052 | 65036200 | chr11 | + | exo | hsa_circ_0022803 | 1.0699 | 0.036578 | POLA2 |
| 156411808 | 156413814 | chr3 | + | exo | hsa_circ_0006999 | 1.3128 | 0.036642 | TIPARP |
| 52729602 | 52752804 | chr4 | + | exo | hsa_circ_0007928 | 0.7990 | 0.036656 | DCUN1D4 |
| 23495059 | 23502883 | chr14 | − | exo | hsa_circ_0031268 | 1.1197 | 0.036728 | PSMB5 |
| 46858233 | 46860223 | chr18 | − | exo | hsa_circ_0047650 | 0.7633 | 0.036750 | DYM |
| 36636707 | 36637214 | chr19 | + | exo | hsa_circ_0002356 | 1.3831 | 0.036801 | CAPNS1 |
| 98984307 | 98987635 | chr7 | + | exo | hsa_circ_0081342 | 1.2678 | 0.036830 | ARPC1B |
| 86292641 | 86293514 | chr9 | − | exo | hsa_circ_0001865 | 0.6506 | 0.036842 | UBQLN1 |
| 73100965 | 73101230 | chr7 | + | exo | hsa_circ_0080502 | 1.2759 | 0.036876 | WBSCR22 |
| 81297486 | 81307112 | chr14 | − | exo | hsa_circ_0032825 | 1.1512 | 0.036899 | CEP128 |
| 89611055 | 89614521 | chr16 | + | exo | hsa_circ_0004945 | 1.1388 | 0.036905 | SPG7 |
| 50290691 | 50346517 | chr20 | − | exo | hsa_circ_0060903 | 1.2710 | 0.037018 | ATP9A |
| 70292882 | 70296427 | chr16 | − | exo | hsa_circ_0040188 | 0.8765 | 0.037040 | AARS |
| 23180387 | 23207656 | chr7 | + | exo | hsa_circ_0079573 | 1.0811 | 0.037072 | KLHL7 |
| 83940591 | 83945972 | chr16 | + | exo | | 1.2280 | 0.037116 | MLYCD |
| 44444179 | 44444552 | chr20 | + | exo | hsa_circ_0060551 | 1.1356 | 0.037166 | UBE2C |
| 88091230 | 88116842 | chr4 | − | exo | hsa_circ_0070389 | 0.8585 | 0.037346 | KLHL8 |
| 34895553 | 34895895 | chr19 | + | exo | hsa_circ_0000927 | 0.5331 | 0.037593 | PDCD2L |
| 45340349 | 45363116 | chr1 | − | exo | hsa_circ_0012187 | 0.9082 | 0.037673 | EIF2B3 |
| 44552565 | 44791352 | chr17 | + | s/o | hsa_circ_0004601 | 0.9066 | 0.037771 | NSF |
| 149778518 | 149778894 | chr5 | + | s/o | hsa_circ_0001545 | 1.1403 | 0.037837 | TCOF1 |
| 118422500 | 118430579 | chr11 | − | exo | hsa_circ_0007372 | 0.8607 | 0.037866 | IFT46 |
| 65568074 | 65568341 | chr14 | − | int | | 1.1722 | 0.037908 | MAX |
| 231624673 | 231658046 | chr2 | + | exo | hsa_circ_0003898 | 0.8988 | 0.037984 | CAB39 |
| 36737125 | 36749456 | chr2 | + | exo | hsa_circ_0006422 | 0.8257 | 0.038003 | CRIM1 |
| 9482139 | 9506356 | chr3 | + | exo | hsa_circ_0064136 | 0.7305 | 0.038050 | SETD5 |
| 25477298 | 25481646 | chr20 | − | exo | | 1.1650 | 0.038079 | NINL |
| 69090598 | 69094633 | chr12 | + | exo | hsa_circ_0027470 | 0.9040 | 0.038082 | NUP107 |
| 90355880 | 90356126 | chr7 | + | exo | hsa_circ_0001721 | 1.4756 | 0.038100 | CDK14 |
| 10865809 | 10868382 | chr12 | − | exo | hsa_circ_0025460 | 0.4132 | 0.038106 | YBX3 |
| 30701807 | 30702014 | chr21 | + | exo | hsa_circ_0061396 | 0.5079 | 0.038172 | BACH1 |
| 112116954 | 112121111 | chr12 | − | exo | hsa_circ_0006396 | 0.8975 | 0.038183 | BRAP |
| 20478343 | 20527139 | chr2 | − | exo | hsa_circ_0052852 | 0.8281 | 0.038205 | PUM2 |
| 146209155 | 146216113 | chr6 | − | exo | hsa_circ_0001649 | 0.7686 | 0.038207 | SHPRH |
| 76688498 | 76695050 | chr17 | − | exo | hsa_circ_0000809 | 0.6638 | 0.038261 | CYTH1 |
| 148142 | 150507 | chr16 | − | exo | hsa_circ_0037130 | 1.4124 | 0.038396 | NPRL3 |
| 15615497 | 15652371 | chr6 | − | exo | hsa_circ_0075681 | 1.2451 | 0.038413 | DTNBP1 |
| 77611362 | 77632364 | chr9 | − | exo | hsa_circ_0087220 | 0.8223 | 0.038422 | C9orf41 |
| 6859013 | 6859932 | chr12 | − | exo | hsa_circ_0007845 | 1.5255 | 0.038442 | MLF2 |
| 40842016 | 40842314 | chr19 | − | exo | hsa_circ_0004952 | 1.1133 | 0.038462 | C19orf47 |
| 27143382 | 27156323 | chr12 | − | exo | hsa_circ_0005824 | 0.8772 | 0.038503 | TM7SF3 |
| 66006274 | 66006748 | chr11 | + | exo | hsa_circ_0006857 | 1.1431 | 0.038601 | PACS1 |
| 34300940 | 34313077 | chr20 | − | exo | hsa_circ_0008143 | 0.8664 | 0.038613 | RBM39 |
| 133740043 | 133769540 | chr8 | − | exo | hsa_circ_0006050 | 0.6561 | 0.038653 | TMEM71 |
| 57523349 | 57526305 | chr15 | + | exo | hsa_circ_0006380 | 0.8796 | 0.038668 | TCF12 |
| 46988169 | 47006422 | chr17 | + | exo | hsa_circ_0044375 | 1.1434 | 0.038697 | UBE2Z |
| 10423846 | 10426634 | chr5 | + | exo | hsa_circ_0071869 | 0.8683 | 0.038721 | MARCH6 |
| 175777615 | 175782752 | chr5 | − | exo | hsa_circ_0008823 | 1.1742 | 0.038728 | KIAA1191 |
| 22160138 | 22162135 | chr22 | − | exo | hsa_circ_0008870 | 0.7721 | 0.038767 | MAPK1 |
| 179043126 | 179043219 | chr5 | − | exo | hsa_circ_0075281 | 1.5634 | 0.038841 | HNRNPH1 |
| 175913355 | 175916028 | chr5 | + | exo | | 1.1746 | 0.038845 | FAF2 |
| 179250857 | 179252226 | chr5 | + | exo | | 1.6508 | 0.038857 | SQSTM1 |
| 50059614 | 50062337 | chr12 | − | exo | hsa_circ_0006802 | 1.1891 | 0.038867 | FMNL3 |
| 61632920 | 61633008 | chr2 | + | as | hsa_circ_0001015 | 1.2400 | 0.038891 | USP34 |
| 161332118 | 161336094 | chr1 | + | s/o | | 1.3699 | 0.038906 | SDHC |
| 183841620 | 183888644 | chr2 | − | exo | hsa_circ_0057283 | 1.0789 | 0.038935 | NCKAP1 |
| 1918802 | 1919370 | chr19 | − | as | | 0.5778 | 0.038995 | SCAMP4 |
| 12422775 | 12557685 | chr1 | + | exo | hsa_circ_0010009 | 1.1460 | 0.039045 | VPS13D |
| 71913593 | 71921687 | chr10 | − | exo | hsa_circ_0018634 | 0.8135 | 0.039060 | SAR1A |
| 38463710 | 38466898 | chr22 | + | exo | hsa_circ_0063280 | 1.1115 | 0.039099 | PICK1 |
| 51926762 | 51930998 | chr1 | − | exo | hsa_circ_0007330 | 0.8135 | 0.039159 | EPS15 |
| 47134948 | 47136316 | chr2 | − | exo | hsa_circ_0008979 | 0.6904 | 0.039196 | MCFD2 |
| 29472702 | 29473271 | chr16 | + | exo | | 0.5131 | 0.039360 | SULT1A3 |
| 23836176 | 23836434 | chr1 | − | s/o | hsa_circ_0000030 | 1.0753 | 0.039429 | E2F2 |
| 18974246 | 18975060 | chr19 | + | exo | hsa_circ_0005706 | 1.1252 | 0.039479 | UPF1 |
| 19286766 | 19305525 | chr9 | + | exo | hsa_circ_0005684 | 0.8236 | 0.039509 | DENND4C |
| 44624185 | 44630515 | chr15 | + | exo | hsa_circ_0000596 | 0.8178 | 0.039553 | CASC4 |
| 33576158 | 33596165 | chr5 | − | exo | hsa_circ_0004807 | 0.7956 | 0.039710 | ADAMTS12 |
| 25650607 | 25654354 | chr15 | − | exo | hsa_circ_0034168 | 1.2128 | 0.039719 | UBE3A |
| 148968042 | 148968202 | chr4 | + | exo | hsa_circ_0071124 | 1.0978 | 0.039733 | ARHGAP10 |
| 116018393 | 116019465 | chr9 | + | exo | hsa_circ_0088128 | 1.1207 | 0.039770 | SLC31A1 |
| 140611077 | 140646860 | chr9 | + | exo | hsa_circ_0002647 | 1.1288 | 0.039842 | EHMT1 |
| 77779390 | 77818086 | chr13 | − | exo | hsa_circ_0030509 | 0.8832 | 0.039849 | MYCBP2 |
| 158850767 | 158852695 | chr6 | + | s/o | hsa_circ_0001660 | 1.2856 | 0.039877 | TULP4 |
| 35029495 | 35053317 | chr7 | − | exo | hsa_circ_0079845 | 0.8561 | 0.039920 | DPY19L1 |
| 21979948 | 21987564 | chr16 | + | exo | hsa_circ_0006485 | 1.1845 | 0.039935 | UQCRC2 |
| 31764759 | 31766195 | chr1 | − | exo | hsa_circ_0011267 | 1.2557 | 0.039975 | SNRNP40 |
| 158850767 | 158873300 | chr6 | + | exo | hsa_circ_0002987 | 1.1989 | 0.039987 | TULP4 |
| 15057658 | 15076316 | chr3 | + | exo | hsa_circ_0064455 | 0.9418 | 0.040033 | NR2C2 |
| 73015794 | 73017356 | chr17 | + | exo | hsa_circ_0045581 | 1.1186 | 0.040142 | ICT1 |
| 72318470 | 72342087 | chr16 | − | s/o | | 0.7773 | 0.040175 | LINC01572 |
| 89856134 | 89857938 | chr15 | + | exo | hsa_circ_0008843 | 1.1009 | 0.040240 | FANCI |
| 116730126 | 116734144 | chr10 | + | exo | hsa_circ_0020092 | 0.8755 | 0.040280 | TRUB1 |
| 53907697 | 53968021 | chr16 | + | exo | hsa_circ_0039400 | 1.2255 | 0.040361 | FTO |
| 57058639 | 57075243 | chr6 | − | exo | hsa_circ_0004119 | 0.7597 | 0.040365 | RAB23 |
| 213046019 | 213061936 | chr1 | + | exo | hsa_circ_0016413 | 0.8313 | 0.040402 | FLVCR1 |
| 70100940 | 70115158 | chr10 | − | s/o | | 1.1062 | 0.040406 | RUFY2 |
| 222803446 | 222803648 | chr1 | + | int | hsa_circ_0000186 | 1.2364 | 0.040420 | MIA3 |
| 19646733 | 19646920 | chr12 | + | exo | | 0.7487 | 0.040457 | AEBP2 |
| 32673862 | 32735061 | chr2 | + | exo | hsa_circ_0053642 | 0.8866 | 0.040508 | BIRC6 |
| 100953713 | 101101559 | chr13 | + | exo | hsa_circ_0030764 | 0.6658 | 0.040564 | PCCA |
| 83276456 | 83280792 | chr4 | − | exo | hsa_circ_0001420 | 1.1872 | 0.040621 | HNRNPD |
| 80071512 | 80088663 | chr5 | + | exo | hsa_circ_0001508 | 0.6922 | 0.040643 | MSH3 |
| 6947713 | 6948261 | chr1 | + | exo | hsa_circ_0009544 | 1.1053 | 0.040706 | CAMTA1 |
| 115949574 | 115950528 | chr7 | − | int | | 1.2842 | 0.040779 | AC073130.3 |
| 37614541 | 37625720 | chr13 | − | exo | hsa_circ_0000475 | 0.8931 | 0.040824 | SUPT20H |
| 18528403 | 18531212 | chr11 | − | exo | hsa_circ_0021461 | 1.1072 | 0.040828 | TSG101 |
| 43008874 | 43011764 | chr15 | + | s/o | | 1.7298 | 0.040831 | STARD9 |
| 13233298 | 13234568 | chr10 | + | exo | hsa_circ_0004477 | 1.1065 | 0.040861 | MCM10 |
| 128715231 | 128719864 | chr4 | + | exo | hsa_circ_0070911 | 1.1208 | 0.040875 | HSPA4L |
| 122237752 | 122239714 | chr12 | − | exo | hsa_circ_0029021 | 0.6373 | 0.040902 | LINC01089 |
| 140064207 | 140069482 | chr7 | − | exo | hsa_circ_0082711 | 0.8629 | 0.040951 | SLC37A3 |
| 87114895 | 87115116 | chr2 | + | exo | hsa_circ_0005528 | 1.1715 | 0.040977 | ANAPC1P1 |
| 112498073 | 112499192 | chr12 | − | exo | | 0.5860 | 0.041051 | NAA25 |
| 32372021 | 32385259 | chr1 | − | exo | hsa_circ_0011333 | 0.5138 | 0.041142 | PTP4A2 |
| 138741982 | 138742307 | chr9 | − | exo | hsa_circ_0089486 | 0.8320 | 0.041172 | CAMSAP1 |
| 76910271 | 76910864 | chr10 | + | exo | hsa_circ_0006148 | 0.7342 | 0.041197 | SAMD8 |
| 37623043 | 37632999 | chr8 | + | exo | hsa_circ_0083932 | 0.8827 | 0.041214 | PROSC |
| 47691321 | 47707559 | chr20 | + | exo | hsa_circ_0001168 | 0.8169 | 0.041278 | CSE1L |
| 228401141 | 228402799 | chr1 | + | exo | hsa_circ_0016771 | 1.3800 | 0.041286 | OBSCN |
| 9905173 | 9905725 | chrX | + | exo | hsa_circ_0089863 | 0.7014 | 0.041302 | SHROOM2 |
| 125472197 | 125474142 | chr11 | + | exo | hsa_circ_0024766 | 0.5059 | 0.041319 | STT3A |
| 62531536 | 62566219 | chr8 | − | exo | hsa_circ_0084606 | 0.8670 | 0.041319 | ASPH |
| 74474399 | 74474581 | chr7 | − | exo | hsa_circ_0080646 | 1.2450 | 0.041360 | WBSCR16 |
| 114069120 | 114619189 | chr3 | − | exo | hsa_circ_0066845 | 0.5085 | 0.041406 | ZBTB20 |
| 185165576 | 185169183 | chr3 | + | exo | | 0.8819 | 0.041432 | MAP3K13 |
| 24416624 | 24418806 | chr6 | + | exo | hsa_circ_0075835 | 0.8988 | 0.041447 | MRS2 |
| 37838091 | 37838774 | chr19 | + | exo | | 1.1649 | 0.041497 | HKR1 |
| 179146668 | 179146782 | chr5 | + | exo | hsa_circ_0075303 | 1.8506 | 0.041500 | CANX |
| 49042408 | 49103656 | chr22 | + | exo | | 1.1368 | 0.041527 | FAM19A5 |
| 40120400 | 40120586 | chr17 | − | as | hsa_circ_0000763 | 1.4107 | 0.041560 | CNP |
| 9367485 | 9388138 | chr18 | + | int | | 1.0820 | 0.041562 | TWSG1 |
| 80961340 | 80976031 | chr10 | + | exo | hsa_circ_0003605 | 1.2837 | 0.041599 | ZMIZ1 |
| 30693541 | 30693835 | chr21 | + | exo | hsa_circ_0001180 | 0.6785 | 0.041620 | BACH1 |
| 138892846 | 138893198 | chr6 | − | int | | 1.3863 | 0.041630 | NHSL1 |
| 39648294 | 39648666 | chr14 | + | exo | | 0.9274 | 0.041639 | PNN |
| 20480076 | 20481656 | chr6 | + | exo | hsa_circ_0075805 | 1.1045 | 0.041644 | E2F3 |
| 158850767 | 158882761 | chr6 | + | exo | hsa_circ_0078465 | 1.2119 | 0.041760 | TULP4 |
| 138400808 | 138407816 | chr3 | − | exo | hsa_circ_0067524 | 0.9630 | 0.041800 | PIK3CB |
| 33463187 | 33466333 | chr17 | − | exo | hsa_circ_0043101 | 1.0825 | 0.041843 | NLE1 |
| 31335607 | 31354680 | chr22 | − | exo | hsa_circ_0062865 | 1.0908 | 0.041871 | MORC2 |
| 77930940 | 77932010 | chr14 | + | exo | hsa_circ_0008404 | 1.2972 | 0.041876 | AHSA1 |
| 233225806 | 233231609 | chr1 | − | exo | hsa_circ_0005994 | 0.8116 | 0.041887 | PCNXL2 |
| 185146284 | 185155418 | chr3 | + | exo | | 0.8069 | 0.041998 | MAP3K13 |
| 79942864 | 79946065 | chr13 | − | exo | hsa_circ_0030541 | 0.8904 | 0.042014 | RBM26 |
| 134312006 | 134314448 | chr9 | + | exo | hsa_circ_0005871 | 1.1792 | 0.042115 | PRRC2B |
| 66288740 | 66291353 | chr11 | + | exo | hsa_circ_0022997 | 1.1555 | 0.042116 | BBS1 |
| 9804597 | 9804897 | chr3 | − | exo | | 1.3754 | 0.042133 | CAMK1 |
| 1437392 | 1461853 | chr17 | − | exo | hsa_circ_0041252 | 1.3612 | 0.042137 | PITPNA |
| 9484660 | 9484764 | chr2 | + | exo | hsa_circ_0052523 | 1.1307 | 0.042155 | ASAP2 |
| 1433137 | 1435777 | chr20 | − | exo | hsa_circ_0008198 | 1.1843 | 0.042244 | NSFL1C |
| 185638891 | 185641772 | chr3 | − | exo | hsa_circ_0006248 | 1.3292 | 0.042259 | TRA2B |
| 48744511 | 48760158 | chr20 | − | exo | hsa_circ_0060828 | 1.1117 | 0.042265 | TMEM189 |
| 1902352 | 1920350 | chr4 | + | exo | hsa_circ_0004156 | 0.7683 | 0.042311 | WHSC1 |
| 67431907 | 67490392 | chr14 | + | exo | hsa_circ_0032253 | 1.2632 | 0.042326 | GPHN |
| 14938844 | 14939516 | chr10 | + | exo | hsa_circ_0006780 | 0.8222 | 0.042390 | SUV39H2 |
| 213037066 | 213058738 | chr1 | + | exo | hsa_circ_0000182 | 0.7884 | 0.042402 | FLVCR1 |
| 108984657 | 108986092 | chr6 | + | exo | hsa_circ_0006404 | 0.8405 | 0.042410 | FOXO3 |
| 85180577 | 85181708 | chr15 | + | exo | hsa_circ_0036592 | 4.5259 | 0.042469 | SCAND2P |
| 73614502 | 73614814 | chr14 | + | exo | hsa_circ_0003848 | 1.1373 | 0.042561 | PSEN1 |
| 73418464 | 73429763 | chr11 | − | exo | hsa_circ_0000339 | 0.8043 | 0.042610 | RAB6A |
| 89192884 | 89212627 | chr14 | − | exo | hsa_circ_0032876 | 0.8881 | 0.042614 | EML5 |
| 130928324 | 130928494 | chrX | − | exo | hsa_circ_0091558 | 0.8714 | 0.042648 | LOC286467 |
| 118280253 | 118280344 | chr5 | − | exo | hsa_circ_0073649 | 0.8152 | 0.042658 | DTWD2 |
| 15508205 | 15531345 | chr8 | + | exo | hsa_circ_0006410 | 0.7609 | 0.042731 | TUSC3 |
| 64483842 | 64769779 | chr5 | − | exo | hsa_circ_0072665 | 1.1073 | 0.042749 | ADAMTS6 |
| 157150420 | 157150544 | chr6 | − | as | hsa_circ_0001656 | 1.0991 | 0.042763 | ARID1B |
| 89961445 | 89962491 | chr16 | + | exo | hsa_circ_0006661 | 1.2903 | 0.042787 | TCF25 |
| 6263945 | 6312055 | chr18 | − | exo | hsa_circ_0000824 | 1.3866 | 0.042884 | L3MBTL4 |
| 52234731 | 52249379 | chr13 | + | exo | hsa_circ_0030303 | 1.0711 | 0.042909 | WDFY2 |
| 161469647 | 161471011 | chr6 | + | exo | hsa_circ_0078619 | 0.7582 | 0.043034 | MAP3K4 |
| 104128452 | 104145855 | chr14 | + | exo | hsa_circ_0033502 | 1.1665 | 0.043078 | KLC1 |
| 171830241 | 171851336 | chr3 | + | exo | hsa_circ_0001361 | 1.2458 | 0.043078 | FNDC3B |
| 120995348 | 120995485 | chr12 | + | exo | hsa_circ_0028904 | 1.2428 | 0.043090 | RNF10 |
| 120276826 | 120278532 | chr11 | + | exo | hsa_circ_0024604 | 1.4387 | 0.043117 | ARHGEF12 |
| 80714040 | 80758872 | chr17 | + | exo | hsa_circ_0046449 | 1.1373 | 0.043121 | TBCD |
| 3658711 | 3681737 | chr7 | + | exo | | 0.6987 | 0.043132 | SDK1 |
| 2607703 | 2616454 | chr16 | + | exo | hsa_circ_0005466 | 0.6774 | 0.043172 | PDPK1 |
| 59981820 | 59996874 | chr17 | − | exo | | 0.8154 | 0.043228 | INTS2 |
| 98660130 | 98691146 | chr9 | + | exo | hsa_circ_0087605 | 0.9040 | 0.043246 | ERCC6L2 |
| 2012497 | 2012657 | chr16 | − | exo | hsa_circ_0037377 | 1.2073 | 0.043282 | RPS2 |
| 74481714 | 74489999 | chr9 | − | exo | hsa_circ_0006383 | 0.8470 | 0.043315 | ABHD17B |
| 7468756 | 7477996 | chr17 | + | exo | hsa_circ_0041905 | 1.0932 | 0.043351 | SENP3-EIF4A1 |
| 3197902 | 3215945 | chr3 | − | exo | hsa_circ_0003400 | 0.8153 | 0.043353 | CRBN |
| 10784445 | 10808849 | chr2 | − | exo | hsa_circ_0000977 | 0.7837 | 0.043376 | NOL10 |
| 34376033 | 34378572 | chr18 | − | int | hsa_circ_0047463 | 0.8471 | 0.043376 | TPGS2 |
| 73775150 | 73775265 | chr17 | − | int | hsa_circ_0000803 | 0.5860 | 0.043387 | H3F3B |
| 58996272 | 58996401 | chr1 | − | exo | hsa_circ_0012722 | 0.8376 | 0.043418 | OMA1 |
| 228496799 | 228497260 | chr1 | + | exo | hsa_circ_0016781 | 1.1031 | 0.043424 | OBSCN |
| 172325397 | 172325966 | chr2 | + | s/o | hsa_circ_0007529 | 0.9264 | 0.043481 | DCAF17 |
| 86284099 | 86293514 | chr9 | − | exo | hsa_circ_0087352 | 0.7085 | 0.043497 | UBQLN1 |
| 47380394 | 47381591 | chr11 | − | exo | hsa_circ_0000302 | 1.2511 | 0.043498 | SPI1 |
| 41015599 | 41016415 | chr4 | − | exo | hsa_circ_0005991 | 0.8375 | 0.043564 | APBB2 |
| 130915557 | 130916831 | chr8 | − | exo | hsa_circ_0085567 | 1.1684 | 0.043647 | FAM49B |
| 85723323 | 85742653 | chr11 | − | exo | hsa_circ_0023940 | 0.5928 | 0.043650 | PICALM |
| 38050167 | 38084515 | chr6 | + | exo | hsa_circ_0076251 | 1.3791 | 0.043674 | ZFAND3 |
| 76396757 | 76396936 | chr17 | + | exo | hsa_circ_0045897 | 1.1627 | 0.043717 | PGS1 |
| 203817281 | 203826149 | chr2 | + | exo | hsa_circ_0003747 | 0.7782 | 0.043744 | CARF |
| 37700501 | 37723145 | chr17 | + | s/o | | 1.1090 | 0.043752 | CDK12 |
| 242195015 | 242195594 | chr2 | + | as | hsa_circ_0004528 | 1.2334 | 0.043780 | HDLBP |
| 39411599 | 39414050 | chr22 | + | exo | hsa_circ_0063369 | 1.1110 | 0.043953 | APOBEC3C |
| 121220457 | 121229360 | chr12 | − | exo | hsa_circ_0004479 | 0.9015 | 0.043968 | SPPL3 |
| 183245098 | 183268082 | chr4 | + | exo | hsa_circ_0071475 | 0.7934 | 0.043971 | TENM3 |
| 150990287 | 151001420 | chr1 | + | exo | hsa_circ_0014085 | 1.1470 | 0.043982 | PRUNE |
| 38794512 | 38798121 | chr15 | − | exo | hsa_circ_0034414 | 1.4068 | 0.044007 | RASGRP1 |
| 129367080 | 129367205 | chr7 | + | exo | hsa_circ_0082314 | 0.8408 | 0.044087 | NRF1 |
| 123798166 | 123800207 | chr12 | − | exo | hsa_circ_0005310 | 0.8560 | 0.044104 | SBNO1 |
| 14568842 | 14569187 | chr19 | + | exo | hsa_circ_0008950 | 1.0872 | 0.044151 | PKN1 |
| 150496687 | 150502572 | chr5 | − | exo | hsa_circ_0074595 | 1.8204 | 0.044153 | ANXA6 |
| 155457868 | 155473602 | chr7 | + | exo | hsa_circ_0001771 | 0.7414 | 0.044160 | RBM33 |
| 5157293 | 5159602 | chr20 | + | exo | hsa_circ_0007888 | 1.2894 | 0.044176 | CDS2 |
| 111664114 | 111681159 | chr3 | + | exo | hsa_circ_0005128 | 0.8955 | 0.044185 | PHLDB2 |
| 44026199 | 44027487 | chr7 | − | exo | hsa_circ_0079988 | 1.4261 | 0.044207 | POLR2J4 |
| 160205697 | 160209175 | chr6 | − | exo | hsa_circ_0005388 | 1.0646 | 0.044211 | TCP1 |
| 127447537 | 127569382 | chr7 | + | exo | hsa_circ_0082142 | 0.8512 | 0.044227 | SND1 |
| 133324417 | 133331719 | chr12 | − | exo | hsa_circ_0008567 | 1.0826 | 0.044242 | ANKLE2 |
| 27151596 | 27156076 | chr8 | − | exo | hsa_circ_0083757 | 1.1891 | 0.044279 | TRIM35 |
| 99071202 | 99080651 | chr12 | + | exo | hsa_circ_0004901 | 0.9247 | 0.044321 | APAF1 |
| 71761715 | 71762097 | chr15 | + | int | | 0.8963 | 0.044352 | THSD4 |
| 25505309 | 25537030 | chr2 | − | exo | | 1.2656 | 0.044372 | DNMT3A |
| 101813725 | 101838883 | chr7 | + | exo | hsa_circ_0001732 | 1.0602 | 0.044401 | CUX1 |
| 48049356 | 48050499 | chr2 | − | exo | hsa_circ_0006794 | 1.0820 | 0.044419 | FBXO11 |
| 25925267 | 25926061 | chr15 | − | exo | | 1.1281 | 0.044540 | ATP10A |
| 36912135 | 36920478 | chr17 | + | exo | hsa_circ_0043368 | 0.8365 | 0.044556 | PSMB3 |
| 20570899 | 20576425 | chr18 | + | exo | hsa_circ_0047135 | 1.0964 | 0.044576 | RBBP8 |
| 888054 | 905704 | chr7 | + | exo | hsa_circ_0079071 | 1.1080 | 0.044588 | SUN1 |
| 15691616 | 15698758 | chr2 | − | exo | hsa_circ_0003287 | 1.1064 | 0.044589 | NBAS |
| 81929411 | 81942196 | chr16 | + | exo | hsa_circ_0000721 | 1.2259 | 0.044598 | PLCG2 |
| 9048750 | 9098771 | chr2 | − | exo | hsa_circ_0000972 | 0.7887 | 0.044681 | MBOAT2 |
| 43023282 | 43024183 | chr6 | − | exo | hsa_circ_0001608 | 1.1076 | 0.044692 | MRPL2 |
| 92347635 | 92347762 | chr14 | − | exo | hsa_circ_0032971 | 0.8087 | 0.044719 | FBLN5 |
| 59376327 | 59381958 | chr15 | + | exo | hsa_circ_0005763 | 1.0921 | 0.044811 | RNF111 |
| 80361812 | 80364358 | chr17 | − | exo | hsa_circ_0000815 | 1.1145 | 0.044867 | OGFOD3 |
| 60648117 | 60656722 | chr15 | − | exo | hsa_circ_0035554 | 1.1774 | 0.044920 | ANXA2 |
| 15510546 | 15510663 | chr19 | + | as | hsa_circ_0000902 | 1.1653 | 0.044946 | AKAP8L |
| 88008653 | 88017865 | chr16 | + | exo | hsa_circ_0040809 | 1.1953 | 0.045014 | BANP |
| 31725125 | 31774903 | chr3 | − | exo | | 1.2765 | 0.045073 | OSBPL10 |
| 70387756 | 70409129 | chr2 | − | exo | hsa_circ_0004975 | 0.9199 | 0.045158 | C2orf42 |
| 23356961 | 23385660 | chr1 | + | exo | hsa_circ_0007822 | 0.6821 | 0.045182 | KDM1A |
| 183245098 | 183245405 | chr4 | + | exo | hsa_circ_0002840 | 0.7289 | 0.045235 | TENM3 |
| 162890861 | 162891808 | chr5 | + | exo | | 0.9185 | 0.045264 | HMMR |
| 37751111 | 37764294 | chr2 | + | s/o | | 1.0947 | 0.045271 | XLOC_001429 |
| 132841908 | 132854660 | chr9 | + | exo | | 0.6503 | 0.045271 | GPR107 |
| 118982215 | 119033695 | chr9 | + | exo | hsa_circ_0088222 | 0.7877 | 0.045278 | PAPPA |
| 44257772 | 44303983 | chr1 | + | exo | hsa_circ_0012103 | 0.5104 | 0.045331 | ST3GAL3 |
| 26059596 | 26070495 | chr22 | + | exo | hsa_circ_0062649 | 0.8003 | 0.045368 | ADRBK2 |
| 35842009 | 35854588 | chr6 | − | exo | hsa_circ_0076177 | 0.8812 | 0.045371 | SRPK1 |
| 79748439 | 79750787 | chr15 | + | exo | | 0.8157 | 0.045410 | KIAA1024 |
| 35826799 | 35842268 | chr20 | + | exo | | 1.1711 | 0.045418 | RPN2 |
| 6420911 | 6434173 | chr9 | + | exo | hsa_circ_0002359 | 0.7677 | 0.045419 | UHRF2 |
| 36685939 | 36694658 | chr20 | + | s/o | hsa_circ_0007673 | 0.9430 | 0.045471 | RPRD1B |
| 73142760 | 73144766 | chr17 | − | exo | hsa_circ_0045602 | 1.2134 | 0.045512 | HN1 |
| 32197099 | 32199491 | chr10 | − | exo | hsa_circ_0000231 | 0.6094 | 0.045544 | ARHGAP12 |
| 77282987 | 77285143 | chr7 | + | ing | | 1.0975 | 0.045565 | |
| 116879948 | 116889297 | chr10 | + | exo | hsa_circ_0020093 | 0.7333 | 0.045589 | ATRNL1 |
| 53672262 | 53681075 | chrX | − | exo | hsa_circ_0001922 | 0.7628 | 0.045664 | HUWE1 |
| 133326545 | 133328709 | chr5 | − | exo | hsa_circ_0073932 | 1.3717 | 0.045677 | VDAC1 |
| 33185853 | 33217203 | chr7 | + | exo | hsa_circ_0003162 | 0.7833 | 0.045726 | BBS9 |
| 161443041 | 161455481 | chr6 | + | int | | 0.7843 | 0.045748 | MAP3K4 |
| 102398300 | 102401858 | chr7 | + | exo | hsa_circ_0007520 | 0.8348 | 0.045756 | FAM185A |
| 3317796 | 3344780 | chr4 | + | exo | hsa_circ_0006091 | 1.2336 | 0.045792 | RGS12 |
| 120830753 | 120836157 | chr2 | + | exo | hsa_circ_0001064 | 0.8477 | 0.045818 | EPB41L5 |
| 37427243 | 37431350 | chr8 | − | ing | | 0.8937 | 0.045821 | |
| 56104880 | 56105000 | chr2 | − | exo | hsa_circ_0054656 | 1.1256 | 0.045854 | EFEMP1 |
| 118920468 | 118925606 | chrX | − | exo | hsa_circ_0091420 | 1.6849 | 0.045974 | RPL39 |
| 1480998 | 1553916 | chr12 | + | exo | hsa_circ_0006747 | 0.7493 | 0.045984 | ERC1 |
| 179315102 | 179321614 | chr5 | − | exo | | 1.1767 | 0.046013 | TBC1D9B |
| 30921774 | 30951837 | chr4 | + | exo | | 0.7864 | 0.046031 | PCDH7 |
| 90313570 | 90315152 | chr9 | + | exo | hsa_circ_0087421 | 1.1518 | 0.046046 | DAPK1 |
| 36211599 | 36212059 | chr9 | + | exo | hsa_circ_0087004 | 1.0811 | 0.046107 | CLTA |
| 108286344 | 108286691 | chr9 | + | int | | 0.5778 | 0.046132 | FSD1L |
| 6150755 | 6155154 | chr7 | + | exo | hsa_circ_0007823 | 0.8612 | 0.046165 | USP42 |
| 53150260 | 53158544 | chr17 | + | exo | hsa_circ_0044699 | 0.8558 | 0.046233 | STXBP4 |
| 42761315 | 42798588 | chr8 | + | exo | hsa_circ_0084143 | 0.8998 | 0.046259 | HOOK3 |
| 1118055 | 1126416 | chr10 | + | exo | hsa_circ_0017438 | 1.0946 | 0.046267 | WDR37 |
| 42041214 | 42050989 | chr1 | − | exo | hsa_circ_0011950 | 0.7736 | 0.046334 | HIVEP3 |
| 78865519 | 78867665 | chr17 | + | exo | hsa_circ_0000813 | 0.6497 | 0.046342 | RPTOR |
| 131666045 | 131666176 | chr10 | − | exo | hsa_circ_0020496 | 0.8040 | 0.046388 | EBF3 |
| 514676 | 527802 | chr12 | + | exo | hsa_circ_0024951 | 1.1448 | 0.046391 | CCDC77 |
| 105953535 | 105955124 | chr14 | + | exo | hsa_circ_0033628 | 0.4406 | 0.046396 | CRIP1 |
| 82267507 | 82268990 | chr9 | + | exo | hsa_circ_0087300 | 1.4413 | 0.046399 | TLE4 |
| 24181170 | 24199945 | chr2 | + | exo | hsa_circ_0005970 | 0.7272 | 0.046417 | UBXN2A |
| 629834 | 637861 | chr6 | − | exo | hsa_circ_0075445 | 0.9221 | 0.046432 | EXOC2 |
| 82267504 | 82268990 | chr9 | + | exo | hsa_circ_0087293 | 1.4677 | 0.046458 | TLE4 |
| 76902524 | 76903190 | chr4 | − | exo | hsa_circ_0003534 | 0.4372 | 0.046463 | SDAD1 |
| 217363992 | 217366188 | chr2 | + | exo | hsa_circ_0058192 | 1.2701 | 0.046485 | RPL37A |
| 2297336 | 2298748 | chr17 | − | exo | | 1.1719 | 0.046514 | MNT |
| 8043537 | 8110761 | chr7 | + | exo | | 0.8510 | 0.046587 | GLCCI1 |
| 19308915 | 19310043 | chr19 | + | exo | hsa_circ_0050212 | 1.5313 | 0.046610 | RFXANK |
| 143412261 | 143436124 | chr8 | − | exo | hsa_circ_0085803 | 1.3118 | 0.046613 | TSNARE1 |
| 34868407 | 34887562 | chr19 | + | exo | hsa_circ_0050490 | 1.2163 | 0.046665 | GPI |
| 111857635 | 111896641 | chr13 | + | exo | hsa_circ_0002680 | 0.7787 | 0.046727 | ARHGEF7 |
| 42579888 | 42603044 | chr15 | + | exo | hsa_circ_0034805 | 0.8463 | 0.046736 | GANC |
| 82780581 | 82796909 | chr12 | + | exo | | 0.7997 | 0.046754 | METTL25 |
| 26786527 | 26786635 | chr1 | + | exo | hsa_circ_0011002 | 1.2056 | 0.046774 | DHDDS |
| 64878082 | 64879797 | chr12 | + | exo | hsa_circ_0000413 | 0.8952 | 0.046777 | TBK1 |
| 35603741 | 35609962 | chr17 | − | exo | hsa_circ_0002404 | 0.9215 | 0.046803 | ACACA |
| 114483003 | 114484081 | chr1 | + | exo | hsa_circ_0013617 | 0.7821 | 0.046807 | HIPK1 |
| 101718878 | 101719225 | chr8 | − | exo | hsa_circ_0006623 | 0.8331 | 0.046946 | PABPC1 |
| 48239625 | 48239790 | chr19 | + | exo | hsa_circ_0051700 | 1.6113 | 0.046954 | EHD2 |
| 9931806 | 9937063 | chr18 | + | exo | hsa_circ_0006990 | 0.8989 | 0.046959 | VAPA |
| 65473657 | 65492309 | chr2 | + | exo | hsa_circ_0008529 | 0.6099 | 0.046982 | ACTR2 |
| 15514731 | 15567918 | chr2 | − | exo | hsa_circ_0008261 | 0.8454 | 0.047029 | NBAS |
| 16727794 | 16727995 | chr9 | − | exo | hsa_circ_0086418 | 1.0821 | 0.047054 | BNC2 |
| 19498259 | 19498474 | chr3 | + | exo | | 0.7648 | 0.047173 | KCNH8 |
| 61912498 | 61913166 | chr11 | + | s/o | hsa_circ_0000315 | 1.6535 | 0.047198 | INCENP |
| 47531309 | 47537001 | chr16 | + | exo | hsa_circ_0039261 | 0.6794 | 0.047270 | PHKB |
| 72406762 | 72423384 | chr11 | − | exo | hsa_circ_0023461 | 1.0841 | 0.047291 | ARAP1 |
| 73950965 | 73958017 | chr4 | − | exo | hsa_circ_0001417 | 0.5646 | 0.047299 | ANKRD17 |
| 69548132 | 69553668 | chr15 | + | exo | | 0.9134 | 0.047385 | GLCE |
| 159238977 | 159239240 | chr6 | − | s/o | hsa_circ_0001664 | 1.1495 | 0.047388 | EZR |
| 30818047 | 30831190 | chr7 | + | exo | | 1.1349 | 0.047401 | FAM188B |
| 107236366 | 107254190 | chr12 | + | exo | hsa_circ_0028021 | 0.7858 | 0.047464 | RIC8B |
| 77210743 | 77221573 | chr7 | + | exo | hsa_circ_0002458 | 0.7566 | 0.047545 | PTPN12 |
| 5643126 | 5643661 | chr7 | + | exo | hsa_circ_0079280 | 1.3747 | 0.047557 | FSCN1 |
| 73521339 | 73523366 | chr7 | + | exo | hsa_circ_0001715 | 1.2910 | 0.047618 | LIMK1 |
| 44739739 | 44741214 | chr7 | + | exo | hsa_circ_0009092 | 1.4393 | 0.047629 | OGDH |
| 45954567 | 45959775 | chr10 | − | exo | hsa_circ_0018273 | 0.7621 | 0.047651 | MARCH8 |
| 24164288 | 24190184 | chr13 | + | exo | hsa_circ_0004196 | 0.8929 | 0.047657 | TNFRSF19 |
| 53670403 | 53671744 | chr12 | + | exo | hsa_circ_0008229 | 1.0828 | 0.047678 | ESPL1 |
| 31849672 | 31852977 | chr14 | − | exo | hsa_circ_0031570 | 0.7662 | 0.047768 | HEATR5A |
| 35169641 | 35174143 | chr19 | + | exo | | 0.8556 | 0.047809 | ZNF302 |
| 105197771 | 105204439 | chr10 | + | exo | hsa_circ_0019857 | 1.1199 | 0.047852 | PDCD11 |
| 72420914 | 72422544 | chr11 | − | exo | hsa_circ_0003557 | 1.1206 | 0.047890 | ARAP1 |
| 135639656 | 135644462 | chr6 | − | exo | | 0.8415 | 0.047896 | AHI1 |
| 17907047 | 17953998 | chr1 | + | exo | hsa_circ_0010210 | 1.1723 | 0.047899 | ARHGEF10L |
| 24679553 | 24679974 | chr14 | − | exo | hsa_circ_0007326 | 1.1446 | 0.047962 | CHMP4A |
| 5711031 | 5717503 | chr11 | − | int | hsa_circ_0000276 | 0.2871 | 0.047973 | TRIM5 |
| 120276826 | 120302620 | chr11 | + | exo | hsa_circ_0024609 | 1.1783 | 0.048011 | ARHGEF12 |
| 155233740 | 155239507 | chr1 | − | exo | hsa_circ_0014522 | 1.0578 | 0.048036 | CLK2 |
| 63631182 | 63667005 | chr2 | − | exo | hsa_circ_0007387 | 0.7791 | 0.048041 | WDPCP |
| 119109304 | 119130033 | chr9 | + | exo | hsa_circ_0088251 | 0.9072 | 0.048066 | PAPPA |
| 33356266 | 33356389 | chr20 | − | exo | hsa_circ_0059998 | 1.1332 | 0.048166 | NCOA6 |
| 31841015 | 31844174 | chr14 | − | exo | hsa_circ_0031569 | 0.7145 | 0.048186 | HEATR5A |
| 6461357 | 6464466 | chr7 | − | exo | hsa_circ_0004330 | 1.1077 | 0.048200 | DAGLB |
| 129021835 | 129022690 | chr8 | + | exo | hsa_circ_0008849 | 1.0638 | 0.048277 | PVT1 |
| 54292038 | 54294350 | chr4 | + | exo | hsa_circ_0069748 | 0.7994 | 0.048289 | FIP1L1 |
| 17311447 | 17313692 | chr19 | + | exo | hsa_circ_0049948 | 1.3002 | 0.048338 | MYO9B |
| 91142866 | 91252672 | chr14 | − | exo | hsa_circ_0005422 | 0.9112 | 0.048413 | TTC7B |
| 6021853 | 6038473 | chr1 | − | exo | hsa_circ_0009468 | 1.1436 | 0.048432 | NPHP4 |
| 140109659 | 140109825 | chr9 | + | int | | 1.1273 | 0.048437 | NDOR1 |
| 42553155 | 42560230 | chr15 | − | exo | hsa_circ_0034803 | 0.7686 | 0.048487 | TMEM87A |
| 76854879 | 76856033 | chrX | − | exo | hsa_circ_0001931 | 1.1116 | 0.048493 | ATRX |
| 34685382 | 34706566 | chr19 | + | exo | hsa_circ_0008030 | 1.6690 | 0.048496 | LSM14A |
| 21983243 | 21994668 | chr16 | + | exo | hsa_circ_0038479 | 1.2839 | 0.048505 | UQCRC2 |
| 151130894 | 151131115 | chrX | − | exo | hsa_circ_0091710 | 0.7910 | 0.048525 | GABRE |
| 44986659 | 45000952 | chr3 | − | exo | hsa_circ_0065052 | 1.1418 | 0.048526 | ZDHHC3 |
| 67873091 | 67893087 | chr15 | + | exo | hsa_circ_0005498 | 0.9196 | 0.048544 | MAP2K5 |
| 127965678 | 127983624 | chr3 | + | exo | hsa_circ_0067209 | 1.3871 | 0.048558 | EEFSEC |
| 223946971 | 223959941 | chr1 | + | exo | hsa_circ_0004879 | 0.8411 | 0.048628 | CAPN2 |
| 42481721 | 42524149 | chr13 | − | exo | hsa_circ_0030123 | 0.8180 | 0.048692 | VWA8 |
| 38610179 | 38613982 | chr17 | + | exo | hsa_circ_0043552 | 1.1146 | 0.048774 | IGFBP4 |
| 46804823 | 46810323 | chr11 | − | exo | | 0.9499 | 0.048794 | CKAP5 |
| 183361267 | 183369064 | chr3 | + | exo | hsa_circ_0001367 | 0.7089 | 0.048803 | KLHL24 |
| 139815688 | 139815842 | chr5 | + | exo | hsa_circ_0074243 | 1.1721 | 0.048838 | ANKHD1 |
| 84765030 | 84772711 | chr6 | + | exo | | 1.1491 | 0.048844 | MRAP2 |
| 69583082 | 69589077 | chr14 | − | exo | hsa_circ_0007392 | 1.0923 | 0.048844 | DCAF5 |
| 48196616 | 48206619 | chr8 | + | exo | hsa_circ_0002157 | 0.8189 | 0.048859 | SPIDR |
| 13604092 | 13610799 | chr20 | − | exo | hsa_circ_0059476 | 0.7547 | 0.048922 | TASP1 |
| 102180052 | 102182109 | chr7 | − | exo | hsa_circ_0081683 | 1.6231 | 0.049074 | POER2J3 |
| 69169490 | 69171146 | chr5 | + | exo | hsa_circ_0072803 | 1.2195 | 0.049193 | SMA4 |
| 5653125 | 5654467 | chr19 | + | exo | hsa_circ_0000881 | 1.1404 | 0.049211 | SAFB |
| 169863210 | 169867032 | chr3 | − | exo | hsa_circ_0001360 | 0.6963 | 0.049314 | PHC3 |
| 121000747 | 121001746 | chr12 | + | exo | hsa_circ_0006390 | 1.1187 | 0.049413 | RNF10 |
| 145161990 | 145162515 | chr8 | + | exo | hsa_circ_0085973 | 1.1990 | 0.049452 | MAF1 |
| 197541778 | 197547301 | chr3 | + | exo | hsa_circ_0002266 | 0.7982 | 0.049464 | LRCH3 |
| 171785557 | 171785759 | chr5 | − | int | | 1.1423 | 0.049609 | SH3PXD2B |
| 128691933 | 128697886 | chr9 | + | exo | hsa_circ_0004804 | 0.7112 | 0.049644 | PBX3 |
| 33714053 | 33719586 | chr20 | − | exo | | 0.3996 | 0.049789 | EDEM2 |
| 40656443 | 40668286 | chr1 | + | exo | hsa_circ_0007302 | 0.8522 | 0.049802 | RLF |
| 170688866 | 170695542 | chr1 | + | exo | hsa_circ_0004370 | 0.6658 | 0.049816 | PRRX1 |
| 22332466 | 22333137 | chr8 | + | exo | hsa_circ_0083619 | 1.3723 | 0.049831 | PPP3CC |
| 57842331 | 57851246 | chr17 | + | exo | hsa_circ_0004027 | 0.7161 | 0.049841 | VMP1 |
| 17933230 | 17934761 | chr20 | − | exo | hsa_circ_0006704 | 0.8110 | 0.049950 | SNX5 |
| 185320068 | 185340722 | chr4 | − | exo | | 0.8399 | 0.049979 | IRF2 |

TABLE 3

| | | | | circRNA | | Fold | p-value | |
|---|---|---|---|---|---|---|---|---|
| Tx Start | Tx End | Chrom | Strand | type | circRNA Alias | BD OFC | BD OFC | GeneSymbol |
| 136389287 | 136396692 | chr2 | + | exo | hsa_circ_0001069 | 0.8515 | 0.000565 | R3HDM1 |
| 5213834 | 5214425 | chr3 | + | exo | hsa_circ_0064080 | 0.9294 | 0.000857 | ARL8B |
| 82917099 | 82947551 | chr11 | + | exo | hsa_circ_0023858 | 0.9041 | 0.001039 | ANKRD42 |
| 79996891 | 80022523 | chr9 | + | exo | hsa_circ_0008075 | 0.8962 | 0.001097 | VPS13A |
| 154184795 | 154186422 | chr1 | − | exo | hsa_circ_0014364 | 0.9310 | 0.001356 | C1orf43 |
| 53188358 | 53191453 | chr16 | + | s/o | hsa_circ_0000701 | 0.8756 | 0.001534 | CHD9 |
| 34595458 | 34596350 | chr14 | − | exo | | 1.1038 | 0.001599 | EGLN3 |
| 29483000 | 29497015 | chr17 | + | exo | hsa_circ_0007542 | 0.8590 | 0.001816 | NF1 |
| 35343915 | 35348156 | chr17 | + | exo | hsa_circ_0003532 | 1.0992 | 0.001853 | AATF |
| 48152822 | 48155648 | chr17 | + | exo | hsa_circ_0044468 | 1.2025 | 0.002623 | ITGA3 |
| 44441761 | 44443967 | chr1 | + | exo | hsa_circ_0012123 | 0.7392 | 0.002665 | ATP6V0B |
| 4848946 | 4849292 | chr17 | − | exo | hsa_circ_0041630 | 1.0960 | 0.002700 | PFN1 |
| 127670655 | 127674305 | chr9 | − | exo | hsa_circ_0002883 | 1.0752 | 0.003151 | GOEGA1 |
| 58499574 | 58500089 | chr19 | − | exo | hsa_circ_0052320 | 1.1111 | 0.003337 | ZNF606 |
| 52773707 | 52789638 | chr20 | − | exo | hsa_circ_0060929 | 1.0699 | 0.003375 | CYP24A1 |
| 53410256 | 53416411 | chr12 | + | exo | hsa_circ_0026497 | 0.8916 | 0.003444 | EIF4B |
| 171482591 | 171484477 | chr5 | − | exo | hsa_circ_0001555 | 0.9458 | 0.003574 | STK10 |
| 40409395 | 40486735 | chr12 | − | s/o | hsa_circ_0000391 | 0.8164 | 0.003639 | SLC2A13 |
| 128902834 | 128951922 | chr8 | + | exo | hsa_circ_0085540 | 0.8800 | 0.003644 | PVT1 |
| 6657590 | 6657991 | chr12 | − | exo | hsa_circ_0000375 | 0.7920 | 0.003713 | IFFO1 |
| 151509199 | 151520283 | chr4 | − | exo | hsa_circ_0004780 | 0.9281 | 0.004273 | LRBA |
| 54940142 | 54940318 | chr20 | + | exo | | 1.0912 | 0.004501 | FAM210B |
| 145620479 | 145620570 | chr8 | − | exo | hsa_circ_0086088 | 0.9415 | 0.004803 | CPSF1 |
| 239184383 | 239186596 | chr2 | − | exo | hsa_circ_0003854 | 0.9373 | 0.004940 | PER2 |
| 79555969 | 79575848 | chr17 | − | exo | hsa_circ_0046188 | 1.1415 | 0.004961 | NPLOC4 |
| 149781199 | 149784743 | chr5 | − | exo | hsa_circ_0074527 | 0.9131 | 0.005391 | CD74 |
| 5413685 | 5417655 | chr7 | − | exo | hsa_circ_0006824 | 0.9344 | 0.005504 | TNRC18 |
| 169863210 | 169890500 | chr3 | − | exo | hsa_circ_0067913 | 1.3251 | 0.005555 | PHC3 |
| 93676169 | 93677054 | chr14 | + | exo | hsa_circ_0004080 | 1.0598 | 0.005798 | UBR7 |
| 227702788 | 227779067 | chr2 | + | exo | | 0.9410 | 0.005929 | RHBDD1 |
| 76444310 | 76446890 | chr12 | − | exo | hsa_circ_0027610 | 1.1960 | 0.005936 | NAP1L1 |
| 114388277 | 114411482 | chr11 | + | s/o | | 0.9424 | 0.006073 | snoU13 |
| 94114814 | 94248681 | chr5 | − | exo | hsa_circ_0073379 | 0.8886 | 0.006393 | MCTP1 |
| 303021 | 303397 | chr20 | − | s/o | | 0.9078 | 0.006579 | NRSN2-AS1 |
| 40102424 | 40127930 | chr7 | + | exo | hsa_circ_0079939 | 1.0787 | 0.006594 | CDK13 |
| 111274562 | 111279894 | chr13 | + | exo | hsa_circ_0030885 | 1.0940 | 0.006631 | CARKD |
| 51957464 | 51963580 | chr13 | − | exo | hsa_circ_0030296 | 1.2256 | 0.006721 | INTS6 |
| 46096161 | 46125470 | chr22 | + | exo | hsa_circ_0008472 | 0.7334 | 0.006861 | ATXN10 |
| 13054580 | 13054850 | chr19 | + | s/o | hsa_circ_0000897 | 1.2037 | 0.006871 | CALR |
| 15753646 | 15766031 | chr3 | − | exo | | 1.1634 | 0.006916 | ANKRD28 |
| 24523658 | 24524213 | chr20 | + | s/o | | 0.9042 | 0.007084 | SYNDIG1 |
| 39893421 | 39895194 | chr6 | − | exo | | 0.9371 | 0.007113 | MOCS1 |
| 22046315 | 22097363 | chr9 | + | exo | hsa_circ_0004297 | 1.0856 | 0.007119 | CDKN2B-AS1 |
| 27642756 | 27689252 | chr7 | − | s/o | | 1.0492 | 0.007149 | HIBADH |
| 79563141 | 79575848 | chr17 | − | exo | hsa_circ_0005221 | 1.1767 | 0.007160 | NPLOC4 |
| 35236117 | 35236221 | chr20 | + | exo | hsa_circ_0001151 | 0.9030 | 0.007390 | C20orf24 |
| 29328755 | 29329131 | chr17 | + | ing | | 1.0859 | 0.007409 | |
| 30029633 | 30032700 | chr3 | + | exo | hsa_circ_0064651 | 1.1400 | 0.007428 | RBMS3 |
| 28970311 | 28970421 | chr16 | + | exo | hsa_circ_0038885 | 1.2273 | 0.007679 | NFATC2IP |
| 126799558 | 126811437 | chr10 | − | int | hsa_circ_0004875 | 1.0634 | 0.007691 | CTBP2 |
| 38610760 | 38612940 | chr21 | − | exo | hsa_circ_0061687 | 1.0885 | 0.007783 | DSCR3 |
| 64863339 | 64868113 | chr5 | + | exo | hsa_circ_0072697 | 0.7769 | 0.007801 | PPWD1 |
| 153227786 | 153228670 | chrX | − | int | | 1.1111 | 0.007840 | HCFC1 |
| 151735592 | 151735871 | chr1 | − | s/o | hsa_circ_0000132 | 0.8937 | 0.008006 | MRPL9 |
| 38463710 | 38466898 | chr22 | + | exo | hsa_circ_0063280 | 0.7194 | 0.008055 | PICK1 |
| 58130779 | 58132861 | chr19 | + | s/o | | 1.0634 | 0.008068 | ZNF134 |
| 19063007 | 19068019 | chr16 | + | exo | hsa_circ_0038314 | 1.2286 | 0.008106 | TMC7 |
| 133242062 | 133244088 | chr12 | − | int | | 0.9411 | 0.008214 | POLE |
| 147438785 | 147460753 | chr1 | + | exo | hsa_circ_0013966 | 1.0741 | 0.008263 | GPR89B |
| 80847543 | 80869665 | chr17 | + | exo | hsa_circ_0046547 | 0.9077 | 0.008393 | TBCD |
| 32689634 | 32696620 | chr1 | + | exo | hsa_circ_0011381 | 1.1304 | 0.008702 | EIF3I |
| 133341934 | 133343019 | chr3 | − | exo | hsa_circ_0008069 | 1.0749 | 0.008714 | TOPBP1 |
| 116926635 | 116944277 | chr1 | + | exo | | 0.9064 | 0.008874 | ATP1A1 |
| 113568990 | 113569830 | chr4 | + | int | hsa_circ_0092348 | 1.1430 | 0.008922 | LARP7 |
| 70301561 | 70302282 | chr16 | − | exo | hsa_circ_0040203 | 0.9100 | 0.008925 | AARS |
| 5307418 | 5320002 | chr17 | − | exo | hsa_circ_0041700 | 0.9173 | 0.008934 | NUP88 |
| 61552509 | 61561106 | chr2 | − | exo | | 1.1285 | 0.009017 | USP34 |
| 201786215 | 201786413 | chr1 | + | exo | hsa_circ_0008264 | 0.8853 | 0.009035 | NAV1 |
| 98530863 | 98533298 | chr7 | + | exo | hsa_circ_0005493 | 1.0703 | 0.009096 | TRRAP |
| 113976596 | 113977741 | chr13 | + | exo | hsa_circ_0031006 | 1.1252 | 0.009114 | LAMP1 |
| 12332743 | 12356334 | chr12 | − | exo | hsa_circ_0025489 | 0.9404 | 0.009136 | LRP6 |
| 38439561 | 38441924 | chr21 | − | exo | hsa_circ_0008160 | 1.2275 | 0.009238 | PIGP |
| 3976434 | 3976629 | chr19 | − | int | hsa_circ_0000872 | 0.8383 | 0.009243 | EEF2 |
| 94066434 | 94068129 | chr6 | − | exo | hsa_circ_0077398 | 1.1161 | 0.009325 | EPHA7 |
| 37162982 | 37163182 | chr3 | − | exo | hsa_circ_0064832 | 1.1945 | 0.009412 | LRRFIP2 |
| 116966876 | 116982009 | chr6 | − | exo | hsa_circ_0077705 | 1.1837 | 0.009625 | ZUFSP |
| 23650789 | 23651172 | chr7 | + | exo | hsa_circ_0001684 | 0.9237 | 0.009706 | CCDC126 |
| 97318671 | 97343323 | chr8 | + | exo | hsa_circ_0008303 | 1.1007 | 0.010148 | PTDSS1 |
| 127660827 | 127662826 | chr9 | − | exo | | 1.0597 | 0.010206 | GOLGA1 |
| 77282987 | 77295385 | chr7 | + | as | | 1.0789 | 0.010234 | RSBN1L-AS1 |
| 127683985 | 127697119 | chr10 | + | exo | | 0.8600 | 0.010665 | FANK1 |
| 79813017 | 79813462 | chr17 | − | exo | hsa_circ_0046263 | 1.1557 | 0.010767 | P4HB |
| 68321225 | 68321778 | chr16 | + | int | | 1.0934 | 0.010850 | SLC7A6 |
| 187627716 | 187630999 | chr4 | − | exo | hsa_circ_0001461 | 0.9064 | 0.010889 | FAT1 |
| 87765294 | 87797542 | chr7 | + | exo | hsa_circ_0080968 | 0.5247 | 0.011017 | ADAM22 |
| 20067587 | 20077417 | chr13 | − | exo | hsa_circ_0029601 | 1.1179 | 0.011194 | TPTE2 |
| 41521867 | 41542820 | chr22 | + | exo | hsa_circ_0063481 | 0.9447 | 0.011365 | EP300 |
| 61458751 | 61459055 | chr8 | + | int | hsa_circ_0001804 | 0.8820 | 0.011484 | RAB2A |
| 101874252 | 101886778 | chr2 | + | exo | hsa_circ_0055894 | 1.1379 | 0.011640 | CNOT11 |
| 151372040 | 151374412 | chr1 | + | exo | hsa_circ_0014157 | 1.1057 | 0.011644 | PSMB4 |
| 107866903 | 107867544 | chr1 | + | exo | hsa_circ_0002286 | 0.8536 | 0.011704 | NTNG1 |
| 29677910 | 29678100 | chr16 | + | int | hsa_circ_0000686 | 0.7298 | 0.011736 | QPRT |
| 186504915 | 186505373 | chr3 | + | exo | hsa_circ_0068464 | 0.6346 | 0.011791 | EIF4A2 |
| 5212187 | 5216099 | chr3 | + | exo | hsa_circ_0005046 | 1.0446 | 0.011954 | ARL8B |
| 31816241 | 31830172 | chr22 | + | exo | hsa_circ_0062901 | 1.0668 | 0.012200 | DRG1 |
| 161455290 | 161471011 | chr6 | + | exo | hsa_circ_0078617 | 0.9122 | 0.012333 | MAP3K4 |
| 50571209 | 50618904 | chr13 | − | exo | hsa_circ_0006475 | 1.0675 | 0.012447 | DLEU2 |
| 118682698 | 118693460 | chr12 | − | exo | hsa_circ_0028671 | 0.7231 | 0.012457 | TAOK3 |
| 102201526 | 102249725 | chr5 | + | exo | hsa_circ_0073492 | 0.9587 | 0.012477 | PAM |
| 152771320 | 152775004 | chrX | + | exo | hsa_circ_0091743 | 1.0983 | 0.012555 | BGN |
| 48779302 | 48780572 | chrX | − | exo | hsa_circ_0090533 | 1.2270 | 0.012585 | OTUD5 |
| 235582787 | 235590554 | chr1 | + | exo | hsa_circ_0006474 | 1.1327 | 0.012630 | TBCE |
| 53365044 | 53365148 | chr6 | − | exo | hsa_circ_0076798 | 1.0684 | 0.012638 | GCLC |
| 19260034 | 19261573 | chr19 | − | exo | hsa_circ_0050205 | 1.2893 | 0.012964 | MEF2BNB-MEF2B |
| 144771373 | 144773883 | chr8 | + | exo | hsa_circ_0085859 | 0.9477 | 0.012990 | ZNF707 |
| 40544231 | 40546159 | chr1 | − | exo | hsa_circ_0011883 | 1.3679 | 0.013004 | PPT1 |
| 52524247 | 52524687 | chr3 | + | int | hsa_circ_0092276 | 1.1544 | 0.013180 | NISCH |
| 138994281 | 139003046 | chr5 | + | exo | hsa_circ_0007452 | 0.9206 | 0.013261 | UBE2D2 |
| 6620218 | 6632869 | chr5 | − | exo | hsa_circ_0071760 | 0.9313 | 0.013274 | NSUN2 |
| 120916382 | 120924441 | chr11 | + | exo | hsa_circ_0000365 | 0.8210 | 0.013385 | TBCEL |
| 19574204 | 19574328 | chr19 | + | int | hsa_circ_0000916 | 1.1948 | 0.013404 | GATAD2A |
| 121662310 | 121663789 | chr10 | + | exo | hsa_circ_0020197 | 1.0944 | 0.013510 | SEC23IP |
| 20518297 | 20532824 | chr2 | − | s/o | hsa_circ_0004642 | 0.9389 | 0.013551 | PUM2 |
| 42284534 | 42284717 | chr2 | + | int | | 0.8688 | 0.013763 | PKDCC |
| 115024714 | 115060196 | chr9 | − | exo | hsa_circ_0008192 | 0.8820 | 0.013807 | PTBP3 |
| 169863210 | 169863309 | chr3 | − | exo | hsa_circ_0067911 | 1.3235 | 0.013883 | PHC3 |
| 61655830 | 61657304 | chr17 | + | exo | hsa_circ_0045202 | 0.9131 | 0.013945 | DCAF7 |
| 64878082 | 64879797 | chr12 | + | exo | hsa_circ_0000413 | 0.9403 | 0.014083 | TBK1 |
| 46878860 | 46879831 | chr19 | + | exo | hsa_circ_0003356 | 0.8890 | 0.014233 | PPP5C |
| 45781180 | 45781873 | chr19 | + | exo | hsa_circ_0000941 | 0.9152 | 0.014309 | MARK4 |
| 4700365 | 4707364 | chr16 | + | exo | hsa_circ_0007032 | 0.8834 | 0.014325 | MGRN1 |
| 128655032 | 128657110 | chr7 | − | exo | hsa_circ_0082275 | 0.9140 | 0.014364 | TNPO3 |
| 115202366 | 115205825 | chr5 | + | exo | hsa_circ_0002919 | 0.7422 | 0.014389 | AP3S1 |
| 231885671 | 231906816 | chr1 | + | exo | | 1.0512 | 0.014508 | DISCI |
| 179136873 | 179143295 | chr5 | + | exo | hsa_circ_0001565 | 0.9284 | 0.014511 | CANX |
| 43552465 | 43555513 | chr17 | − | exo | hsa_circ_0003434 | 0.9193 | 0.014616 | PLEKHM1 |
| 6776706 | 6777567 | chr7 | + | exo | hsa_circ_0007607 | 0.9351 | 0.014665 | PMS2CL |
| 88977237 | 88989213 | chr4 | + | exo | | 0.9091 | 0.014939 | PKD2 |
| 46567153 | 46567326 | chr11 | − | exo | hsa_circ_0021866 | 0.8862 | 0.014975 | AMBRA1 |
| 79571573 | 79575848 | chr17 | − | exo | hsa_circ_0000814 | 1.1951 | 0.015050 | NPLOC4 |
| 45506682 | 45509814 | chr21 | + | exo | hsa_circ_0061936 | 0.8974 | 0.015220 | TRAPPC10 |
| 156294762 | 156304709 | chr1 | − | exo | hsa_circ_0005131 | 0.9413 | 0.015370 | CCT3 |
| 45587230 | 45599993 | chr14 | − | s/o | | 0.9039 | 0.015581 | FKBP3 |
| 101716524 | 101717901 | chr8 | − | exo | hsa_circ_0085133 | 0.9125 | 0.015864 | PABPC1 |
| 32190706 | 32190881 | chr16 | − | exo | hsa_circ_0039187 | 0.7250 | 0.016001 | HERC2P4 |
| 117109316 | 117110576 | chr11 | + | exo | hsa_circ_0024402 | 0.9167 | 0.016005 | RNF214 |
| 60613530 | 60642498 | chr17 | + | exo | hsa_circ_0045128 | 0.9163 | 0.016022 | TLK2 |
| 51123962 | 51364771 | chr10 | − | exo | hsa_circ_0018343 | 0.9067 | 0.016042 | PARG |
| 34376033 | 34409158 | chr18 | − | s/o | hsa_circ_0047467 | 1.1837 | 0.016141 | TPGS2 |
| 116335174 | 116444414 | chr10 | − | exo | hsa_circ_0020076 | 0.9467 | 0.016205 | ABLIM1 |
| 56958592 | 56985413 | chr15 | − | exo | | 1.1077 | 0.016208 | ZNF280D |
| 102486229 | 102489217 | chr14 | + | exo | hsa_circ_0008683 | 0.8030 | 0.016304 | DYNC1H1 |
| 16327848 | 16345099 | chr3 | + | exo | hsa_circ_0001273 | 0.9302 | 0.016355 | OXNAD1 |
| 18285849 | 18286507 | chr19 | + | exo | hsa_circ_0005571 | 0.9534 | 0.016384 | IFI30 |
| 1417498 | 1422395 | chr19 | + | exo | hsa_circ_0048232 | 0.9260 | 0.016493 | DAZAP1 |
| 27829360 | 27840440 | chr12 | + | exo | hsa_circ_0025731 | 1.1385 | 0.016605 | PPFIBP1 |
| 68059317 | 68072052 | chr16 | + | exo | hsa_circ_0039908 | 0.5068 | 0.016611 | DUS2 |
| 153635690 | 153636950 | chr1 | − | exo | hsa_circ_0014263 | 1.0716 | 0.016667 | ILF2 |
| 78575577 | 78575863 | chr15 | − | exo | hsa_circ_0036450 | 1.0531 | 0.016669 | WDR61 |
| 100830613 | 100833198 | chr11 | + | exo | | 1.1080 | 0.016900 | ARHGAP42 |
| 117788564 | 117788924 | chrX | + | exo | hsa_circ_0007733 | 1.3030 | 0.016941 | DOCK11 |
| 42941740 | 42942776 | chr6 | − | exo | hsa_circ_0005038 | 0.9350 | 0.017021 | PEX6 |
| 4845635 | 4848517 | chr17 | + | exo | hsa_circ_0041623 | 0.8757 | 0.017043 | RNF167 |
| 72082738 | 72091059 | chr9 | − | exo | hsa_circ_0004129 | 0.9557 | 0.017099 | APBA1 |
| 6620185 | 6628610 | chr7 | + | exo | hsa_circ_0079381 | 1.1602 | 0.017109 | ZDHHC4 |
| 6021853 | 6029319 | chr1 | − | exo | hsa_circ_0009135 | 0.7767 | 0.017415 | NPHP4 |
| 37617884 | 37620116 | chr6 | − | exo | | 1.2906 | 0.017424 | MDGA1 |
| 25398740 | 25405963 | chr20 | + | exo | | 1.0521 | 0.017438 | GINS1 |
| 85309700 | 85337797 | chr11 | − | exo | | 0.9206 | 0.017447 | DLG2 |
| 37250990 | 37382367 | chr7 | − | exo | hsa_circ_0079891 | 1.1735 | 0.017477 | ELMO1 |
| 2184780 | 2209644 | chrX | − | exo | hsa_circ_0007582 | 0.9384 | 0.017535 | DHRSX |
| 29311641 | 29311945 | chr17 | + | int | | 1.1128 | 0.017646 | RNF135 |
| 32479446 | 32483505 | chr3 | + | s/o | hsa_circ_0005414 | 0.7347 | 0.017850 | CMTM7 |
| 129280642 | 129280746 | chr3 | − | exo | hsa_circ_0067301 | 0.5266 | 0.017859 | PLXND1 |
| 56492459 | 56496546 | chr16 | + | exo | hsa_circ_0039444 | 1.0843 | 0.017902 | OGFOD1 |
| 58594115 | 58594266 | chr16 | − | exo | hsa_circ_0007079 | 1.1051 | 0.018022 | CNOT1 |
| 11210898 | 11213462 | chr19 | + | exo | hsa_circ_0049377 | 0.9256 | 0.018044 | LDLR |
| 220375617 | 220406205 | chr1 | − | exo | hsa_circ_0016513 | 0.9192 | 0.018236 | RAB3GAP2 |
| 76729417 | 76739097 | chr10 | + | exo | | 1.0983 | 0.018252 | KAT6B |
| 64466443 | 64484047 | chr5 | − | exo | hsa_circ_0003503 | 0.9482 | 0.018373 | ADAMTS6 |
| 94653105 | 94669388 | chr10 | + | exo | hsa_circ_0019171 | 0.9036 | 0.018414 | EXOC6 |
| 90670648 | 90670832 | chr6 | + | as | hsa_circ_0001623 | 0.9184 | 0.018421 | BACH2 |
| 116091160 | 116094330 | chr9 | − | exo | | 1.0464 | 0.018563 | WDR31 |
| 87808910 | 87812746 | chr4 | − | exo | hsa_circ_0004262 | 0.8284 | 0.018588 | C4orf36 |
| 184114751 | 184130178 | chr4 | + | exo | hsa_circ_0003437 | 1.1277 | 0.018743 | WWC2 |
| 64084777 | 64100213 | chr5 | + | exo | hsa_circ_0072654 | 0.8506 | 0.018768 | CWC27 |
| 77327195 | 77348851 | chr11 | − | exo | hsa_circ_0023691 | 0.7614 | 0.018773 | CLNS1A |
| 29068914 | 29070498 | chr1 | + | exo | hsa_circ_0000039 | 0.9507 | 0.018812 | YTHDF2 |
| 110764194 | 110765822 | chr12 | + | exo | hsa_circ_0005279 | 1.1252 | 0.018822 | ATP2A2 |
| 34969052 | 34991833 | chr11 | + | exo | hsa_circ_0006254 | 0.9049 | 0.018989 | PDHX |
| 28420334 | 28445911 | chr11 | + | s/o | | 1.0673 | 0.019169 | RP11-22P4.1 |
| 34974539 | 34985912 | chr21 | − | exo | | 0.8230 | 0.019271 | CRYZL1 |
| 43142254 | 43142429 | chr1 | + | exo | hsa_circ_0011977 | 1.3831 | 0.019309 | PPIH |
| 30661008 | 30662078 | chr7 | + | exo | hsa_circ_0009000 | 0.9450 | 0.019339 | GARS |
| 89054662 | 89071017 | chr15 | − | s/o | | 0.8830 | 0.019342 | DET1 |
| 215259710 | 215298093 | chr1 | + | exo | hsa_circ_0016459 | 1.3911 | 0.019396 | KCNK2 |
| 7760002 | 7760161 | chr17 | − | exo | hsa_circ_0041959 | 1.1468 | 0.019529 | NAA38 |
| 74570202 | 74632392 | chr11 | − | exo | hsa_circ_0023598 | 1.2042 | 0.019545 | XRRA1 |
| 25497809 | 25505580 | chr2 | − | exo | hsa_circ_0053028 | 0.8993 | 0.019603 | DNMT3A |
| 86308723 | 86310291 | chr2 | − | exo | hsa_circ_0001038 | 0.9092 | 0.019620 | POLR1A |
| 34614144 | 34635942 | chr21 | + | s/o | | 1.2222 | 0.019704 | IFNAR2 |
| 39595439 | 39613921 | chr18 | + | exo | hsa_circ_0000843 | 0.9131 | 0.019904 | PIK3C3 |
| 28907071 | 28907741 | chr1 | − | exo | hsa_circ_0005174 | 0.9294 | 0.019913 | SNHG12 |
| 152480271 | 152522207 | chr7 | + | exo | hsa_circ_0005181 | 0.8937 | 0.019976 | ACTR3B |
| 33632307 | 33637797 | chr20 | − | exo | | 0.9328 | 0.020188 | TRPC4AP |
| 82533610 | 82545116 | chr15 | − | exo | hsa_circ_0036559 | 1.1296 | 0.020257 | EFTUD1 |
| 134308003 | 134330553 | chr9 | + | exo | hsa_circ_0005708 | 0.9379 | 0.020298 | PRRC2B |
| 88211723 | 88221044 | chr10 | − | exo | hsa_circ_0019005 | 1.0619 | 0.020317 | WAPAL |
| 33603810 | 33609159 | chr20 | − | exo | hsa_circ_0060035 | 1.1719 | 0.020344 | TRPC4AP |
| 25848915 | 25849486 | chr4 | − | exo | | 1.0758 | 0.020387 | SEL1L3 |
| 97820925 | 97823884 | chr7 | + | exo | hsa_circ_0081207 | 0.8846 | 0.020474 | LMTK2 |
| 101996606 | 101997988 | chr10 | − | exo | hsa_circ_0019504 | 1.0435 | 0.020590 | CWF19L1 |
| 146432295 | 146451816 | chrX | − | ing | | 1.1291 | 0.020713 | |
| 1219147 | 1235307 | chr4 | − | exo | hsa_circ_0001386 | 0.8922 | 0.020773 | CTBP1 |
| 43628823 | 43652158 | chr15 | + | s/o | | 1.1162 | 0.020799 | ADAL |
| 41006257 | 41007382 | chr1 | + | exo | | 1.1321 | 0.020839 | ZNF684 |
| 100826890 | 100828258 | chr14 | − | exo | hsa_circ_0033191 | 1.1603 | 0.020851 | WARS |
| 53289511 | 53302038 | chr16 | + | exo | hsa_circ_0039365 | 0.9131 | 0.020868 | CHD9 |
| 42365180 | 42375484 | chr19 | + | exo | hsa_circ_0051244 | 1.0903 | 0.020874 | RPS19 |
| 102697158 | 102710510 | chr10 | + | exo | hsa_circ_0002045 | 1.2912 | 0.020935 | FAM178A |
| 110764194 | 110780253 | chr12 | + | exo | hsa_circ_0028173 | 1.1626 | 0.020957 | ATP2A2 |
| 7164499 | 7170346 | chr2 | + | exo | hsa_circ_0052491 | 0.8807 | 0.020998 | RNF144A |
| 167754623 | 167766153 | chr3 | − | exo | hsa_circ_0067871 | 0.9474 | 0.021069 | GOLIM4 |
| 14038705 | 14039001 | chr19 | + | exo | hsa_circ_0049704 | 1.1673 | 0.021095 | CC2D1A |
| 70263118 | 70265962 | chr11 | + | exo | hsa_circ_0023339 | 0.9110 | 0.021227 | CTTN |
| 170667931 | 170669824 | chr5 | + | exo | hsa_circ_0004738 | 0.7582 | 0.021254 | RANBP17 |
| 47716964 | 47727660 | chr3 | − | exo | hsa_circ_0001295 | 0.9246 | 0.021342 | SMARCC1 |
| 31789187 | 31803431 | chr13 | + | exo | hsa_circ_0002635 | 0.7498 | 0.021458 | B3GALTL |
| 6026173 | 6040252 | chr19 | − | exo | hsa_circ_0000883 | 1.0951 | 0.021544 | RFX2 |
| 220253068 | 220263134 | chr1 | − | s/o | hsa_circ_0000185 | 0.9258 | 0.021624 | BPNT1 |
| 100006372 | 100009557 | chr6 | − | exo | hsa_circ_0003326 | 1.1654 | 0.021664 | CCNC |
| 72398922 | 72400649 | chr7 | + | exo | hsa_circ_0080451 | 1.2633 | 0.021792 | POM121 |
| 189022209 | 189026744 | chr4 | − | exo | | 0.9112 | 0.021809 | TRIML2 |
| 47584769 | 47585552 | chr19 | − | exo | | 0.9173 | 0.021889 | ZC3H4 |
| 44564901 | 44605080 | chr19 | + | s/o | hsa_circ_0000938 | 1.2564 | 0.021925 | ZNF223 |
| 618892 | 647089 | chr7 | − | exo | hsa_circ_0079040 | 0.8411 | 0.022118 | PRKAR1B |
| 189357857 | 189377916 | chr4 | + | s/o | | 1.0776 | 0.022161 | LINC01060 |
| 33450727 | 33467233 | chr3 | − | exo | hsa_circ_0001985 | 0.8263 | 0.022358 | UBP1 |
| 156619298 | 156619438 | chr7 | − | exo | hsa_circ_0083126 | 0.8976 | 0.022438 | LMBR1 |
| 50405399 | 50408891 | chr20 | − | exo | hsa_circ_0060909 | 1.0848 | 0.022459 | SALL4 |
| 183388821 | 183390272 | chr3 | + | exo | | 0.7865 | 0.022463 | KLHL24 |
| 104352338 | 104377185 | chr10 | + | exo | hsa_circ_0019756 | 1.0537 | 0.022641 | SUFU |
| 171537385 | 171544267 | chr1 | + | exo | hsa_circ_0004412 | 1.1369 | 0.022647 | PRRC2C |
| 63821212 | 63829355 | chr15 | + | exo | hsa_circ_0008104 | 1.0963 | 0.022662 | USP3 |
| 151934651 | 151936377 | chrX | − | exo | hsa_circ_0091722 | 1.2944 | 0.022759 | MAGEA3 |
| 115013208 | 115060196 | chr9 | − | exo | hsa_circ_0003458 | 1.3212 | 0.022844 | PTBP3 |
| 99718694 | 99761618 | chr8 | − | exo | hsa_circ_0085045 | 1.0529 | 0.022921 | STK3 |
| 1093881 | 1168869 | chr2 | + | exo | | 1.2516 | 0.023152 | SNTG2 |
| 19049161 | 19049858 | chr19 | − | exo | hsa_circ_0008135 | 1.0941 | 0.023157 | HOMER3 |
| 66367959 | 66373490 | chr11 | + | exo | hsa_circ_0023006 | 1.1442 | 0.023171 | CCS |
| 39746137 | 39796226 | chr14 | + | exo | hsa_circ_0031741 | 1.1321 | 0.023296 | CTAGE5 |
| 140458885 | 140459606 | chr9 | − | exo | hsa_circ_0006502 | 0.6977 | 0.023349 | DPH7 |
| 10368897 | 10369361 | chr19 | + | exo | hsa_circ_0008762 | 1.0638 | 0.023465 | MRPL4 |
| 33397466 | 33427756 | chr7 | + | exo | hsa_circ_0008336 | 0.8803 | 0.023567 | BBS9 |
| 57148183 | 57161450 | chr17 | − | exo | hsa_circ_0004090 | 1.1937 | 0.023626 | TRIM37 |
| 110321942 | 110343415 | chr2 | − | exo | hsa_circ_0003065 | 1.0764 | 0.023738 | SEPT10 |
| 193181194 | 193205486 | chr1 | + | exo | hsa_circ_0000167 | 0.8206 | 0.023803 | CDC73 |
| 9517036 | 9520937 | chr2 | + | exo | hsa_circ_0052535 | 1.1339 | 0.024035 | ASAP2 |
| 40745835 | 40750331 | chr22 | + | exo | hsa_circ_0002811 | 1.0655 | 0.024068 | ADSL |
| 1902352 | 1920350 | chr4 | + | exo | hsa_circ_0004156 | 0.8410 | 0.024423 | WHSC1 |
| 3424822 | 3427945 | chr11 | + | exo | hsa_circ_0020846 | 1.2822 | 0.024476 | TSSC2 |
| 30207591 | 30214332 | chr17 | − | exo | hsa_circ_0000757 | 0.9286 | 0.024561 | UTP6 |
| 46934593 | 46950619 | chr16 | + | exo | hsa_circ_0039219 | 1.1436 | 0.024650 | GPT2 |
| 27830711 | 27830906 | chr2 | + | exo | hsa_circ_0008911 | 1.1732 | 0.024688 | ZNF512 |
| 5118219 | 5126751 | chr17 | − | exo | hsa_circ_0041671 | 1.0939 | 0.024795 | SCIMP |
| 33954359 | 33962059 | chr20 | − | exo | hsa_circ_0060063 | 1.1671 | 0.024858 | UQCC1 |
| 45153949 | 45163688 | chr21 | + | exo | hsa_circ_0061891 | 0.8117 | 0.025111 | PDXK |
| 117423010 | 117448255 | chr12 | + | exo | hsa_circ_0028613 | 1.0834 | 0.025126 | FBXW8 |
| 62892698 | 62895478 | chr12 | + | exo | hsa_circ_0027364 | 0.9308 | 0.025160 | MON2 |
| 34793786 | 34805178 | chr21 | + | exo | hsa_circ_0002113 | 1.2612 | 0.025186 | IFNGR2 |
| 14153264 | 14153601 | chr19 | + | exo | | 0.9390 | 0.025354 | IL27RA |
| 66923610 | 66990707 | chr12 | − | exo | | 0.8005 | 0.025425 | GRIP1 |
| 30693541 | 30724134 | chr21 | + | exo | | 0.9469 | 0.025491 | BACH1 |
| 77823695 | 77832220 | chr11 | − | exo | hsa_circ_0003615 | 1.1177 | 0.025549 | ALG8 |
| 2986239 | 2994019 | chr4 | + | exo | | 0.9463 | 0.025607 | GRK4 |
| 755878 | 760253 | chr11 | + | exo | hsa_circ_0003307 | 1.0735 | 0.025645 | TALDO1 |
| 110134684 | 110135599 | chr11 | − | exo | hsa_circ_0002773 | 0.9040 | 0.025678 | RDX |
| 60601596 | 60613698 | chr17 | + | exo | hsa_circ_0006250 | 0.9451 | 0.025755 | TLK2 |
| 148851036 | 148851432 | chr7 | + | exo | hsa_circ_0004351 | 0.8938 | 0.025892 | ZNF398 |
| 42373100 | 42375484 | chr19 | + | exo | hsa_circ_0051245 | 1.0853 | 0.025906 | RPS19 |
| 11223953 | 11224438 | chr19 | + | exo | hsa_circ_0002579 | 1.1528 | 0.025986 | LDLR |
| 16758226 | 16764505 | chr5 | − | exo | hsa_circ_0072008 | 1.0774 | 0.026075 | MYO10 |
| 118583263 | 118583380 | chr12 | − | as | hsa_circ_0000445 | 1.0960 | 0.026138 | PEBP1 |
| 98231032 | 98232213 | chr9 | − | exo | | 1.3600 | 0.026164 | PTCH1 |
| 33160465 | 33161645 | chr1 | − | exo | hsa_circ_0004895 | 1.1382 | 0.026187 | SYNC |
| 42919243 | 42932507 | chr8 | + | exo | hsa_circ_0008397 | 0.9457 | 0.026312 | FNTA |
| 169279207 | 169293738 | chr1 | − | exo | hsa_circ_0000156 | 0.9561 | 0.026388 | NME7 |
| 44970017 | 44970136 | chr11 | − | int | hsa_circ_0000293 | 1.1833 | 0.026531 | TP53I11 |
| 120276826 | 120351139 | chr11 | + | exo | hsa_circ_0024615 | 0.9238 | 0.026593 | ARHGEF12 |
| 61484604 | 61504528 | chr8 | + | exo | hsa_circ_0007581 | 0.9433 | 0.026636 | RAB2A |
| 140646904 | 140812121 | chr4 | − | exo | hsa_circ_0071000 | 0.7839 | 0.026649 | MAML3 |
| 14588570 | 14589379 | chr19 | − | exo | hsa_circ_0049753 | 1.0501 | 0.026694 | GIPC1 |
| 31994030 | 32006486 | chr1 | + | ing | hsa_circ_0000046 | 1.1644 | 0.026767 | |
| 228388478 | 228401709 | chr2 | + | exo | hsa_circ_0058522 | 1.0435 | 0.026772 | AGFG1 |
| 78115117 | 78115647 | chr17 | − | exo | hsa_circ_0045989 | 1.0764 | 0.026922 | EIF4A3 |
| 45560357 | 45568558 | chr17 | − | exo | | 0.9503 | 0.026959 | MRPL45P2 |
| 36936478 | 36950106 | chr15 | + | exo | hsa_circ_0034398 | 1.1955 | 0.026983 | C15orf41 |
| 2903852 | 2945848 | chr20 | + | exo | hsa_circ_0001126 | 1.2765 | 0.027174 | PTPRA |
| 79810631 | 79810766 | chr5 | − | as | hsa_circ_0001507 | 1.0559 | 0.027231 | FAM151B |
| 110062421 | 110068928 | chr9 | + | exo | hsa_circ_0087855 | 0.9197 | 0.027372 | RAD23B |
| 46832463 | 46834530 | chr19 | + | exo | | 1.4055 | 0.027466 | HIF3A |
| 56989531 | 57025950 | chr6 | + | exo | | 1.0827 | 0.027470 | ZNF451 |
| 42191638 | 42202061 | chr15 | − | exo | hsa_circ_0034776 | 1.0736 | 0.027627 | EHD4 |
| 215645283 | 215661841 | chr2 | − | exo | hsa_circ_0058055 | 0.9210 | 0.027720 | BARD1 |
| 39064972 | 39065915 | chr2 | − | s/o | | 1.0823 | 0.027780 | DHX57 |
| 44229519 | 44237812 | chr22 | − | exo | hsa_circ_0063697 | 0.8994 | 0.027802 | SULT4A1 |
| 45885784 | 45889438 | chr19 | − | exo | | 0.9270 | 0.027812 | PPP1R13L |
| 25264266 | 25266713 | chr16 | − | exo | hsa_circ_0038702 | 1.1251 | 0.027944 | ZKSCAN2 |
| 35846859 | 35854683 | chr1 | + | exo | hsa_circ_0000051 | 0.9032 | 0.027980 | ZMYM4 |
| 26789747 | 26802589 | chr10 | + | exo | | 1.1104 | 0.027987 | APBB1IP |
| 24416624 | 24418806 | chr6 | + | exo | hsa_circ_0075835 | 0.9384 | 0.028018 | MRS2 |
| 125939261 | 125939875 | chr5 | + | exo | | 0.9206 | 0.028133 | PHAX |
| 23999828 | 24135302 | chr16 | + | exo | hsa_circ_0038645 | 0.8140 | 0.028232 | PRKCB |
| 64096074 | 64100213 | chr5 | + | exo | | 0.8704 | 0.028318 | CWC27 |
| 29927157 | 29927575 | chr8 | − | exo | hsa_circ_0004283 | 1.0604 | 0.028332 | SARAF |
| 36953719 | 36976504 | chr5 | + | exo | hsa_circ_0001472 | 0.9469 | 0.028368 | NIPBL |
| 106888840 | 106938791 | chr7 | − | exo | hsa_circ_0081888 | 1.1360 | 0.028407 | COG5 |
| 43090856 | 43091077 | chr15 | − | int | | 1.2124 | 0.028466 | TTBK2 |
| 115030328 | 115060196 | chr9 | − | exo | hsa_circ_0003500 | 1.1806 | 0.028512 | PTBP3 |
| 130773149 | 130776616 | chr11 | − | exo | hsa_circ_0024895 | 1.1830 | 0.028862 | SNX19 |
| 168233450 | 168250336 | chr5 | − | exo | hsa_circ_0074930 | 0.9084 | 0.028866 | SLIT3 |
| 139993019 | 139994721 | chr4 | − | exo | hsa_circ_0002594 | 1.0372 | 0.028878 | ELF2 |
| 158688265 | 158703410 | chr6 | + | s/o | | 1.2182 | 0.028908 | RP11-732M18.3 |
| 35195356 | 35201078 | chr6 | + | exo | hsa_circ_0076092 | 1.0818 | 0.028909 | SCUBE3 |
| 71369656 | 71370884 | chr11 | + | ing | | 1.1663 | 0.028954 | |
| 59204761 | 59209198 | chr15 | − | exo | hsa_circ_0000605 | 0.8771 | 0.028994 | SLTM |
| 56419830 | 56423287 | chr16 | − | exo | hsa_circ_0000704 | 1.1089 | 0.029022 | AMFR |
| 27825691 | 27828418 | chr13 | + | exo | hsa_circ_0029813 | 1.1093 | 0.029113 | RPL21 |
| 35802664 | 35804870 | chr17 | + | exo | hsa_circ_0043284 | 0.7708 | 0.029296 | TADA2A |
| 183515710 | 183519316 | chr3 | + | exo | hsa_circ_0001371 | 1.1532 | 0.029349 | YEATS2 |
| 43295853 | 43297268 | chr5 | − | exo | hsa_circ_0072391 | 0.9458 | 0.029446 | HMGCS1 |
| 30366964 | 30375041 | chr22 | + | exo | hsa_circ_0006303 | 0.9316 | 0.029483 | MTMR3 |
| 51712030 | 51716483 | chr14 | + | exo | hsa_circ_0031900 | 0.7255 | 0.029598 | TMX1 |
| 24021007 | 24046439 | chr2 | − | exo | hsa_circ_0052941 | 0.9468 | 0.029861 | ATAD2B |
| 119413806 | 119414072 | chr9 | − | exo | hsa_circ_0088266 | 1.0544 | 0.029877 | ASTN2 |
| 60888143 | 60888306 | chr20 | − | exo | hsa_circ_0061081 | 1.1389 | 0.029920 | LAMA5 |
| 138203933 | 138255748 | chr7 | + | exo | hsa_circ_0082582 | 1.2675 | 0.029931 | TRIM24 |
| 39108038 | 39130809 | chr3 | + | exo | hsa_circ_0001283 | 1.1379 | 0.029968 | WDR48 |
| 17543067 | 17551835 | chr6 | + | exo | | 0.9438 | 0.030060 | CAP2 |
| 26784281 | 26786635 | chr1 | + | exo | hsa_circ_0007537 | 1.0522 | 0.030102 | DHDDS |
| 45289532 | 45333038 | chr6 | − | exo | | 0.9135 | 0.030275 | SUPT3H |
| 50647289 | 50667802 | chr14 | − | exo | hsa_circ_0031830 | 1.0720 | 0.030346 | SOS2 |
| 43679047 | 43680248 | chr7 | − | exo | hsa_circ_0001700 | 1.0749 | 0.030401 | COA1 |
| 47147486 | 47158244 | chr3 | − | exo | | 0.9415 | 0.030441 | SETD2 |
| 138784244 | 138842320 | chr5 | − | exo | hsa_circ_0074216 | 1.1852 | 0.030600 | ECSCR |
| 46925682 | 46930378 | chr7 | − | s/o | hsa_circ_0001704 | 0.9088 | 0.030653 | AC004901.1 |
| 136770511 | 136771563 | chr5 | − | int | | 0.8567 | 0.030677 | SPOCK1 |
| 47768925 | 47769734 | chr21 | + | exo | hsa_circ_0002903 | 0.8924 | 0.030681 | PCNT |
| 53441706 | 53442118 | chrX | − | exo | | 1.1381 | 0.030694 | SMC1A |
| 47444334 | 47446190 | chrX | + | exo | hsa_circ_0090449 | 1.1026 | 0.030740 | TIMP1 |
| 66426136 | 66430767 | chr17 | − | exo | hsa_circ_0045502 | 1.1028 | 0.030847 | WIPI1 |
| 51825603 | 51826337 | chr13 | + | exo | hsa_circ_0030292 | 1.1621 | 0.030902 | FAM124A |
| 136035782 | 136048826 | chr3 | + | exo | | 0.8809 | 0.030945 | PCCB |
| 5827104 | 5842668 | chr10 | − | exo | hsa_circ_0005379 | 0.8271 | 0.030960 | GDI2 |
| 109687716 | 109697315 | chr6 | − | exo | hsa_circ_0077582 | 1.2295 | 0.031030 | CD164 |
| 99324974 | 99327765 | chr9 | − | exo | hsa_circ_0002780 | 1.1895 | 0.031053 | CDC14B |
| 62515438 | 62519013 | chr17 | + | exo | hsa_circ_0045306 | 1.1631 | 0.031159 | CEP95 |
| 28970037 | 28970421 | chr16 | + | exo | hsa_circ_0038883 | 1.1136 | 0.031236 | NFATC2IP |
| 42501716 | 42513913 | chr17 | − | exo | hsa_circ_0003508 | 0.8449 | 0.031238 | GPATCH8 |
| 71855409 | 71860053 | chr10 | + | exo | | 1.0535 | 0.031263 | H2AFY2 |
| 137940767 | 137942575 | chr3 | + | exo | hsa_circ_0007912 | 1.1400 | 0.031296 | ARMC8 |
| 17267718 | 17278874 | chr19 | + | exo | hsa_circ_0049925 | 1.0670 | 0.031322 | MYO9B |
| 151214598 | 151215043 | chr1 | + | exo | hsa_circ_0014132 | 1.1960 | 0.031389 | PIP5K1A |
| 196050614 | 196051311 | chr3 | − | exo | hsa_circ_0068641 | 1.1123 | 0.031421 | TM4SF19-TCTEX1D2 |
| 6426842 | 6431672 | chr7 | + | exo | hsa_circ_0004349 | 1.0872 | 0.031432 | RAC1 |
| 100645330 | 100645426 | chr15 | + | as | hsa_circ_0000661 | 0.9326 | 0.031439 | ADAMTS17 |
| 73808192 | 73809959 | chr17 | + | exo | hsa_circ_0045714 | 1.1679 | 0.031697 | UNK |
| 24786772 | 24807429 | chr2 | + | s/o | hsa_circ_0000983 | 1.0470 | 0.031744 | NCOA1 |
| 130685988 | 130688240 | chr3 | + | exo | | 0.9591 | 0.031764 | ATP2C1 |
| 160181332 | 160185162 | chr1 | + | exo | hsa_circ_0014865 | 1.1449 | 0.031791 | PEA15 |
| 145992374 | 146007432 | chr6 | − | exo | | 1.0741 | 0.031808 | EPM2A |
| 20811436 | 20811559 | chr14 | − | s/o | hsa_circ_0000520 | 0.7332 | 0.031858 | RPPH1 |
| 101997616 | 102031004 | chr13 | − | exo | hsa_circ_0030788 | 0.7692 | 0.031915 | NALCN |
| 32902351 | 32907027 | chr18 | + | s/o | hsa_circ_0003785 | 1.4346 | 0.031918 | XLOC_012658 |
| 104135847 | 104139768 | chr14 | + | exo | hsa_circ_0007737 | 0.9278 | 0.032012 | KLC1 |
| 38879442 | 38891942 | chr22 | − | exo | hsa_circ_0063313 | 0.9137 | 0.032129 | DDX17 |
| 116879948 | 116925405 | chr10 | + | exo | hsa_circ_0020094 | 0.7679 | 0.032223 | ATRNL1 |
| 43565432 | 43568828 | chr6 | + | exo | hsa_circ_0005451 | 1.0382 | 0.032263 | POLH |
| 67546221 | 67559327 | chr3 | − | exo | hsa_circ_0004276 | 1.0456 | 0.032267 | SUCLG2 |
| 62160368 | 62162206 | chr1 | − | s/o | | 0.9590 | 0.032281 | TM2D1 |
| 82836502 | 82836622 | chr5 | + | int | hsa_circ_0001510 | 1.1506 | 0.032322 | VCAN |
| 77210743 | 77221573 | chr7 | + | exo | hsa_circ_0002458 | 0.9063 | 0.032387 | PTPN12 |
| 78177431 | 78178966 | chr1 | − | exo | hsa_circ_0012989 | 1.2310 | 0.032440 | USP33 |
| 98262520 | 98264657 | chr2 | + | exo | hsa_circ_0055754 | 1.2133 | 0.032494 | COX5B |
| 58339410 | 58347472 | chr12 | + | exo | | 1.1552 | 0.032570 | XRCC6BP1 |
| 247319707 | 247323115 | chr1 | − | s/o | | 0.9237 | 0.032623 | ZNF124 |
| 135624111 | 135626611 | chr2 | − | s/o | | 0.9044 | 0.032789 | CCNT2-AS1 |
| 32987845 | 32995095 | chr11 | + | exo | | 0.9038 | 0.032908 | QSER1 |
| 71243446 | 71244971 | chr10 | + | exo | hsa_circ_0002758 | 1.1426 | 0.033045 | TSPAN15 |
| 43819869 | 43861614 | chr11 | + | exo | | 1.0830 | 0.033067 | HSD17B12 |
| 7409610 | 7423911 | chr10 | − | exo | hsa_circ_0017648 | 0.9441 | 0.033189 | SFMBT2 |
| 18108412 | 18108601 | chr11 | − | exo | hsa_circ_0021417 | 1.1129 | 0.033199 | SAAL1 |
| 85692787 | 85695016 | chr11 | − | exo | hsa_circ_0008773 | 1.1208 | 0.033348 | PICALM |
| 77616277 | 77620283 | chr8 | + | exo | hsa_circ_0084789 | 0.8483 | 0.033374 | ZFHX4 |
| 93118615 | 93118705 | chr14 | + | s/o | hsa_circ_0000561 | 1.1638 | 0.033504 | RIN3 |
| 99301359 | 99304174 | chr9 | − | exo | hsa_circ_0087643 | 0.9274 | 0.033546 | CDC14B |
| 22326248 | 22328860 | chr22 | − | exo | hsa_circ_0062456 | 1.0576 | 0.033573 | TOP3B |
| 16632297 | 16641930 | chr1 | − | exo | hsa_circ_0010146 | 1.1306 | 0.033633 | FBXO42 |
| 97715747 | 97720938 | chr6 | − | exo | hsa_circ_0077409 | 1.1513 | 0.033642 | MMS22L |
| 28249524 | 28269803 | chr22 | − | s/o | | 1.2298 | 0.033650 | PITPNB |
| 122237752 | 122239714 | chr12 | − | exo | hsa_circ_0029021 | 0.8947 | 0.033683 | LINC01089 |
| 74655296 | 74657969 | chr16 | − | exo | hsa_circ_0040474 | 0.9425 | 0.033771 | RFWD3 |
| 210003452 | 210004388 | chr1 | + | exo | hsa_circ_0016330 | 0.8841 | 0.033804 | DIEXF |
| 175716656 | 175717958 | chr5 | + | exo | hsa_circ_0075092 | 1.1058 | 0.033892 | SIMC1 |
| 203155045 | 203160560 | chr2 | + | exo | hsa_circ_0008327 | 1.0678 | 0.033960 | NOP58 |
| 21461899 | 21470081 | chr18 | + | exo | hsa_circ_0047232 | 1.0722 | 0.033981 | LAMA3 |
| 40655612 | 40657441 | chr2 | − | exo | hsa_circ_0005232 | 0.6772 | 0.034010 | SLC8A1 |
| 30748452 | 30756180 | chr2 | + | exo | hsa_circ_0000987 | 0.9305 | 0.034035 | LCLAT1 |
| 236626200 | 236659132 | chr2 | + | exo | hsa_circ_0058794 | 0.6669 | 0.034066 | AGAP1 |
| 179310431 | 179319578 | chr3 | − | exo | hsa_circ_0005292 | 1.0479 | 0.034131 | MRPL47 |
| 32480394 | 32481489 | chr12 | + | exo | hsa_circ_0025841 | 0.9229 | 0.034209 | BICD1 |
| 40567269 | 40567355 | chr3 | + | exo | hsa_circ_0064924 | 1.2093 | 0.034229 | ZNF621 |
| 3424822 | 3426193 | chr11 | + | exo | hsa_circ_0020845 | 1.2549 | 0.034348 | TSSC2 |
| 156619298 | 156626486 | chr7 | − | exo | hsa_circ_0083127 | 0.9029 | 0.034426 | LMBR1 |
| 10286215 | 10288043 | chr19 | − | exo | hsa_circ_0049224 | 0.9472 | 0.034440 | DNMT1 |
| 42807412 | 42807742 | chr22 | − | exo | hsa_circ_0001240 | 1.2030 | 0.034451 | NFAM1 |
| 20744988 | 20749278 | chr16 | − | exo | hsa_circ_0038383 | 1.0578 | 0.034528 | THUMPD1 |
| 38297823 | 38315052 | chr8 | − | exo | hsa_circ_0084010 | 0.9257 | 0.034572 | FGFR1 |
| 40655612 | 40657444 | chr2 | − | exo | hsa_circ_0000994 | 0.6588 | 0.034601 | SLC8A1 |
| 3660963 | 3661999 | chr19 | − | exo | hsa_circ_0000871 | 0.9265 | 0.034629 | PIP5K1C |
| 74948073 | 74948409 | chr15 | − | exo | hsa_circ_0004401 | 0.9361 | 0.034712 | EDC3 |
| 174229522 | 174232559 | chr2 | + | s/o | | 1.0739 | 0.034760 | CDCA7 |
| 805012 | 805187 | chr19 | + | exo | hsa_circ_0048104 | 0.8403 | 0.034934 | PTBP1 |
| 102076623 | 102080295 | chr11 | + | exo | hsa_circ_0006286 | 1.0745 | 0.035047 | YAP1 |
| 58965500 | 58979345 | chr14 | + | exo | | 1.1291 | 0.035139 | KIAA0586 |
| 30104674 | 30110339 | chr13 | − | exo | hsa_circ_0006290 | 1.0633 | 0.035161 | SLC7A1 |
| 69304539 | 69318051 | chr2 | + | exo | hsa_circ_0055033 | 0.8275 | 0.035221 | ANTXR1 |
| 9020437 | 9021799 | chr12 | + | exo | hsa_circ_0025388 | 1.1050 | 0.035261 | A2ML1 |
| 168007608 | 168014465 | chr1 | + | exo | hsa_circ_0000155 | 0.9126 | 0.035301 | DCAF6 |
| 21178613 | 21193019 | chr22 | − | exo | hsa_circ_0062397 | 0.8918 | 0.035343 | PI4KA |
| 52157280 | 52161651 | chr5 | + | exo | hsa_circ_0004840 | 1.0553 | 0.035345 | ITGA1 |
| 52861717 | 52863535 | chr1 | − | exo | hsa_circ_0003494 | 1.1870 | 0.035388 | ORC1 |
| 111406810 | 111419418 | chr2 | − | exo | hsa_circ_0056037 | 1.1437 | 0.035439 | BUB1 |
| 43479368 | 43490707 | chr18 | − | exo | | 1.0758 | 0.035455 | EPG5 |
| 121675707 | 121732604 | chr4 | − | exo | hsa_circ_0005654 | 0.7201 | 0.035573 | PRDM5 |
| 62297840 | 62298224 | chr11 | − | int | hsa_circ_0000320 | 1.0898 | 0.035615 | AHNAK |
| 89570990 | 89579642 | chr4 | + | exo | hsa_circ_0006387 | 1.0598 | 0.035625 | HERC3 |
| 12826218 | 12826553 | chr19 | − | exo | hsa_circ_0049587 | 0.9288 | 0.035651 | TNPO2 |
| 212977661 | 212977993 | chr1 | + | exo | hsa_circ_0016404 | 0.7795 | 0.035666 | TATDN3 |
| 5365638 | 5367009 | chr17 | − | exo | | 0.8553 | 0.035748 | DHX33 |
| 139865173 | 139866645 | chr5 | + | exo | hsa_circ_0074264 | 1.0814 | 0.035862 | ANKHD1 |
| 15858833 | 15889942 | chr10 | − | exo | hsa_circ_0000219 | 0.8842 | 0.036103 | FAM188A |
| 114823280 | 114841741 | chr12 | − | exo | hsa_circ_0003176 | 1.0592 | 0.036217 | TBX5 |
| 82984689 | 82989872 | chr11 | − | exo | | 1.0377 | 0.036226 | CCDC90B |
| 57430575 | 57430887 | chr17 | + | exo | hsa_circ_0005600 | 0.9130 | 0.036242 | YPEL2 |
| 25628813 | 25631937 | chr17 | + | exo | hsa_circ_0042488 | 0.8734 | 0.036245 | WSB1 |
| 67720920 | 67722005 | chr14 | + | int | | 0.9185 | 0.036341 | MPP5 |
| 201790558 | 201796171 | chr2 | − | exo | | 1.2028 | 0.036390 | ORC2 |
| 176085759 | 176118210 | chr1 | − | exo | hsa_circ_0015374 | 0.9192 | 0.036480 | RFWD2 |
| 3712576 | 3716836 | chr11 | − | exo | hsa_circ_0006430 | 1.1258 | 0.036562 | NUP98 |
| 40301605 | 40301685 | chr13 | + | exo | hsa_circ_0030039 | 0.9083 | 0.036775 | COG6 |
| 43106586 | 43107263 | chr6 | + | exo | hsa_circ_0076479 | 1.0767 | 0.036828 | PTK7 |
| 1118055 | 1151207 | chr10 | + | exo | hsa_circ_0017441 | 0.9456 | 0.036866 | WDR37 |
| 34925772 | 34929671 | chr19 | + | exo | hsa_circ_0050525 | 0.9229 | 0.036875 | UBA2 |
| 55731669 | 55742565 | chr15 | − | exo | | 0.8691 | 0.036978 | DYX1C1 |
| 18419661 | 18462483 | chr3 | − | exo | hsa_circ_0064555 | 0.7852 | 0.036979 | SATB1 |
| 135106002 | 135116484 | chr10 | − | exo | | 1.0443 | 0.037154 | TUBGCP2 |
| 3761470 | 3764177 | chr1 | − | exo | hsa_circ_0009435 | 1.1337 | 0.037244 | CEP104 |
| 38894089 | 38894578 | chr22 | − | exo | hsa_circ_0063329 | 0.8166 | 0.037312 | DDX17 |
| 20101609 | 20104725 | chr11 | + | exo | hsa_circ_0004819 | 1.1204 | 0.037326 | NAV2 |
| 147581750 | 147612308 | chr6 | + | exo | hsa_circ_0078162 | 0.9197 | 0.037379 | STXBP5 |
| 72181471 | 72188374 | chr10 | + | exo | hsa_circ_0018657 | 1.0767 | 0.037382 | EIF4EBP2 |
| 38571895 | 38610522 | chr19 | + | exo | hsa_circ_0050792 | 0.9292 | 0.037475 | SIPA1L3 |
| 93714717 | 93722752 | chr3 | + | exo | hsa_circ_0006135 | 0.9120 | 0.037726 | ARL13B |
| 65592690 | 65599361 | chr7 | + | exo | hsa_circ_0004604 | 0.9561 | 0.037862 | CRCP |
| 148543561 | 148547617 | chr7 | − | s/o | hsa_circ_0004930 | 0.9529 | 0.037905 | EZH2 |
| 127295459 | 127298715 | chr3 | − | exo | hsa_circ_0067161 | 1.2338 | 0.037945 | TPRA1 |
| 50724534 | 50731374 | chr15 | + | exo | | 1.0942 | 0.038019 | USP8 |
| 74083421 | 74092259 | chr18 | − | exo | hsa_circ_0005140 | 1.0749 | 0.038034 | ZNF516 |
| 94340604 | 94341852 | chr11 | + | exo | | 1.0524 | 0.038048 | PIWIL4 |
| 129917520 | 129943235 | chrX | − | int | | 1.2619 | 0.038061 | ENOX2 |
| 2944917 | 2945848 | chr20 | + | exo | hsa_circ_0006117 | 1.3094 | 0.038191 | PTPRA |
| 16727794 | 16738483 | chr9 | − | exo | hsa_circ_0008732 | 1.0475 | 0.038193 | BNC2 |
| 65078566 | 65088674 | chr12 | + | s/o | hsa_circ_0008087 | 1.0449 | 0.038284 | RASSF3 |
| 131466424 | 131490413 | chr6 | + | exo | | 0.9274 | 0.038318 | AKAP7 |
| 49091920 | 49094856 | chr12 | − | exo | hsa_circ_0026046 | 1.0569 | 0.038333 | CCNT1 |
| 238280374 | 238290142 | chr2 | − | exo | hsa_circ_0002447 | 1.1286 | 0.038434 | COL6A3 |
| 42160912 | 42161222 | chr17 | − | exo | hsa_circ_0044020 | 0.9385 | 0.038456 | HDAC5 |
| 77214837 | 77216311 | chr12 | + | exo | | 1.3047 | 0.038476 | ZDHHC17 |
| 6441946 | 6443598 | chr7 | + | exo | hsa_circ_0079352 | 1.0772 | 0.038524 | RAC1 |
| 204058517 | 204062114 | chr2 | + | exo | hsa_circ_0002412 | 1.0509 | 0.038660 | NBEAL1 |
| 47707278 | 47711500 | chr20 | + | exo | hsa_circ_0060762 | 0.9625 | 0.038699 | CSE1L |
| 77830227 | 77835260 | chr11 | − | exo | hsa_circ_0023787 | 1.1568 | 0.038772 | ALG8 |
| 39191239 | 39200116 | chr19 | + | exo | hsa_circ_0050900 | 0.9354 | 0.038823 | ACTN4 |
| 89206670 | 89206971 | chr1 | + | exo | hsa_circ_0000089 | 0.9411 | 0.038850 | PKN2 |
| 76349039 | 76360251 | chr10 | + | exo | hsa_circ_0018900 | 0.9245 | 0.038882 | ADK |
| 23872209 | 23873495 | chr18 | + | exo | hsa_circ_0047318 | 1.0675 | 0.038966 | TAF4B |
| 216227368 | 216238260 | chr2 | + | as | | 1.2087 | 0.039000 | FN1 |
| 18525058 | 18528978 | chrX | + | s/o | hsa_circ_0001912 | 0.9216 | 0.039069 | CDKL5 |
| 179136873 | 179147561 | chr5 | + | exo | hsa_circ_0002051 | 0.9584 | 0.039211 | CANX |
| 151374292 | 151374412 | chr1 | + | exo | hsa_circ_0014165 | 1.0995 | 0.039341 | PSMB4 |
| 27132716 | 27143560 | chr12 | − | exo | hsa_circ_0025705 | 1.1897 | 0.039406 | TM7SF3 |
| 201786215 | 201796102 | chr1 | + | exo | hsa_circ_0015911 | 1.0972 | 0.039451 | NAV1 |
| 74385612 | 74397235 | chr17 | − | exo | hsa_circ_0045801 | 1.1911 | 0.039493 | UBE2O |
| 96097655 | 96098258 | chr9 | − | exo | hsa_circ_0006570 | 0.8414 | 0.039500 | C9orf129 |
| 170162524 | 170168267 | chr6 | + | exo | hsa_circ_0004237 | 1.2192 | 0.039519 | ERMARD |
| 69745158 | 69747884 | chr15 | + | exo | hsa_circ_0036088 | 1.3525 | 0.039553 | RPLP1 |
| 28223515 | 28224218 | chr1 | − | exo | hsa_circ_0011107 | 1.0574 | 0.039633 | RPA2 |
| 90446883 | 90451554 | chr14 | + | exo | hsa_circ_0032898 | 1.1212 | 0.039689 | TDP1 |
| 101790176 | 101799759 | chr12 | − | exo | hsa_circ_0027902 | 1.1856 | 0.039759 | ARL1 |
| 43514307 | 43516197 | chr6 | − | exo | hsa_circ_0076560 | 0.9428 | 0.039761 | XPO5 |
| 129827640 | 129830977 | chr11 | − | exo | hsa_circ_0024866 | 1.1424 | 0.039815 | PRDM10 |
| 56792449 | 56839544 | chr16 | + | exo | | 1.1128 | 0.040162 | NUP93 |
| 64576517 | 64608670 | chr13 | − | exo | hsa_circ_0030389 | 1.0750 | 0.040191 | AK057471 |
| 21783862 | 21802971 | chr6 | + | exo | hsa_circ_0075819 | 1.0725 | 0.040294 | CASC15 |
| 126310872 | 126311001 | chr10 | − | int | hsa_circ_0000265 | 1.0588 | 0.040446 | RP11-12J10.3 |
| 5711031 | 5717503 | chr11 | − | int | hsa_circ_0000276 | 1.2328 | 0.040475 | TRIM5 |
| 33113327 | 33116872 | chr19 | − | exo | hsa_circ_0050386 | 0.8346 | 0.040542 | ANKRD27 |
| 78692645 | 78752841 | chr5 | − | exo | hsa_circ_0073128 | 0.8167 | 0.040749 | HOMER1 |
| 12608444 | 12626325 | chr17 | + | exo | hsa_circ_0042103 | 1.0467 | 0.040764 | MYOCD |
| 1721833 | 1724750 | chr1 | − | exo | hsa_circ_0009344 | 1.1527 | 0.040921 | GNB1 |
| 24523655 | 24524213 | chr20 | + | exo | | 0.9319 | 0.040930 | SYNDIG1 |
| 17117929 | 17128675 | chr22 | + | exo | hsa_circ_0062142 | 1.3667 | 0.040979 | TPTEP1 |
| 76211490 | 76216231 | chr1 | + | exo | hsa_circ_0012964 | 0.9023 | 0.041002 | ACADM |
| 144086397 | 144086935 | chr6 | + | exo | hsa_circ_0007769 | 1.1334 | 0.041004 | PHACTR2 |
| 30387492 | 30394846 | chr22 | + | exo | hsa_circ_0001219 | 0.9382 | 0.041028 | MTMR3 |
| 17875010 | 17879836 | chr4 | − | int | | 1.0673 | 0.041038 | LCORL |
| 85657103 | 85661070 | chr15 | + | exo | hsa_circ_0005889 | 1.1128 | 0.041052 | PDE8A |
| 121275020 | 121286936 | chr10 | − | exo | hsa_circ_0005516 | 1.1291 | 0.041282 | RGS10 |
| 224868659 | 224922408 | chr1 | + | exo | hsa_circ_0004797 | 1.2567 | 0.041325 | CNIH3 |
| 102962378 | 102963241 | chr7 | − | exo | hsa_circ_0081751 | 1.1019 | 0.041368 | DNAJC2 |
| 68125117 | 68157520 | chr11 | + | exo | | 0.8717 | 0.041378 | LRP5 |
| 172336669 | 172341841 | chr5 | + | exo | hsa_circ_0075048 | 0.9457 | 0.041472 | ERGIC1 |
| 7225183 | 7226185 | chr17 | − | exo | hsa_circ_0007066 | 0.9506 | 0.041576 | NEURL4 |
| 10459084 | 10480201 | chr1 | + | exo | hsa_circ_0009723 | 1.2239 | 0.041764 | PGD |
| 99220665 | 99220823 | chr9 | + | s/o | | 0.8383 | 0.041793 | HABP4 |
| 41519318 | 41521260 | chr8 | − | exo | hsa_circ_0007366 | 0.9417 | 0.041912 | ANK1 |
| 107617009 | 107617704 | chr8 | + | int | | 1.0489 | 0.041951 | OXR1 |
| 44624185 | 44630515 | chr15 | + | exo | hsa_circ_0000596 | 0.8867 | 0.041998 | CASC4 |
| 47278139 | 47280662 | chr19 | − | exo | hsa_circ_0051620 | 1.0703 | 0.042156 | SLC1A5 |
| 117944807 | 117948267 | chr1 | + | exo | hsa_circ_0000116 | 0.7036 | 0.042251 | MAN1A2 |
| 20800729 | 20825802 | chr22 | − | exo | hsa_circ_0062321 | 0.9556 | 0.042268 | KLHL22 |
| 120285606 | 120286575 | chr1 | − | as | hsa_circ_0000121 | 0.7862 | 0.042309 | PHGDH |
| 156286956 | 156287338 | chr1 | − | exo | hsa_circ_0000142 | 0.8982 | 0.042528 | CCT3 |
| 133311561 | 133328709 | chr5 | − | exo | hsa_circ_0073927 | 1.1101 | 0.042720 | VDAC1 |
| 79942864 | 79946065 | chr13 | − | exo | hsa_circ_0030541 | 0.9317 | 0.042895 | RBM26 |
| 60572605 | 60589763 | chr20 | − | exo | | 1.0781 | 0.042910 | TAF4 |
| 16685846 | 16690028 | chr5 | − | exo | hsa_circ_0071991 | 1.0469 | 0.042982 | MYO10 |
| 57048187 | 57069951 | chr18 | − | s/o | | 1.1051 | 0.042987 | XLOC_012858 |
| 2928627 | 2945848 | chr20 | + | exo | hsa_circ_0005265 | 1.2683 | 0.042995 | PTPRA |
| 74649111 | 74659608 | chr18 | + | exo | hsa_circ_0047997 | 1.1532 | 0.043002 | ZNF236 |
| 78734832 | 78752841 | chr5 | − | exo | hsa_circ_0006916 | 0.6440 | 0.043068 | HOMER1 |
| 129811943 | 129813356 | chr3 | + | exo | hsa_circ_0067327 | 1.2673 | 0.043071 | ALG1L2 |
| 20811305 | 20811534 | chr14 | − | s/o | hsa_circ_0000515 | 0.5961 | 0.043083 | RPPH1 |
| 1703150 | 1704318 | chr17 | − | exo | hsa_circ_0004018 | 1.0978 | 0.043140 | SMYD4 |
| 47735306 | 47737913 | chr1 | − | exo | hsa_circ_0012366 | 1.0401 | 0.043281 | STIL |
| 155823066 | 155823597 | chr1 | − | exo | hsa_circ_0000139 | 0.9138 | 0.043339 | GON4L |
| 2120456 | 2124390 | chr16 | + | exo | hsa_circ_0037422 | 1.1628 | 0.043345 | TSC2 |
| 84765030 | 84772711 | chr6 | + | exo | | 0.8780 | 0.043362 | MRAP2 |
| 22319468 | 22320831 | chr16 | + | exo | hsa_circ_0038508 | 1.0742 | 0.043451 | POLR3E |
| 13169744 | 13178897 | chr10 | + | s/o | hsa_circ_0007242 | 0.8857 | 0.043474 | OPIN |
| 37547116 | 37547282 | chr20 | + | s/o | hsa_circ_0001156 | 0.8609 | 0.043502 | PPP1R16B |
| 17370097 | 17370831 | chr19 | − | exo | hsa_circ_0049965 | 0.9168 | 0.043667 | USHBP1 |
| 121726783 | 121739560 | chr5 | + | exo | | 1.1481 | 0.043676 | SNCAIP |
| 24181170 | 24207701 | chr2 | + | exo | hsa_circ_0052967 | 0.9480 | 0.043756 | UBXN2A |
| 31899500 | 31907527 | chr1 | + | exo | hsa_circ_0011286 | 1.3077 | 0.043759 | SERINC2 |
| 165859440 | 165872516 | chr1 | + | exo | hsa_circ_0015081 | 1.1853 | 0.043891 | UCK2 |
| 57050985 | 57054919 | chr16 | + | exo | hsa_circ_0039522 | 1.0743 | 0.044275 | NLRC5 |
| 2659529 | 2661781 | chr4 | + | exo | hsa_circ_0068925 | 0.9235 | 0.044656 | FAM193A |
| 9404716 | 9416330 | chr3 | + | exo | hsa_circ_0064110 | 1.0836 | 0.044712 | THUMPD3 |
| 67974406 | 67976265 | chr16 | − | int | | 0.8645 | 0.044765 | LCAT |
| 48239625 | 48239790 | chr19 | + | exo | hsa_circ_0051700 | 1.0361 | 0.044790 | EHD2 |
| 154762425 | 154766923 | chr6 | − | s/o | | 1.1257 | 0.044954 | CNKSR3 |
| 25831719 | 25836945 | chr4 | − | exo | | 0.9548 | 0.045044 | SEL1L3 |
| 11114049 | 11154879 | chr16 | + | exo | hsa_circ_0000672 | 0.8628 | 0.045099 | CLEC16A |
| 36405746 | 36407737 | chr20 | + | exo | hsa_circ_0060308 | 1.0987 | 0.045117 | CTNNBL1 |
| 39382719 | 39388954 | chr5 | − | exo | hsa_circ_0002649 | 0.9408 | 0.045248 | DAB2 |
| 56846508 | 56857353 | chr6 | + | exo | | 0.9363 | 0.045274 | BEND6 |
| 235634163 | 235647831 | chr1 | − | exo | hsa_circ_0017067 | 0.9558 | 0.045500 | B3GALNT2 |
| 16658006 | 16753578 | chr6 | − | exo | hsa_circ_0004032 | 0.7232 | 0.045509 | ATXN1 |
| 18825435 | 18846381 | chr2 | + | s/o | | 1.0653 | 0.045636 | XLOC_001374 |
| 51497129 | 51517852 | chr3 | − | exo | hsa_circ_0065916 | 1.1877 | 0.045698 | VPRBP |
| 153377362 | 153414527 | chr5 | − | exo | hsa_circ_0003152 | 1.1137 | 0.045724 | FAM114A2 |
| 41736871 | 41737203 | chr19 | + | exo | hsa_circ_0002945 | 1.1626 | 0.045736 | AXL |
| 183361267 | 183369064 | chr3 | + | exo | hsa_circ_0001367 | 0.8217 | 0.045877 | KLHL24 |
| 28827559 | 28828036 | chr8 | + | exo | hsa_circ_0083824 | 0.9243 | 0.045922 | HMBOX1 |
| 70044653 | 70052000 | chr16 | − | exo | hsa_circ_0008142 | 0.9463 | 0.046017 | PDXDC2P |
| 17342693 | 17356151 | chr19 | − | exo | hsa_circ_0049963 | 1.1015 | 0.046083 | NR2F6 |
| 42607267 | 42610565 | chr3 | − | exo | | 1.1390 | 0.046273 | SEC22C |
| 65266939 | 65273363 | chr15 | − | exo | hsa_circ_0004506 | 1.0775 | 0.046310 | SPG21 |
| 36726361 | 36749456 | chr2 | + | exo | hsa_circ_0002938 | 1.0970 | 0.046338 | CRIM1 |
| 95991977 | 96002258 | chr9 | + | exo | | 0.9296 | 0.046353 | WNK2 |
| 62476405 | 62479091 | chr17 | − | exo | | 1.1341 | 0.046374 | POLG2 |
| 69881194 | 69882097 | chr10 | + | exo | | 1.1144 | 0.046379 | MYPN |
| 122669065 | 122670847 | chr12 | + | exo | | 1.1913 | 0.046441 | LRRC43 |
| 113137647 | 113139646 | chr9 | − | exo | | 1.1279 | 0.046479 | SVEP1 |
| 31813101 | 31844174 | chr14 | − | exo | hsa_circ_0031554 | 1.1757 | 0.046637 | HEATR5A |
| 47028897 | 47030400 | chrX | + | int | | 1.0972 | 0.046648 | RBM10 |
| 19358063 | 19359646 | chr18 | + | exo | hsa_circ_0008190 | 0.8688 | 0.046664 | MIB1 |
| 185094075 | 185106928 | chr1 | − | exo | hsa_circ_0015663 | 1.0403 | 0.046725 | TRMT1L |
| 77649696 | 77652297 | chr11 | − | exo | hsa_circ_0002476 | 0.7021 | 0.046788 | INTS4 |
| 69644908 | 69656342 | chr12 | + | exo | hsa_circ_0000417 | 0.8010 | 0.046910 | CPSF6 |
| 31815277 | 31867987 | chr7 | − | exo | | 1.1770 | 0.047023 | PDE1C |
| 22635490 | 22659753 | chr12 | − | exo | | 0.9505 | 0.047040 | C2CD5 |
| 180286875 | 180286984 | chr5 | − | int | | 1.1679 | 0.047117 | ZFP62 |
| 140055467 | 140069482 | chr7 | − | exo | hsa_circ_0082708 | 1.3919 | 0.047126 | SLC37A3 |
| 183695304 | 183696439 | chr3 | − | exo | hsa_circ_0068259 | 1.0853 | 0.047135 | ABCC5 |
| 35199501 | 35201078 | chr6 | + | exo | hsa_circ_0076094 | 1.0826 | 0.047174 | SCUBE3 |
| 73406509 | 73407085 | chr14 | + | exo | hsa_circ_0032482 | 1.0443 | 0.047241 | DCAF4 |
| 103354700 | 103373854 | chr8 | − | exo | hsa_circ_0085258 | 0.8877 | 0.047398 | UBR5 |
| 4760668 | 4766974 | chr20 | − | exo | hsa_circ_0059354 | 0.9345 | 0.047433 | RASSF2 |
| 33660850 | 33661050 | chr6 | + | int | hsa_circ_0092294 | 1.0822 | 0.047499 | ITPR3 |
| 33722243 | 33739978 | chr18 | + | exo | hsa_circ_0007535 | 1.0716 | 0.047514 | ELP2 |
| 16464347 | 16464925 | chr1 | − | exo | hsa_circ_0005240 | 1.0963 | 0.047525 | EPHA2 |
| 148571844 | 148582568 | chrX | − | exo | hsa_circ_0091671 | 1.0855 | 0.047538 | IDS |
| 177652366 | 177652583 | chr5 | − | int | hsa_circ_0001561 | 1.0485 | 0.047600 | PHYKPL |
| 147527106 | 147599340 | chr6 | + | exo | hsa_circ_0078153 | 1.1462 | 0.047699 | STXBP5 |
| 48259473 | 48260829 | chr10 | + | exo | hsa_circ_0018309 | 0.9138 | 0.047736 | ANXA8 |
| 34157357 | 34157545 | chr22 | − | exo | hsa_circ_0063018 | 0.8611 | 0.047783 | LARGE |
| 21788274 | 21810728 | chr12 | − | exo | hsa_circ_0025597 | 0.9398 | 0.047783 | LDHB |
| 21148518 | 21166543 | chr13 | + | exo | | 1.0552 | 0.047800 | IFT88 |
| 118511503 | 118511797 | chr12 | − | exo | | 1.2302 | 0.047829 | VSIG10 |
| 70404454 | 70406762 | chr10 | + | exo | | 1.1239 | 0.047866 | TET1 |
| 18202872 | 18206037 | chr17 | − | exo | hsa_circ_0042331 | 1.0744 | 0.047893 | TOP3A |
| 151330954 | 151358253 | chr6 | + | exo | hsa_circ_0002697 | 1.0969 | 0.047931 | MTHFD1L |
| 238933982 | 238944581 | chr2 | + | exo | hsa_circ_0007713 | 0.8713 | 0.047940 | UBE2F |
| 5129748 | 5131057 | chr16 | + | exo | hsa_circ_0037777 | 1.2290 | 0.047958 | ALG1 |
| 40596333 | 40597123 | chr21 | − | exo | hsa_circ_0001193 | 0.8700 | 0.048047 | BRWD1 |
| 116590610 | 116608496 | chr10 | + | exo | hsa_circ_0020080 | 1.2705 | 0.048160 | FAM160B1 |
| 28362054 | 28384605 | chr1 | − | exo | hsa_circ_0007895 | 0.9033 | 0.048190 | EYA3 |
| 104432491 | 104433310 | chr8 | + | exo | | 1.0983 | 0.048207 | DCAF13 |
| 26587169 | 26606215 | chr2 | + | exo | hsa_circ_0006111 | 0.9147 | 0.048209 | EPT1 |
| 38300748 | 38305830 | chr1 | − | s/o | | 1.0707 | 0.048365 | MTF1 |
| 57832923 | 57843886 | chr3 | + | exo | hsa_circ_0066277 | 1.0399 | 0.048634 | SLMAP |
| 50810448 | 50832564 | chr22 | + | exo | hsa_circ_0001258 | 0.8100 | 0.048646 | PPP6R2 |
| 47603639 | 47604020 | chr11 | + | exo | hsa_circ_0022015 | 1.1548 | 0.048703 | NDUFS3 |
| 48311248 | 48337216 | chr16 | + | exo | hsa_circ_0008558 | 0.8539 | 0.048718 | LONP2 |
| 156956503 | 156979715 | chr7 | + | exo | hsa_circ_0083145 | 1.0702 | 0.048738 | UBE3C |
| 131761180 | 131761386 | chr5 | + | int | hsa_circ_0001525 | 0.8947 | 0.048755 | C5orf56 |
| 23845133 | 23847587 | chr18 | + | exo | | 1.0956 | 0.048783 | TAF4B |
| 37331786 | 37341344 | chr5 | − | exo | hsa_circ_0072264 | 1.0399 | 0.048916 | NUP155 |
| 50737418 | 50742355 | chr7 | − | exo | hsa_circ_0008334 | 0.9101 | 0.048923 | GRB10 |
| 24880815 | 24883968 | chr10 | − | exo | hsa_circ_0017979 | 1.1090 | 0.048925 | ARHGAP21 |
| 3608374 | 3608939 | chr17 | − | s/o | hsa_circ_0006692 | 1.0764 | 0.049002 | XLOC_012358 |
| 121220457 | 121248689 | chr12 | − | exo | hsa_circ_0002179 | 1.1072 | 0.049031 | SPPL3 |
| 155556502 | 155567341 | chr7 | + | s/o | hsa_circ_0005006 | 1.0437 | 0.049131 | RBM33 |
| 141710989 | 141716304 | chr8 | − | exo | hsa_circ_0005273 | 0.7833 | 0.049133 | PTK2 |
| 152497615 | 152498816 | chr7 | + | exo | hsa_circ_0083061 | 0.8017 | 0.049246 | ACTR3B |
| 27432375 | 27436268 | chr1 | − | exo | hsa_circ_0007283 | 1.2002 | 0.049265 | SLC9A1 |
| 2066700 | 2087531 | chr1 | + | exo | hsa_circ_0009366 | 1.2527 | 0.049267 | PRKCZ |
| 59981820 | 59996874 | chr17 | − | exo | | 1.3144 | 0.049299 | INTS2 |
| 125396193 | 125396954 | chr12 | − | exo | hsa_circ_0029352 | 1.2216 | 0.049346 | UBC |
| 74043986 | 74053624 | chr17 | − | exo | | 1.0374 | 0.049353 | SRP68 |
| 183859566 | 183860566 | chr2 | − | exo | hsa_circ_0057289 | 1.1143 | 0.049427 | NCKAP1 |
| 50045984 | 50059076 | chr5 | + | exo | hsa_circ_0005809 | 0.9400 | 0.049439 | PARP8 |
| 79168800 | 79169629 | chr17 | − | int | | 1.1527 | 0.049515 | AZI1 |
| 13424714 | 13466728 | chr11 | − | exo | | 0.9410 | 0.049537 | BTBD10 |
| 114133569 | 114183238 | chr11 | + | exo | hsa_circ_0024326 | 1.1089 | 0.049636 | NNMT |
| 184104555 | 184114861 | chr4 | + | s/o | hsa_circ_0003631 | 1.0771 | 0.049708 | WWC2 |
| 24357988 | 24362320 | chr2 | + | exo | hsa_circ_0007270 | 0.8503 | 0.049708 | FAM228B |
| 114319831 | 114320430 | chr1 | − | exo | hsa_circ_0013607 | 1.0721 | 0.049773 | RSBN1 |

TABLE 4

| | | | | circRNA | | Fold | p-value | |
|---|---|---|---|---|---|---|---|---|
| Tx Start | Tx End | Chrom | Strand | type | circRNA Alias | SCZ OFC | SCZ OFC | GeneSymbol |
| 154184795 | 154186422 | chr1 | − | exo | hsa_circ_0014364 | 0.9386 | 0.001537 | C1orf43 |
| 176132004 | 176153828 | chr1 | − | exo | hsa_circ_0009064 | 0.8955 | 0.001650 | RFWD2 |
| 64509453 | 64510584 | chr17 | + | int | | 0.8795 | 0.001800 | PRKCA |
| 40728446 | 40747121 | chr5 | − | exo | hsa_circ_0007795 | 0.9276 | 0.003082 | TTC33 |
| 74229605 | 74230755 | chr6 | − | exo | hsa_circ_0077007 | 1.0694 | 0.003384 | EEF1A1 |
| 46085591 | 46125470 | chr22 | + | exo | hsa_circ_0002657 | 0.9020 | 0.003947 | ATXN10 |
| 127620157 | 127620346 | chr9 | − | exo | hsa_circ_0088515 | 0.9474 | 0.003961 | RPL35 |
| 60962120 | 60963576 | chr20 | + | exo | hsa_circ_0061107 | 0.8590 | 0.004367 | RPS21 |
| 154006958 | 154019343 | chrX | − | exo | hsa_circ_0092148 | 0.8987 | 0.004677 | MPP1 |
| 156848913 | 156849949 | chr1 | + | exo | hsa_circ_0014798 | 0.9041 | 0.004936 | NTRK1 |
| 65817491 | 65821858 | chr7 | + | exo | hsa_circ_0080346 | 0.8803 | 0.005079 | TPST1 |
| 102521074 | 102527646 | chr13 | − | exo | hsa_circ_0030793 | 0.9029 | 0.005604 | FGF14 |
| 21788274 | 21810728 | chr12 | − | exo | hsa_circ_0025597 | 0.9270 | 0.006179 | LDHB |
| 101183173 | 101183330 | chr7 | + | exo | hsa_circ_0081599 | 0.8880 | 0.006465 | COL26A1 |
| 125895123 | 125923347 | chr9 | − | exo | hsa_circ_0001886 | 1.0473 | 0.007120 | STRBP |
| 58709843 | 58710250 | chr16 | − | exo | hsa_circ_0004541 | 0.9026 | 0.007245 | SLC38A7 |
| 12826218 | 12826553 | chr19 | − | exo | hsa_circ_0049587 | 0.9204 | 0.007879 | TNPO2 |
| 136302868 | 136303486 | chr9 | + | exo | hsa_circ_0089372 | 0.9560 | 0.008010 | ADAMTS13 |
| 47462052 | 47462148 | chr3 | + | as | hsa_circ_0001291 | 0.9517 | 0.008162 | SCAP |
| 29677910 | 29678100 | chr16 | + | int | hsa_circ_0000686 | 0.7558 | 0.008499 | QPRT |
| 4123222 | 4133292 | chr11 | + | exo | hsa_circ_0007923 | 0.9544 | 0.009222 | RRM1 |
| 79996891 | 80022523 | chr9 | + | exo | hsa_circ_0008075 | 0.9246 | 0.009439 | VPS13A |
| 41006257 | 41007382 | chr1 | + | exo | | 1.1105 | 0.009443 | ZNF684 |
| 107216250 | 107230146 | chr4 | − | exo | hsa_circ_0003673 | 0.9157 | 0.010156 | TBCK |
| 36133847 | 36194359 | chr14 | − | exo | hsa_circ_0031677 | 1.0928 | 0.010400 | RALGAPA1 |
| 138994281 | 139003046 | chr5 | + | exo | hsa_circ_0007452 | 0.9283 | 0.010560 | UBE2D2 |
| 29119457 | 29120690 | chr17 | − | exo | hsa_circ_0042860 | 1.0550 | 0.010742 | CRLF3 |
| 421177 | 421699 | chr17 | − | int | | 0.9005 | 0.011096 | VPS53 |
| 80388619 | 80422982 | chr5 | + | exo | hsa_circ_0008226 | 0.8985 | 0.011501 | RASGRF2 |
| 35824525 | 35855699 | chr1 | + | exo | hsa_circ_0011538 | 1.4846 | 0.012143 | ZMYM4 |
| 10685092 | 10685705 | chr19 | − | exo | hsa_circ_0049279 | 0.9057 | 0.012219 | AP1M2 |
| 151169883 | 151170623 | chr5 | + | exo | hsa_circ_0003967 | 0.9015 | 0.012491 | G3BP1 |
| 41721567 | 41721922 | chr22 | + | exo | hsa_circ_0007704 | 0.9215 | 0.012728 | ZC3H7B |
| 47479629 | 47489400 | chr18 | − | exo | | 0.9571 | 0.013603 | MYO5B |
| 50142509 | 50143142 | chr3 | + | exo | hsa_circ_0003795 | 0.9563 | 0.014013 | RBM5 |
| 157174939 | 157178305 | chr7 | + | exo | hsa_circ_0083176 | 0.9478 | 0.014256 | DNAJB6 |
| 38297823 | 38315052 | chr8 | − | exo | hsa_circ_0084010 | 0.9272 | 0.014776 | FGFR1 |
| 34991685 | 35006275 | chr11 | + | exo | hsa_circ_0000290 | 0.9430 | 0.014892 | PDHX |
| 49736908 | 49738142 | chr3 | + | s/o | hsa_circ_0001302 | 0.9581 | 0.015084 | RNF123 |
| 70236227 | 70236759 | chr14 | + | int | hsa_circ_0000549 | 1.1355 | 0.015284 | SRSF5 |
| 5207932 | 5218543 | chr19 | − | exo | hsa_circ_0048728 | 0.8785 | 0.015371 | PTPRS |
| 49094294 | 49095323 | chr3 | − | exo | hsa_circ_0002561 | 0.9166 | 0.015495 | QRICH1 |
| 19835117 | 19866335 | chr17 | − | exo | hsa_circ_0042415 | 0.9031 | 0.015548 | AKAP10 |
| 100623093 | 100628033 | chr2 | − | exo | hsa_circ_0055855 | 0.8270 | 0.015836 | AFF3 |
| 245018859 | 245019265 | chr1 | − | s/o | hsa_circ_0000200 | 1.4447 | 0.016002 | HNRNPU |
| 47474879 | 47481653 | chr7 | − | exo | hsa_circ_0008729 | 0.8582 | 0.016247 | TNS3 |
| 201786215 | 201786413 | chr1 | + | exo | hsa_circ_0008264 | 0.8862 | 0.016604 | NAV1 |
| 119209651 | 119215101 | chr11 | − | exo | hsa_circ_0024588 | 0.9130 | 0.016826 | MFRP |
| 44568737 | 44571186 | chr1 | + | int | | 1.2660 | 0.016965 | KLF17 |
| 44617544 | 44623775 | chr18 | − | s/o | | 1.4947 | 0.017745 | RP11-49K24.4 |
| 2638098 | 2644625 | chr7 | + | exo | hsa_circ_0079201 | 0.8016 | 0.017946 | IQCE |
| 38463710 | 38466898 | chr22 | + | exo | hsa_circ_0063280 | 0.8019 | 0.018321 | PICK1 |
| 86627164 | 86633908 | chr5 | + | exo | hsa_circ_0007507 | 1.7728 | 0.018331 | RASA1 |
| 99589361 | 99607298 | chr9 | − | exo | | 0.8503 | 0.018739 | ZNF782 |
| 50902104 | 50902741 | chr19 | + | exo | hsa_circ_0052011 | 0.9081 | 0.018757 | POLD1 |
| 18252691 | 18258434 | chr19 | + | exo | | 0.8308 | 0.018793 | MAST3 |
| 30212998 | 30226749 | chr17 | − | exo | | 1.0860 | 0.019167 | UTP6 |
| 46085591 | 46136418 | chr22 | + | exo | hsa_circ_0008199 | 0.8920 | 0.019251 | ATXN10 |
| 3185580 | 3209234 | chr10 | − | exo | hsa_circ_0017502 | 0.8452 | 0.019467 | PITRM1 |
| 77327195 | 77348851 | chr11 | − | exo | hsa_circ_0023691 | 0.8066 | 0.019625 | CLNS1A |
| 154245001 | 154247352 | chr5 | + | int | | 1.0788 | 0.020019 | CNOT8 |
| 35814866 | 35815138 | chr1 | + | int | | 0.9367 | 0.020220 | ZMYM4 |
| 3988781 | 4045217 | chr11 | + | exo | hsa_circ_0020927 | 0.9332 | 0.020251 | STIM1 |
| 98231032 | 98232213 | chr9 | − | exo | | 1.3997 | 0.020495 | PTCH1 |
| 23540822 | 23541218 | chr16 | − | exo | hsa_circ_0038608 | 1.1082 | 0.020639 | EARS2 |
| 77930940 | 77932010 | chr14 | + | exo | hsa_circ_0008404 | 0.9093 | 0.020883 | AHSA1 |
| 4947116 | 4951008 | chr19 | + | exo | | 1.3895 | 0.020911 | UHRF1 |
| 99718694 | 99761618 | chr8 | − | exo | hsa_circ_0085045 | 1.0426 | 0.020927 | STK3 |
| 17448955 | 17451907 | chr16 | − | s/o | | 0.8976 | 0.021010 | XYLT1 |
| 1890310 | 1896059 | chr2 | − | exo | hsa_circ_0052455 | 0.8424 | 0.021111 | MYT1L |
| 97535283 | 97563284 | chr9 | + | exo | hsa_circ_0002191 | 0.7820 | 0.021129 | C9orf3 |
| 154291286 | 154292598 | chr5 | − | exo | hsa_circ_0074736 | 0.8763 | 0.021415 | GEMIN5 |
| 45898718 | 45959242 | chr22 | + | exo | hsa_circ_0063739 | 0.8780 | 0.021786 | FBLN1 |
| 108175911 | 108179689 | chr13 | − | int | hsa_circ_0003145 | 0.9255 | 0.022157 | FAM155A |
| 114149816 | 114164739 | chr13 | + | exo | hsa_circ_0002807 | 1.1131 | 0.022249 | TMCO3 |
| 202410265 | 202440049 | chr2 | − | exo | | 1.0541 | 0.022260 | ALS2CR11 |
| 137371579 | 137372196 | chr5 | − | int | hsa_circ_0001536 | 1.4034 | 0.022284 | FAM13B |
| 121036193 | 121038483 | chr2 | + | exo | | 1.1422 | 0.022584 | RALB |
| 131744827 | 131745829 | chr9 | + | exo | | 0.8156 | 0.022667 | NUP188 |
| 158204420 | 158223486 | chr5 | − | exo | hsa_circ_0074817 | 0.8630 | 0.022725 | EBF1 |
| 123471177 | 123512691 | chr3 | − | exo | hsa_circ_0002768 | 0.8943 | 0.023124 | MYLK |
| 9804597 | 9804897 | chr3 | − | exo | | 0.9108 | 0.023302 | CAMK1 |
| 140458885 | 140459606 | chr9 | − | exo | hsa_circ_0006502 | 0.7502 | 0.023410 | DPH7 |
| 12558109 | 12560696 | chr3 | + | exo | hsa_circ_0005028 | 0.9264 | 0.023597 | TSEN2 |
| 45459677 | 45460699 | chr6 | + | int | hsa_circ_0005526 | 1.4252 | 0.023616 | RUNX2 |
| 110321942 | 110343415 | chr2 | − | exo | hsa_circ_0003065 | 1.1063 | 0.023655 | SEPT10 |
| 61118817 | 61128218 | chr2 | + | exo | hsa_circ_0001011 | 1.0493 | 0.023730 | REL |
| 128995614 | 129012667 | chr4 | + | exo | hsa_circ_0070934 | 1.0729 | 0.023885 | LARP1B |
| 7285519 | 7327916 | chr10 | − | exo | hsa_circ_0017636 | 0.8713 | 0.024035 | SFMBT2 |
| 12825860 | 12825983 | chr19 | − | exo | hsa_circ_0049586 | 0.8891 | 0.024063 | TNPO2 |
| 134987410 | 134993491 | chrX | + | exo | hsa_circ_0091611 | 0.9361 | 0.024272 | SAGE1 |
| 78443772 | 78452895 | chr12 | + | exo | hsa_circ_0002419 | 1.0671 | 0.024352 | NAV3 |
| 50689402 | 50689520 | chr22 | − | int | hsa_circ_0001256 | 0.9438 | 0.024376 | HDAC10 |
| 27620262 | 27620746 | chr17 | − | int | hsa_circ_0000751 | 0.8982 | 0.024390 | NUFIP2 |
| 19042277 | 19042456 | chr2 | + | int | hsa_circ_0000979 | 1.5461 | 0.024474 | XLOC_001374 |
| 27041298 | 27043076 | chr17 | − | exo | hsa_circ_0042688 | 0.9033 | 0.024869 | RAB34 |
| 63631182 | 63667005 | chr2 | − | exo | hsa_circ_0007387 | 1.2403 | 0.025143 | WDPCP |
| 150202905 | 150204264 | chr1 | − | exo | hsa_circ_0004300 | 1.1782 | 0.025180 | ANP32E |
| 12366963 | 12367381 | chr18 | − | exo | | 0.9410 | 0.025329 | AFG3L2 |
| 190584297 | 190585513 | chr2 | + | exo | | 0.9049 | 0.025490 | ANKAR |
| 27473614 | 27474913 | chr16 | − | exo | hsa_circ_0038737 | 0.8053 | 0.025602 | GTF3C1 |
| 3160973 | 3162236 | chr10 | + | exo | hsa_circ_0006608 | 0.8515 | 0.025798 | PFKP |
| 49340634 | 49350811 | chr17 | + | exo | | 0.8985 | 0.025874 | UTP18 |
| 43782390 | 43786644 | chr21 | − | exo | hsa_circ_0061825 | 0.9200 | 0.025881 | TFF1 |
| 34789429 | 34791125 | chr6 | + | exo | hsa_circ_0076055 | 0.9104 | 0.025949 | UHRF1BP1 |
| 706921 | 708487 | chr17 | − | exo | hsa_circ_0005351 | 0.9116 | 0.026237 | NXN |
| 88061088 | 88105818 | chr16 | + | exo | hsa_circ_0040825 | 0.9086 | 0.026346 | BANP |
| 96444853 | 96448656 | chr8 | + | int | | 0.9583 | 0.026351 | C8orf37-AS1 |
| 26147969 | 26208379 | chr12 | + | exo | hsa_circ_0025636 | 1.1245 | 0.026459 | RASSF8 |
| 35436177 | 35438558 | chr6 | + | exo | hsa_circ_0076138 | 0.9249 | 0.026573 | RPE10A |
| 11015626 | 11019883 | chr19 | + | exo | hsa_circ_0004552 | 1.5181 | 0.026606 | CARM1 |
| 68208276 | 68217269 | chr16 | + | exo | hsa_circ_0039933 | 0.9217 | 0.026746 | NFATC3 |
| 50902107 | 50902741 | chr19 | + | exo | hsa_circ_0052012 | 0.9127 | 0.026910 | POLD1 |
| 125042721 | 125054119 | chr9 | + | exo | hsa_circ_0003250 | 0.9113 | 0.026959 | MRRF |
| 27862909 | 27870810 | chr2 | + | exo | | 1.4136 | 0.026991 | GPN1 |
| 25282854 | 25290211 | chr20 | − | exo | hsa_circ_0059660 | 0.8323 | 0.027089 | ABHD12 |
| 18622958 | 18662408 | chr8 | − | exo | hsa_circ_0002111 | 0.8876 | 0.027153 | PSD3 |
| 165878068 | 165878169 | chr1 | + | s/o | hsa_circ_0000148 | 1.3800 | 0.027218 | UCK2 |
| 76285013 | 76315227 | chr10 | + | int | | 0.9035 | 0.027255 | ADK |
| 151043647 | 151049345 | chr5 | − | exo | hsa_circ_0004104 | 0.8819 | 0.027261 | SPARC |
| 74551677 | 74551959 | chr14 | + | s/o | hsa_circ_0000554 | 0.8039 | 0.027275 | EIN52 |
| 39514366 | 39532617 | chr14 | − | exo | hsa_circ_0031710 | 0.9468 | 0.027332 | SEC23A |
| 75915260 | 75915723 | chr13 | − | exo | hsa_circ_0030428 | 0.8461 | 0.027369 | TBC1D4 |
| 129752887 | 129773423 | chr4 | + | exo | hsa_circ_0006563 | 1.0917 | 0.027412 | JADE1 |
| 46124699 | 46126897 | chr1 | − | exo | hsa_circ_0000068 | 1.0495 | 0.027485 | GPBP1L1 |
| 72491369 | 72509841 | chr15 | − | exo | hsa_circ_0036186 | 0.9193 | 0.027510 | PKM |
| 137582757 | 137593179 | chr9 | + | exo | hsa_circ_0007482 | 0.8744 | 0.027816 | COL5A1 |
| 99196947 | 99197507 | chr10 | − | exo | hsa_circ_0004896 | 1.4823 | 0.027901 | EXOSC1 |
| 18285849 | 18286507 | chr19 | + | exo | hsa_circ_0005571 | 0.9573 | 0.028283 | IFI30 |
| 71209067 | 71209308 | chr14 | − | exo | hsa_circ_0032377 | 0.8660 | 0.028578 | MAP3K9 |
| 4286037 | 4286435 | chr9 | − | int | hsa_circ_0004822 | 0.9644 | 0.028645 | GLIS3 |
| 151771875 | 151789457 | chr4 | − | exo | hsa_circ_0071224 | 0.8589 | 0.028771 | LRBA |
| 6978421 | 6978937 | chr12 | + | exo | hsa_circ_0025278 | 1.2037 | 0.028847 | TPI1 |
| 87114895 | 87115116 | chr2 | + | exo | hsa_circ_0005528 | 0.8739 | 0.028999 | ANAPC1P1 |
| 50369762 | 50370182 | chr19 | − | int | hsa_circ_0092285 | 0.8336 | 0.029065 | PNKP |
| 110028575 | 110032986 | chr12 | + | exo | hsa_circ_0028143 | 0.8591 | 0.029106 | MVK |
| 5143977 | 5144913 | chr19 | + | exo | | 0.8642 | 0.029225 | KDM4B |
| 43675612 | 43704502 | chr5 | + | s/o | hsa_circ_0005585 | 0.9231 | 0.029570 | NNT |
| 79555969 | 79575848 | chr17 | − | exo | hsa_circ_0046188 | 1.0802 | 0.029594 | NPLOC4 |
| 4452322 | 4452402 | chr4 | − | int | hsa_circ_0001393 | 0.9173 | 0.029627 | STX18 |
| 47001424 | 47002076 | chr16 | − | exo | hsa_circ_0000696 | 1.1694 | 0.029667 | DNAJA2 |
| 88957371 | 88973310 | chr4 | + | exo | hsa_circ_0070400 | 1.1087 | 0.029750 | PKD2 |
| 98923104 | 98923600 | chr13 | + | int | | 1.2790 | 0.029899 | FARP1 |
| 13542212 | 13544480 | chr3 | + | exo | hsa_circ_0064416 | 0.8875 | 0.029906 | HDAC11 |
| 96277951 | 96278551 | chr9 | + | exo | hsa_circ_0007060 | 0.8519 | 0.029961 | FAM120A |
| 33365805 | 33366164 | chr6 | + | exo | | 0.9194 | 0.030055 | KIFC1 |
| 74948073 | 74948409 | chr15 | − | exo | hsa_circ_0004401 | 0.9355 | 0.030132 | EDC3 |
| 99481790 | 99483412 | chr9 | + | s/o | | 1.5973 | 0.030421 | RP11-535M15.1 |
| 100676720 | 100691953 | chr12 | + | exo | hsa_circ_0006258 | 1.0513 | 0.030905 | SCYL2 |
| 74120092 | 74146780 | chr2 | + | exo | hsa_circ_0055257 | 0.9610 | 0.030996 | ACTG2 |
| 6859878 | 6860842 | chr12 | − | exo | hsa_circ_0025249 | 0.8181 | 0.031005 | MLF2 |
| 49054148 | 49063055 | chr12 | − | exo | hsa_circ_0026036 | 0.9308 | 0.031153 | KANSL2 |
| 62579578 | 62582288 | chr17 | − | exo | hsa_circ_0045322 | 1.0972 | 0.031394 | SMURF2 |
| 32695598 | 32718742 | chr7 | − | exo | | 1.1394 | 0.031563 | DPY19L1P1 |
| 242028602 | 242033847 | chr2 | − | exo | | 0.9343 | 0.031635 | MTERFD2 |
| 10527246 | 10529395 | chr1 | − | exo | hsa_circ_0008936 | 0.9093 | 0.032018 | DFFA |
| 116599227 | 116635985 | chr8 | − | exo | hsa_circ_0085362 | 1.0878 | 0.032323 | TRPS1 |
| 57430575 | 57430887 | chr17 | + | exo | hsa_circ_0005600 | 0.9187 | 0.032635 | YPEL2 |
| 93929156 | 93941802 | chr8 | − | int | hsa_circ_0004561 | 1.3013 | 0.032679 | TRIQK |
| 52880957 | 52887181 | chr12 | − | exo | hsa_circ_0026426 | 1.1310 | 0.032706 | KRT6A |
| 6620218 | 6632869 | chr5 | − | exo | hsa_circ_0071760 | 0.9410 | 0.032783 | NSUN2 |
| 70732539 | 70796508 | chr16 | − | exo | hsa_circ_0040319 | 0.9191 | 0.032817 | VAC14 |
| 43819869 | 43861614 | chr11 | + | exo | | 1.1378 | 0.032909 | HSD17B12 |
| 102201526 | 102249725 | chr5 | + | exo | hsa_circ_0073492 | 0.9654 | 0.033229 | PAM |
| 67546221 | 67559327 | chr3 | − | exo | hsa_circ_0004276 | 1.0446 | 0.033787 | SUCLG2 |
| 72361889 | 72362548 | chr2 | − | exo | hsa_circ_0055161 | 0.9392 | 0.034116 | CYP26B1 |
| 39328342 | 39329036 | chr19 | − | int | | 1.2820 | 0.034410 | HNRNPL |
| 53908237 | 53910107 | chr3 | − | exo | hsa_circ_0002554 | 1.2871 | 0.034490 | ACTR8 |
| 52773420 | 52773806 | chr8 | − | exo | hsa_circ_0001802 | 1.0760 | 0.034659 | PCMTD1 |
| 46529740 | 46534363 | chr11 | − | exo | hsa_circ_0003110 | 0.9046 | 0.034692 | AMBRA1 |
| 78865519 | 78867665 | chr17 | + | exo | hsa_circ_0000813 | 1.3148 | 0.034760 | RPTOR |
| 189022209 | 189026744 | chr4 | − | exo | | 0.9125 | 0.034957 | TRIML2 |
| 58095302 | 58112489 | chr3 | + | exo | hsa_circ_0066331 | 0.9105 | 0.035180 | FLNB |
| 156303463 | 156304503 | chr1 | − | int | | 1.6075 | 0.035314 | CCT3 |
| 9190378 | 9238002 | chr5 | − | exo | hsa_circ_0002099 | 1.1792 | 0.035502 | SEMA5A |
| 92078065 | 92083999 | chr7 | + | exo | | 0.9030 | 0.035533 | GATAD1 |
| 78111184 | 78113939 | chr17 | − | exo | hsa_circ_0045986 | 0.8661 | 0.035533 | EIF4A3 |
| 24025911 | 24037704 | chr22 | − | exo | hsa_circ_0062548 | 0.9602 | 0.035562 | GUSBP11 |
| 19371334 | 19379935 | chr18 | + | exo | | 1.3183 | 0.035669 | MIB1 |
| 98923108 | 98923600 | chr13 | + | int | hsa_circ_0003907 | 1.5093 | 0.035796 | FARP1 |
| 45726483 | 45728591 | chr22 | + | exo | hsa_circ_0001245 | 0.9388 | 0.035902 | FAM118A |
| 117109316 | 117110576 | chr11 | + | exo | hsa_circ_0024402 | 0.9308 | 0.035972 | RNF214 |
| 5213834 | 5214425 | chr3 | + | exo | hsa_circ_0064080 | 0.9588 | 0.036132 | ARL8B |
| 5215701 | 5216099 | chr3 | + | exo | hsa_circ_0001263 | 0.9451 | 0.036223 | ARL8B |
| 11354777 | 11356967 | chr3 | + | exo | hsa_circ_0003862 | 0.9407 | 0.036248 | ATG7 |
| 75766029 | 75782642 | chr15 | − | exo | | 0.8823 | 0.036286 | PTPN9 |
| 355970 | 415573 | chr10 | − | exo | | 1.0470 | 0.036412 | DIP2C |
| 140402665 | 140404763 | chr7 | + | exo | hsa_circ_0007518 | 0.9345 | 0.036802 | NDUFB2 |
| 116734383 | 116744352 | chr11 | − | exo | hsa_circ_0000357 | 0.8922 | 0.036822 | SIK3 |
| 111026652 | 111119523 | chr4 | − | exo | | 0.8915 | 0.036906 | ELOVL6 |
| 77055327 | 77065626 | chr4 | − | exo | hsa_circ_0070039 | 1.0655 | 0.036996 | NUP54 |
| 46284289 | 46288060 | chr18 | + | exo | hsa_circ_0003877 | 0.9537 | 0.037187 | CTIF |
| 25312558 | 25351441 | chr15 | + | exo | hsa_circ_0034125 | 0.9172 | 0.037236 | SNURF-SNRPN |
| 35817493 | 35819173 | chr22 | + | int | hsa_circ_0092299 | 0.8329 | 0.037250 | MCM5 |
| 37623043 | 37623873 | chr8 | + | exo | hsa_circ_0001788 | 0.8536 | 0.037337 | PROSC |
| 62407030 | 62422143 | chr20 | − | exo | hsa_circ_0002805 | 0.9324 | 0.037403 | ZBTB46 |
| 109186061 | 109186605 | chr12 | − | exo | hsa_circ_0002957 | 0.9476 | 0.037557 | SSH1 |
| 5859467 | 5867902 | chr9 | − | ing | | 1.4655 | 0.037625 | |
| 4763457 | 4794491 | chr12 | + | exo | | 0.8911 | 0.038009 | NDUFA9 |
| 97999787 | 97999925 | chr10 | − | int | hsa_circ_0000253 | 2.1277 | 0.038105 | BLNK |
| 93424545 | 93460342 | chr14 | − | exo | hsa_circ_0033018 | 0.9331 | 0.038688 | ITPK1 |
| 1475710 | 1489345 | chr17 | − | s/o | hsa_circ_0000731 | 1.0431 | 0.038703 | SLC43A2 |
| 71590282 | 71597135 | chr2 | + | exo | hsa_circ_0001028 | 1.0872 | 0.038748 | ZNF638 |
| 169982931 | 170034621 | chr6 | − | exo | hsa_circ_0078724 | 0.9278 | 0.038940 | WDR27 |
| 49042408 | 49103656 | chr22 | + | exo | | 0.9602 | 0.039048 | FAM19A5 |
| 56651185 | 56651585 | chr12 | − | int | hsa_circ_0092273 | 0.9129 | 0.039098 | ANKRD52 |
| 45883610 | 45893784 | chr11 | + | exo | hsa_circ_0000295 | 0.9426 | 0.039524 | CRY2 |
| 39923846 | 39924209 | chr19 | − | exo | hsa_circ_0050998 | 0.8866 | 0.039560 | RPS16 |
| 25013135 | 25013450 | chr2 | − | exo | hsa_circ_0052996 | 0.9117 | 0.039646 | PTRHD1 |
| 45239084 | 45242364 | chr20 | − | exo | | 0.9313 | 0.039912 | SLC13A3 |
| 242572331 | 242573494 | chr2 | − | exo | hsa_circ_0007647 | 0.8630 | 0.039921 | THAP4 |
| 28748408 | 28752072 | chr13 | + | exo | hsa_circ_0004372 | 0.8681 | 0.039926 | PAN3 |
| 113869912 | 113870043 | chr13 | + | int | hsa_circ_0000508 | 1.4626 | 0.040250 | CUL4A |
| 101450683 | 101451498 | chr3 | + | exo | hsa_circ_0066701 | 0.9047 | 0.040259 | CEP97 |
| 29129325 | 29140861 | chr2 | + | exo | hsa_circ_0053339 | 1.1021 | 0.040310 | WDR43 |
| 33246480 | 33247316 | chr8 | − | exo | hsa_circ_0083902 | 1.1446 | 0.040668 | FUT10 |
| 45506682 | 45509814 | chr21 | + | exo | hsa_circ_0061936 | 0.9187 | 0.040681 | TRAPPC10 |
| 39679868 | 39925459 | chr17 | − | exo | hsa_circ_0043602 | 1.5316 | 0.040689 | JUP |
| 70642966 | 70644088 | chrX | + | exo | hsa_circ_0091025 | 0.9645 | 0.040722 | TAF1 |
| 28712642 | 28835595 | chr13 | + | exo | hsa_circ_0029830 | 0.8751 | 0.040851 | PAN3 |
| 174188262 | 174190302 | chr1 | + | exo | hsa_circ_0008141 | 1.1983 | 0.041104 | RABGAP1L |
| 10961537 | 10964076 | chr6 | + | exo | hsa_circ_0075629 | 0.9234 | 0.041116 | SYCP2L |
| 30557597 | 30560757 | chr8 | − | exo | hsa_circ_0083861 | 0.9261 | 0.041548 | GSR |
| 128637442 | 128641288 | chr7 | − | exo | hsa_circ_0082271 | 0.9568 | 0.041555 | TNPO3 |
| 239184383 | 239186596 | chr2 | − | exo | hsa_circ_0003854 | 0.9545 | 0.041690 | PER2 |
| 70723046 | 70726959 | chr10 | + | exo | hsa_circ_0008523 | 1.0472 | 0.041885 | DDX21 |
| 172305190 | 172314585 | chr2 | + | exo | hsa_circ_0005904 | 0.8131 | 0.041886 | DCAF17 |
| 62421173 | 62422143 | chr20 | − | exo | hsa_circ_0061179 | 0.9068 | 0.041888 | ZBTB46 |
| 49994035 | 49994121 | chr19 | + | exo | hsa_circ_0051899 | 0.9035 | 0.041890 | RPL13A |
| 56492459 | 56496546 | chr16 | + | exo | hsa_circ_0039444 | 1.0502 | 0.041947 | OGFOD1 |
| 64126607 | 64139559 | chr2 | − | exo | | 0.8854 | 0.042193 | VPS54 |
| 76910271 | 76910864 | chr10 | + | exo | hsa_circ_0006148 | 0.8776 | 0.042434 | SAMD8 |
| 99538969 | 99608397 | chr8 | − | exo | hsa_circ_0002798 | 0.8980 | 0.042543 | STK3 |
| 119176864 | 119182471 | chr3 | − | s/o | hsa_circ_0066869 | 1.8702 | 0.042714 | TMEM39A |
| 58774155 | 58774397 | chr19 | + | int | hsa_circ_0000968 | 0.8233 | 0.042733 | ZNF544 |
| 33722243 | 33726344 | chr18 | + | exo | hsa_circ_0047427 | 1.0456 | 0.042796 | ELP2 |
| 233651859 | 233660931 | chr2 | + | exo | hsa_circ_0058733 | 1.0859 | 0.042961 | GIGYF2 |
| 73991926 | 73996813 | chr15 | + | exo | hsa_circ_0036254 | 0.9309 | 0.043013 | CD276 |
| 120881763 | 120884215 | chr12 | − | exo | hsa_circ_0028879 | 0.9648 | 0.043028 | TRIAP1 |
| 210970849 | 211192598 | chr1 | − | exo | hsa_circ_0016346 | 1.4589 | 0.043070 | KCNH1 |
| 79249768 | 79257302 | chr17 | − | exo | hsa_circ_0046131 | 0.9541 | 0.043173 | SLC38A10 |
| 34797903 | 34797990 | chr21 | + | int | hsa_circ_0001184 | 1.1748 | 0.043188 | IFNGR2 |
| 128902834 | 128903244 | chr8 | + | exo | hsa_circ_0001821 | 0.9237 | 0.043208 | PVT1 |
| 56912110 | 56915652 | chr6 | + | exo | hsa_circ_0076875 | 0.8739 | 0.043296 | KIAA1586 |
| 41518947 | 41519459 | chr8 | − | exo | hsa_circ_0001792 | 0.9276 | 0.043637 | ANK1 |
| 41923912 | 41940125 | chr22 | − | s/o | hsa_circ_0001237 | 0.9063 | 0.043676 | POLR3H |
| 34685382 | 34687668 | chr19 | + | exo | hsa_circ_0006446 | 1.4161 | 0.043682 | LSM14A |
| 31806997 | 31830172 | chr22 | + | exo | hsa_circ_0062898 | 0.8448 | 0.043735 | DRG1 |
| 108586605 | 108594189 | chr11 | + | exo | hsa_circ_0024251 | 1.0603 | 0.043836 | DDX10 |
| 53670403 | 53671744 | chr12 | + | exo | hsa_circ_0008229 | 0.9411 | 0.043977 | ESPL1 |
| 27642756 | 27689252 | chr7 | − | s/o | | 1.0527 | 0.044189 | HIBADH |
| 98984307 | 98987635 | chr7 | + | exo | hsa_circ_0081342 | 0.8236 | 0.044617 | ARPC1B |
| 173735284 | 173744981 | chr1 | + | exo | hsa_circ_0015279 | 0.9264 | 0.044642 | KLHL20 |
| 4872866 | 4884696 | chr10 | + | exo | hsa_circ_0017521 | 0.9081 | 0.044924 | AKR1E2 |
| 210003452 | 210004388 | chr1 | + | exo | hsa_circ_0016330 | 0.8919 | 0.044997 | DIEXF |
| 71712657 | 71715808 | chr16 | − | exo | hsa_circ_0000715 | 1.0783 | 0.045058 | PHLPP2 |
| 5413685 | 5417655 | chr7 | − | exo | hsa_circ_0006824 | 0.9535 | 0.045078 | TNRC18 |
| 51467605 | 51467865 | chr12 | − | exo | hsa_circ_0004538 | 0.9210 | 0.045124 | CSRNP2 |
| 42470415 | 42470768 | chr15 | − | exo | hsa_circ_0034786 | 0.8674 | 0.045126 | VPS39 |
| 168007608 | 168014465 | chr1 | + | exo | hsa_circ_0000155 | 0.9175 | 0.045718 | DCAF6 |
| 114170357 | 114175048 | chr13 | + | int | | 0.8762 | 0.045866 | TMCO3 |
| 95027210 | 95027835 | chr9 | − | exo | hsa_circ_0087497 | 1.3144 | 0.045888 | IARS |
| 152004981 | 152009511 | chr1 | − | exo | hsa_circ_0014206 | 0.8956 | 0.046860 | S100A11 |
| 77659913 | 77663079 | chr4 | + | exo | hsa_circ_0070049 | 0.9016 | 0.046940 | SHROOM3 |
| 14081841 | 14113080 | chr9 | − | exo | hsa_circ_0086368 | 0.8886 | 0.046944 | NFIB |
| 41515056 | 41518061 | chr13 | − | exo | hsa_circ_0030051 | 0.9386 | 0.047018 | ELF1 |
| 22404921 | 22413359 | chr1 | + | exo | hsa_circ_0005576 | 1.1781 | 0.047043 | CDC42 |
| 72342922 | 72353078 | chr18 | + | exo | hsa_circ_0047958 | 1.1151 | 0.047211 | ZNF407 |
| 43114355 | 43129457 | chr6 | + | exo | hsa_circ_0076482 | 0.9447 | 0.047262 | PTK7 |
| 33805093 | 33807214 | chr17 | − | exo | | 0.9531 | 0.047357 | SLFN12L |
| 20078221 | 20078421 | chr22 | + | int | hsa_circ_0092365 | 0.8760 | 0.047407 | DGCR8 |
| 73477044 | 73477365 | chr2 | + | int | | 0.9515 | 0.047429 | CCT7 |
| 46945730 | 46957922 | chr21 | − | exo | hsa_circ_0062022 | 0.8853 | 0.047504 | SLC19A1 |
| 11289020 | 11289396 | chr19 | − | exo | hsa_circ_0008554 | 0.9037 | 0.047594 | KANK2 |
| 64863339 | 64868113 | chr5 | + | exo | hsa_circ_0072697 | 0.8293 | 0.047622 | PPWD1 |
| 195997285 | 196010409 | chr3 | − | exo | | 1.1051 | 0.047719 | PCYT1A |
| 155547476 | 155551830 | chr3 | − | exo | hsa_circ_0001350 | 1.0496 | 0.047873 | SLC33A1 |
| 10354443 | 10357007 | chr3 | − | s/o | | 0.9298 | 0.047960 | SEC13 |
| 123078822 | 123109235 | chr12 | + | exo | | 0.8715 | 0.047981 | KNTC1 |
| 15564438 | 15567918 | chr2 | − | exo | hsa_circ_0052760 | 0.9367 | 0.048004 | NBAS |
| 173662 | 173882 | chr5 | + | int | hsa_circ_0092371 | 0.7970 | 0.048014 | PLEKHG4B |
| 42980912 | 42981094 | chr6 | − | s/o | hsa_circ_0001606 | 0.9001 | 0.048176 | MEA1 |
| 24982756 | 25002883 | chr12 | − | exo | hsa_circ_0007579 | 0.9203 | 0.048237 | BCAT1 |
| 171969049 | 172025291 | chr3 | + | exo | hsa_circ_0067991 | 1.0640 | 0.048292 | FNDC3B |
| 36872779 | 36872903 | chr22 | − | exo | hsa_circ_0063158 | 1.1518 | 0.048389 | TXN2 |
| 160206924 | 160231148 | chr1 | − | exo | hsa_circ_0014879 | 0.8186 | 0.048396 | DCAF8 |
| 102121473 | 102122174 | chr4 | − | int | | 0.8594 | 0.048501 | PPP3CA |
| 58733959 | 58734403 | chr17 | + | exo | hsa_circ_0002677 | 1.0429 | 0.048536 | PPM1D |
| 20800729 | 20825802 | chr22 | − | exo | hsa_circ_0062321 | 0.9606 | 0.048717 | KLHL22 |
| 12022357 | 12022903 | chr12 | + | exo | hsa_circ_0004123 | 0.9235 | 0.048754 | ETV6 |
| 65041198 | 65043837 | chr15 | − | exo | | 0.8911 | 0.048779 | RBPMS2 |
| 14821320 | 14821476 | chrY | + | exo | hsa_circ_0092232 | 0.9133 | 0.048857 | USP9Y |
| 34243644 | 34252790 | chr22 | − | int | | 0.9412 | 0.048899 | LARGE |
| 52675969 | 52677359 | chr3 | − | exo | hsa_circ_0066083 | 0.9566 | 0.048948 | PBRM1 |
| 629834 | 637861 | chr6 | − | exo | hsa_circ_0075445 | 1.0598 | 0.049124 | EXOC2 |
| 38829400 | 38869928 | chr7 | − | exo | hsa_circ_0001698 | 1.0428 | 0.049288 | VPS41 |
| 72167171 | 72168707 | chr18 | + | exo | hsa_circ_0000855 | 1.0760 | 0.049465 | CNDP2 |
| 128715231 | 128719864 | chr4 | + | exo | hsa_circ_0070911 | 1.0414 | 0.049510 | HSPA4L |
| 96412293 | 96416207 | chr13 | + | exo | | 1.0807 | 0.049680 | DNAJC3 |
| 100275475 | 100276304 | chrX | − | exo | hsa_circ_0004957 | 0.8022 | 0.049931 | TRMT2B |

What is claimed is:

1. A composition comprising a panel of between 1 and 10 circRNAs, where the circRNAs are brain disorder-associated circRNA biomarkers.

2. The composition of claim 1 wherein at least one of the circRNAs is circHomer1a.

3. The composition of claim 1 wherein at last one of the circRNAs is circHomer1b.

4. The composition of claim 1 wherein at least one of the circRNAs is circCUL4A.

5. The composition of claim 1 wherein at least one of the circRNAs is circADAM22.

6. An assay for detecting brain disorders comprising: one or more probes configured to identify the presence of at least one brain disorder-associated circRNA biomarker.

7. The assay of claim 6 wherein the probe comprises a sequence selected from the group of SEQ ID NOS. 13, 14, 15, 16, 17, 18 and 19.

8. The assay of claim 6 wherein the probe comprises a sequence that spans the splice junction of the circRNA in an asymmetric manner.

9. The assay of claim 6 wherein the brain disorder-associated circRNA biomarker is selected from the group consisting of circHomer1a, circHomer1b, circCUL4A, and circADAM22.

10. The assay of claim 6 further comprising primers configured to amplify at least one brain disorder-associated circRNA biomarker.

11. The assay of claim 10 wherein the primer includes a sequence that spans the splice junction of the circRNA in an asymmetric manner.

12. The assay of claim 10 wherein at least one of the primers comprises a sequence that is selected from the group of SEQ ID NOS. 2, 3, 5, 6, 8, 9, 11 or 12.

13. The assay of claim 6 wherein the brain disorder is selected from the group consisting of:
bipolar disorder (BD), schizophrenia (SCZ), depression, Attention-Deficit/Hyperactivity Disorder (ADHD), Obsessive-compulsive disorder (OCD), Anxiety Disorders Autism, Asperger's Syndrome, and other Autism Spectrum Disorders (ASD), pervasive developmental disorders not otherwise specified (PDD-NOS).

14. A method for assessing one or more brain disorders in a subject comprising:
obtaining a sample from the patient;
assaying the sample using probes and/or primers configured to identify the presence of and/or amplify one or more brain disorder-associated circRNA biomarkers;
producing an expression profile of the one or more brain disorder-associated circRNA biomarkers based on the assay; and
comparing the expression profile to a baseline.

15. The method of claim 14 wherein the brain disorder is selected from the group consisting of bipolar disorder (BD), schizophrenia (SCZ), depression, Attention-Deficit/Hyperactivity Disorder (ADHD), Obsessive-compulsive disorder (OCD), and Anxiety Disorders.

16. The method of claim 14 wherein the step of assaying comprises one or more probes or primers that include a sequence that spans the splice junction of the circRNA in an asymmetric manner.

17. The method of claim 15 wherein the brain disorder-associated circRNA biomarker is selected from the group consisting of circHomer1a, circHomer1b, circCUL4A, and circADAM22.

18. The method of claim 16 wherein the step of assaying comprises one or more probes that are complementary to the group consisting of circHomer1a, circHomer1b, circCUL4A, and circADAM22.

19. The method of claim 15 wherein the step of assaying comprises primers configured to amplify at least a portion of the group consisting of circHomer1a, circHomer1b, circCUL4A, and circADAM22.

20. The method of claim 14 wherein at least one of the primers comprises a sequence that is selected from the group consisting of SEQ ID NOS. 2, 3, 5, 6, 8, 9, 11 or 12 or at least one of the probes comprises a sequence that is selected from the group of SEQ ID NOS. 13, 14, 15, 16, 17, 18 and 19.


SEQUENCE LISTING (20 sequences)

SEQ ID NO: 1 - DNA, 522 bp, human
ggaacaacctatcttcagcactcgagctcatgtcttccaaattgacccaaacacaaagaagaactgggtacccaccagcaagcatgcagttactgtgtcttatttctatgacagcacaagaaatgtgtataggataatcagtttagatggctcaaaggcaataataaatagtaccatcaccccaaacatgacatttactaaaacatctcagaagtttggccagtgggctgatagccgggcaaacaccgtttatggattgggattctcctctgagcatcatctttcgaaatttgcagaaaagtttcaggaatttaaagaagctgctcgactagcaaaggaaaaatcacaagagaagatggaacttaccagtacaccttcacaggaatccgcaggcggggatcttcagtctcctttaacaccggaaagtatcaacgggacagatgatgaaagaacacctgatgtgacacagaactcagagccaagggctgaaccaactcagaatgcattgccattttcacatag

SEQ ID NO: 2 - DNA, 20 bp, human
tcaacgggacagatgatgaa

SEQ ID NO: 3 - DNA, 20 bp, human
ttgtgtttgggtcaatttgg

SEQ ID NO: 4 - DNA, 289 bp, human
ggaacaacctatcttcagcactcgagctcatgtcttccaaattgacccaaacacaaagaagaactgggtacccaccagcaagcatgcagttactgtgtcttatttctatgacagcacaagaaatgtgtataggataatcagtttagatggctcaaaggcaataataaatagtaccatcaccccaaacatgacatttactaaaacatctcagaagtttggccagtgggctgatagccgggcaaacaccgtttatggattgggattctcctctgagcatcatctttcgaaa

SEQ ID NO: 5 - DNA, 20 bp, human
cgggcaaacaccgtttatgg

SEQ ID NO: 6 - DNA, 21 bp, human
catgagctcgagtgctgaaga

SEQ ID NO: 7 - DNA, 1114 bp, human
gtgaatgtaaatgcgaggacacgtggtccgggtgcataatgggagacactggctattatcttcctaaaaagttcacccagtgtaatattgaagagtatcatgacttcctgaatagtggaggtggtgcctgccttttcaacaaaccttctaagcttcttgatcctcctgagtgtggcaatggcttcattgaaactggagaggagtgtgattgtggaaccccggccgaatgtgtccttgaaggagcagagtgttgtaagaaatgcaccttgactcaagactctcaatgcagtgacggtctttgctgtaaaaagtgcaagtttcagcctatgggcactgtgtgccgagaagcagtaaatgattgtgatattcgtgaaacgtgctcaggaaattcaagccagtgtgcccctaatattcataaaatggatggatattcatgtgatggtgttcagggaatttgctttggaggaagatgcaaaaccagagatagacaatgcaaatacatttgggggcaaaaggtgacagcatcagacaaatattgctatgagaaactgaatattgaagggacggagaagggtaactgtgggaaagacaaagacacatggatacagtgcaacaaacgggatgtgctttgtggttaccttttgtgtaccaatattggcaatatcccaaggcttggagaactcgatggtgaaatcacatctactttagttgtgcagcaaggaagaacattaaactgcagtggtgggcatgttaagcttgaagaagatgtagatcttggctatgtggaagatgggacaccttgtggtccccaaatgatgtgcttagaacacaggtgtcttcctgtggcttctttcaactttagtacttgcttgagcagtaaagaaggcactatttgctcaggaaatggagtttgcagtaatgagctgaagtgtgtgtgtaacagacactggataggttctgattgcaacacttacttccctcacaatgatgatgcaaagactggtatcactctgtctggcaatggtgttgctggcaccaatatcataataggcataattgctggcaccattttagtgctggccctcatattaggaataactgcgtggggttataa

SEQ ID NO: 8 - DNA, 23 bp, human
tgttgctggcaccaatatcataa

SEQ ID NO: 9 - DNA, 21 bp, human
agtgtctcccattatgcaccc

SEQ ID NO: 10 - DNA, 131 bp, human
gtcacgtcccgtgtggctgtggagatctctgtgtgactatggaggtttcatccatgtggctctgaaggtctctgtgtgactatggaggtcgcgtcccatgtggctgtggaggtcgctgtgtggctatggag

SEQ ID NO: 11 - DNA, 19 bp, human
ctatggaggtcacgtcccg

SEQ ID NO: 12 - DNA, 21 bp, human
agagccacatggatgaaacct

SEQ ID NO: 13 - DNA, 20 bp, human
gccattttcacatagggaac

SEQ ID NO: 14 - DNA, 21 bp, mouse
gccatttccacatagggagca

SEQ ID NO: 15 - DNA, 20 bp, rat
gccattttcacatagggaac

SEQ ID NO: 16 - DNA, 10 bp, mouse
ttccacatag

SEQ ID NO: 17 - DNA, 10 bp, mouse
ggagcaacct

SEQ ID NO: 18 - DNA, 10 bp, human
tttcacatag

SEQ ID NO: 19 - DNA, 10 bp, human
ggaacaacct

SEQ ID NO: 20 - DNA, 32 bp, human
cattgccattttcacatagggaacaacctatc